(19) United States
(12) Patent Application Publication
(10) Pub. No.: US 2025/0155801 A1
(43) Pub. Date: May 15, 2025

(54) RESIST COMPOSITION AND PATTERN FORMING PROCESS

(71) Applicant: Shin-Etsu Chemical Co., Ltd., Tokyo (JP)

(72) Inventor: Jun Hatakeyama, Joetsu-shi (JP)

(73) Assignee: Shin-Etsu Chemical Co., Ltd., Tokyo (JP)

(21) Appl. No.: 18/930,232

(22) Filed: Oct. 29, 2024

(30) Foreign Application Priority Data
Nov. 14, 2023 (JP) 2023-193456

(51) Int. Cl. G03F 7/004; C08K 5/03; C08K 5/42; C08K 5/45; G03F 7/00; G03F 7/038; G03F 7/039; G03F 7/16

(52) U.S. Cl. CPC G03F 7/0042; C08K 5/03; C08K 5/42; C08K 5/45; G03F 7/0046; G03F 7/0382; G03F 7/0388; G03F 7/0397; G03F 7/162; G03F 7/168; G03F 7/70025; G03F 7/70033

(57) ABSTRACT

A resist composition is provided comprising a bis-onium salt consisting of a divalent anion having a sulfonic acid anion structure directly bonded to an iodized aromatic group and a sulfonimide or sulfonamide anion structure bonded to the aromatic group directly or via a linking group, and onium cations. It exhibits a high sensitivity and forms a pattern with reduced LWR or improved CDU independent of whether it is of positive or negative type.

### CROSS-REFERENCE TO RELATED APPLICATIONS

This non-provisional application claims priority under 35 U.S.C. § 119(a) on Patent Application No. 2023-193456 filed in Japan on Nov. 14, 2023, the entire contents of which are hereby incorporated by reference.

### TECHNICAL FIELD

This invention relates to a resist composition and a patterning process.

### BACKGROUND ART

To meet the demand for higher integration density and operating speed of LSIs, the effort to reduce the pattern rule is in rapid progress. As the use of 5G high-speed communications and artificial intelligence (AI) is widely spreading, high-performance devices are needed for their processing. As the advanced miniaturization technology, manufacturing of microelectronic devices at the 5 or 3-nm node by the lithography using EUV of wavelength 13.5 nm has been implemented in a mass scale. Studies are made on the application of EUV lithography to 2 nm node devices of the next generation and 14 Å node devices of the next-but-one generation. IMEC in Belgium announced its successful development of 2 Å node devices.

As the feature size reduces, image blurs due to acid diffusion become a problem. To insure resolution for fine patterns with a size of 45 nm et seq., not only an improvement in dissolution contrast is important as previously reported, but control of acid diffusion is also important as reported in Non-Patent Document 1. Since chemically amplified resist compositions are designed such that sensitivity and contrast are enhanced by acid diffusion, an attempt to minimize acid diffusion by reducing the temperature and/or time of post-exposure bake (PEB) fails, resulting in drastic reductions of sensitivity and contrast.

The EUV lithography resist must meet high sensitivity, high resolution and low line width roughness (LWR) at the same time. As the acid diffusion distance is reduced, LWR or CDU is improved, but sensitivity becomes lower. For example, as the PEB temperature is lowered, the outcome is an improved LWR or CDU, but a lower sensitivity. As the amount of quencher added is increased, the outcome is an improved LWR or CDU, but a lower sensitivity. It is necessary to overcome the tradeoff relation between sensitivity and LWR.

Patent Documents 1 to 4 disclose resist materials to which an onium salt containing an iodized or brominated anion is added as the acid generator. Since iodine having high EUV absorption or bromine having high ionization efficiency is included, the efficiency of decomposition of the acid generator during exposure is increased, leading to a higher sensitivity. Since the number of photons absorbed is increased, the physical contrast is enhanced.

The irradiation energy of EUV of wavelength 13.5 nm is higher than that of ArF excimer laser of wavelength 193 nm because the wavelength is shorter by more than one order. This suggests that a variation of photon number has a stronger influence (see Non-Patent Document 2). LWR is thus degraded as pointed out in Non-Patent Document 3. With the advance of miniaturization, the degradation of LWR is also affected by variations (known as resist stochastics) of components such as polymer, acid generator (PAG) and quencher (PDQ) in resist material, as pointed out in Non-Patent Document 4.

Patent Document 5 proposes a resist material comprising a polymer having PAG and PDQ combined together. This intends to improve LWR or CDU by integrating the polymer with PAG and PDQ to minimize their distribution variation. Patent Documents 6 and 7 propose resist materials comprising an additive having PAG and PDQ combined together.

The potential hazard to health of perfluoroalkyl substances (PFASs) is pointed out. The REACH Regulation of EU aims to put restrictions on the manufacture and sales of PFASs. A number of substances including PFASs are currently used in the field of semiconductor lithography. For example, materials containing PFASs are applied to surfactants, acid generators, and the like.

In Non-Patent Document 5, a resist material comprising an acid generator capable of generating a fluorinated anion bonded to the polymer backbone is compared with a resist material comprising an acid generator capable of generating a fluorine-free anion bonded to the polymer backbone. It is described that the polymer-bound acid generator capable of generating a fluorinated anion exhibits a higher resolution. The sulfonic acid having a high acid strength has a high efficiency of deprotection reaction. That is, the introduction of fluorine is effective for enhancing acid strength.

In the resist material described in Non-Patent Document 6, an anion having a high acidity is generated by introducing a nitro group or chlorine rather than fluorine. Although the resist material comprising an acid generator capable of generating a nitro or chlorine-substituted anion sometimes shows a high rectangularity as compared with the resist material comprising an acid generator capable of generating a fluorine-substituted anion, the former resist material has drawbacks of low sensitivity and large MEEF as seen from Table 2 in the literature. Such drawbacks are caused by low dissolution contrast due to the low acidity of the anion and the low efficiency of deprotection reaction.

### CITATION LIST

Patent Document 1: JP-A 2018-159744 (U.S. Pat. No. 10,613,436)
Patent Document 2: JP-A 2018-005224 (U.S. Pat. No. 10,323,113)
Patent Document 3: JP-A 2018-025789 (U.S. Pat. No. 10,101,653)
Patent Document 4: JP-A 2019-003175 (U.S. Pat. No. 10,725,378)
Patent Document 5: JP-A 2022-115072 (U.S. Pat. No. 11,953,832)
Patent Document 6: JP-A 2015-024989
Patent Document 7: WO 2020/158313
Non-Patent Document 1: SPIE Vol. 6520 65203L-1 (2007)
Non-Patent Document 2: SPIE Vol. 3331 535 (1998)
Non-Patent Document 3: SPIE Vol. 7273 727343-1 (2009)
Non-Patent Document 4: SPIE Vol. 9776 97760V-1 (2016)
Non-Patent Document 5: SPIE Vol. 6519 65191F-1 (2007)
Non-Patent Document 6: SPIE Vol. 7639 76390D-1 (2010)

### SUMMARY OF THE INVENTION

It is desired to have a resist material having a higher sensitivity than prior art resist materials and capable of reducing the LWR of line patterns and improving the CDU of hole patterns.

An object of the invention is to provide a resist composition which exhibits a high sensitivity, reduced LWR, and improved CDU independent of whether it is of positive or negative type, and a pattern forming process using the same.

The inventor has found that when a bis-onium salt consisting of a divalent anion having a sulfonic acid anion structure directly bonded to an iodized aromatic group and a sulfonimide or sulfonamide anion structure bonded to the aromatic group directly or via a linking group containing at least one atom, and onium cations is used as the acid generator and quencher, the highly absorptive acid generator is directly excited upon exposure to radiation, the influence of diffusion of secondary electrons is eliminated, the acid diffusion is controlled by the neighboring quencher, and the influence of image blur due to diffusion is minimized. There is thus obtained a resist composition which exhibits a high sensitivity, reduced LWR, improved CDU, high contrast, high resolution, and wide process margin.

In one aspect, the invention provides a resist composition comprising a bis-onium salt consisting of a divalent anion having a sulfonic acid anion structure directly bonded to an iodized aromatic group and a sulfonimide or sulfonamide anion structure bonded to the aromatic group directly or via a linking group containing at least one atom, and onium cations.

In one preferred embodiment, the bis-onium salt has the formula (1).

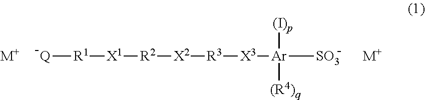

Herein p is an integer of 1 to 5, q is an integer of 0 to 7,
X¹ to X³ are each independently a single bond, ether bond, ester bond or amide bond,
R¹ to R³ are each independently a single bond or C₁-C₃₀ hydrocarbylene group which may contain at least one element selected from oxygen, nitrogen, sulfur and halogen, the total number of carbon atoms in R¹ to R³ being up to 30,
R⁴ is hydrogen, hydroxy, carboxy, fluorine, chlorine, bromine, amino, nitro, cyano, C₁-C₂₀ hydrocarbyl group, C₁-C₂₀ hydrocarbyloxy group, C₂-C₂₀ hydrocarbyloxycarbonyl group, C₂-C₂₀ hydrocarbylcarbonyloxy group, C₁-C₂₀ hydrocarbylsulfonyloxy group, —N(R^{4A})—C(═O)—R^{4B}, —N(R^{4A})—C(═O)—O—R^{4B}, or —N(R^{4A})—S(═O)₂—R^{4B}, the hydrocarbyl, hydrocarbyloxy, hydrocarbyloxycarbonyl, hydrocarbylcarbonyloxy, and hydrocarbylsulfonyloxy groups may contain at least one moiety selected from fluorine, chlorine, bromine, iodine, hydroxy, amino, ester bond, ether bond, urethane bond, urea bond, carbonate bond, amide bond, sulfonate ester bond, carbonyl, sulfide, and sulfonyl, R^{4A} is hydrogen or a C₁-C₆ saturated hydrocarbyl group which may contain halogen, hydroxy, a C₁-C₆ saturated hydrocarbyloxy moiety, C₂-C₆ saturated hydrocarbylcarbonyl moiety, or C₂-C₆ saturated hydrocarbylcarbonyloxy moiety, R^{4B} is a C₁-C₁₆ aliphatic hydrocarbyl group or C₆-C₁₂ aryl group, which may contain halogen, hydroxy, a C₁-C₆ saturated hydrocarbyloxy moiety, C₂-C₆ saturated hydrocarbylcarbonyl moiety, or C₂-C₆ saturated hydrocarbylcarbonyloxy moiety,
Ar is a C₆-C₁₆ (p+q+1)-valent aromatic hydrocarbon group,
Q⁻ is a group having a sulfonimide or sulfonamide anion structure, and
M⁺ is a sulfonium or iodonium cation.

In a preferred embodiment, Q⁻ is a group having any one of the following formulae (Q-1) to (Q-4).

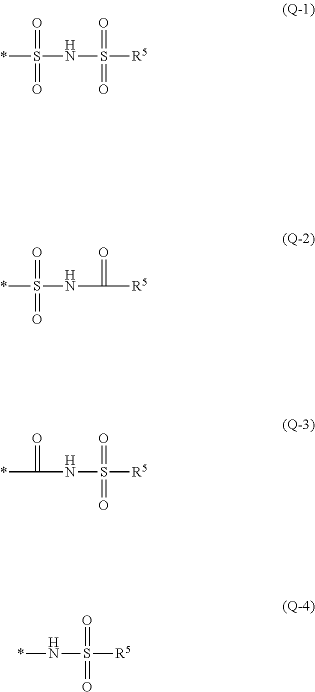

Herein R⁵ is each independently a C₁-C₁₂ saturated hydrocarbyl group or C₆-C₁₂ aryl group, which may be substituted with at least one moiety selected from halogen, cyano, nitro, hydroxy, C₁-C₈ saturated hydrocarbyl moiety, C₂-C₈ saturated hydrocarbylcarbonyloxy moiety, C₂-C₈ saturated hydrocarbyloxycarbonyl moiety, trifluoromethoxy, difluoromethoxy, trifluoromethylthio, and trifluoromethyl, and * designates a point of attachment to X¹.

In a preferred embodiment, the resist composition further comprises a base polymer.

In a preferred embodiment, the base polymer comprises repeat units having formula (a1) or a2).

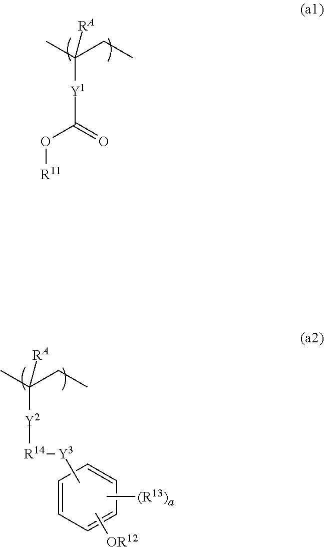

Herein R^{A} is each independently hydrogen or methyl,
Y¹ is a single bond, phenylene group, naphthylene group or a C₁-C₁₂ linking group which contains at least one moiety selected from an ester bond, ether bond and lactone ring, the phenylene group, naphthylene group and linking group may contain at least one moiety selected from hydroxy, C₁-C₈ saturated hydrocarbyloxy moiety and C₂-C₈ saturated hydrocarbylcarbonyloxy moiety,
Y² is a single bond or ester bond,
Y³ is a single bond, ether bond or ester bond,
R¹¹ and R¹² are each independently an acid labile group,
R¹³ is a C₁-C₄ saturated hydrocarbyl group, halogen, C₂-C₅ saturated hydrocarbylcarbonyl group, cyano, or C₂-C₅ saturated hydrocarbyloxycarbonyl group,
R¹⁴ is a single bond or a C₁-C₆ alkanediyl group which may contain an ether bond or ester bond, and
a is an integer of 0 to 4.

Typically, the resist composition is a chemically amplified positive resist composition.

In another embodiment, the base polymer is free of an acid labile group. In this embodiment, the resist composition is a chemically amplified negative resist composition.

The resist composition may further comprise an organic solvent, a quencher, an acid generator, and/or a surfactant.

In another aspect, the invention provides a pattern forming process comprising the steps of applying the resist composition defined herein onto a substrate to form a resist film thereon, exposing the resist film to high-energy radiation, and developing the exposed resist film in a developer.

Typically, the high-energy radiation is ArF excimer laser of wavelength 193 nm, KrF excimer laser of wavelength 248 nm, EB or EUV of wavelength 3 to 15 nm.

### Advantageous Effects of Invention

The onium salt of an iodized arylsulfonic acid shows greater absorption of EUV, higher acid strength and less (or restrained) acid diffusion than unsubstituted arylsulfonic acids and even fluorinated arylsulfonic acids. The high absorption leads to an increase in direct excitation reaction, which restrains diffusion of secondary electrons. The bis-onium salt having an anion which is structured such that a quencher is further bonded to the iodized arylsulfonic acid is also effective for restraining acid diffusion. Thus the image blur due to diffusion of both secondary electrons and acid is minimized. The bis-onium salt in which an acid generator in the form of an onium salt capable of generating sulfonic acid and a quencher in the form of an onium salt are bonded together suggests that the acid generator and the quencher are always located at a certain distance. The resist film containing such a bis-onium salt is improved in the resist stochastics, which leads to an improvement in LWR or CDU. That is, using the bis-onium salt, a resist composition having a high sensitivity, reduced LWR, and improved CDU can be designed.

### DETAILED DESCRIPTION OF THE INVENTION

As used herein, the singular forms “a,” “an” and “the” include plural referents unless the context clearly dictates otherwise. “Optional” or “optionally” means that the subsequently described event or circumstances may or may not occur, and that description includes instances where the event or circumstance occurs and instances where it does not. The notation (Cn-Cm) means a group containing from n to m carbon atoms per group. In chemical formulae, the broken line (---) or asterisk (*) designates a point of attachment or valence bond. As used herein, the term “fluorinated” refers to a fluorine-substituted or fluorine-containing compound or group, and the term “iodized” refers to an iodine-substituted or iodine-containing compound or group. The terms “group” and “moiety” are interchangeable.

The abbreviations and acronyms have the following meaning.
EB: electron beam
EUV: extreme ultraviolet
Mw: weight average molecular weight
Mn: number average molecular weight
Mw/Mn: molecular weight distribution or dispersity
GPC: gel permeation chromatography
PEB: post-exposure bake
PAG: photoacid generator
LWR: line width roughness
CDU: critical dimension uniformity

### [Resist Composition]

One embodiment of the invention is a resist composition comprising a bis-onium salt consisting of a divalent anion having a sulfonic acid anion structure directly bonded to an iodized aromatic group and a sulfonimide or sulfonamide anion structure bonded to the aromatic group directly or via a linking group containing at least one atom, and onium cations. Due to the cooperation of the high absorption of iodine, the high acidity of sulfonic acid and concomitant high reactivity, and a high level of acid diffusion control owing to the consistent location of the quencher in vicinity, the bis-onium salt is characterized by low acid diffusion and a uniform diffusion distance. Then LWR or CDU is improved.

The bis-onium salt exerts the effect of improving LWR or CDU, which is valid in positive or negative tone pattern formation by aqueous alkaline development and in negative tone pattern formation by organic solvent development.

### [Bis-Onium Salt]

The bis-onium salt preferably has the formula (1).

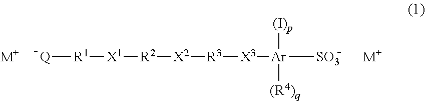

In formula (1), p is an integer of 1 to 5, and q is an integer of 0 to 7.

In formula (1), X¹ to X³ are each independently a single bond, ether bond, ester bond or amide bond.

In formula (1), R¹ to R³ are each independently a single bond or C₁-C₃₀ hydrocarbylene group. The hydrocarbylene group may contain at least one element selected from oxygen, nitrogen, sulfur and halogen. The total number of carbon atoms in R¹ to R³ is up to 30.

The hydrocarbylene group R¹ to R³ may be saturated or unsaturated and straight, branched or cyclic. Also included are C₁-C₂₄ hydrocarbylene groups in which some or all of the hydrogen atoms are substituted by iodine or bromine. Examples of the C₁-C₂₄ hydrocarbylene group include alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, heptadecane-1,17-diyl, octadecane-1,18-diyl, nonadecane-1,19-diyl, and eicosane-1,20-diyl; cyclic saturated hydrocarbylene groups such as cyclopropanediyl, cyclobutanediyl, cyclopentanediyl, methylcyclopentanediyl, dimethylcyclopentanediyl, trimethylcyclopentanediyl, tetramethylcyclopentanediyl, cyclohexanediyl, methylcyclohexanediyl, dimethylcyclohexanediyl, trimethylcyclohexanediyl, tetramethylcyclohexanediyl, norbornanediyl and adamantanediyl; C₂-C₂₀ alkenediyl groups such as ethenediyl, propenediyl and butenediyl; C₂-C₂₀ alkynediyl groups such as ethynediyl, propynediyl and butynediyl; arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene, tert-butylnaphthylene, tetrahydronaphthylene, biphenyldiyl, methylbiphenyldiyl and dimethylbiphenyldiyl; and combinations thereof. In the hydrocarbylene group, some or all of the hydrogen atoms may be substituted by a moiety containing at least one element selected from oxygen, nitrogen, sulfur and halogen, or some —CH₂— may be replaced by a moiety containing at least one element selected from oxygen, nitrogen and sulfur, so that the group may contain a hydroxy moiety, ester bond, ether bond, amide bond, carbamate bond or urea bond.

In formula (1), R⁴ is hydrogen, hydroxy, carboxy, fluorine, chlorine, bromine, amino, nitro, cyano, a C₁-C₂₀ hydrocarbyl group, C₁-C₂₀ hydrocarbyloxy group, C₂-C₂₀ hydrocarbyloxycarbonyl group, C₂-C₂₀ hydrocarbylcarbonyloxy group, C₁-C₂₀ hydrocarbylsulfonyloxy group, —N(R^{4A})—C(═O)—R^{4B}, —N(R^{4A})—C(═O)—O—R^{4B}, or —N(R^{4A})—S(═O)₂—R^{4B}. The hydrocarbyl, hydrocarbyloxy, hydrocarbyloxycarbonyl, hydrocarbylcarbonyloxy, and hydrocarbylsulfonyloxy groups may contain at least one moiety selected from fluorine, chlorine, bromine, iodine, hydroxy, amino, ester bond, ether bond, urethane bond, urea bond, carbonate bond, amide bond, sulfonate ester bond, carbonyl, sulfide, and sulfonyl. R^{4A} is hydrogen or a C₁-C₆ saturated hydrocarbyl group which may contain halogen, hydroxy, C₁-C₆ saturated hydrocarbyloxy, C₂-C₆ saturated hydrocarbylcarbonyl, or C₂-C₆ saturated hydrocarbylcarbonyloxy moiety. R^{4B} is a C₁-C₁₆ aliphatic hydrocarbyl group or C₆-C₁₂ aryl group, which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety.

The hydrocarbyl group and hydrocarbyl moiety in the hydrocarbyloxy, hydrocarbyloxycarbonyl, hydrocarbylcarbonyloxy, and hydrocarbylsulfonyloxy groups, represented by R⁴, may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, propenyl, butenyl and hexenyl; C₂-C₂₀ alkynyl groups such as ethynyl, propynyl and butynyl; C₃-C₂₀ cyclic unsaturated aliphatic hydrocarbyl groups such as cyclohexenyl and norbornenyl; C₆-C₂₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, and tert-butylnaphthyl; C₇-C₂₀ aralkyl groups such as benzyl and phenethyl; and combinations thereof.

In formula (1), Ar is a C₆-C₁₆ (p+q+1)-valent aromatic hydrocarbon group. Examples of the aromatic hydrocarbon group include groups obtained by eliminating (p+q+1) number of hydrogen atoms from aromatic hydrocarbons such as benzene, naphthalene, anthracene, and pyrene.

In formula (1), Q⁻ is a group having a sulfonimide or sulfonamide anion structure. A group having any one of the following formulae (Q-1) to (Q-4) is preferred.

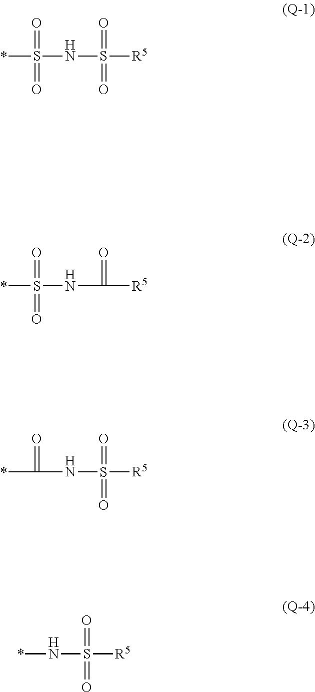

In formulae (Q-1) to (Q-4), R⁵ is each independently a C₁-C₁₂ saturated hydrocarbyl group or C₆-C₁₂ aryl group, which may be substituted with at least one moiety selected from halogen, cyano, nitro, hydroxy, C₁-C₈ saturated hydrocarbyl moiety, C₂-C₈ saturated hydrocarbylcarbonyloxy moiety, C₂-C₈ saturated hydrocarbyloxycarbonyl moiety, trifluoromethoxy, difluoromethoxy, trifluoromethylthio, and trifluoromethyl. The asterisk (*) designates a point of attachment to X¹.

The C₁-C₁₂ saturated hydrocarbyl group R⁵ may be straight, branched or cyclic. Examples thereof include C₁-C₁₂ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, n-decyl, undecyl, and dodecyl; and C₃-C₁₂ cyclic saturated hydrocarbyl groups such as cyclopentyl, methylcyclopentyl, ethylcyclopentyl, propylcyclopentyl, isopropylcyclopentyl, n-butylcyclopentyl, isobutylcyclopentyl, sec-butylcyclopentyl, tert-butylcyclopentyl, cyclohexyl, methylcyclohexyl, ethylcyclohexyl, propylcyclohexyl, isopropylcyclohexyl, n-butylcyclohexyl, isobutylcyclohexyl, sec-butylcyclohexyl, tert-butylcyclohexyl, cyclopentylmethyl, cyclopentylethyl, cyclopentylbutyl, cyclohexylmethyl, cyclohexylethyl, cyclohexylbutyl, norbornyl, and adamantyl.

Examples of the C₆-C₁₂ aryl group R⁵ include phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, and ethylnaphthyl.

Examples of the anion in the bis-onium salt are shown below, but not limited thereto.

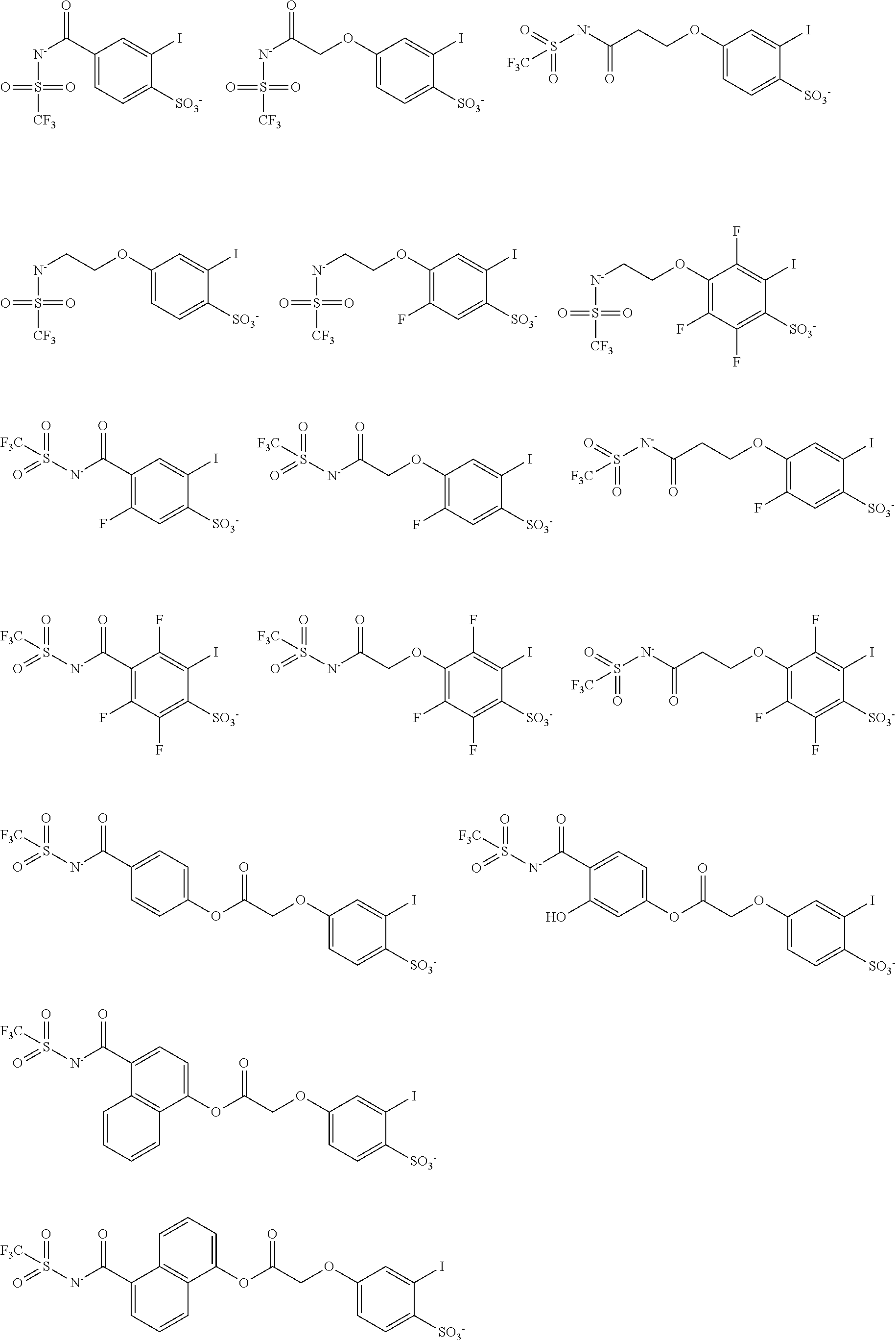

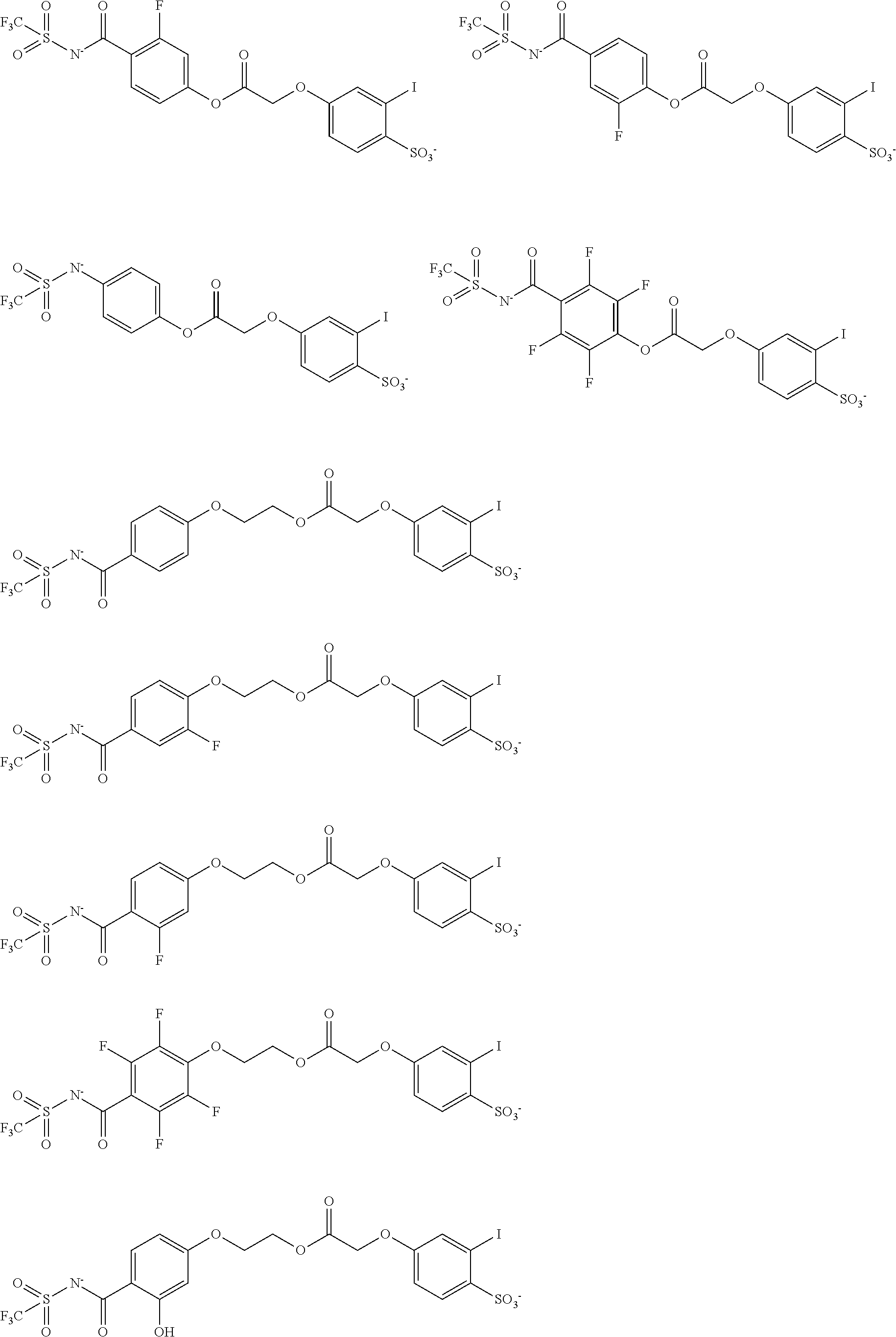

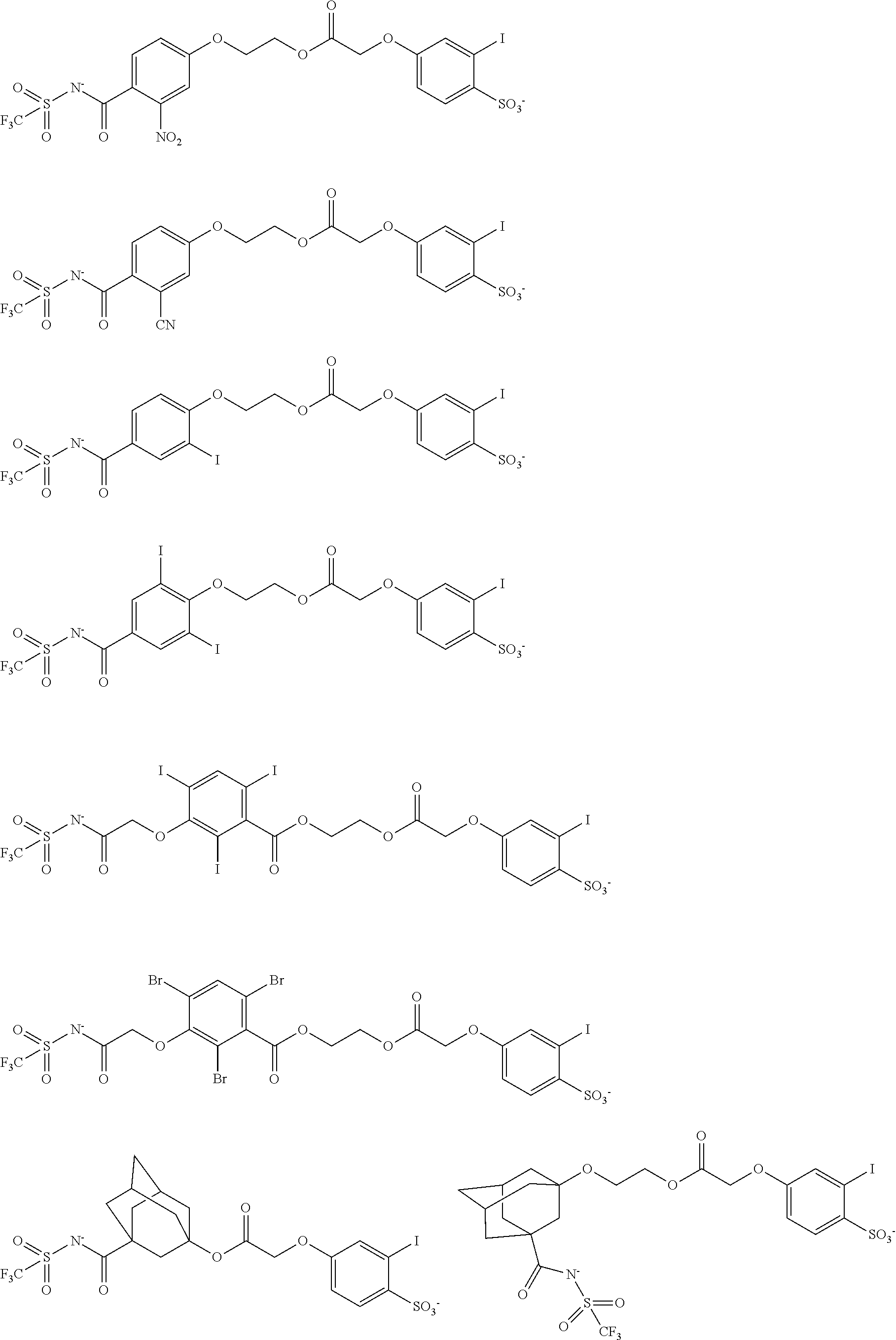

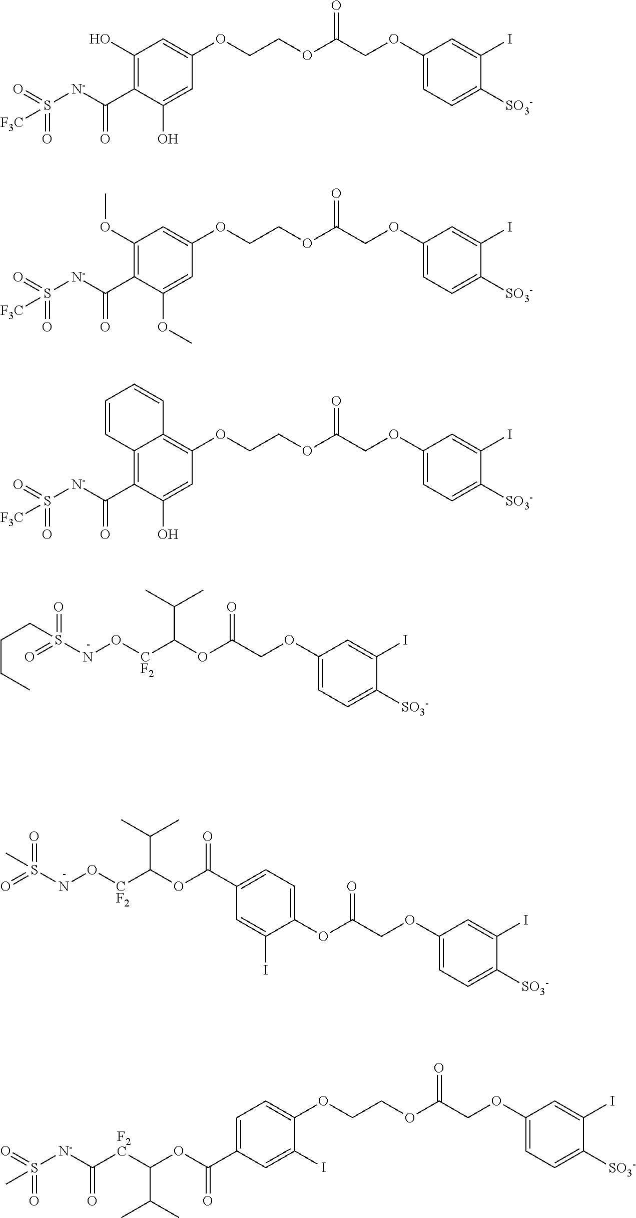

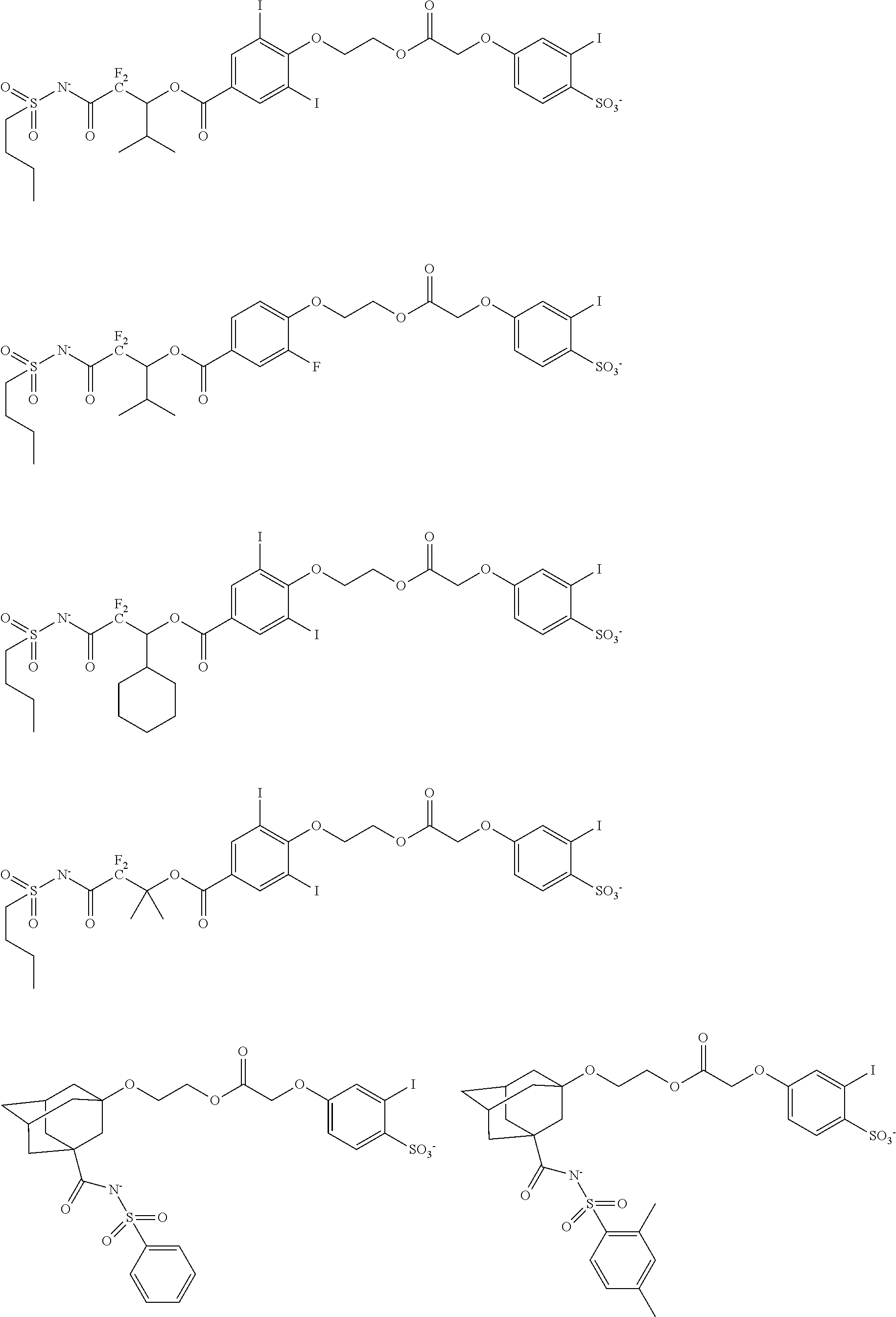

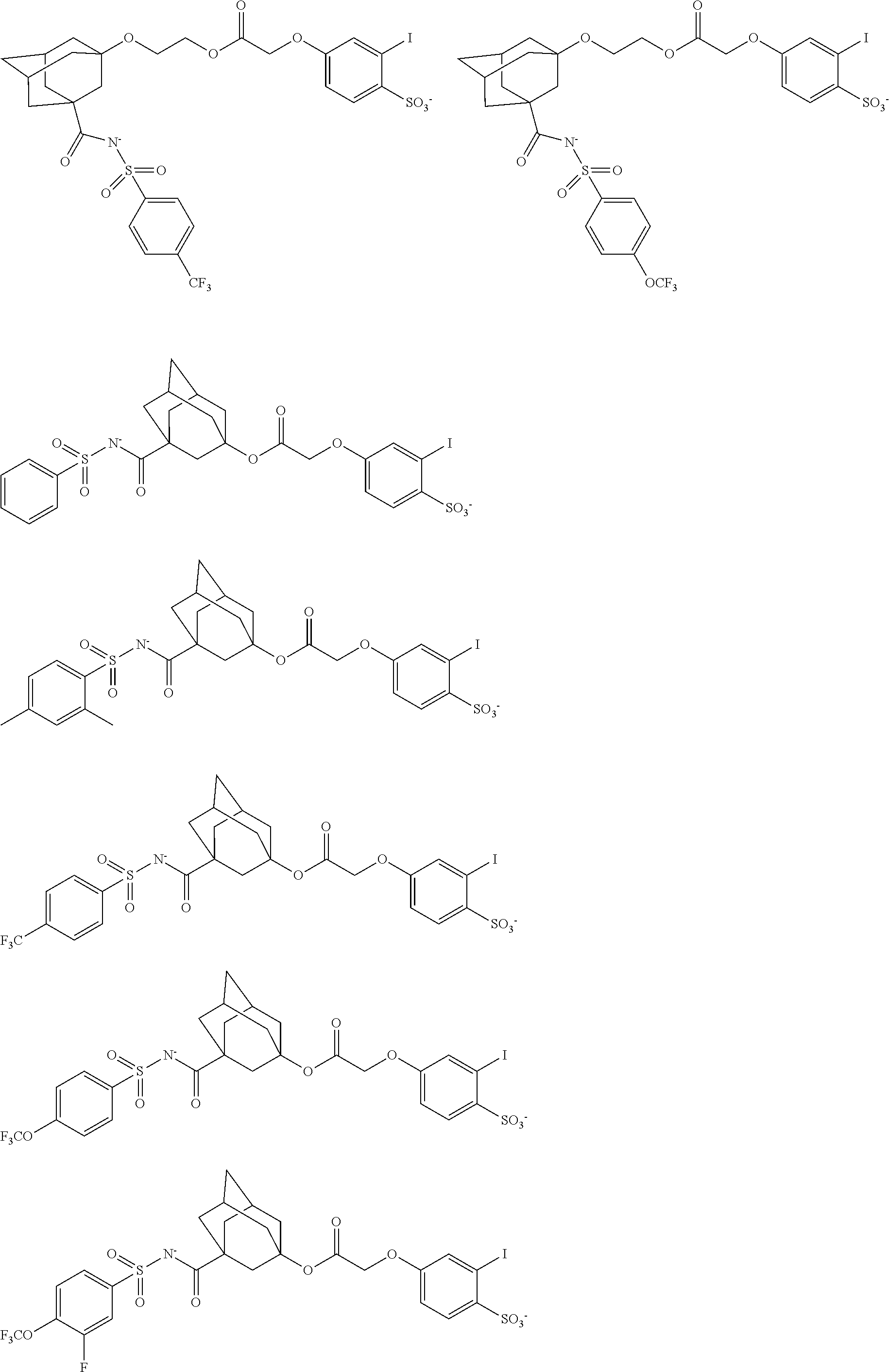

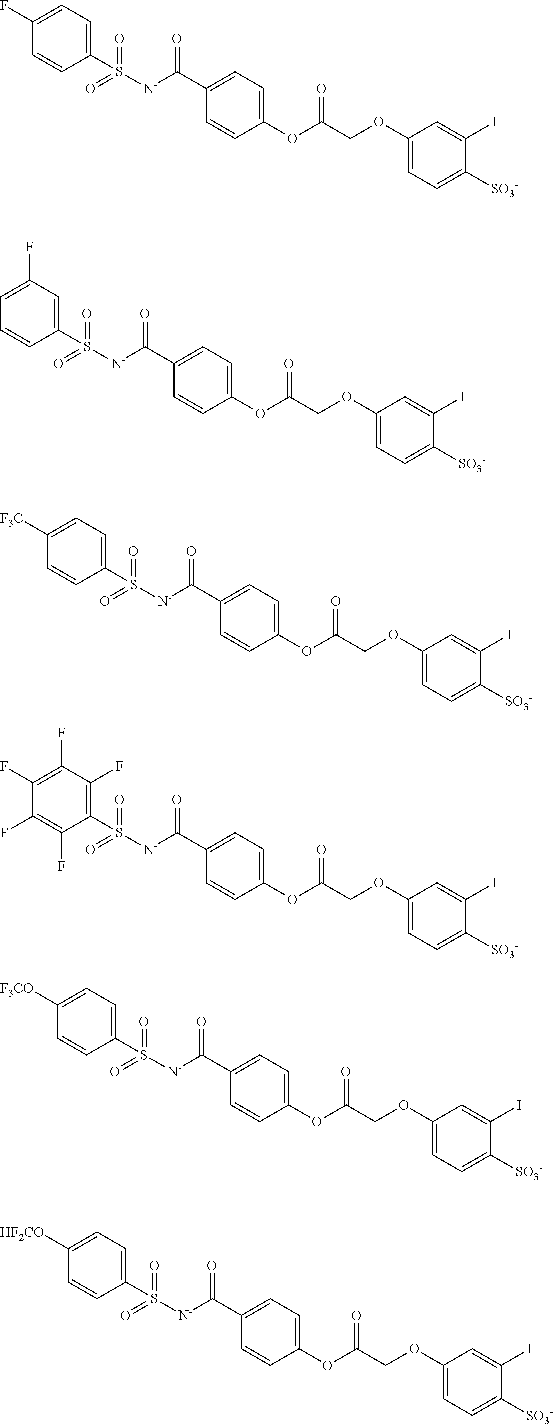

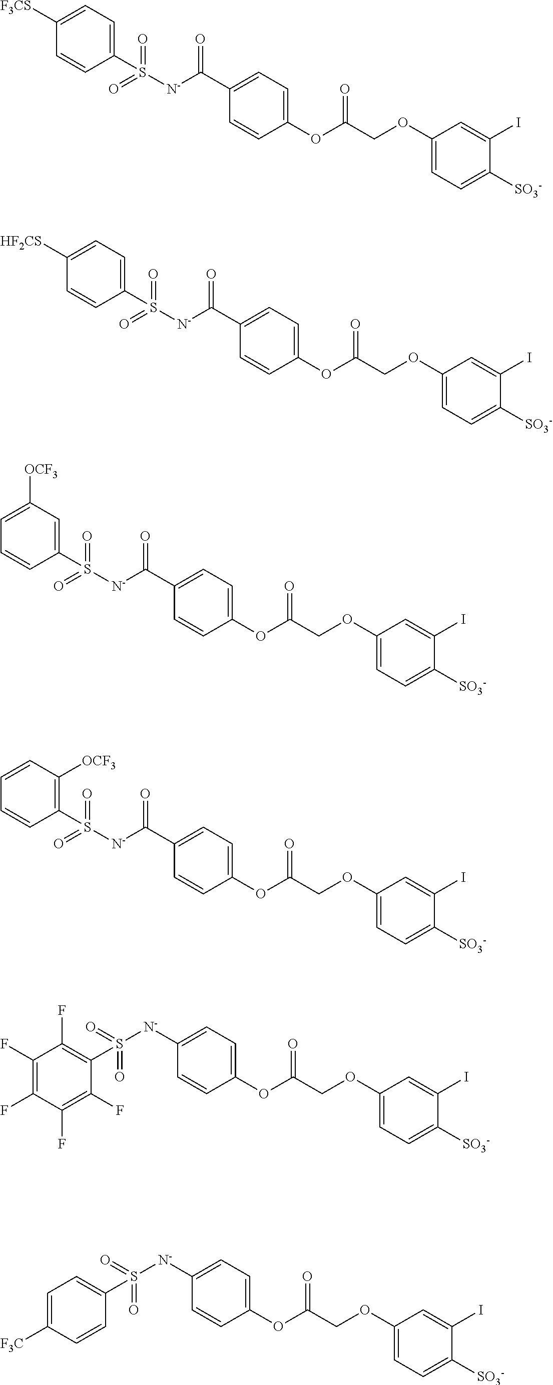

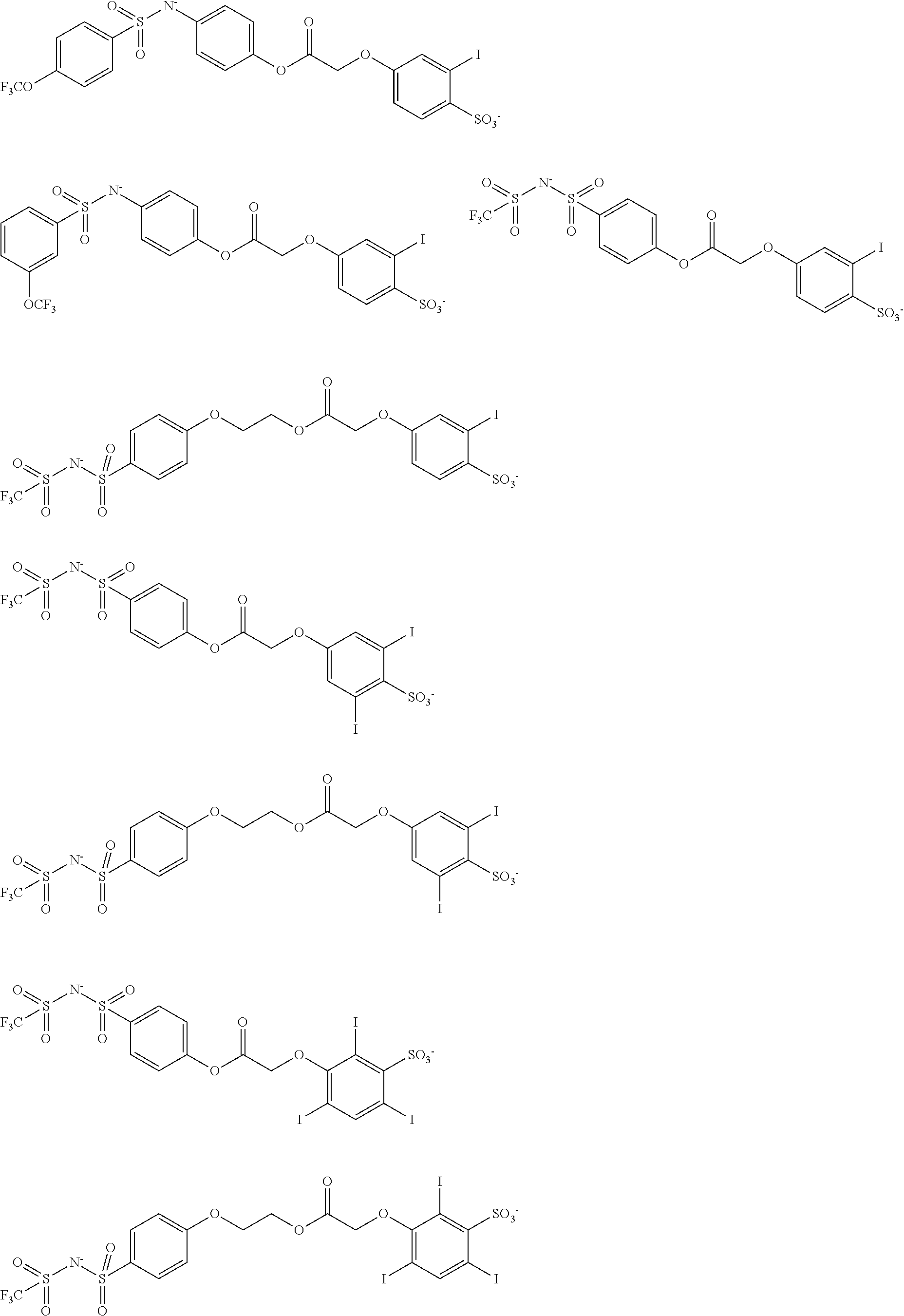

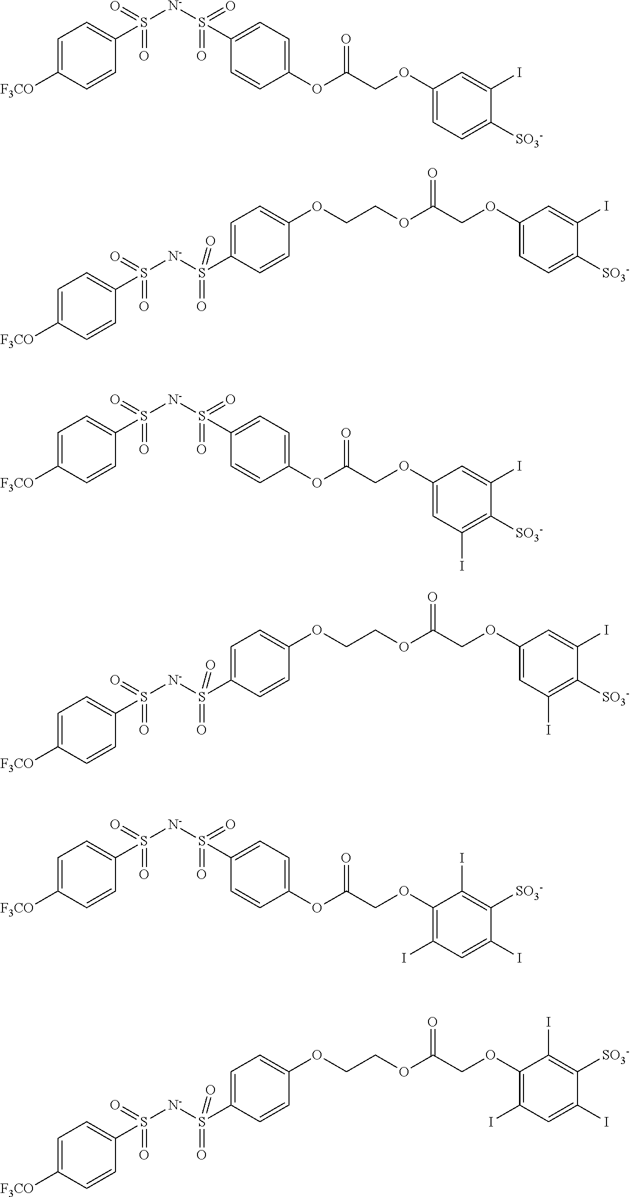

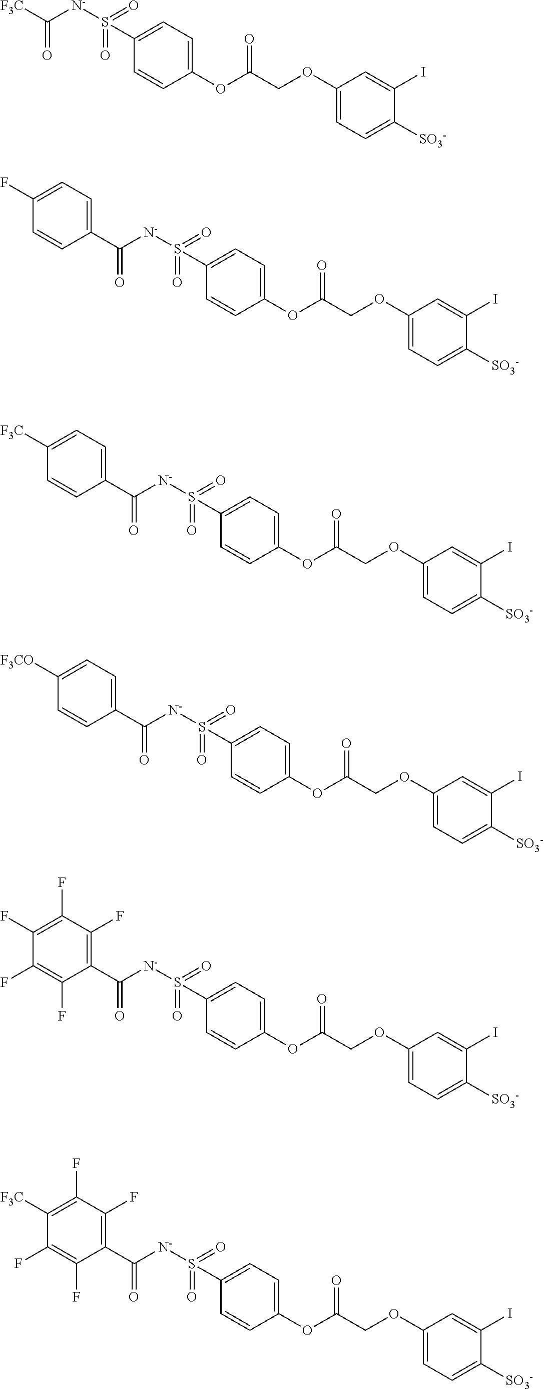

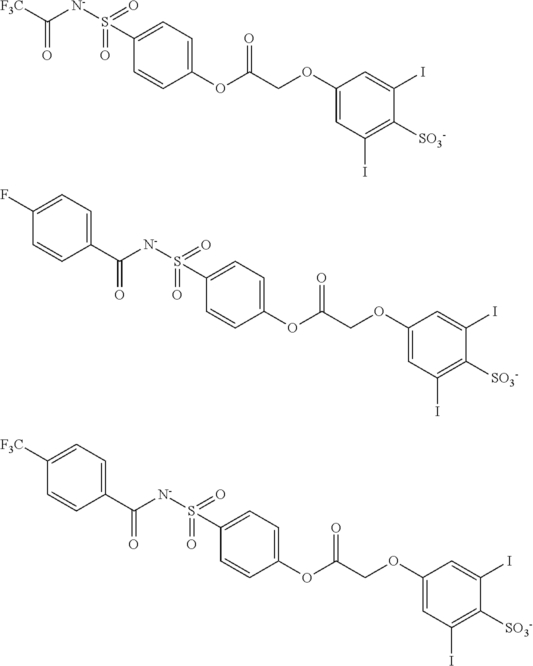

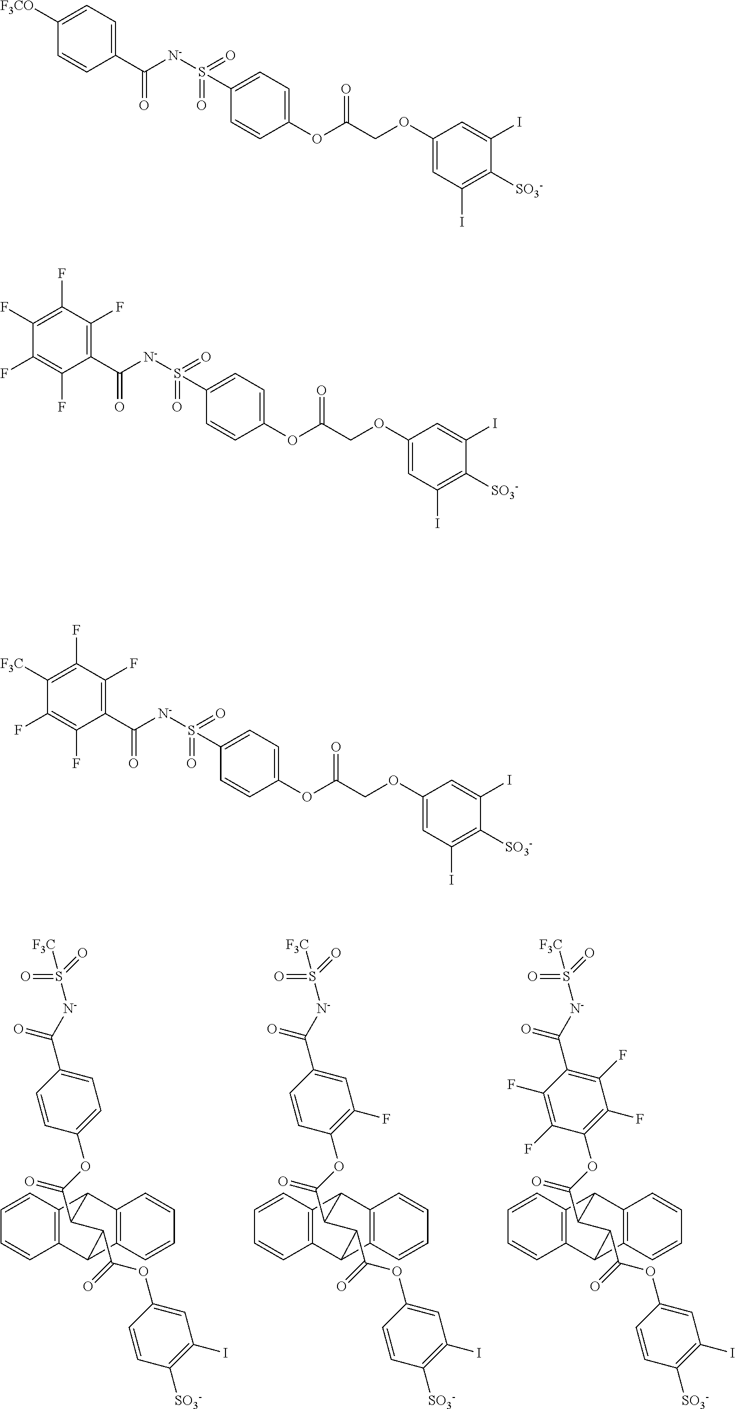

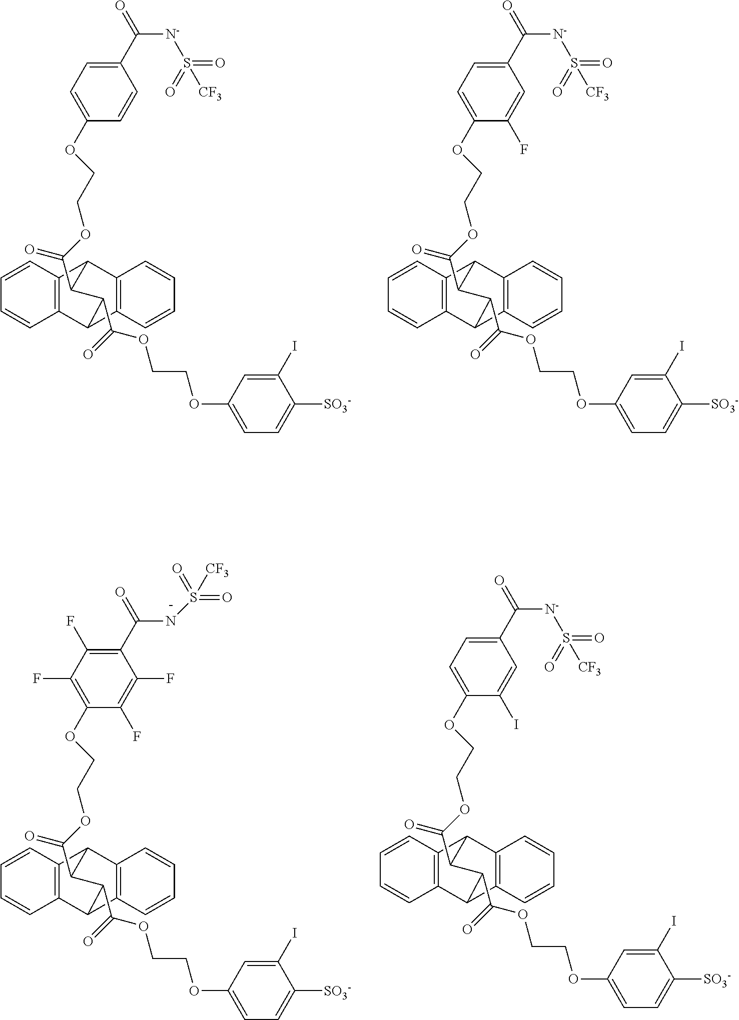

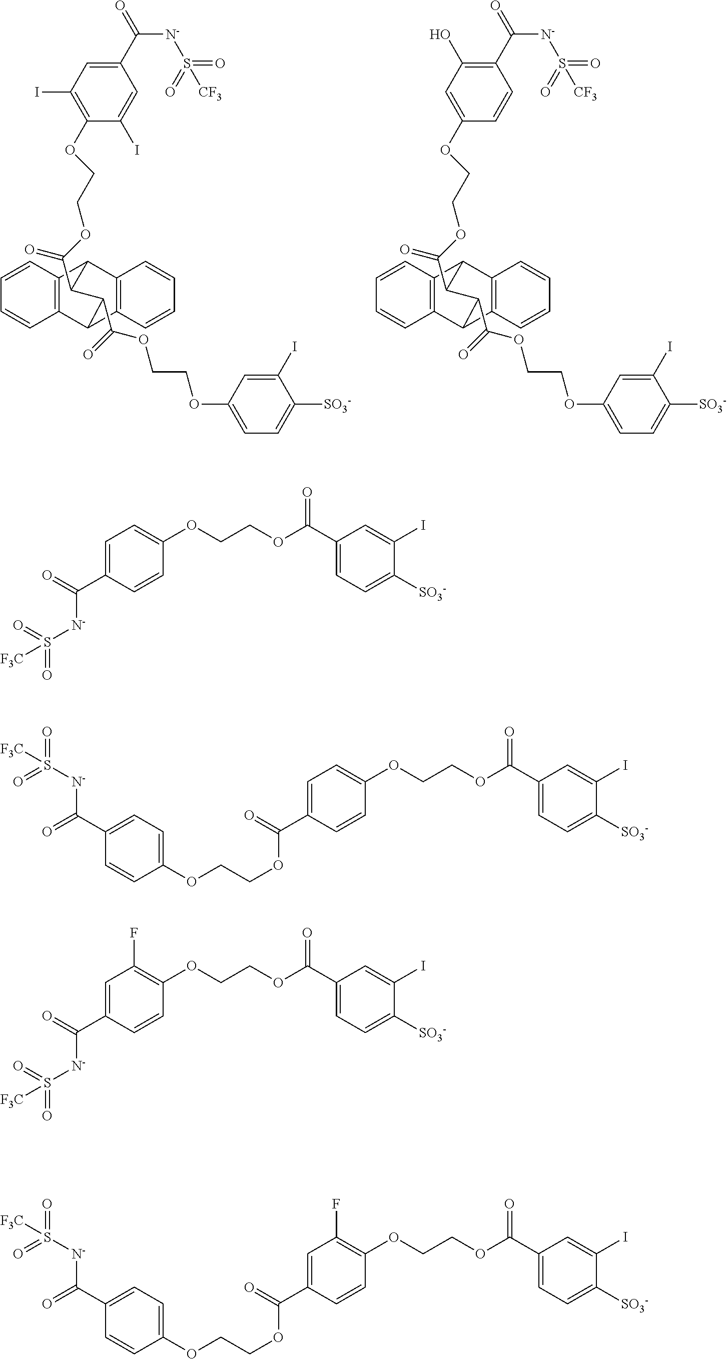

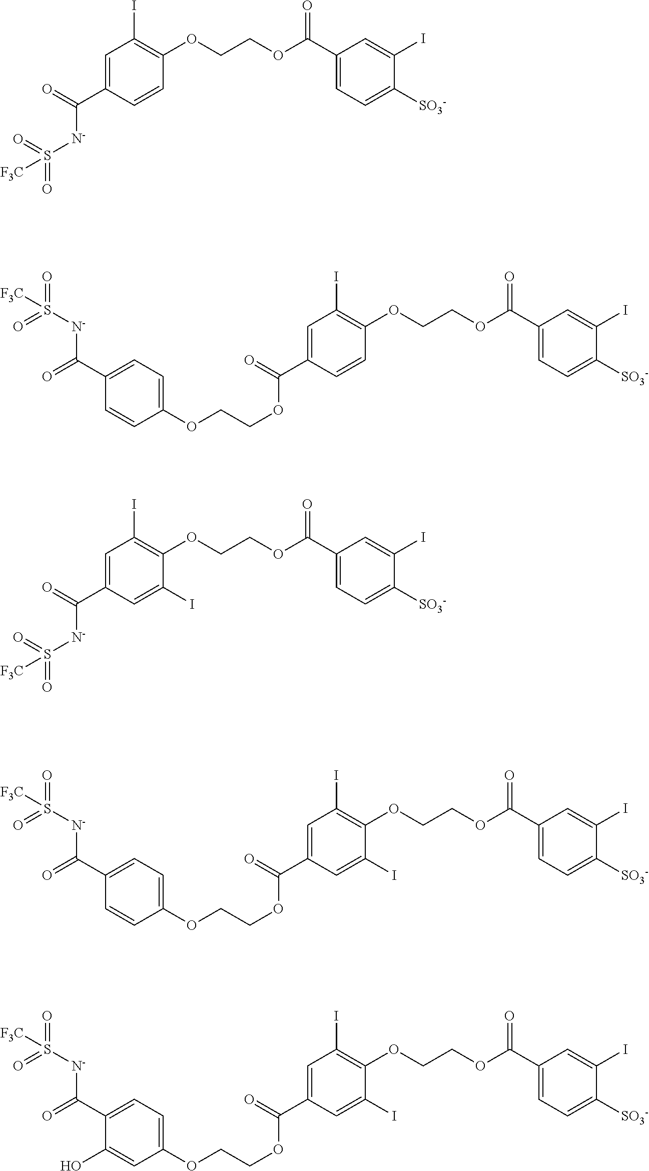

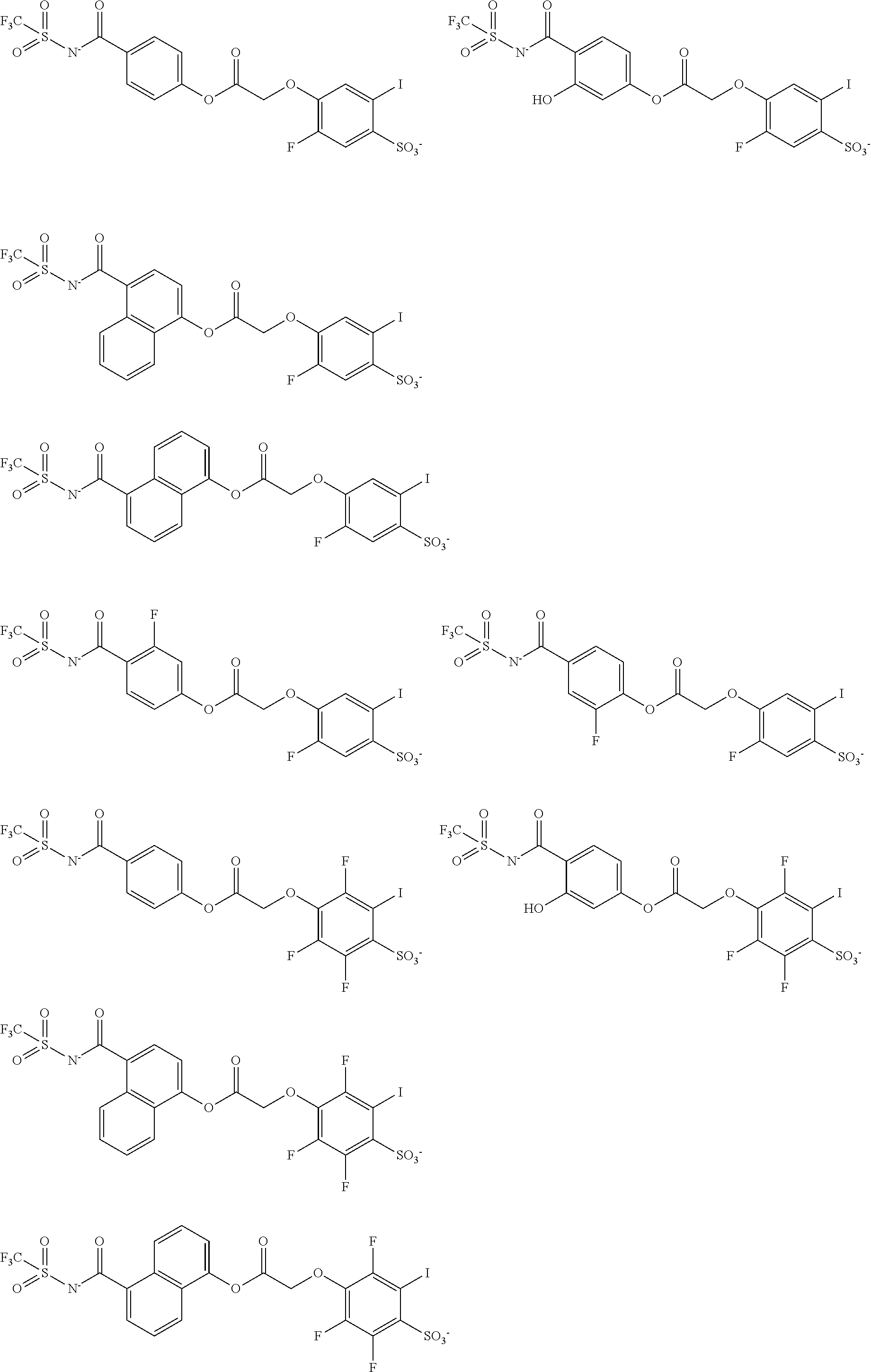

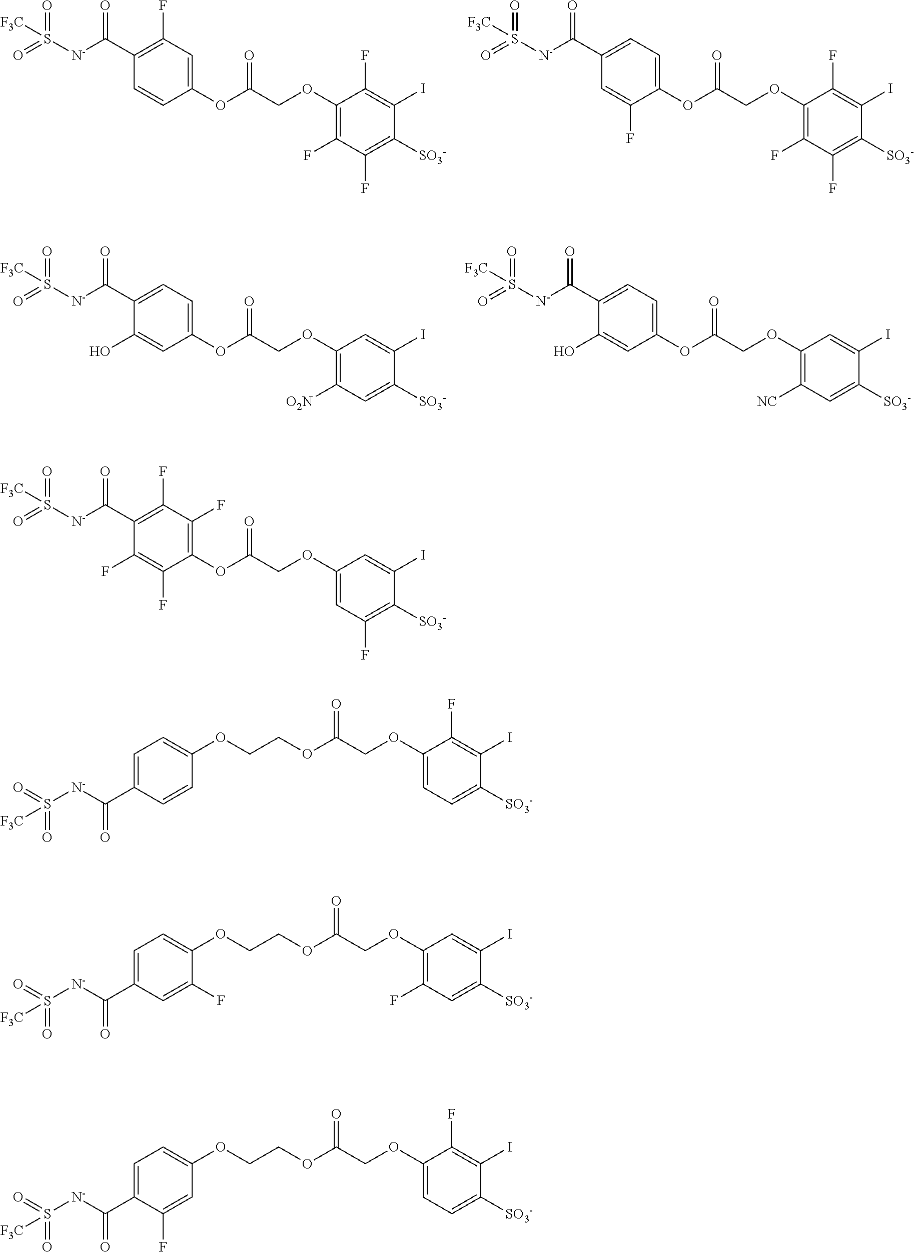

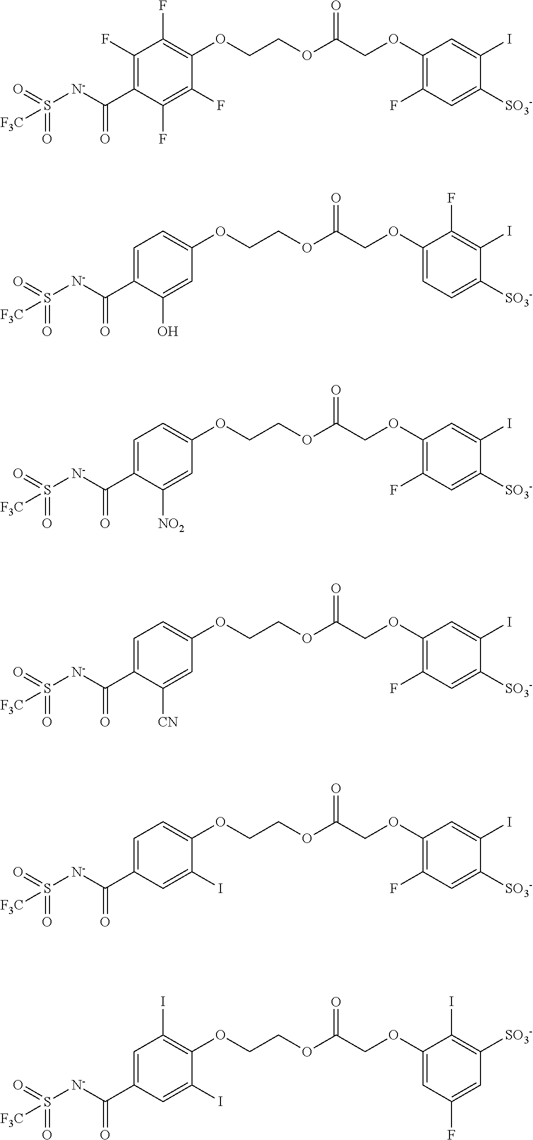

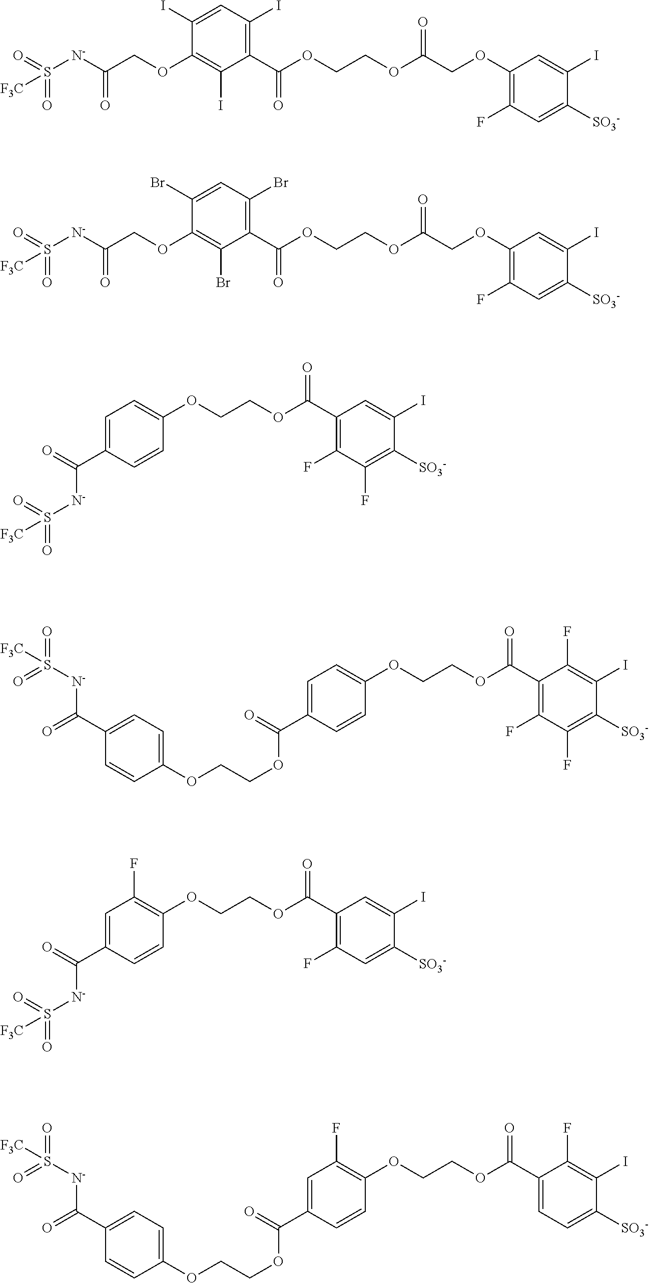

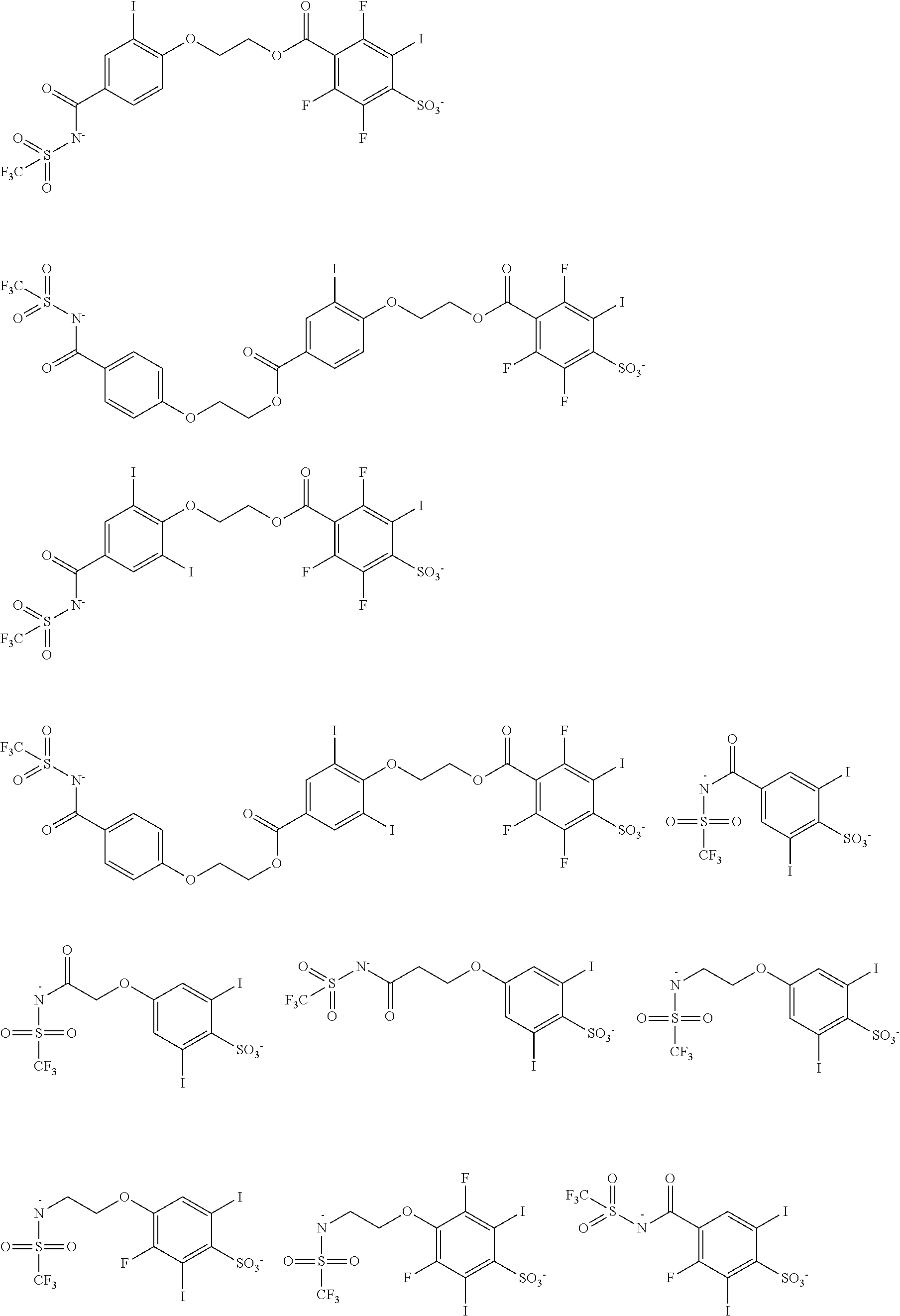

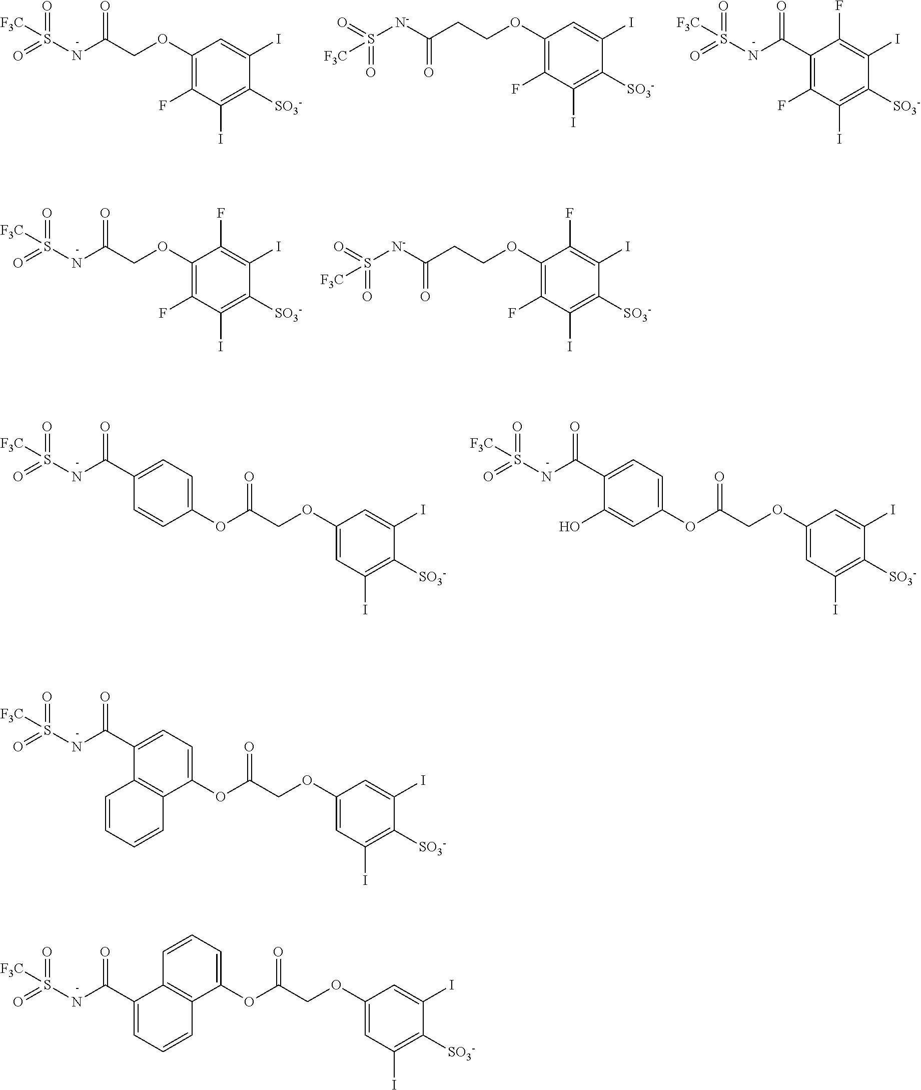

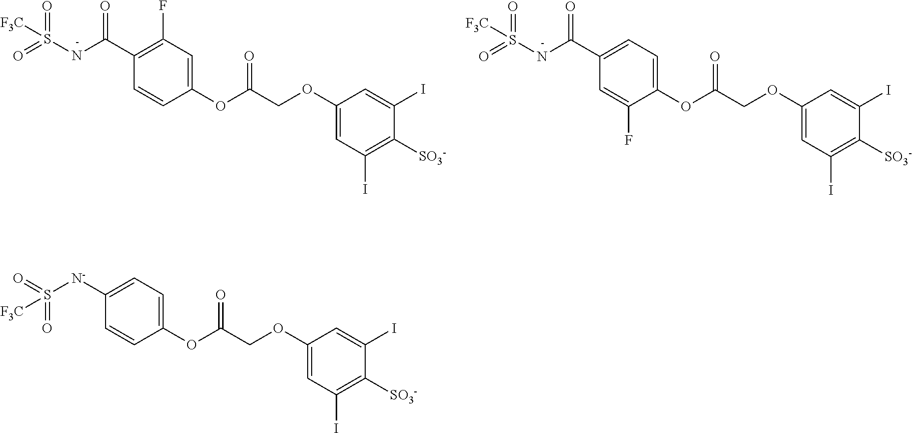

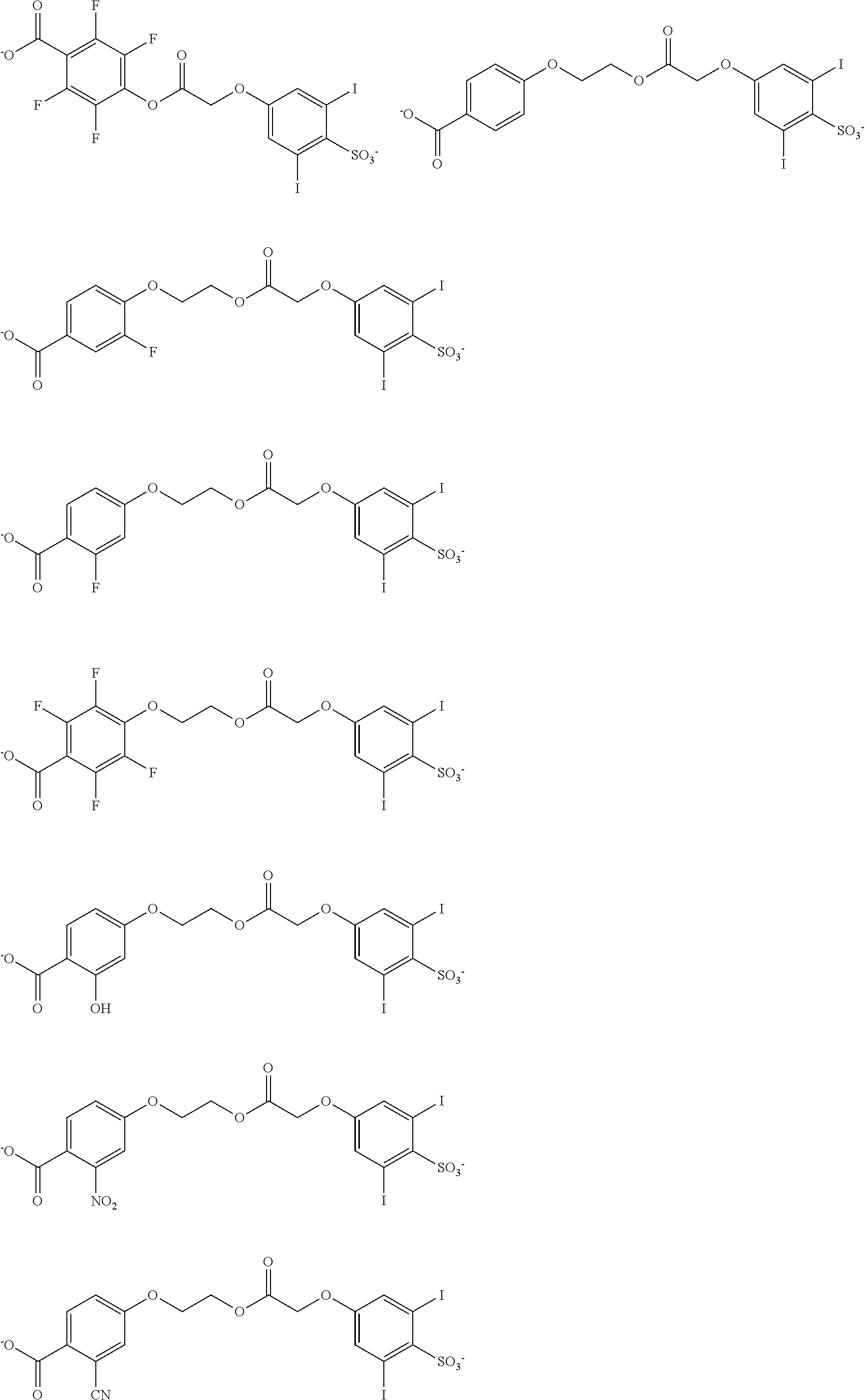

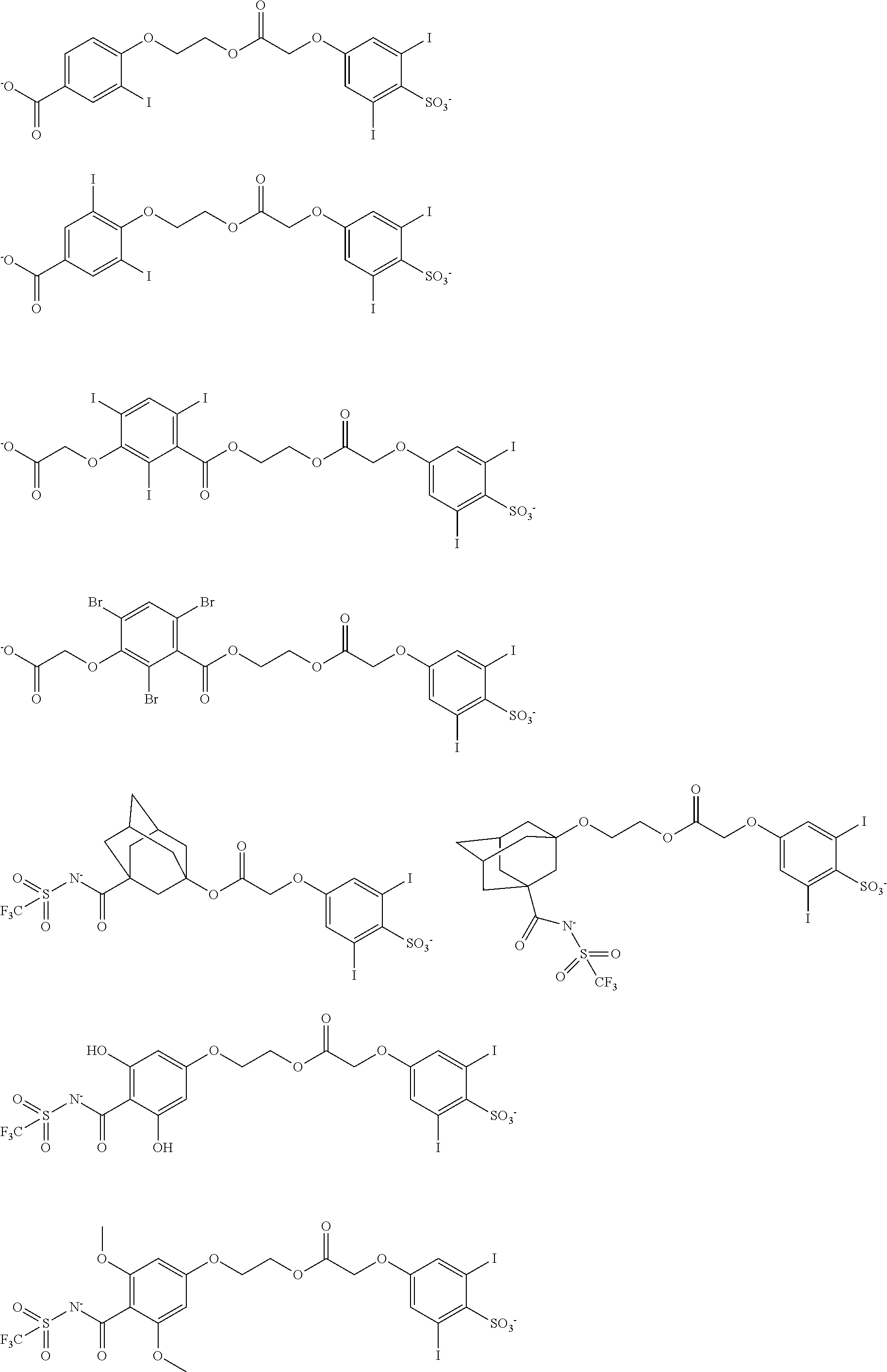

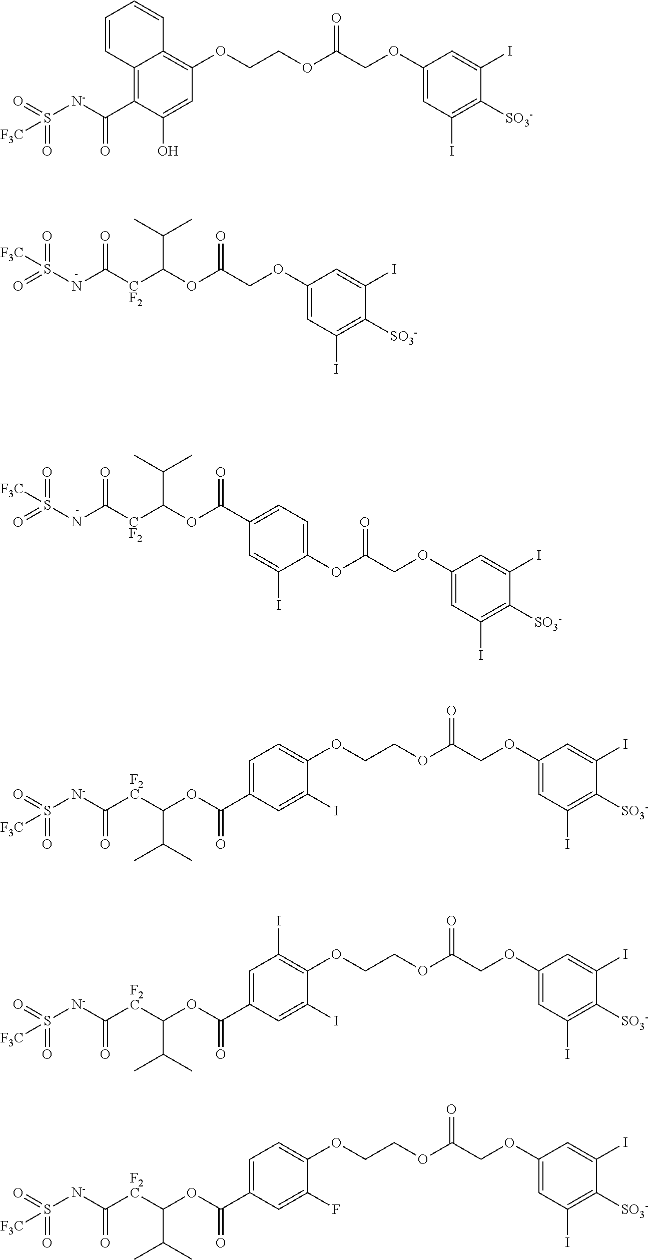

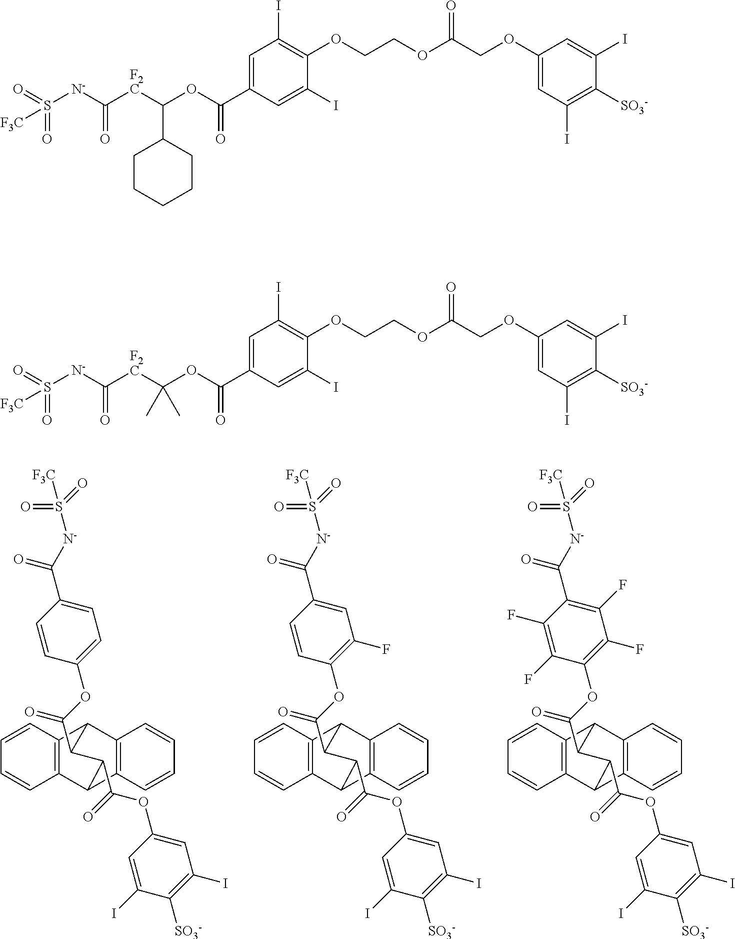

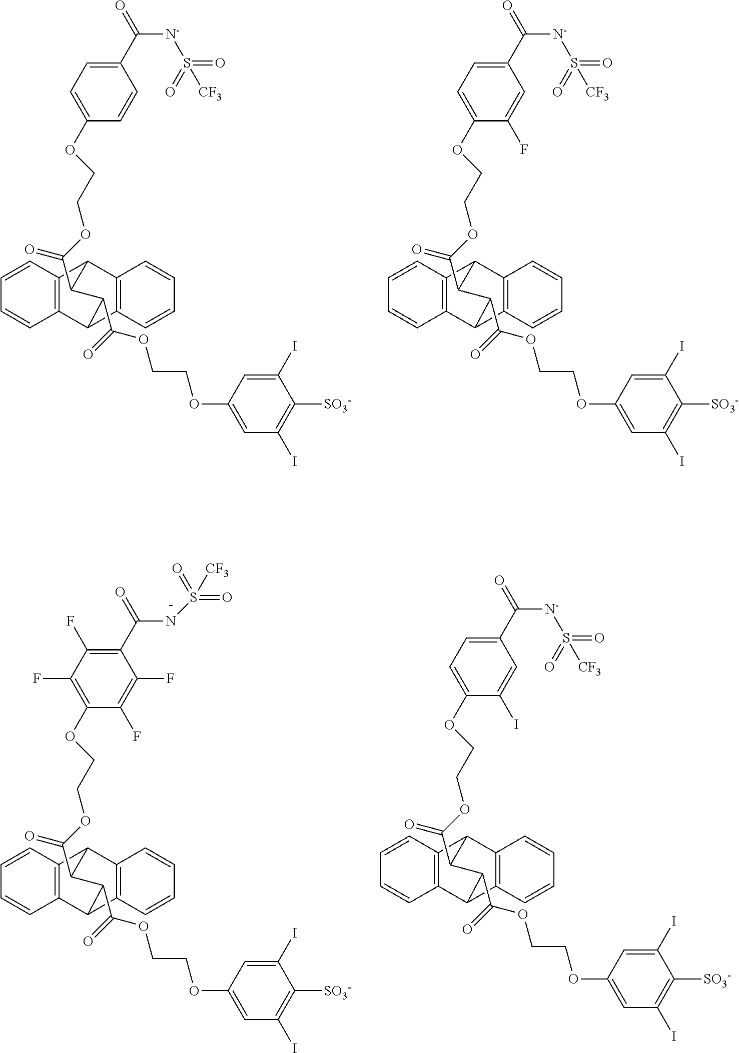

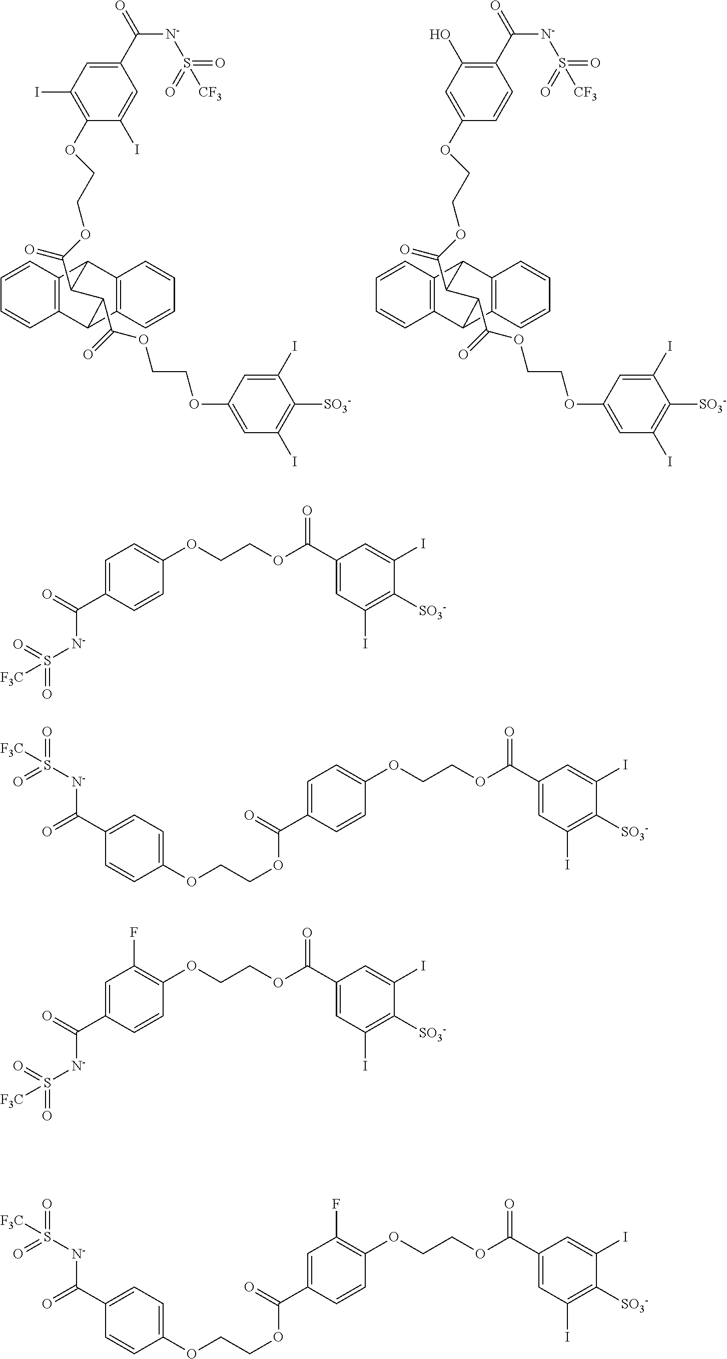

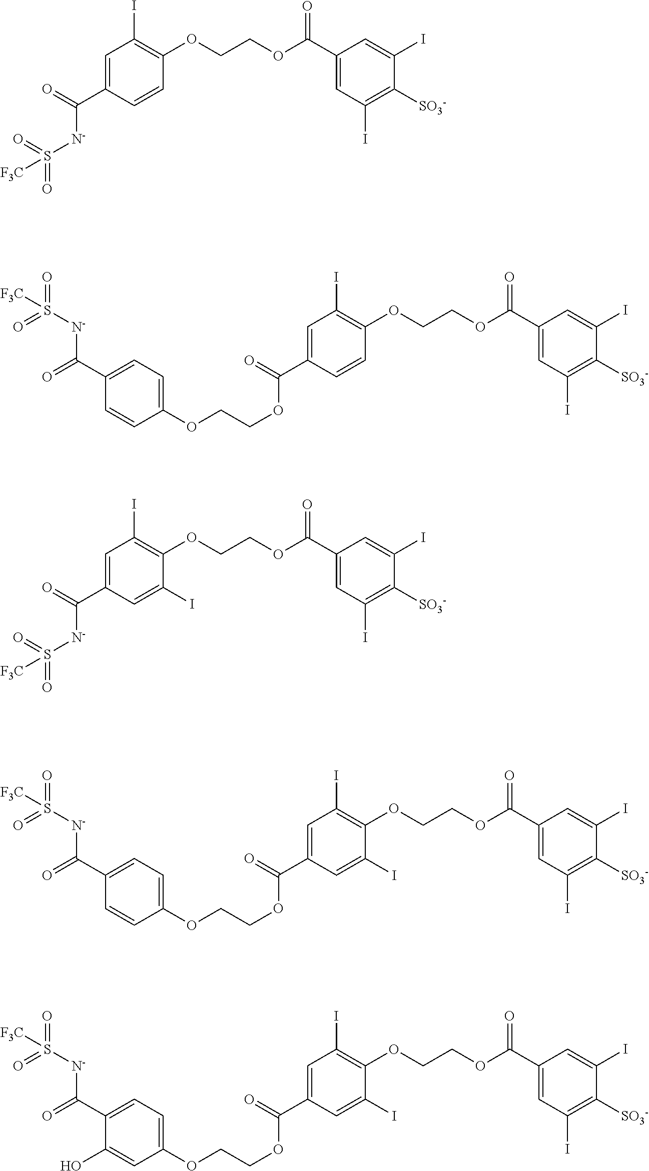

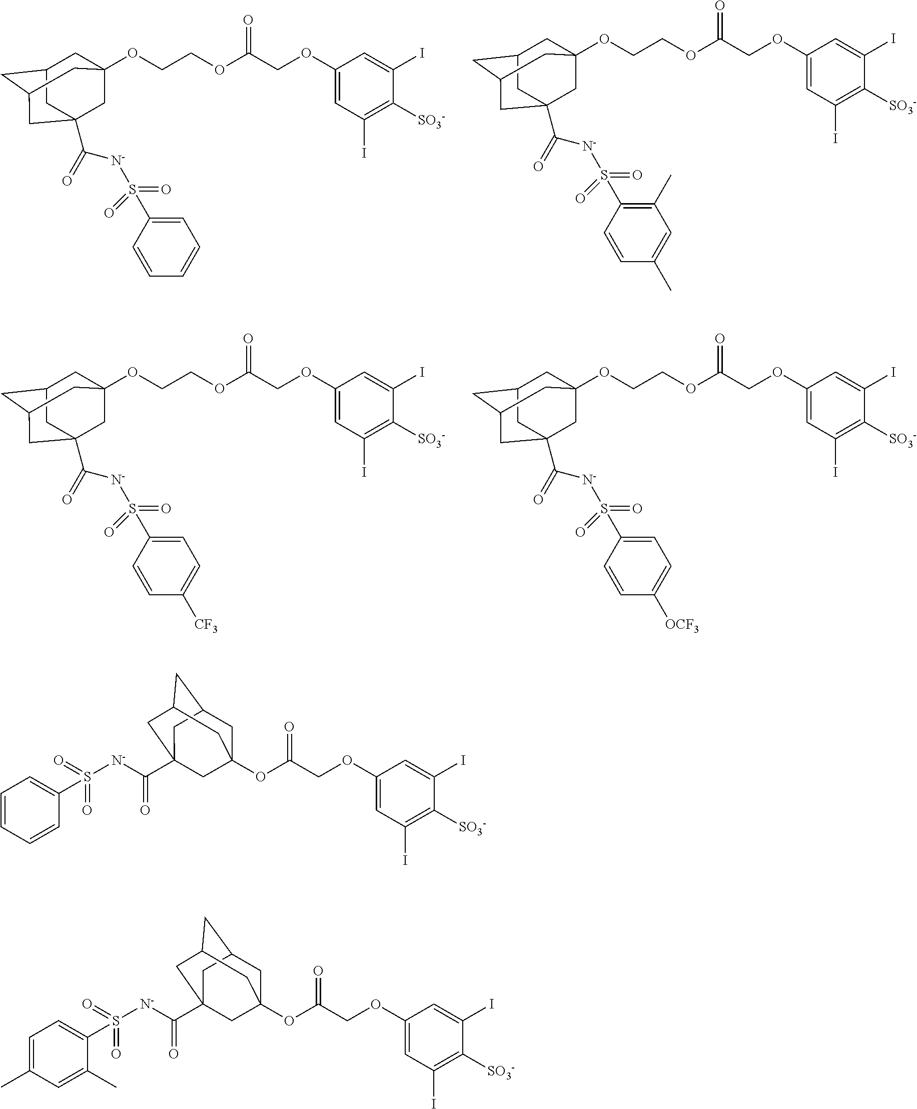

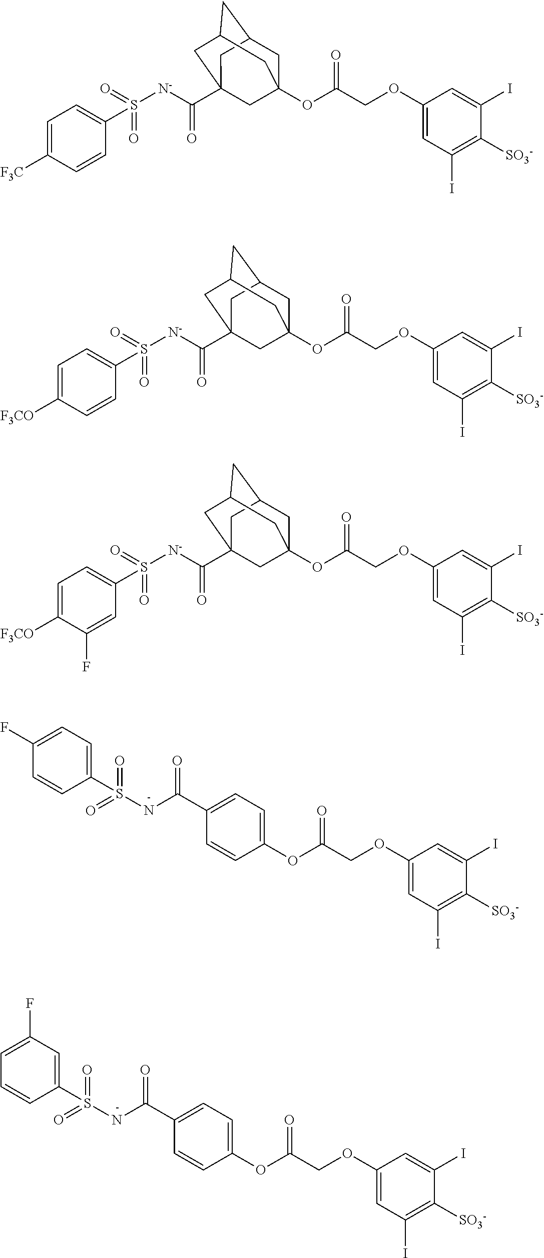

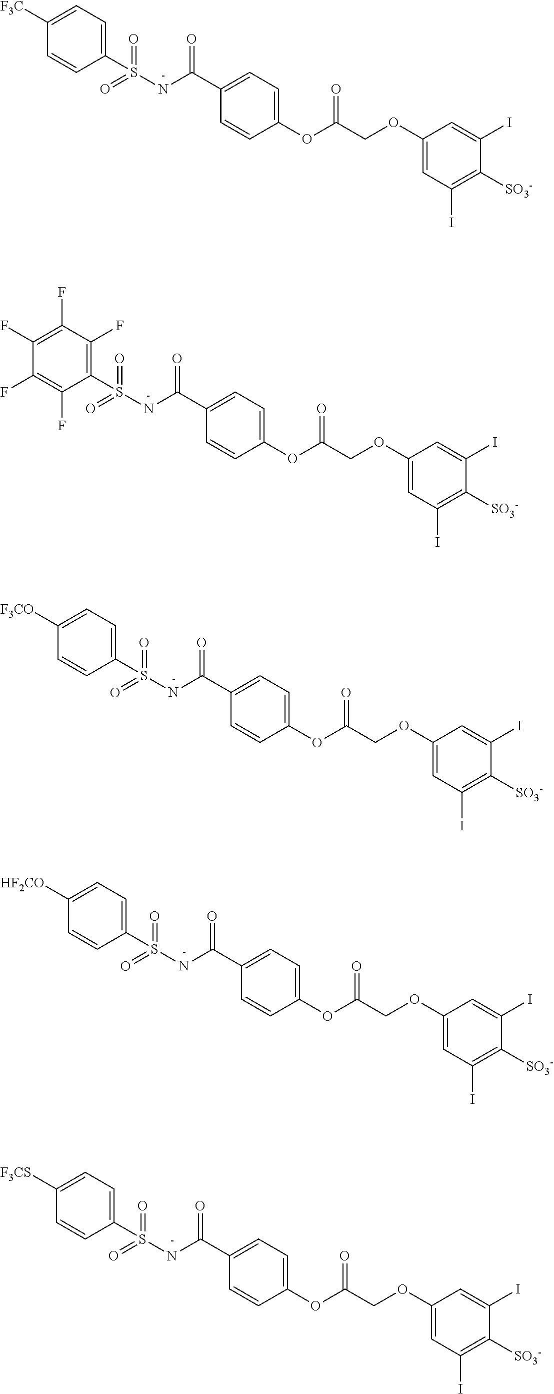

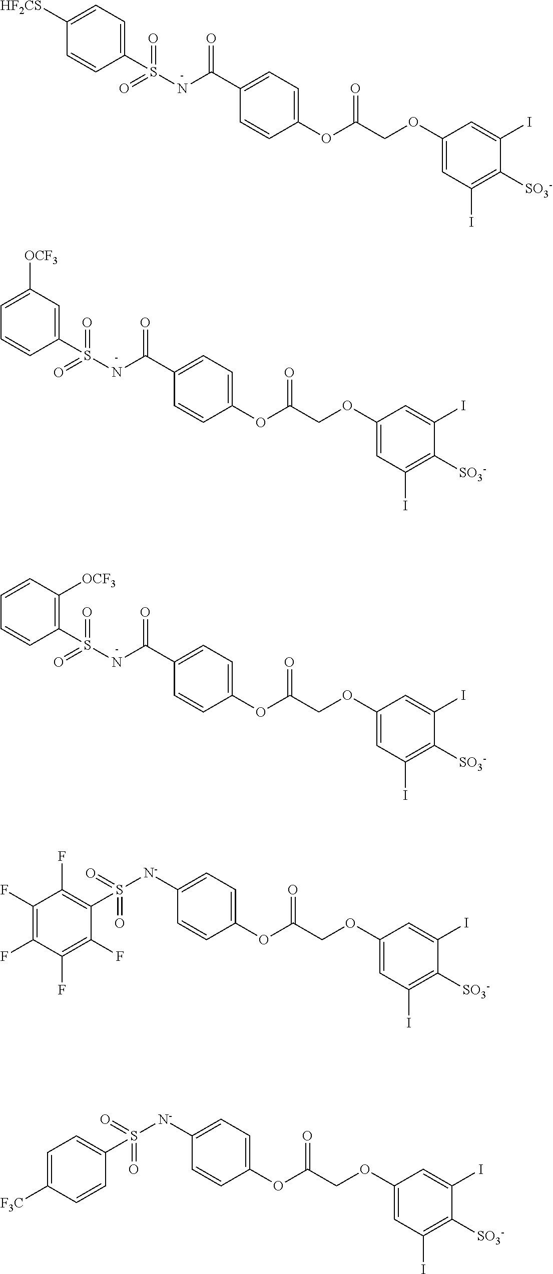

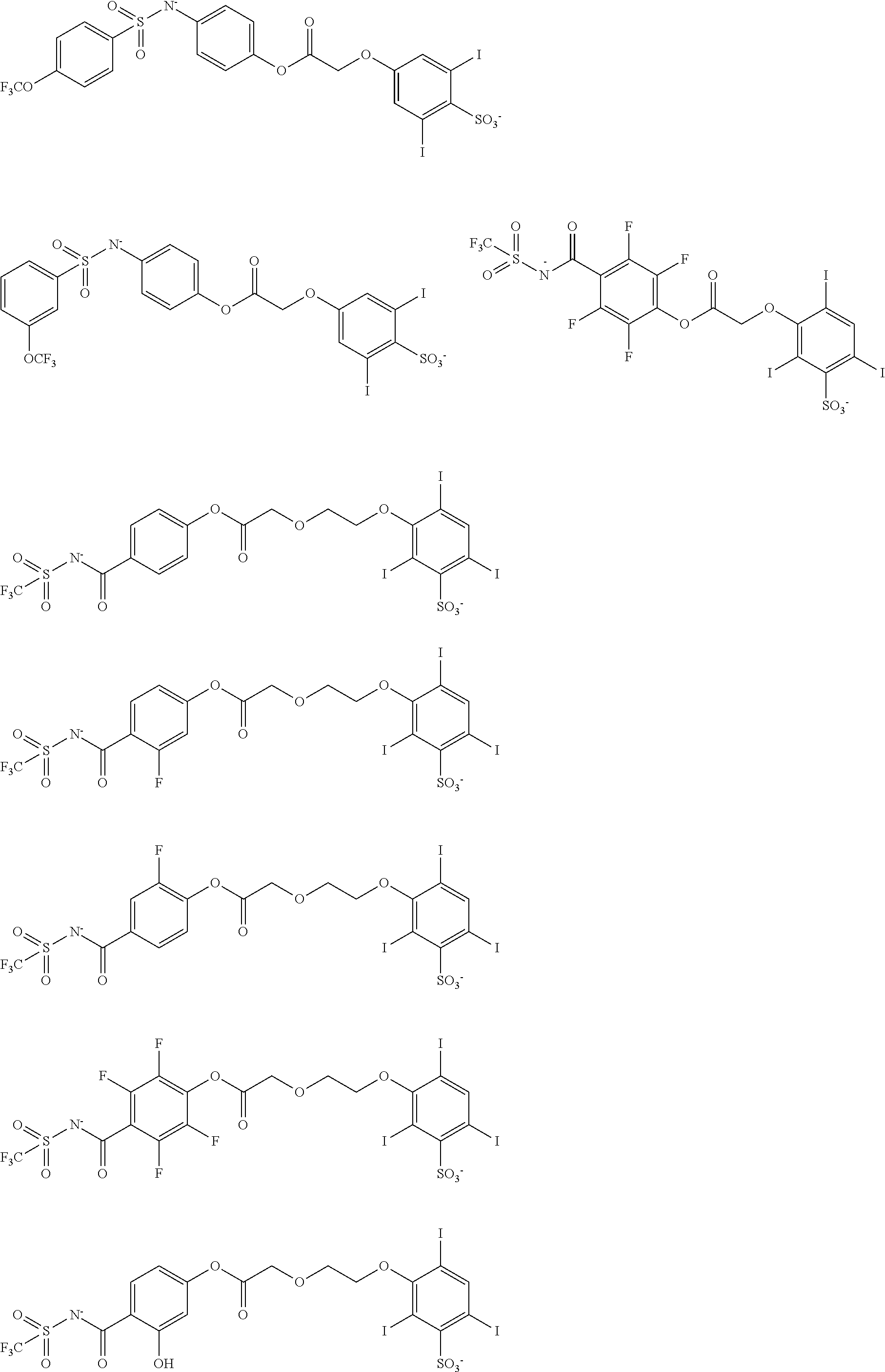

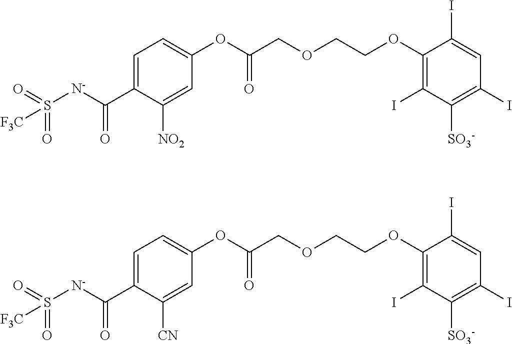

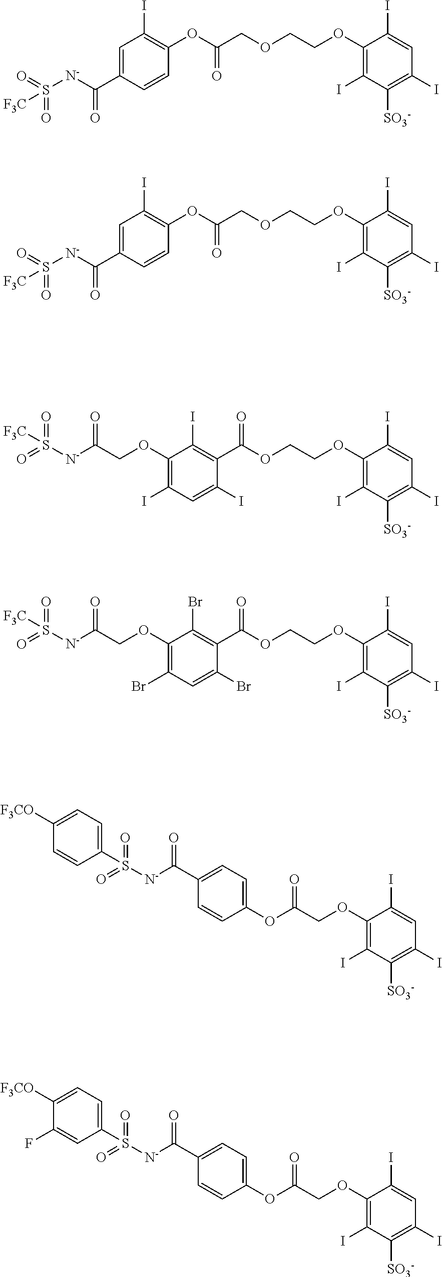

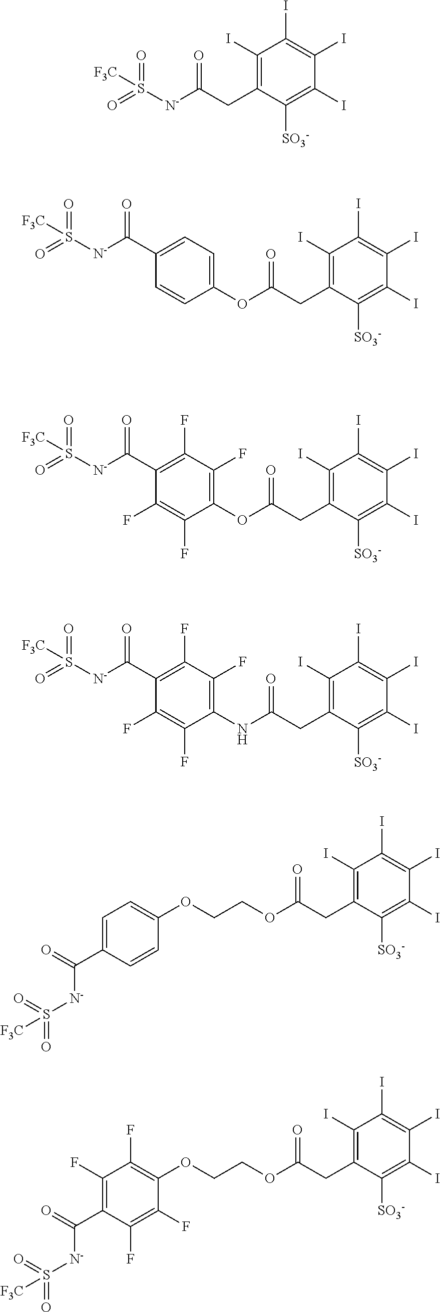

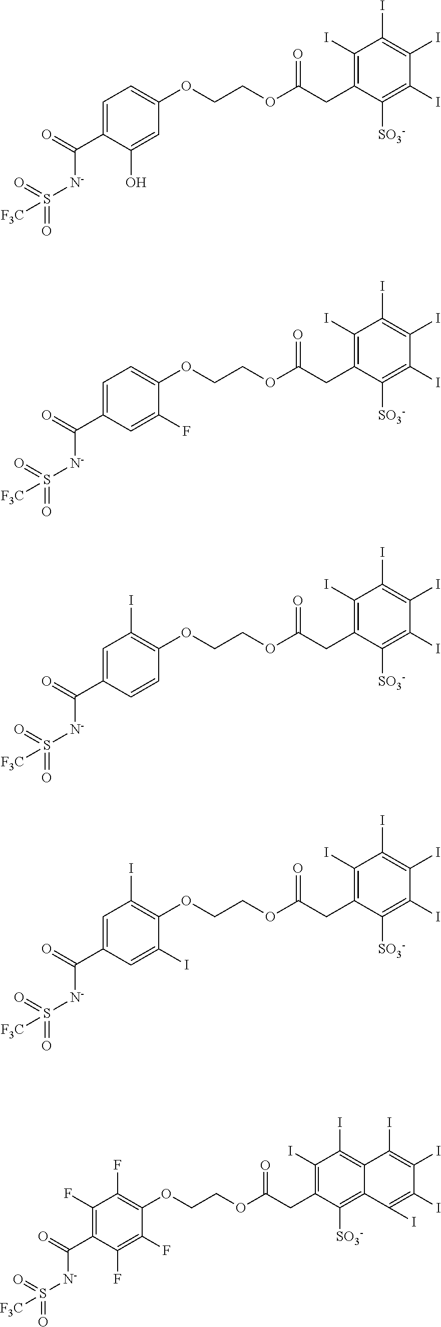

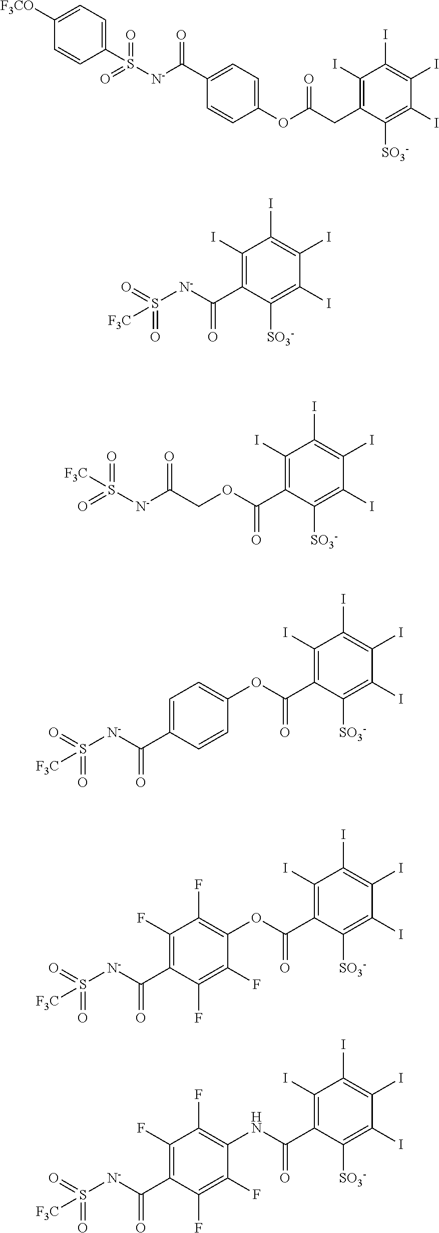

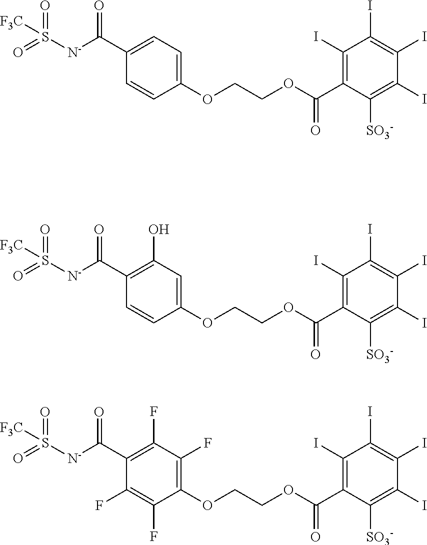

In formula (1), M⁺ is a sulfonium or iodonium cation. It is acceptable that of two M⁺, one is a sulfonium cation and the other is an iodonium cation, both are sulfonium cations, or both are iodonium cations. When two M⁺ are both sulfonium cations, they may be identical or different. When two M⁺ are both iodonium cations, they may be identical or different.

The preferred sulfonium cation has the formula (2) and the preferred iodonium cation has the formula (3).

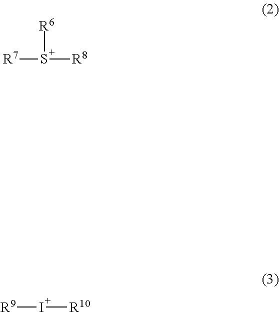

In formulae (2) and (3), R⁶ to R¹⁰ are each independently halogen or a C₁-C₂₀ hydrocarbyl group which may contain a heteroatom. Suitable halogen atoms include fluorine, chlorine, bromine and iodine.

The C₁-C₂₀ hydrocarbyl group represented by R⁶ to R¹⁰ may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, propenyl, butenyl, and hexenyl; C₂-C₂₀ alkynyl groups such as ethynyl, propynyl and butynyl; C₃-C₂₀ cyclic unsaturated aliphatic hydrocarbyl groups such as cyclohexenyl and norbornenyl; C₆-C₂₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, and tert-butylnaphthyl; C₇-C₂₀ aralkyl groups such as benzyl and phenethyl, and combinations thereof.

In the hydrocarbyl group, some or all of the hydrogen atoms may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, fluorine, chlorine, bromine, iodine, cyano, nitro, mercapto, pentafluorosulfanyl, carbonyl, ether bond, ester bond, sulfonate ester bond, carbonate bond, lactone ring, sultone ring, carboxylic anhydride (—C(═O)—O—C(═O)—) or haloalkyl moiety.

Also, R⁶ and R⁷ may bond together to form a ring with the sulfur atom to which they are attached. Preferred structures of the ring are shown below.

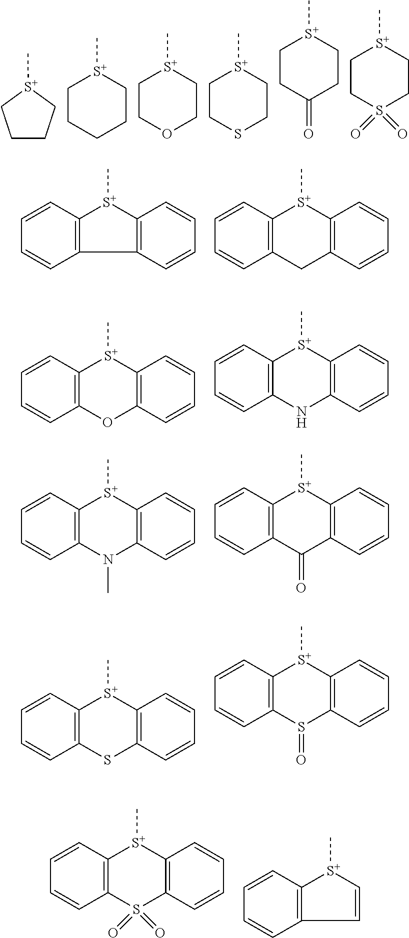

Examples of the sulfonium cation M⁺ are shown below, but not limited thereto.

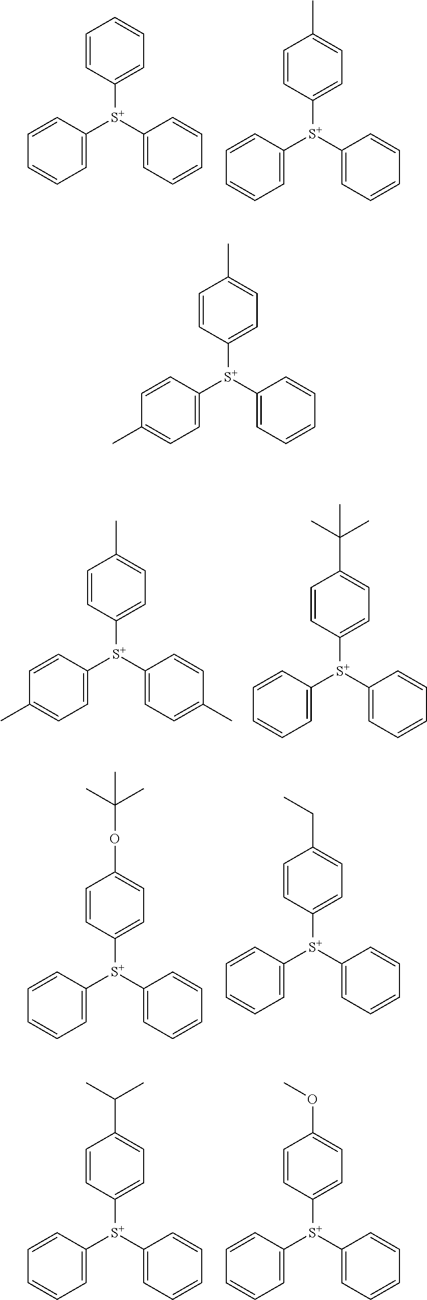

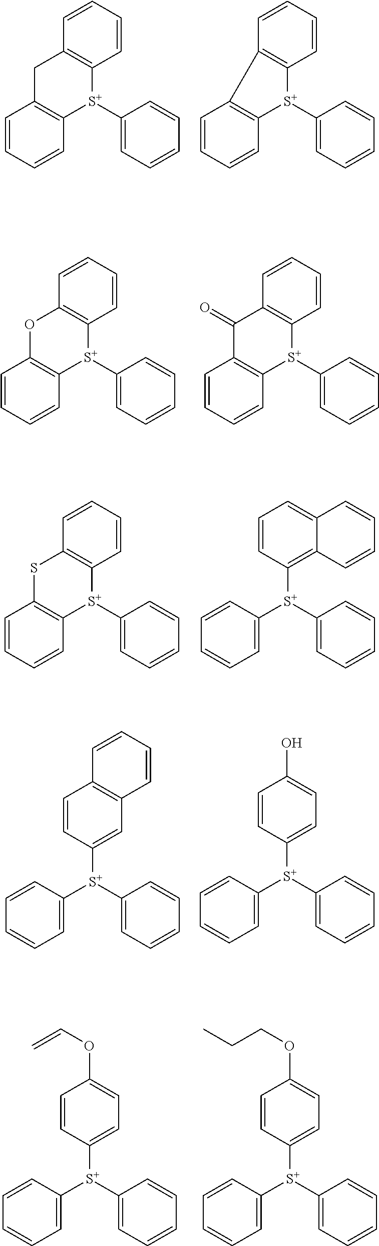

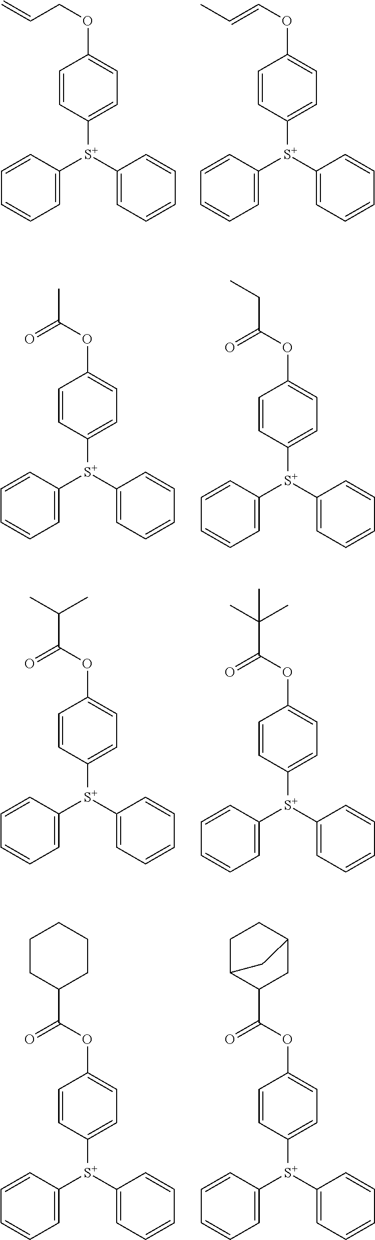

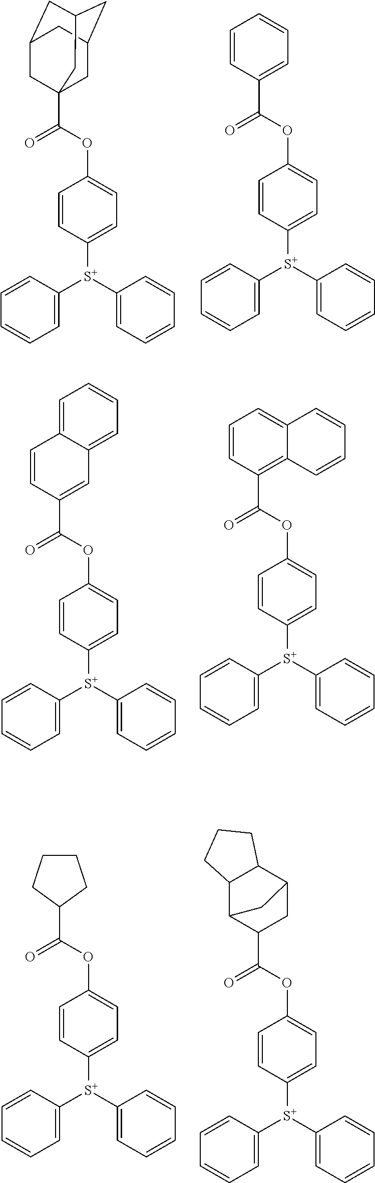 [SMILES: O=C(Oc1ccc([S+](c2ccccc2)c2ccccc2)cc1)C12CC3CC(CC(C3)C1)C2 ; O=C(Oc1ccc([S+](c2ccccc2)c2ccccc2)cc1)c1ccccc1 ; O=C(Oc1ccc([S+](c2ccccc2)c2ccccc2)cc1)c1ccc2ccccc2c1 ; O=C(Oc1ccc([S+](c2ccccc2)c2ccccc2)cc1)c1cccc2ccccc12 ; O=C(Oc1ccc([S+](c2ccccc2)c2ccccc2)cc1)C1CCCC1 ; O=C(Oc1ccc([S+](c2ccccc2)c2ccccc2)cc1)C1CC2CC1C1CCCC21]

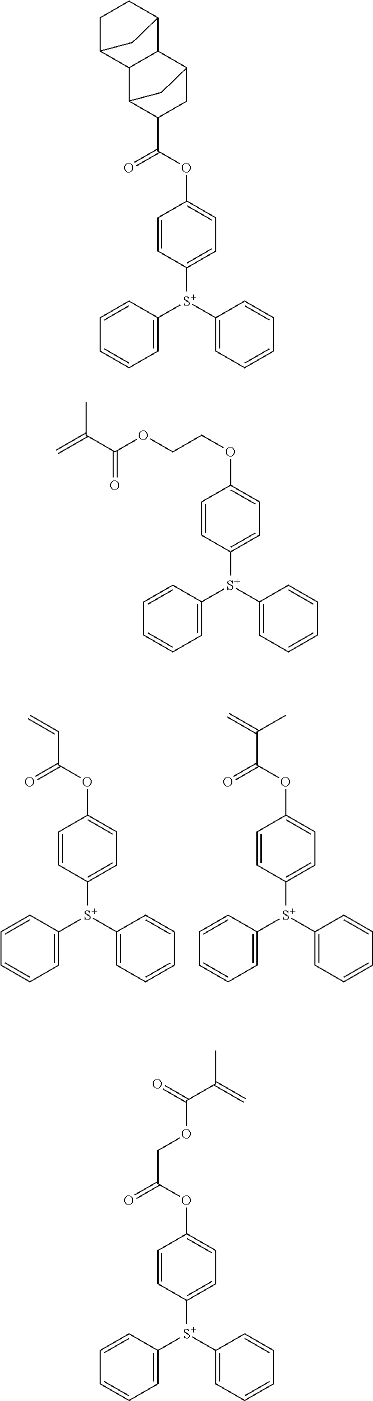 [SMILES: O=C(Oc1ccc([S+](c2ccccc2)c2ccccc2)cc1)C1CC2CC1C1C3CCC(C3)C21 ; C=C(C)C(=O)OCCOc1ccc([S+](c2ccccc2)c2ccccc2)cc1 ; C=CC(=O)Oc1ccc([S+](c2ccccc2)c2ccccc2)cc1 ; C=C(C)C(=O)Oc1ccc([S+](c2ccccc2)c2ccccc2)cc1 ; C=C(C)C(=O)OCC(=O)Oc1ccc([S+](c2ccccc2)c2ccccc2)cc1]

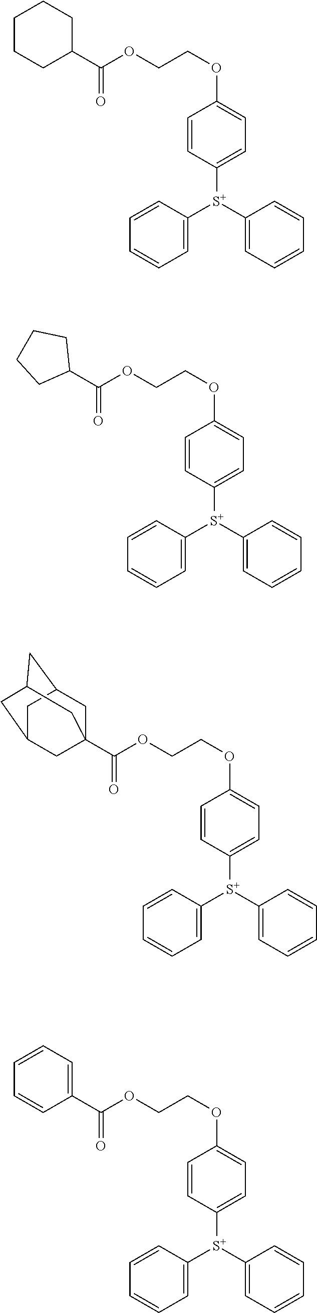 [SMILES: O=C(OCCOc1ccc([S+](c2ccccc2)c2ccccc2)cc1)C1CCCCC1 ; O=C(OCCOc1ccc([S+](c2ccccc2)c2ccccc2)cc1)C1CCCC1 ; O=C(OCCOc1ccc([S+](c2ccccc2)c2ccccc2)cc1)C12CC3CC(CC(C3)C1)C2 ; O=C(OCCOc1ccc([S+](c2ccccc2)c2ccccc2)cc1)c1ccccc1]

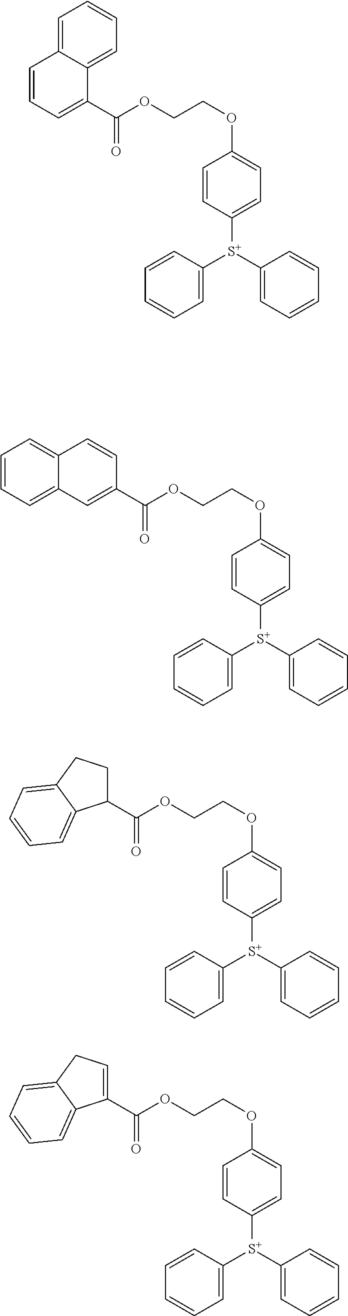 [SMILES: O=C(OCCOc1ccc([S+](c2ccccc2)c2ccccc2)cc1)c1cccc2ccccc12 ; O=C(OCCOc1ccc([S+](c2ccccc2)c2ccccc2)cc1)c1ccc2ccccc2c1 ; O=C(OCCOc1ccc([S+](c2ccccc2)c2ccccc2)cc1)C1CCc2ccccc21 ; O=C(OCCOc1ccc([S+](c2ccccc2)c2ccccc2)cc1)C1=CCc2ccccc21]

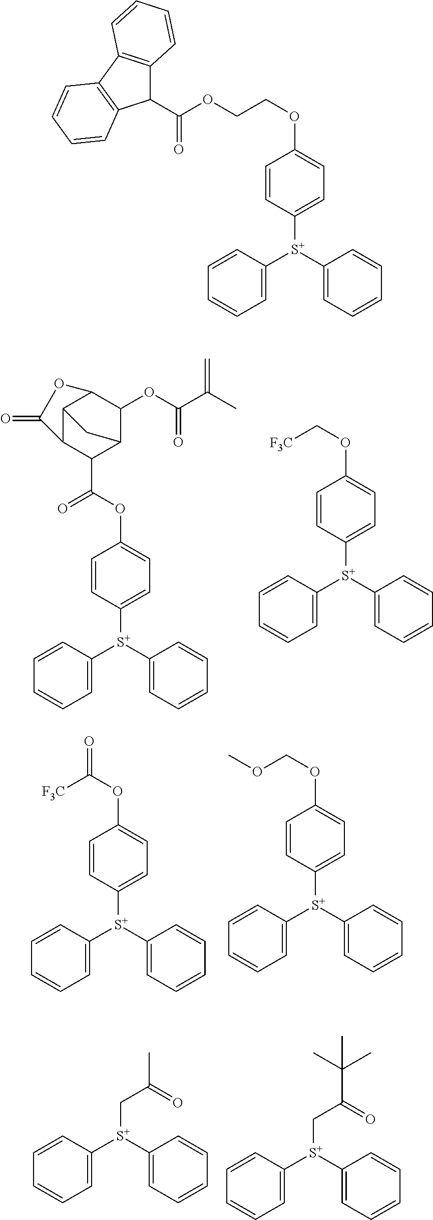

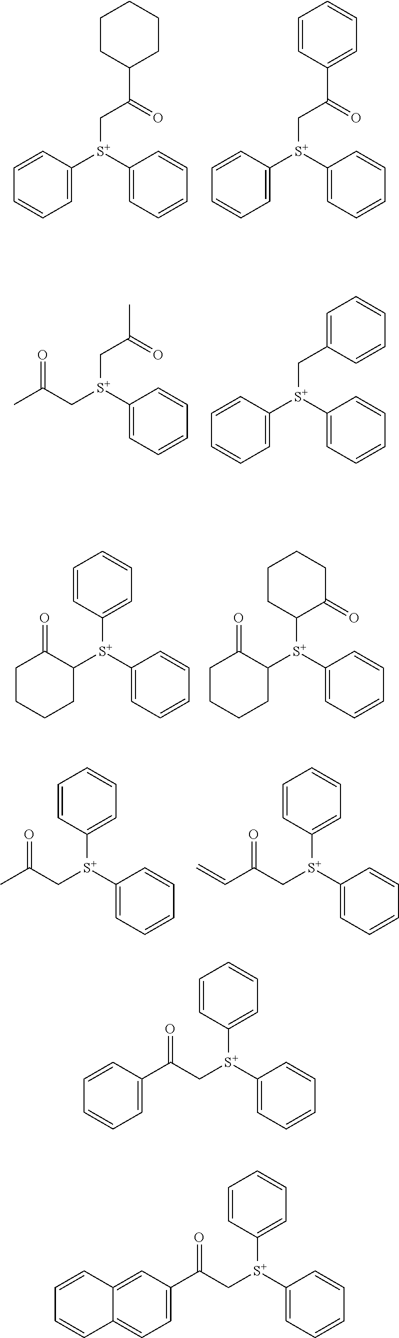

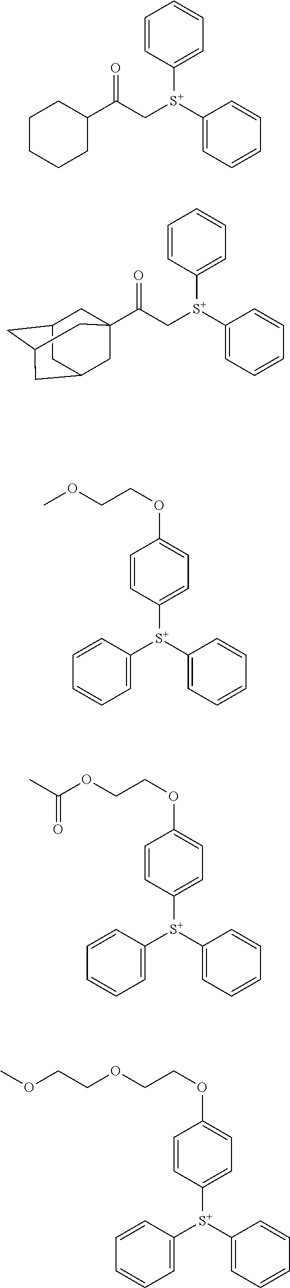 [SMILES: O=C(C[S+](c1ccccc1)c1ccccc1)C1CCCCC1 ; O=C(C[S+](c1ccccc1)c1ccccc1)C12CC3CC(CC(C3)C1)C2 ; COCCOc1ccc([S+](c2ccccc2)c2ccccc2)cc1 ; CC(=O)OCCOc1ccc([S+](c2ccccc2)c2ccccc2)cc1 ; COCCOCCOc1ccc([S+](c2ccccc2)c2ccccc2)cc1]

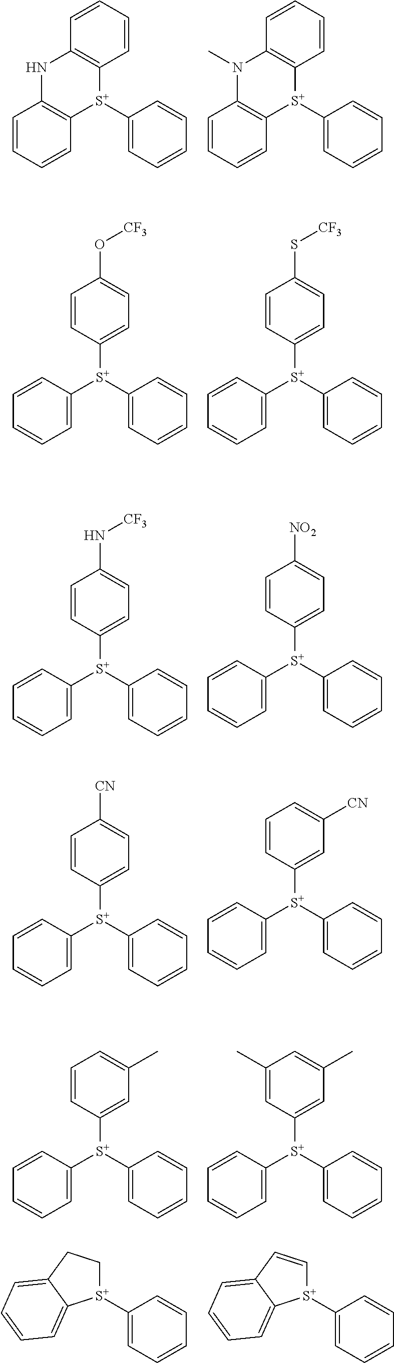

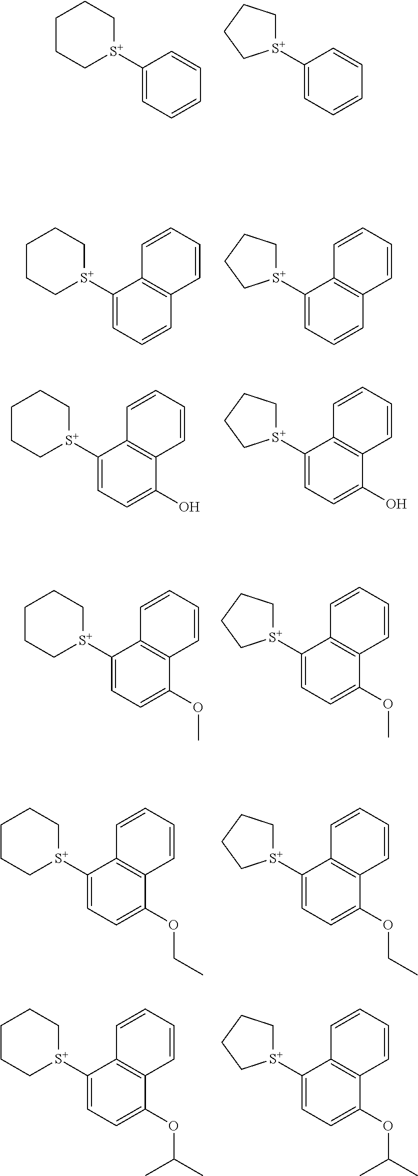

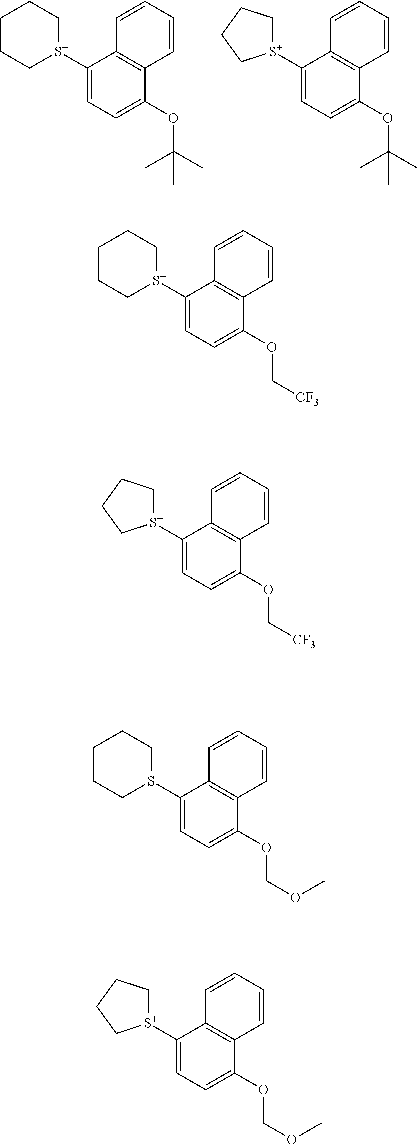 [SMILES: CC(C)(C)Oc1ccc([S+]2CCCCC2)c2ccccc12 ; CC(C)(C)Oc1ccc([S+]2CCCC2)c2ccccc12 ; FC(F)(F)COc1ccc([S+]2CCCCC2)c2ccccc12 ; FC(F)(F)COc1ccc([S+]2CCCC2)c2ccccc12 ; COCOc1ccc([S+]2CCCCC2)c2ccccc12 ; COCOc1ccc([S+]2CCCC2)c2ccccc12]

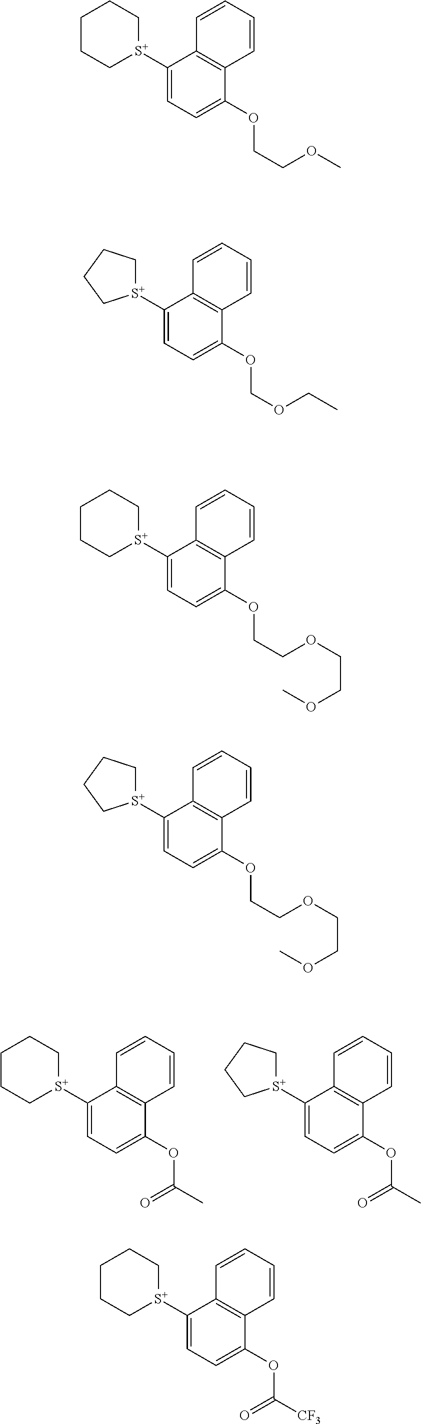

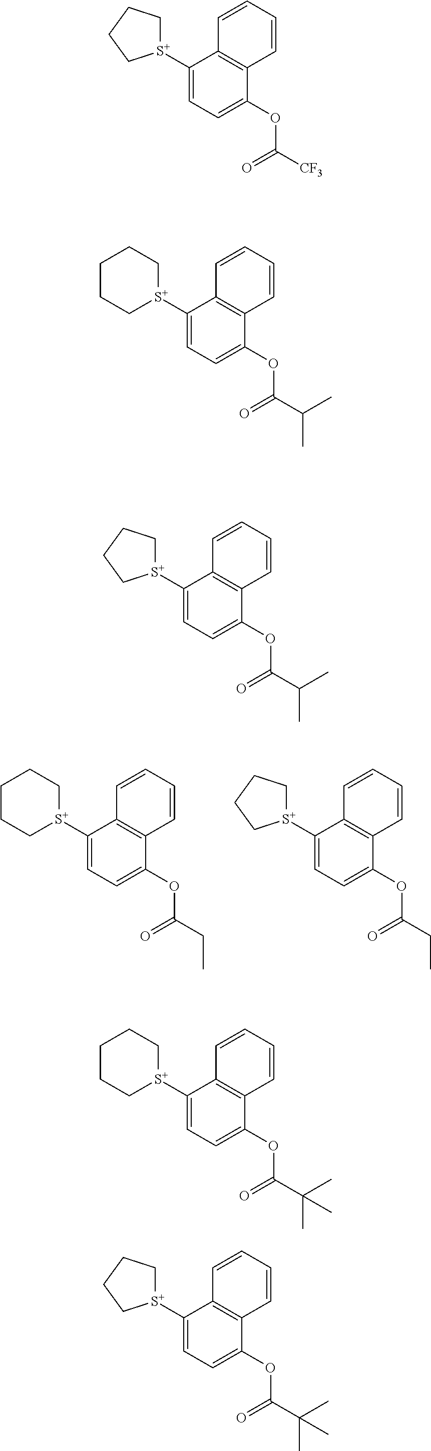

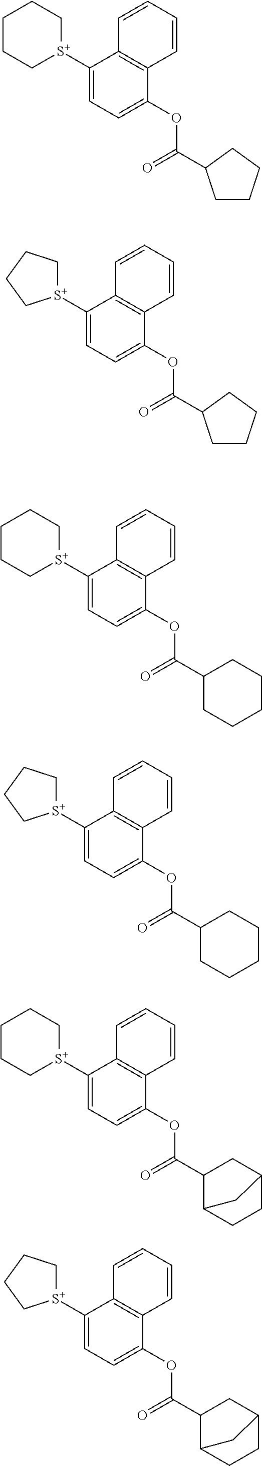 [SMILES: O=C(Oc1ccc([S+]2CCCCC2)c2ccccc12)C1CCCC1 ; O=C(Oc1ccc([S+]2CCCC2)c2ccccc12)C1CCCC1 ; O=C(Oc1ccc([S+]2CCCCC2)c2ccccc12)C1CCCCC1 ; O=C(Oc1ccc([S+]2CCCC2)c2ccccc12)C1CCCCC1 ; O=C(Oc1ccc([S+]2CCCCC2)c2ccccc12)C1CC2CCC1C2 ; O=C(Oc1ccc([S+]2CCCC2)c2ccccc12)C1CC2CCC1C2]

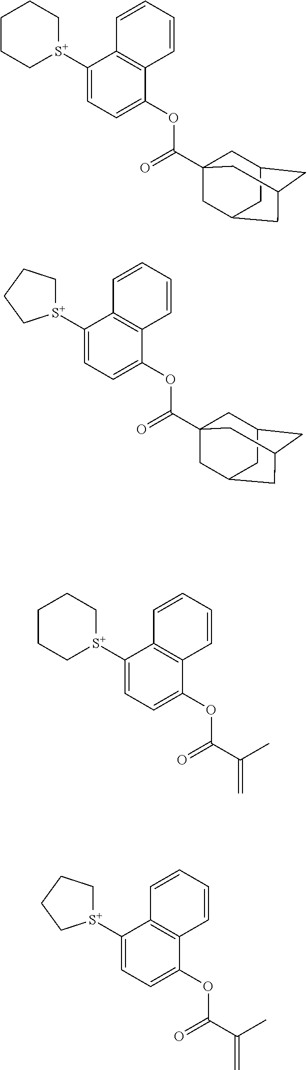 [SMILES: O=C(Oc1ccc([S+]2CCCCC2)c2ccccc12)C12CC3CC(CC(C3)C1)C2 ; O=C(Oc1ccc([S+]2CCCC2)c2ccccc12)C12CC3CC(CC(C3)C1)C2 ; C=C(C)C(=O)Oc1ccc([S+]2CCCCC2)c2ccccc12 ; C=C(C)C(=O)Oc1ccc([S+]2CCCC2)c2ccccc12]

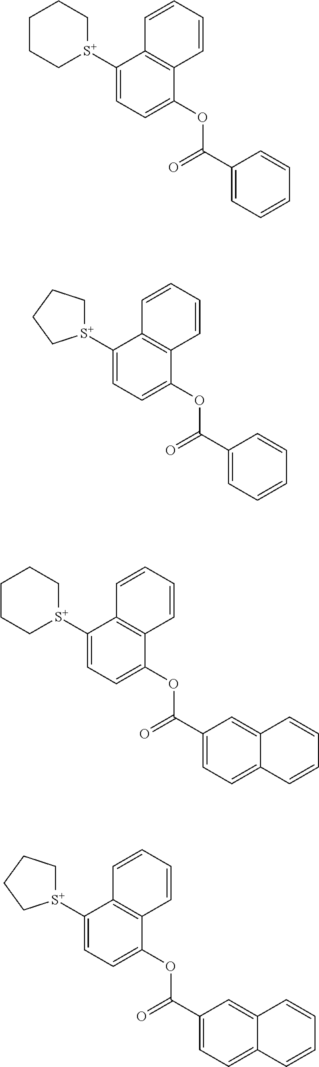 [SMILES: O=C(Oc1ccc([S+]2CCCCC2)c2ccccc12)c1ccccc1 ; O=C(Oc1ccc([S+]2CCCC2)c2ccccc12)c1ccccc1 ; O=C(Oc1ccc([S+]2CCCCC2)c2ccccc12)c1ccc2ccccc2c1 ; O=C(Oc1ccc([S+]2CCCC2)c2ccccc12)c1ccc2ccccc2c1]

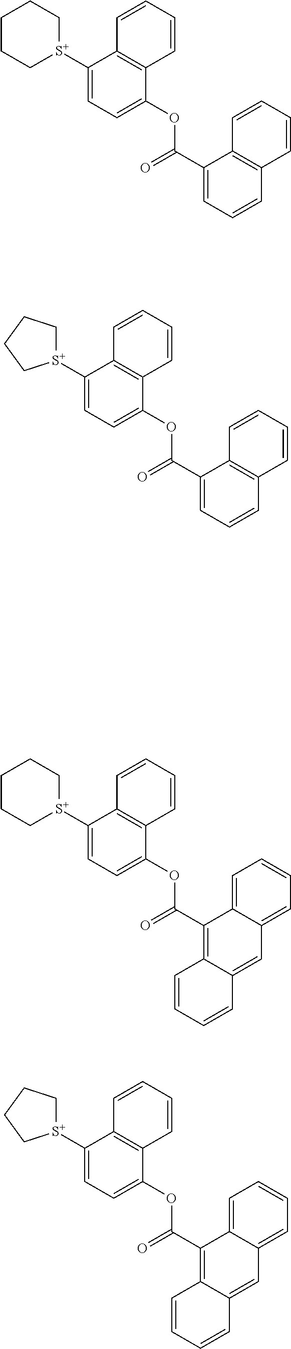 [SMILES: O=C(Oc1ccc([S+]2CCCCC2)c2ccccc12)c1cccc2ccccc12 ; O=C(Oc1ccc([S+]2CCCC2)c2ccccc12)c1cccc2ccccc12 ; O=C(Oc1ccc([S+]2CCCCC2)c2ccccc12)c1c2ccccc2cc2ccccc12 ; O=C(Oc1ccc([S+]2CCCC2)c2ccccc12)c1c2ccccc2cc2ccccc12]

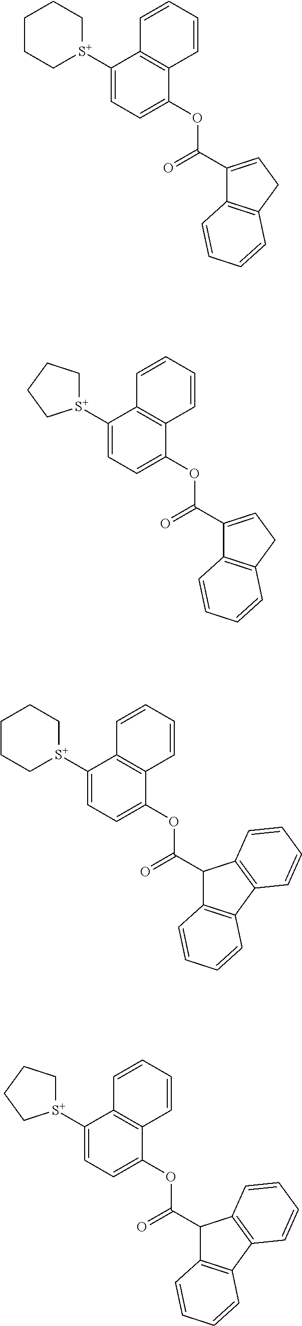 [SMILES: O=C(Oc1ccc([S+]2CCCCC2)c2ccccc12)C1=CCc2ccccc21 ; O=C(Oc1ccc([S+]2CCCC2)c2ccccc12)C1=CCc2ccccc21 ; O=C(Oc1ccc([S+]2CCCCC2)c2ccccc12)C1c2ccccc2-c2ccccc21 ; O=C(Oc1ccc([S+]2CCCC2)c2ccccc12)C1c2ccccc2-c2ccccc21]

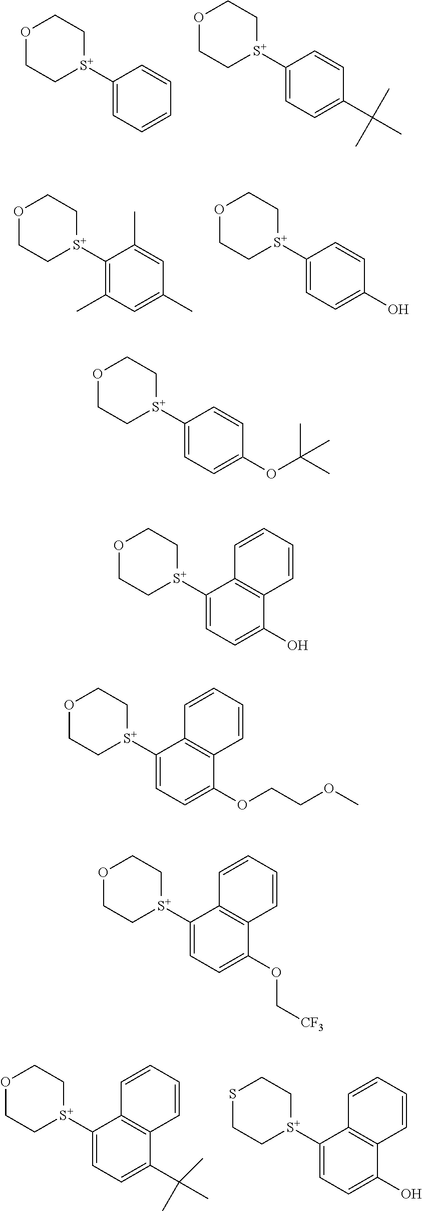

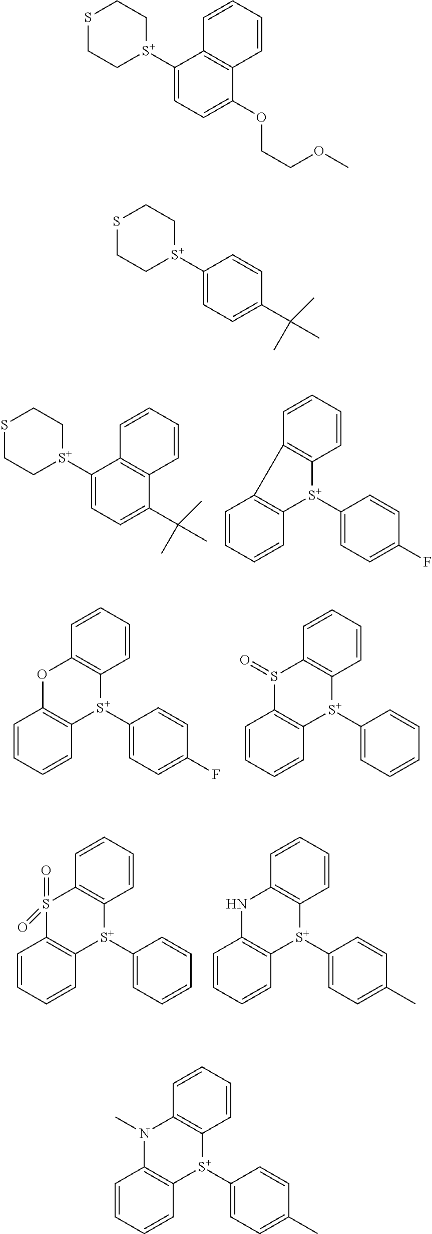

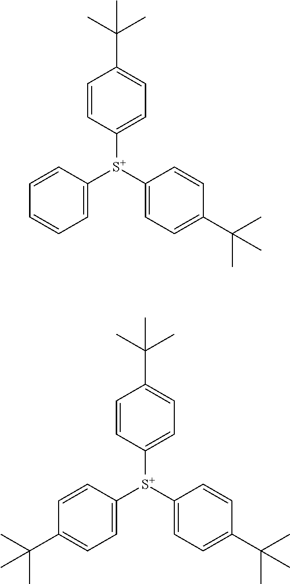

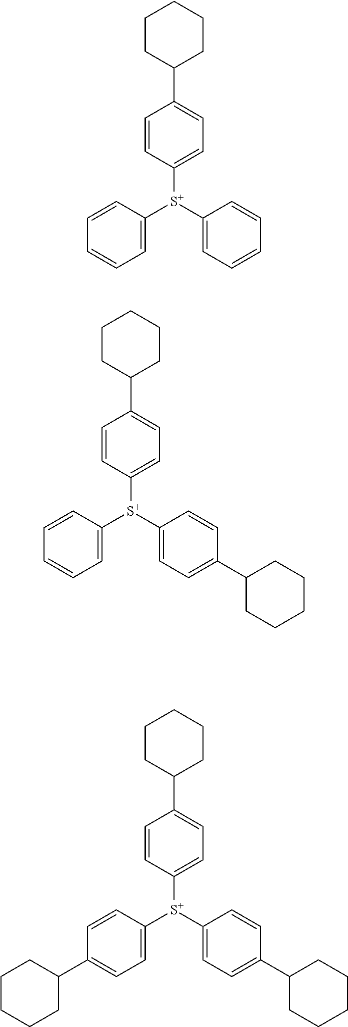 [SMILES: c1ccc([S+](c2ccccc2)c2ccc(C3CCCCC3)cc2)cc1 ; c1ccc([S+](c2ccc(C3CCCCC3)cc2)c2ccc(C3CCCCC3)cc2)cc1 ; c1cc([S+](c2ccc(C3CCCCC3)cc2)c2ccc(C3CCCCC3)cc2)ccc1C1CCCCC1]

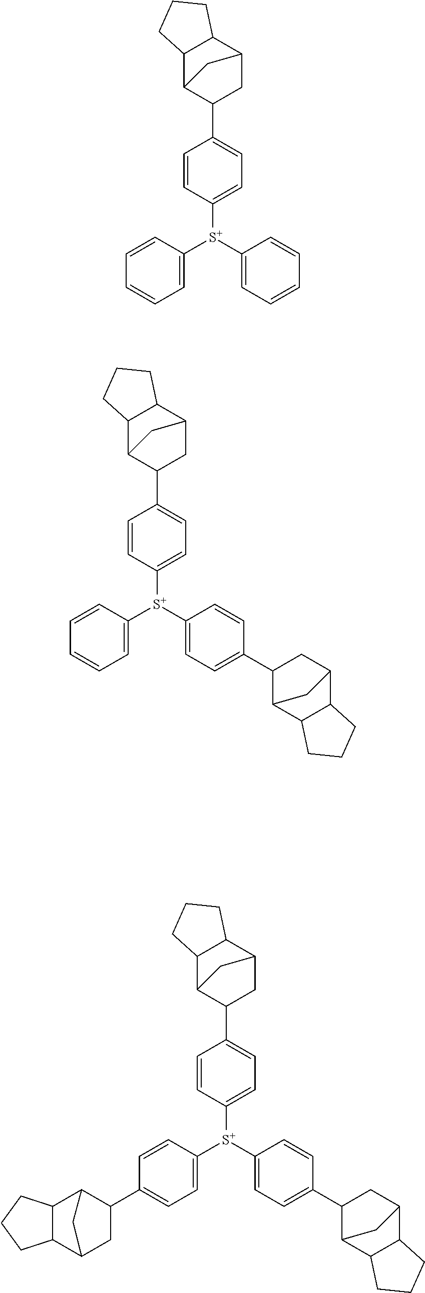 [SMILES: c1ccc([S+](c2ccccc2)c2ccc(C3CC4CC3C3CCCC43)cc2)cc1 ; c1ccc([S+](c2ccc(C3CC4CC3C3CCCC43)cc2)c2ccc(C3CC4CC3C3CCCC43)cc2)cc1 ; c1cc([S+](c2ccc(C3CC4CC3C3CCCC43)cc2)c2ccc(C3CC4CC3C3CCCC43)cc2)ccc1C1CC2CC1C1CCCC21]

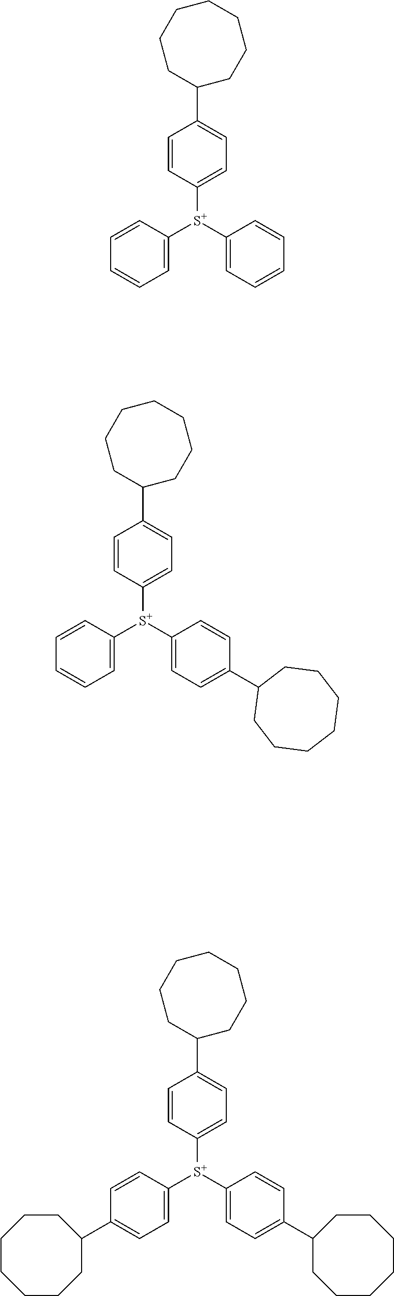 [SMILES: c1ccc([S+](c2ccccc2)c2ccc(C3CCCCCCC3)cc2)cc1 ; c1ccc([S+](c2ccc(C3CCCCCCC3)cc2)c2ccc(C3CCCCCCC3)cc2)cc1 ; c1cc([S+](c2ccc(C3CCCCCCC3)cc2)c2ccc(C3CCCCCCC3)cc2)ccc1C1CCCCCCC1]

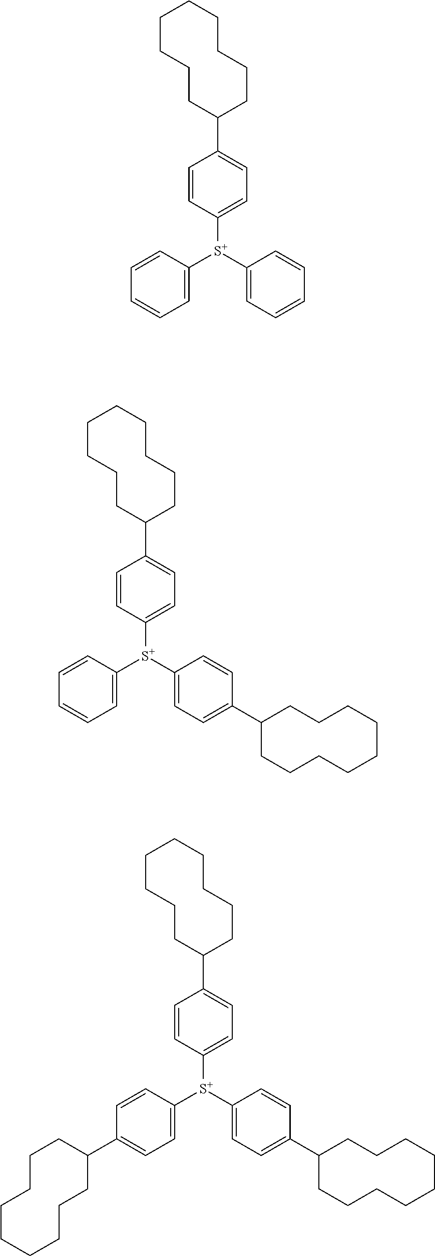 [SMILES: c1ccc([S+](c2ccccc2)c2ccc(C3CCCCCCCCC3)cc2)cc1 ; c1ccc([S+](c2ccc(C3CCCCCCCCC3)cc2)c2ccc(C3CCCCCCCCC3)cc2)cc1 ; c1cc([S+](c2ccc(C3CCCCCCCCC3)cc2)c2ccc(C3CCCCCCCCC3)cc2)ccc1C1CCCCCCCCC1]

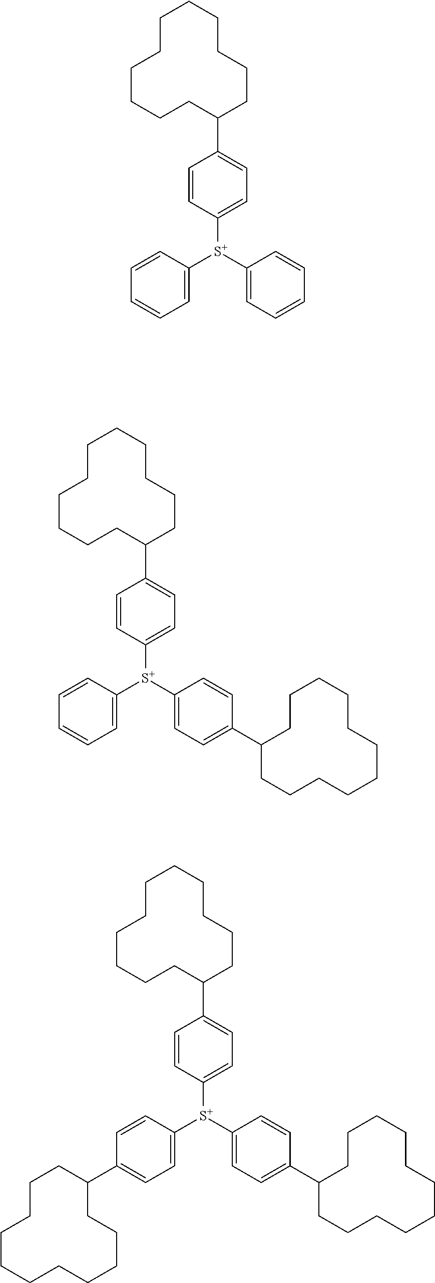 [SMILES: c1ccc([S+](c2ccccc2)c2ccc(C3CCCCCCCCCCC3)cc2)cc1 ; c1ccc([S+](c2ccc(C3CCCCCCCCCCC3)cc2)c2ccc(C3CCCCCCCCCCC3)cc2)cc1 ; c1cc([S+](c2ccc(C3CCCCCCCCCCC3)cc2)c2ccc(C3CCCCCCCCCCC3)cc2)ccc1C1CCCCCCCCCCC1]

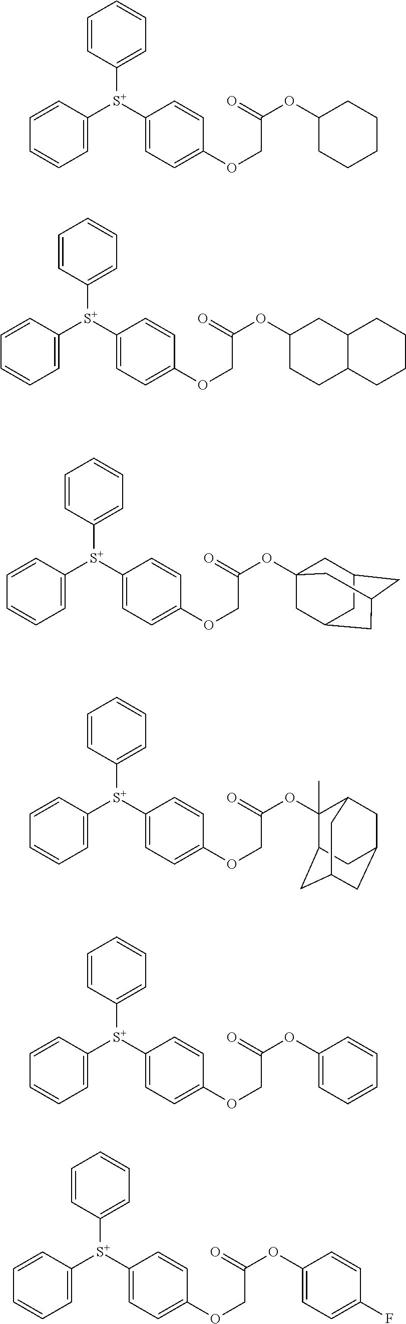 [SMILES: O=C(COc1ccc([S+](c2ccccc2)c2ccccc2)cc1)OC1CCCCC1 ; O=C(COc1ccc([S+](c2ccccc2)c2ccccc2)cc1)OC1CCC2CCCCC2C1 ; O=C(COc1ccc([S+](c2ccccc2)c2ccccc2)cc1)OC12CC3CC(CC(C3)C1)C2 ; CC1(OC(=O)COc2ccc([S+](c3ccccc3)c3ccccc3)cc2)C2CC3CC(C2)CC1C3 ; O=C(COc1ccc([S+](c2ccccc2)c2ccccc2)cc1)Oc1ccccc1 ; O=C(COc1ccc([S+](c2ccccc2)c2ccccc2)cc1)Oc1ccc(F)cc1]

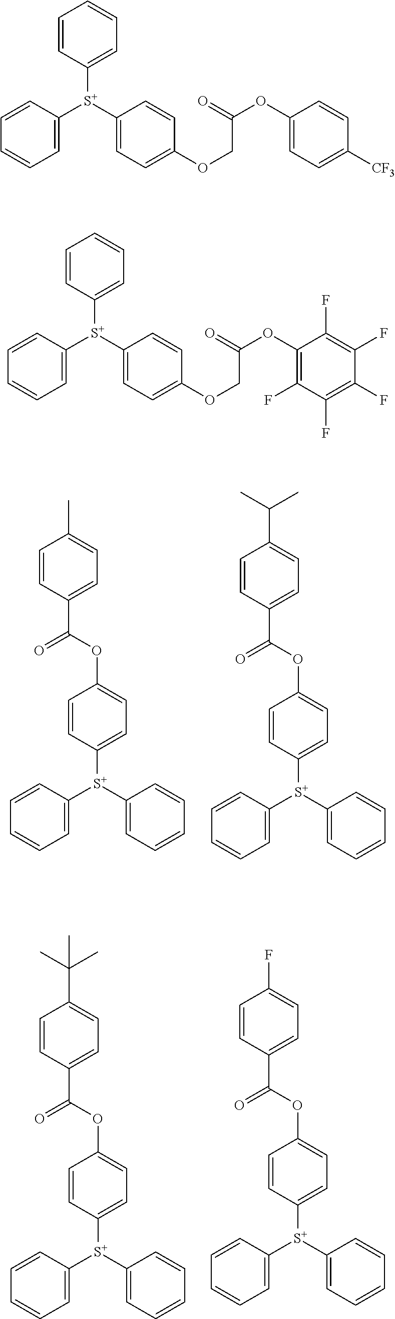 [SMILES: O=C(COc1ccc([S+](c2ccccc2)c2ccccc2)cc1)Oc1ccc(C(F)(F)F)cc1 ; O=C(COc1ccc([S+](c2ccccc2)c2ccccc2)cc1)Oc1c(F)c(F)c(F)c(F)c1F ; Cc1ccc(C(=O)Oc2ccc([S+](c3ccccc3)c3ccccc3)cc2)cc1 ; CC(C)c1ccc(C(=O)Oc2ccc([S+](c3ccccc3)c3ccccc3)cc2)cc1 ; CC(C)(C)c1ccc(C(=O)Oc2ccc([S+](c3ccccc3)c3ccccc3)cc2)cc1 ; O=C(Oc1ccc([S+](c2ccccc2)c2ccccc2)cc1)c1ccc(F)cc1]

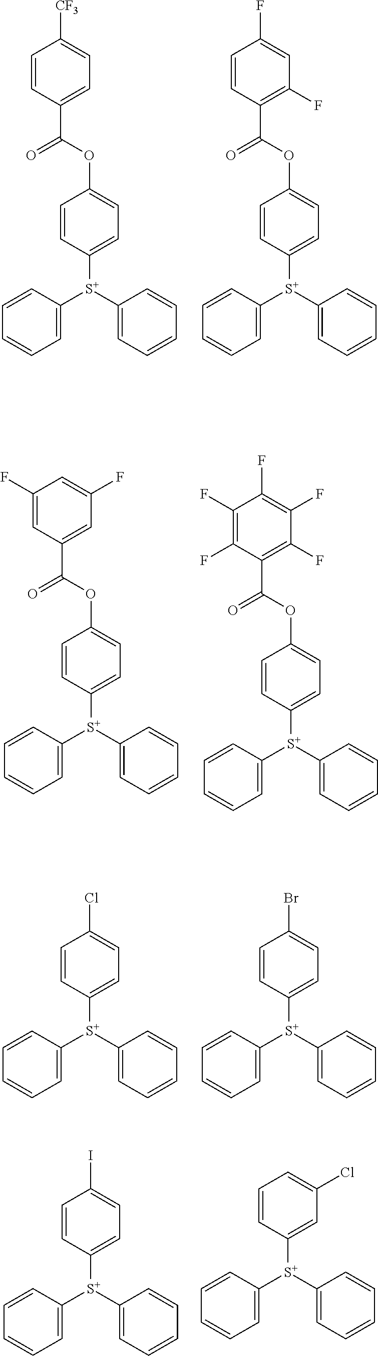

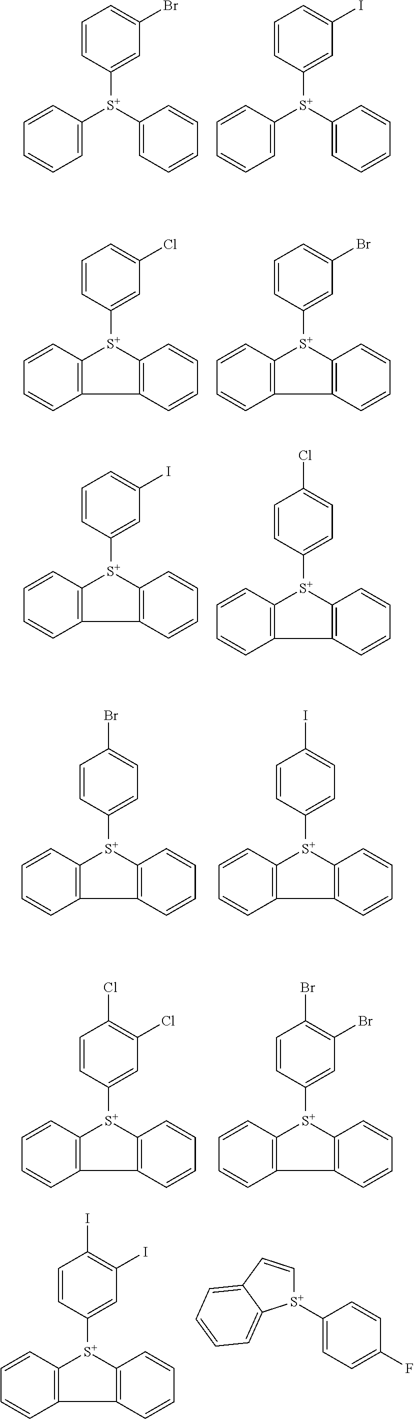

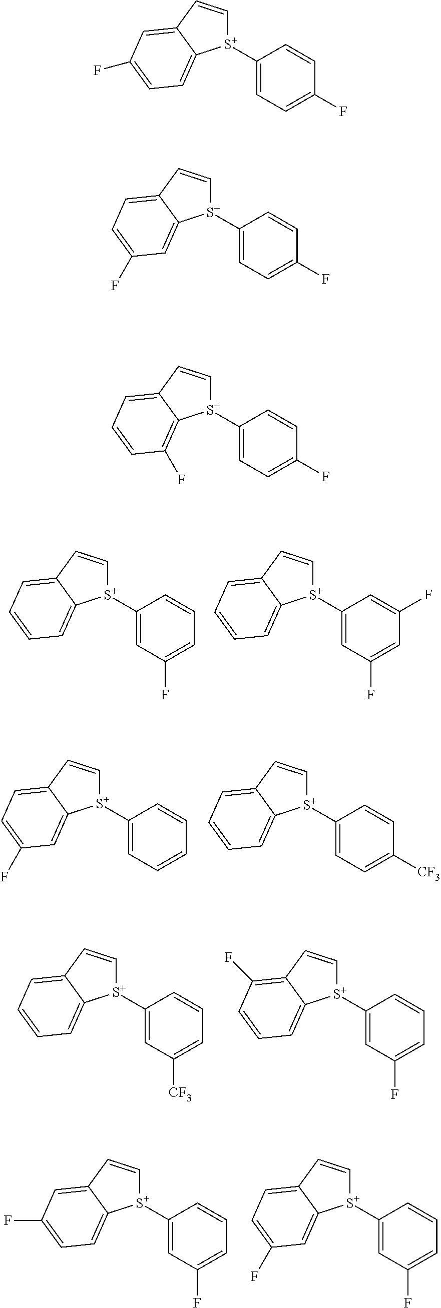

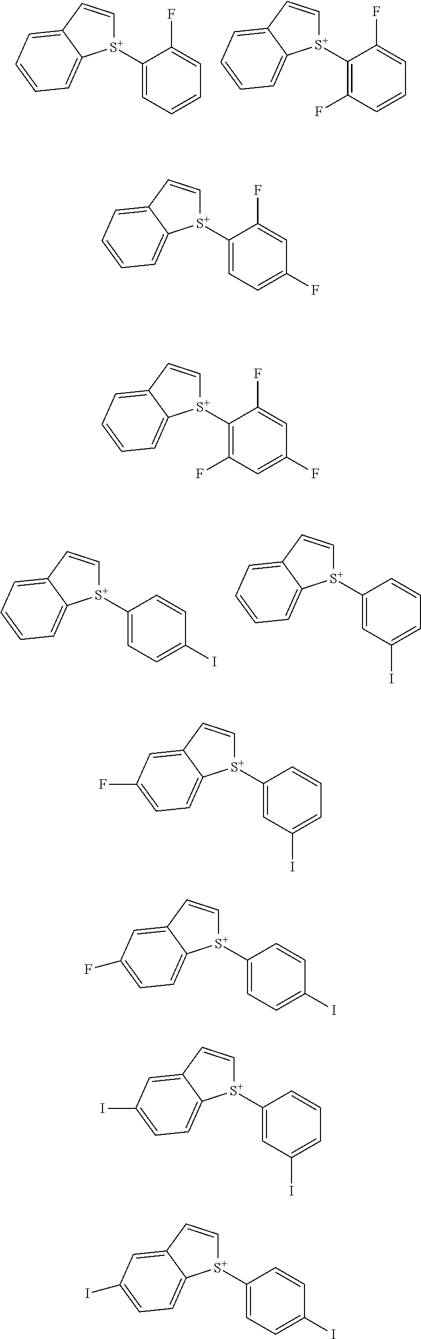

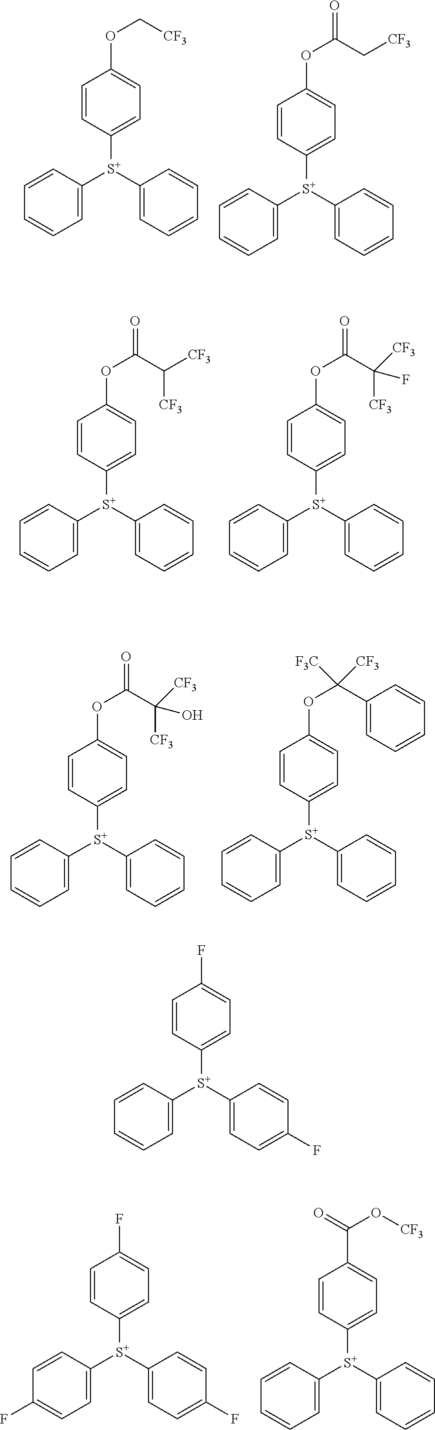

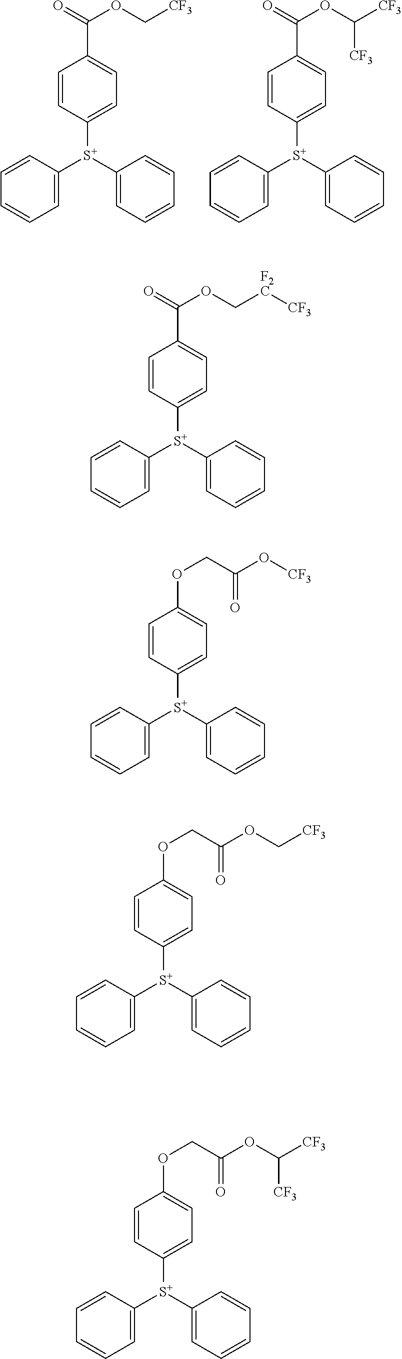 [SMILES: O=C(OCC(F)(F)F)c1ccc([S+](c2ccccc2)c2ccccc2)cc1 ; O=C(OC(C(F)(F)F)C(F)(F)F)c1ccc([S+](c2ccccc2)c2ccccc2)cc1 ; O=C(OCC(F)(F)C(F)(F)F)c1ccc([S+](c2ccccc2)c2ccccc2)cc1 ; O=C(COc1ccc([S+](c2ccccc2)c2ccccc2)cc1)OC(F)(F)F ; O=C(COc1ccc([S+](c2ccccc2)c2ccccc2)cc1)OCC(F)(F)F ; O=C(COc1ccc([S+](c2ccccc2)c2ccccc2)cc1)OC(C(F)(F)F)C(F)(F)F]

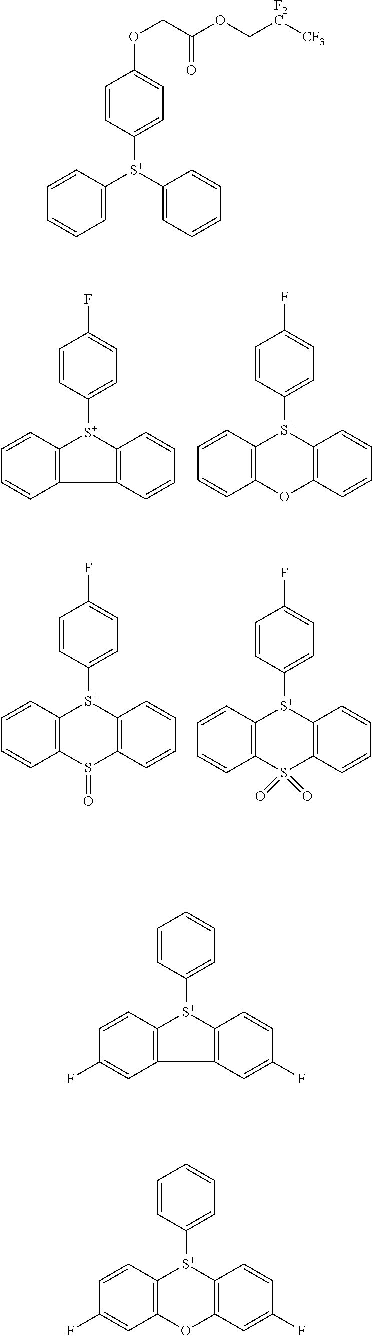

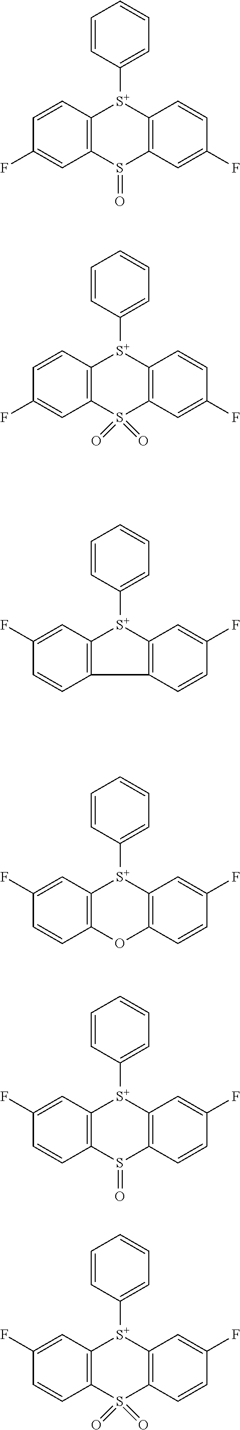 [SMILES: O=S1c2cc(F)ccc2[S+](c2ccccc2)c2ccc(F)cc21 ; O=S1(=O)c2cc(F)ccc2[S+](c2ccccc2)c2ccc(F)cc21 ; Fc1ccc2c3ccc(F)cc3[s+](-c3ccccc3)c2c1 ; Fc1ccc2c(c1)[S+](c1ccccc1)c1cc(F)ccc1O2 ; O=S1c2ccc(F)cc2[S+](c2ccccc2)c2cc(F)ccc21 ; O=S1(=O)c2ccc(F)cc2[S+](c2ccccc2)c2cc(F)ccc21]

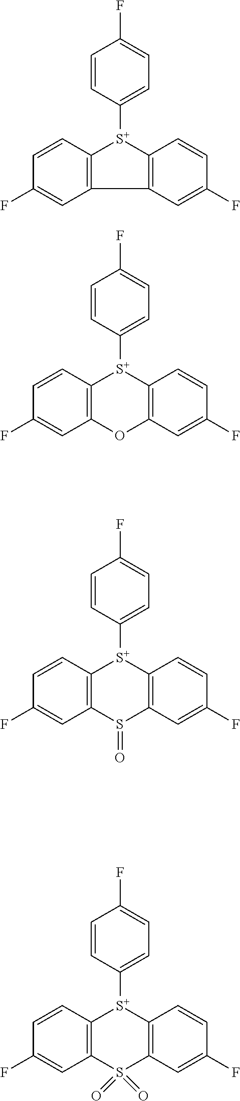 [SMILES: Fc1ccc(-[s+]2c3ccc(F)cc3c3cc(F)ccc32)cc1 ; Fc1ccc([S+]2c3ccc(F)cc3Oc3cc(F)ccc32)cc1 ; O=S1c2cc(F)ccc2[S+](c2ccc(F)cc2)c2ccc(F)cc21 ; O=S1(=O)c2cc(F)ccc2[S+](c2ccc(F)cc2)c2ccc(F)cc21]

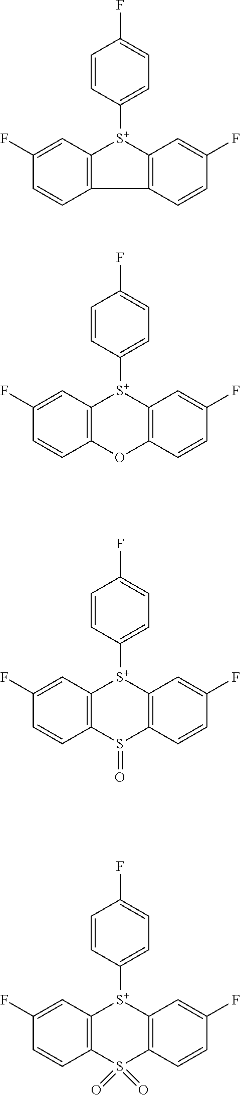 [SMILES: Fc1ccc(-[s+]2c3cc(F)ccc3c3ccc(F)cc32)cc1 ; Fc1ccc([S+]2c3cc(F)ccc3Oc3ccc(F)cc32)cc1 ; O=S1c2ccc(F)cc2[S+](c2ccc(F)cc2)c2cc(F)ccc21 ; O=S1(=O)c2ccc(F)cc2[S+](c2ccc(F)cc2)c2cc(F)ccc21]

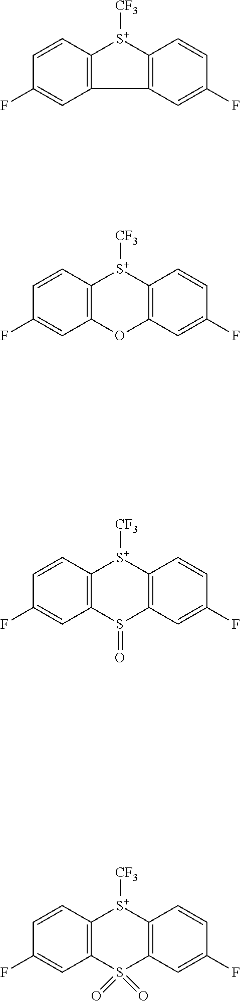 [SMILES: Fc1ccc2c(c1)c1cc(F)ccc1[s+]2C(F)(F)F ; Fc1ccc2c(c1)Oc1cc(F)ccc1[S+]2C(F)(F)F ; O=S1c2cc(F)ccc2[S+](C(F)(F)F)c2ccc(F)cc21 ; O=S1(=O)c2cc(F)ccc2[S+](C(F)(F)F)c2ccc(F)cc21]

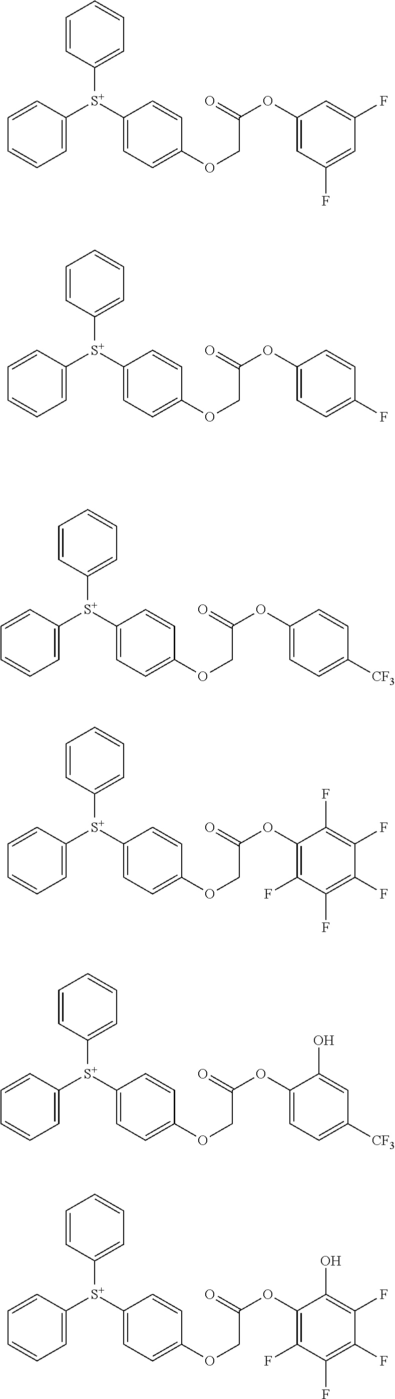 [SMILES: O=C(COc1ccc([S+](c2ccccc2)c2ccccc2)cc1)Oc1cc(F)cc(F)c1 ; O=C(COc1ccc([S+](c2ccccc2)c2ccccc2)cc1)Oc1ccc(F)cc1 ; O=C(COc1ccc([S+](c2ccccc2)c2ccccc2)cc1)Oc1ccc(C(F)(F)F)cc1 ; O=C(COc1ccc([S+](c2ccccc2)c2ccccc2)cc1)Oc1c(F)c(F)c(F)c(F)c1F ; O=C(COc1ccc([S+](c2ccccc2)c2ccccc2)cc1)Oc1ccc(C(F)(F)F)cc1O ; O=C(COc1ccc([S+](c2ccccc2)c2ccccc2)cc1)Oc1c(O)c(F)c(F)c(F)c1F]

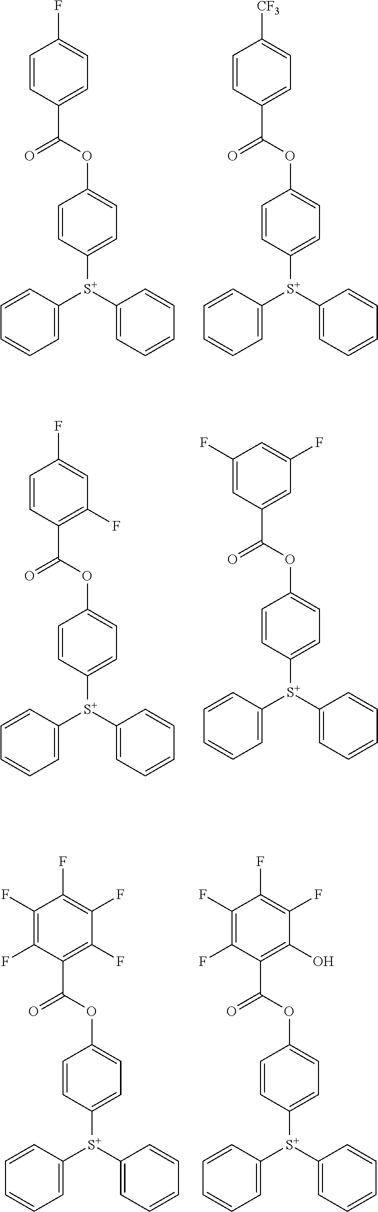 [SMILES: O=C(Oc1ccc([S+](c2ccccc2)c2ccccc2)cc1)c1ccc(F)cc1 ; O=C(Oc1ccc([S+](c2ccccc2)c2ccccc2)cc1)c1ccc(C(F)(F)F)cc1 ; O=C(Oc1ccc([S+](c2ccccc2)c2ccccc2)cc1)c1ccc(F)cc1F ; O=C(Oc1ccc([S+](c2ccccc2)c2ccccc2)cc1)c1cc(F)cc(F)c1 ; O=C(Oc1ccc([S+](c2ccccc2)c2ccccc2)cc1)c1c(F)c(F)c(F)c(F)c1F ; O=C(Oc1ccc([S+](c2ccccc2)c2ccccc2)cc1)c1c(O)c(F)c(F)c(F)c1F]

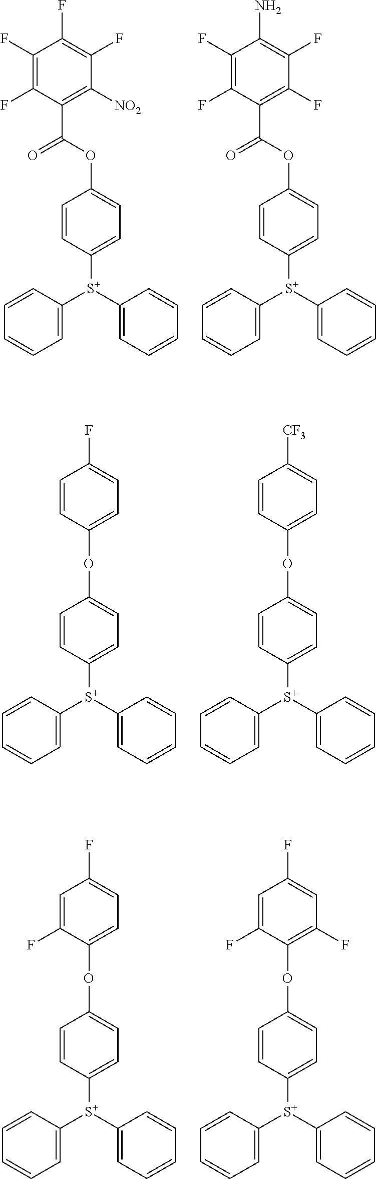 [SMILES: O=C(Oc1ccc([S+](c2ccccc2)c2ccccc2)cc1)c1c(F)c(F)c(F)c(F)c1[N+](=O)[O-] ; Nc1c(F)c(F)c(C(=O)Oc2ccc([S+](c3ccccc3)c3ccccc3)cc2)c(F)c1F ; Fc1ccc(Oc2ccc([S+](c3ccccc3)c3ccccc3)cc2)cc1 ; FC(F)(F)c1ccc(Oc2ccc([S+](c3ccccc3)c3ccccc3)cc2)cc1 ; Fc1ccc(Oc2ccc([S+](c3ccccc3)c3ccccc3)cc2)c(F)c1 ; Fc1cc(F)c(Oc2ccc([S+](c3ccccc3)c3ccccc3)cc2)c(F)c1]

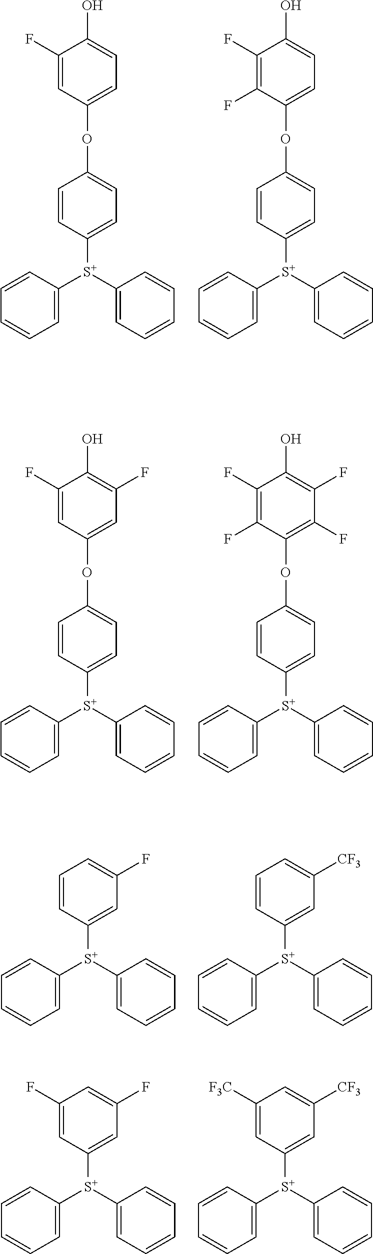

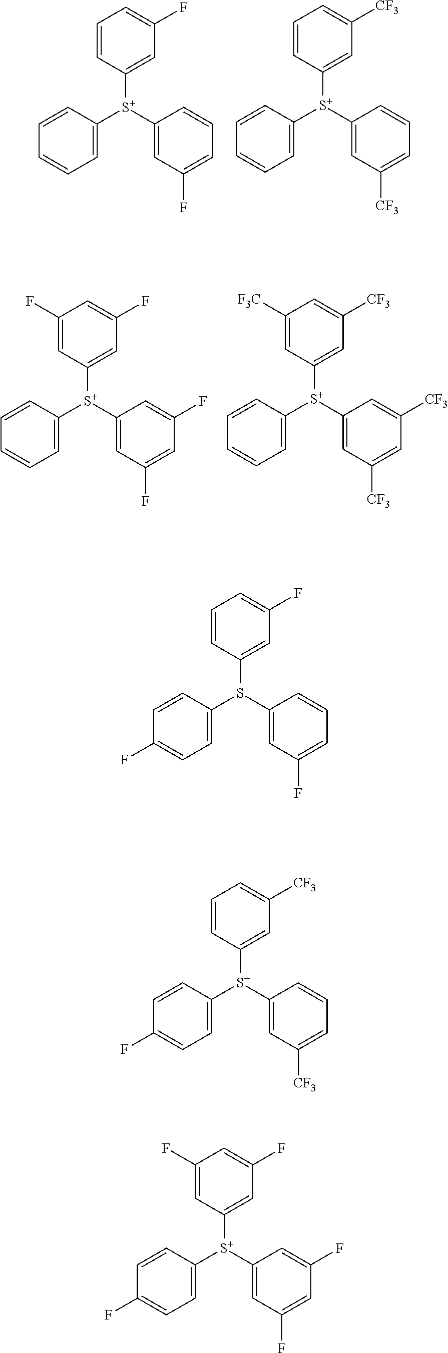

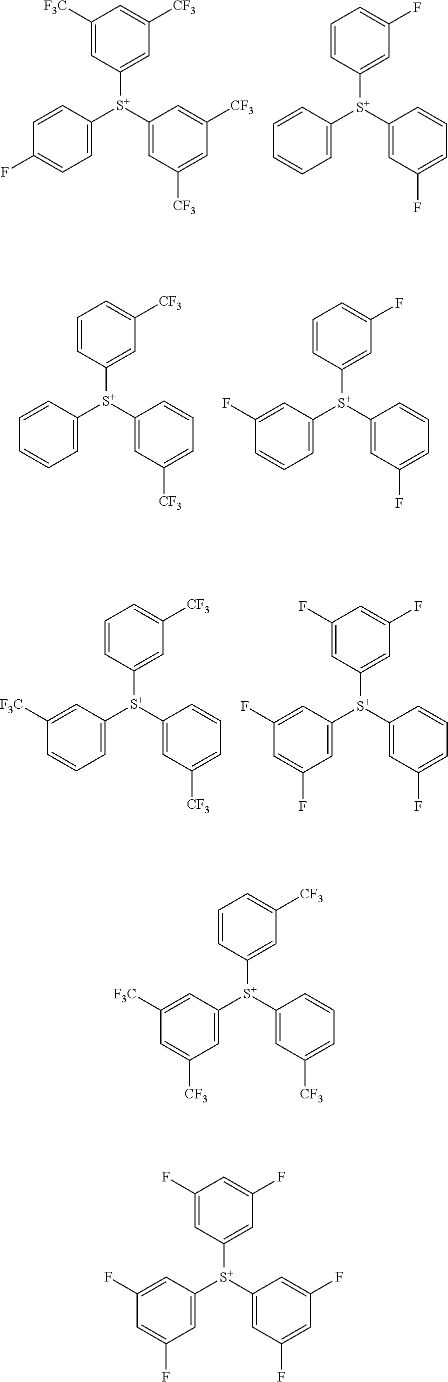

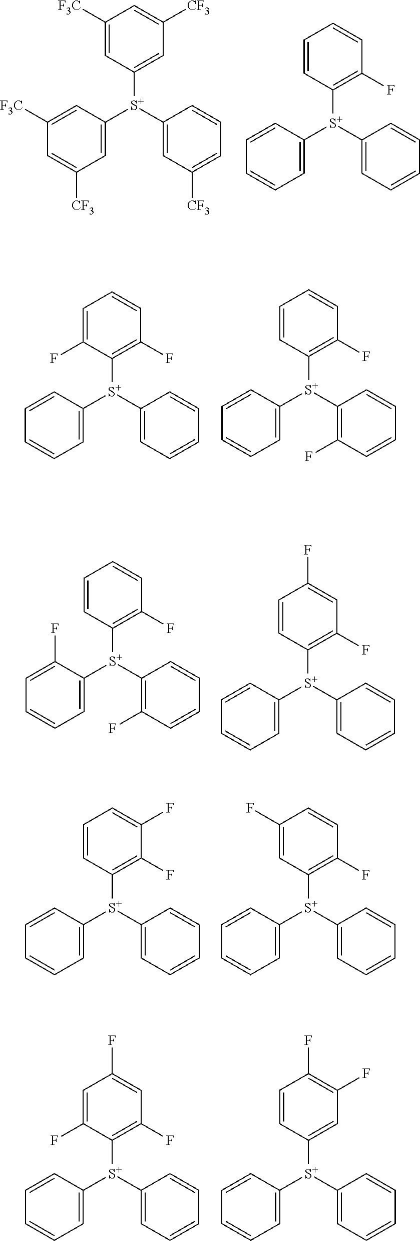

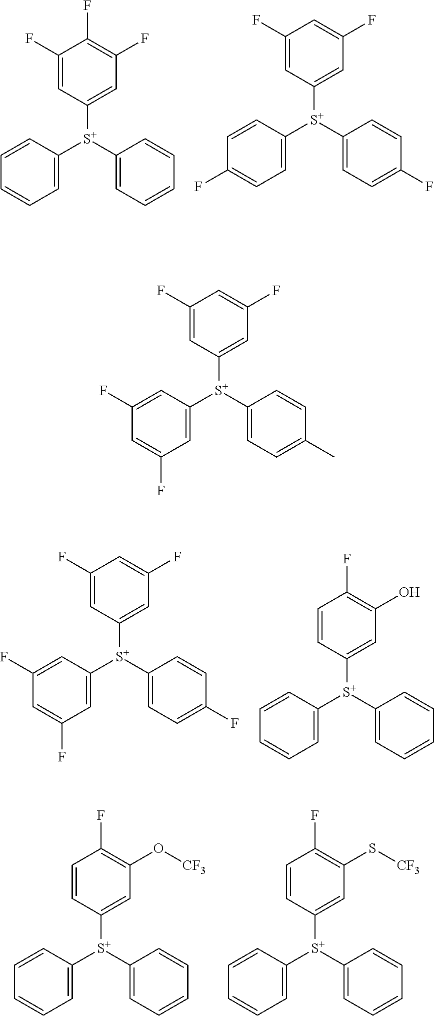

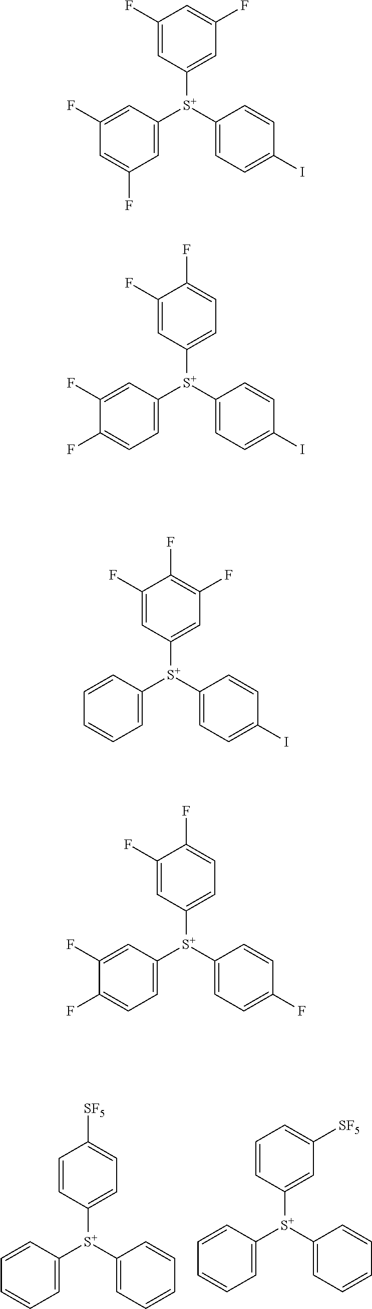

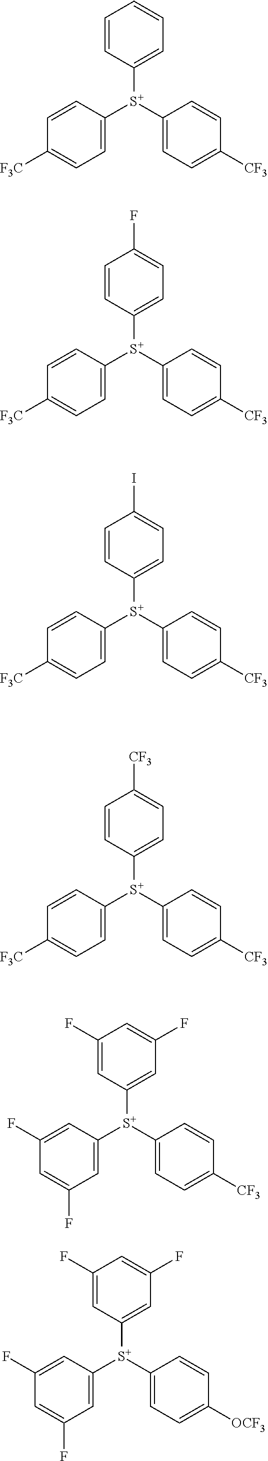 [SMILES: FC(F)(F)c1ccc([S+](c2ccccc2)c2ccc(C(F)(F)F)cc2)cc1 ; Fc1ccc([S+](c2ccc(C(F)(F)F)cc2)c2ccc(C(F)(F)F)cc2)cc1 ; FC(F)(F)c1ccc([S+](c2ccc(I)cc2)c2ccc(C(F)(F)F)cc2)cc1 ; FC(F)(F)c1ccc([S+](c2ccc(C(F)(F)F)cc2)c2ccc(C(F)(F)F)cc2)cc1 ; Fc1cc(F)cc([S+](c2ccc(C(F)(F)F)cc2)c2cc(F)cc(F)c2)c1 ; Fc1cc(F)cc([S+](c2ccc(OC(F)(F)F)cc2)c2cc(F)cc(F)c2)c1]

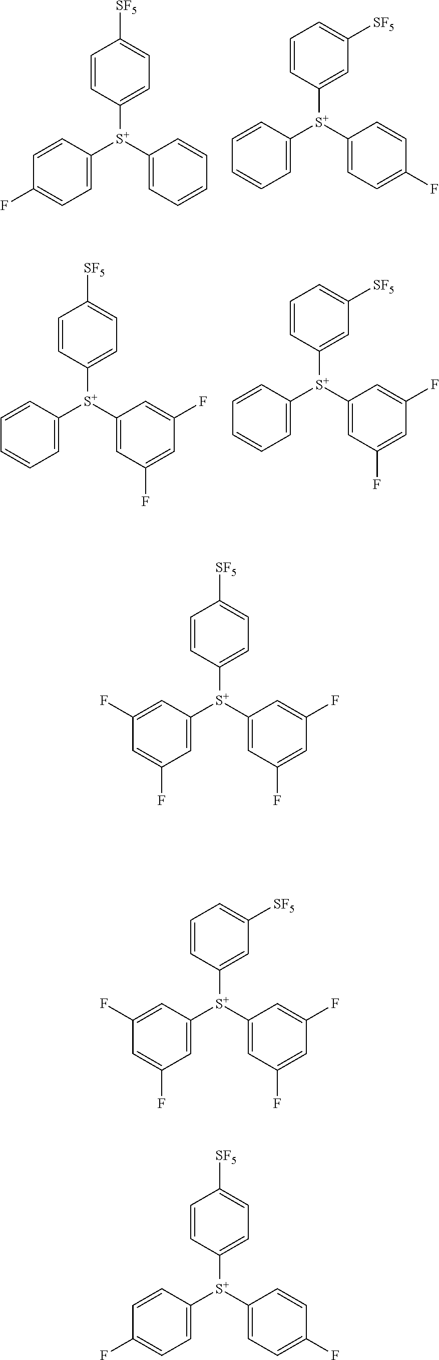

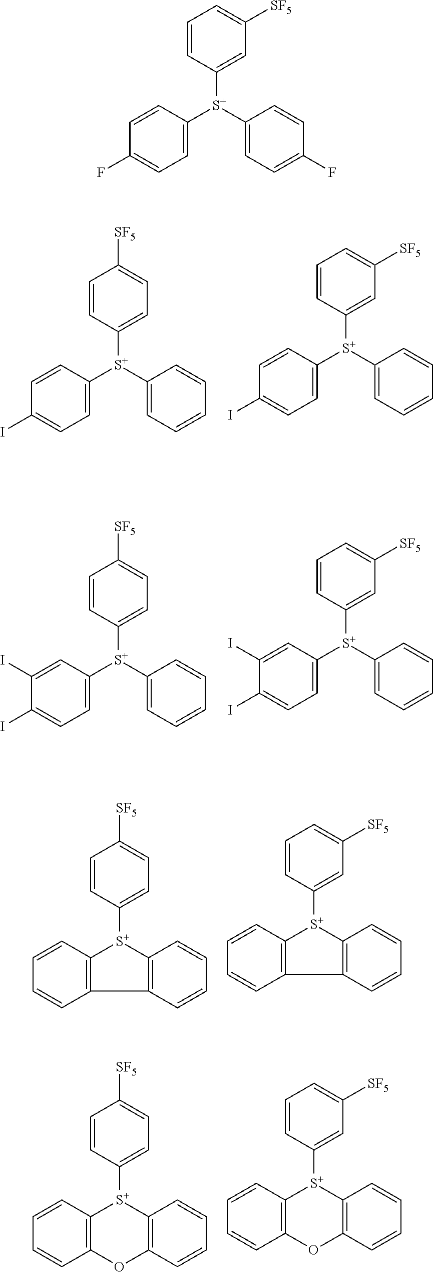

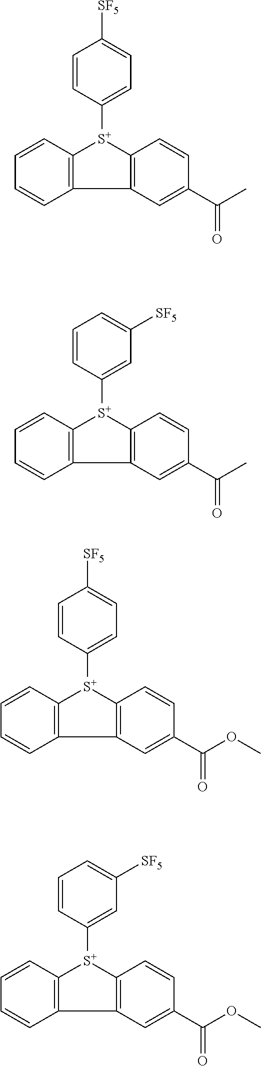

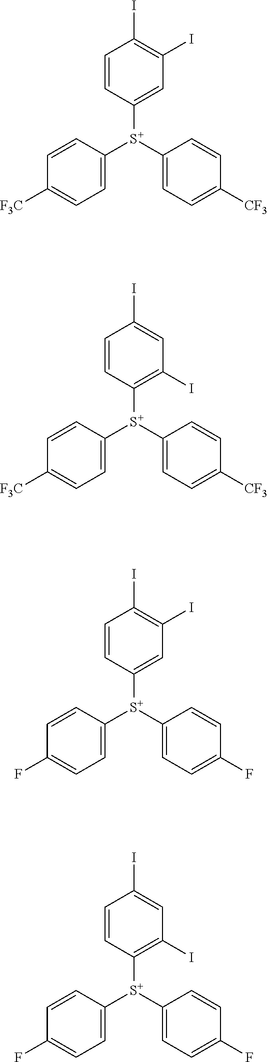 [SMILES: FC(F)(F)c1ccc([S+](c2ccc(C(F)(F)F)cc2)c2ccc(I)c(I)c2)cc1 ; FC(F)(F)c1ccc([S+](c2ccc(C(F)(F)F)cc2)c2ccc(I)cc2I)cc1 ; Fc1ccc([S+](c2ccc(F)cc2)c2ccc(I)c(I)c2)cc1 ; Fc1ccc([S+](c2ccc(F)cc2)c2ccc(I)cc2I)cc1]

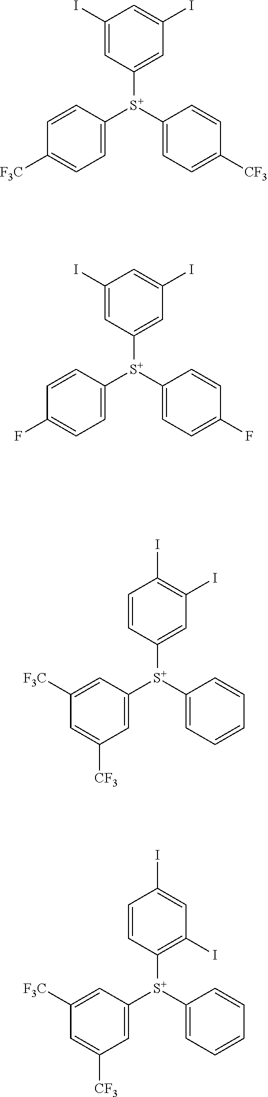 [SMILES: FC(F)(F)c1ccc([S+](c2ccc(C(F)(F)F)cc2)c2cc(I)cc(I)c2)cc1 ; Fc1ccc([S+](c2ccc(F)cc2)c2cc(I)cc(I)c2)cc1 ; FC(F)(F)c1cc([S+](c2ccccc2)c2ccc(I)c(I)c2)cc(C(F)(F)F)c1 ; FC(F)(F)c1cc([S+](c2ccccc2)c2ccc(I)cc2I)cc(C(F)(F)F)c1]

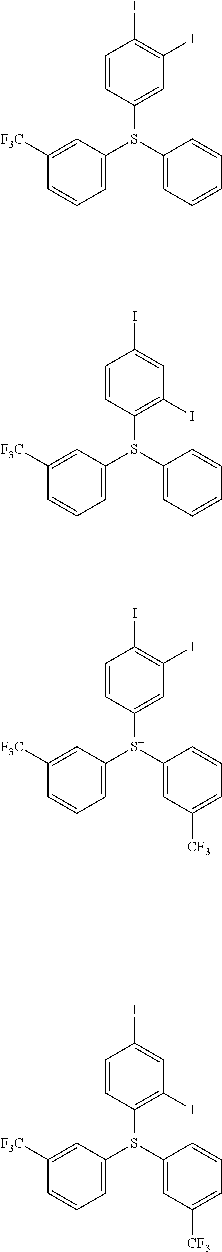 [SMILES: FC(F)(F)c1cccc([S+](c2ccccc2)c2ccc(I)c(I)c2)c1 ; FC(F)(F)c1cccc([S+](c2ccccc2)c2ccc(I)cc2I)c1 ; FC(F)(F)c1cccc([S+](c2cccc(C(F)(F)F)c2)c2ccc(I)c(I)c2)c1 ; FC(F)(F)c1cccc([S+](c2cccc(C(F)(F)F)c2)c2ccc(I)cc2I)c1]

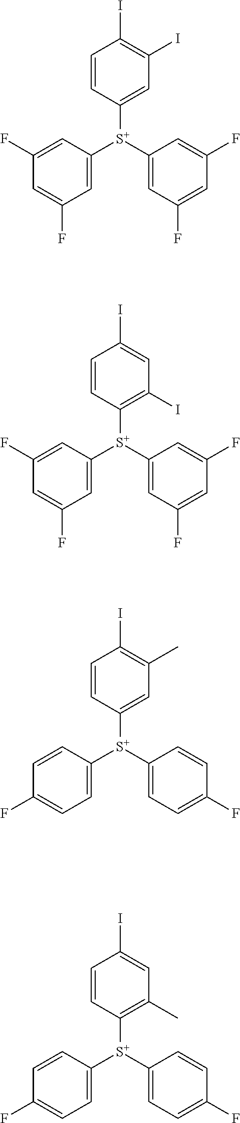 [SMILES: Fc1cc(F)cc([S+](c2cc(F)cc(F)c2)c2ccc(I)c(I)c2)c1 ; Fc1cc(F)cc([S+](c2cc(F)cc(F)c2)c2ccc(I)cc2I)c1 ; Cc1cc([S+](c2ccc(F)cc2)c2ccc(F)cc2)ccc1I ; Cc1cc(I)ccc1[S+](c1ccc(F)cc1)c1ccc(F)cc1]

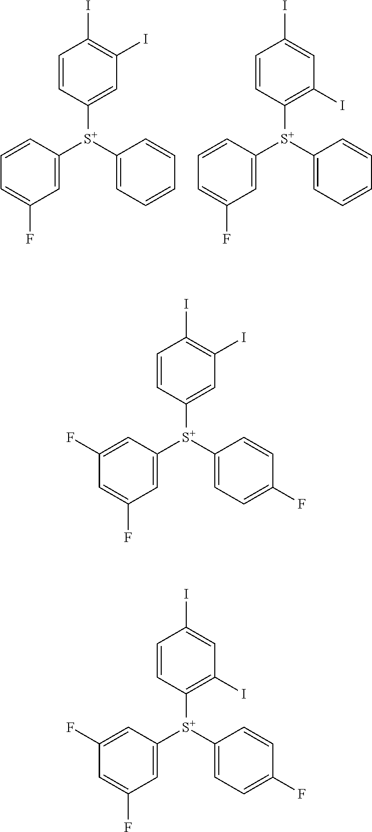 [SMILES: Fc1cccc([S+](c2ccccc2)c2ccc(I)c(I)c2)c1 ; Fc1cccc([S+](c2ccccc2)c2ccc(I)cc2I)c1 ; Fc1ccc([S+](c2cc(F)cc(F)c2)c2ccc(I)c(I)c2)cc1 ; Fc1ccc([S+](c2cc(F)cc(F)c2)c2ccc(I)cc2I)cc1]

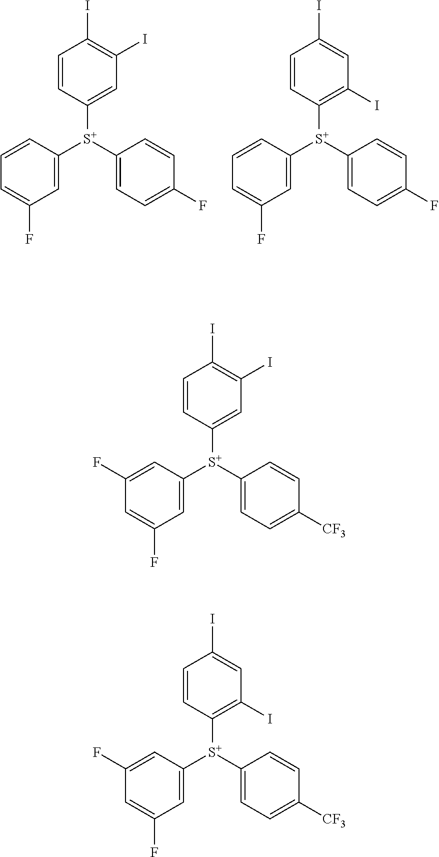 [SMILES: Fc1ccc([S+](c2cccc(F)c2)c2ccc(I)c(I)c2)cc1 ; Fc1ccc([S+](c2cccc(F)c2)c2ccc(I)cc2I)cc1 ; Fc1cc(F)cc([S+](c2ccc(C(F)(F)F)cc2)c2ccc(I)c(I)c2)c1 ; Fc1cc(F)cc([S+](c2ccc(C(F)(F)F)cc2)c2ccc(I)cc2I)c1]

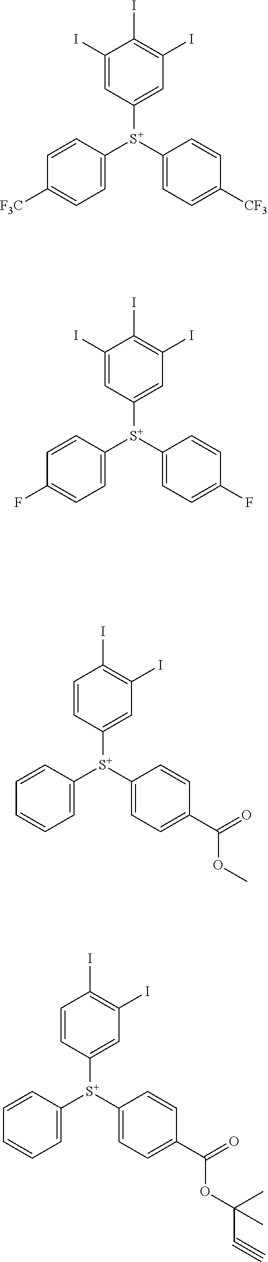 [SMILES: FC(F)(F)c1ccc([S+](c2ccc(C(F)(F)F)cc2)c2cc(I)c(I)c(I)c2)cc1 ; Fc1ccc([S+](c2ccc(F)cc2)c2cc(I)c(I)c(I)c2)cc1 ; COC(=O)c1ccc([S+](c2ccccc2)c2ccc(I)c(I)c2)cc1 ; C#CC(C)(C)OC(=O)c1ccc([S+](c2ccccc2)c2ccc(I)c(I)c2)cc1]

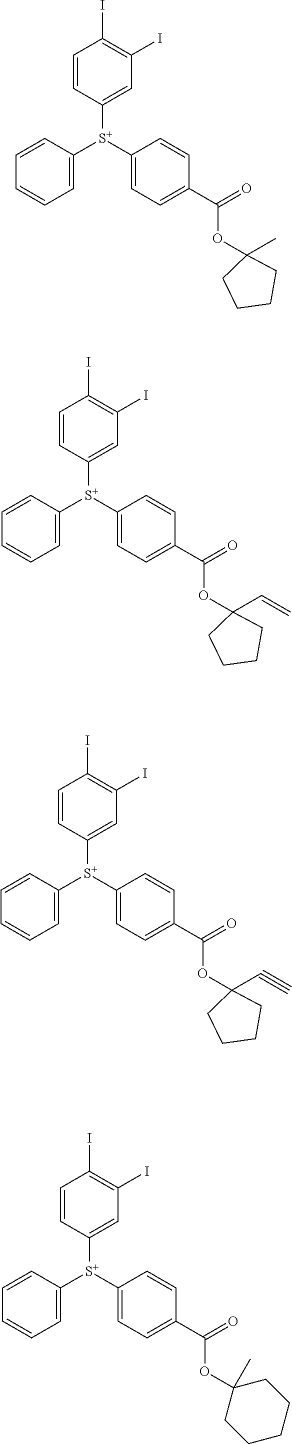 [SMILES: CC1(OC(=O)c2ccc([S+](c3ccccc3)c3ccc(I)c(I)c3)cc2)CCCC1 ; C=CC1(OC(=O)c2ccc([S+](c3ccccc3)c3ccc(I)c(I)c3)cc2)CCCC1 ; C#CC1(OC(=O)c2ccc([S+](c3ccccc3)c3ccc(I)c(I)c3)cc2)CCCC1 ; CC1(OC(=O)c2ccc([S+](c3ccccc3)c3ccc(I)c(I)c3)cc2)CCCCC1]

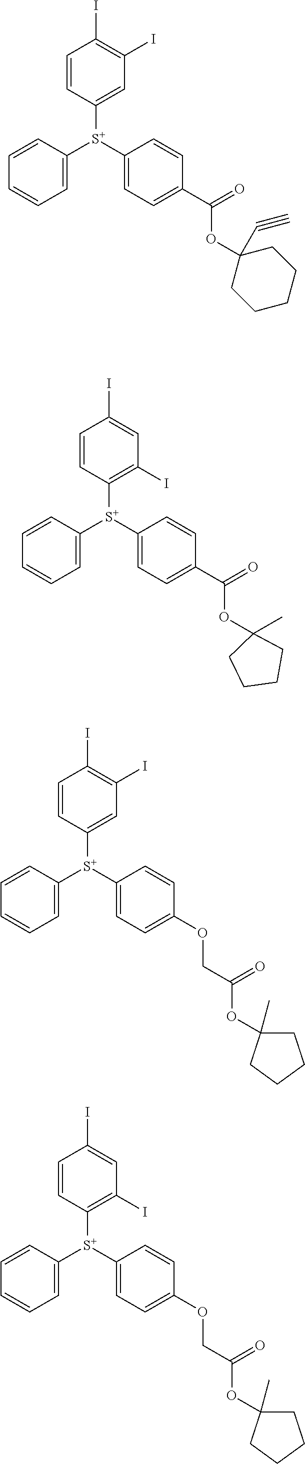 [SMILES: C#CC1(OC(=O)c2ccc([S+](c3ccccc3)c3ccc(I)c(I)c3)cc2)CCCCC1 ; CC1(OC(=O)c2ccc([S+](c3ccccc3)c3ccc(I)cc3I)cc2)CCCC1 ; CC1(OC(=O)COc2ccc([S+](c3ccccc3)c3ccc(I)c(I)c3)cc2)CCCC1 ; CC1(OC(=O)COc2ccc([S+](c3ccccc3)c3ccc(I)cc3I)cc2)CCCC1]

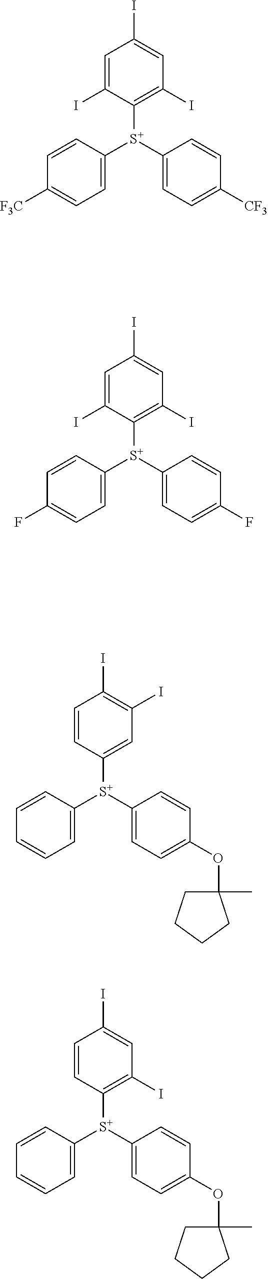 [SMILES: FC(F)(F)c1ccc([S+](c2ccc(C(F)(F)F)cc2)c2c(I)cc(I)cc2I)cc1 ; Fc1ccc([S+](c2ccc(F)cc2)c2c(I)cc(I)cc2I)cc1 ; CC1(Oc2ccc([S+](c3ccccc3)c3ccc(I)c(I)c3)cc2)CCCC1 ; CC1(Oc2ccc([S+](c3ccccc3)c3ccc(I)cc3I)cc2)CCCC1]

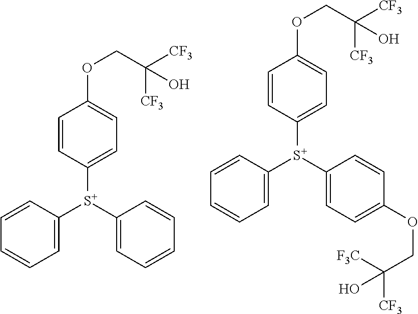 [SMILES: OC(COc1ccc([S+](c2ccccc2)c2ccccc2)cc1)(C(F)(F)F)C(F)(F)F ; OC(COc1ccc([S+](c2ccccc2)c2ccc(OCC(O)(C(F)(F)F)C(F)(F)F)cc2)cc1)(C(F)(F)F)C(F)(F)F]

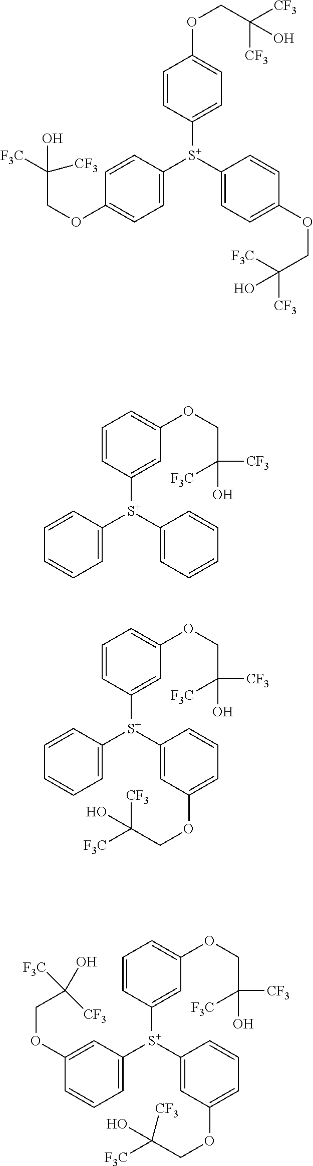 [SMILES: OC(COc1ccc([S+](c2ccc(OCC(O)(C(F)(F)F)C(F)(F)F)cc2)c2ccc(OCC(O)(C(F)(F)F)C(F)(F)F)cc2)cc1)(C(F)(F)F)C(F)(F)F ; OC(COc1cccc([S+](c2ccccc2)c2ccccc2)c1)(C(F)(F)F)C(F)(F)F ; OC(COc1cccc([S+](c2ccccc2)c2cccc(OCC(O)(C(F)(F)F)C(F)(F)F)c2)c1)(C(F)(F)F)C(F)(F)F ; OC(COc1cccc([S+](c2cccc(OCC(O)(C(F)(F)F)C(F)(F)F)c2)c2cccc(OCC(O)(C(F)(F)F)C(F)(F)F)c2)c1)(C(F)(F)F)C(F)(F)F]

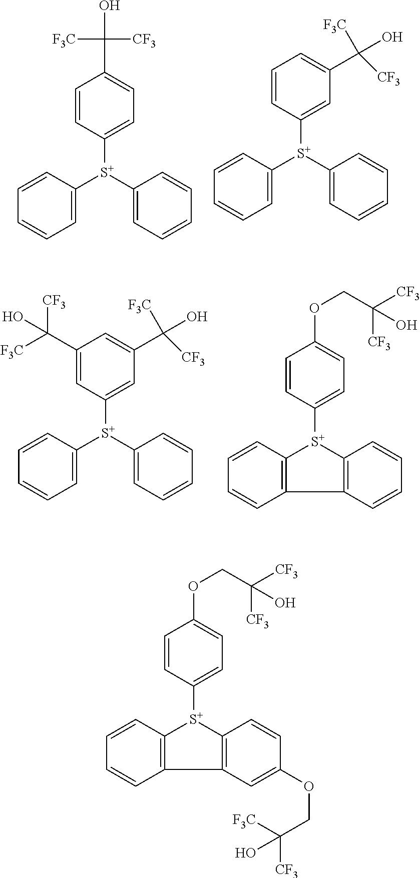 [SMILES: OC(c1ccc([S+](c2ccccc2)c2ccccc2)cc1)(C(F)(F)F)C(F)(F)F ; OC(c1cccc([S+](c2ccccc2)c2ccccc2)c1)(C(F)(F)F)C(F)(F)F ; OC(c1cc([S+](c2ccccc2)c2ccccc2)cc(C(O)(C(F)(F)F)C(F)(F)F)c1)(C(F)(F)F)C(F)(F)F ; OC(COc1ccc(-[s+]2c3ccccc3c3ccccc32)cc1)(C(F)(F)F)C(F)(F)F ; OC(COc1ccc(-[s+]2c3ccccc3c3cc(OCC(O)(C(F)(F)F)C(F)(F)F)ccc32)cc1)(C(F)(F)F)C(F)(F)F]

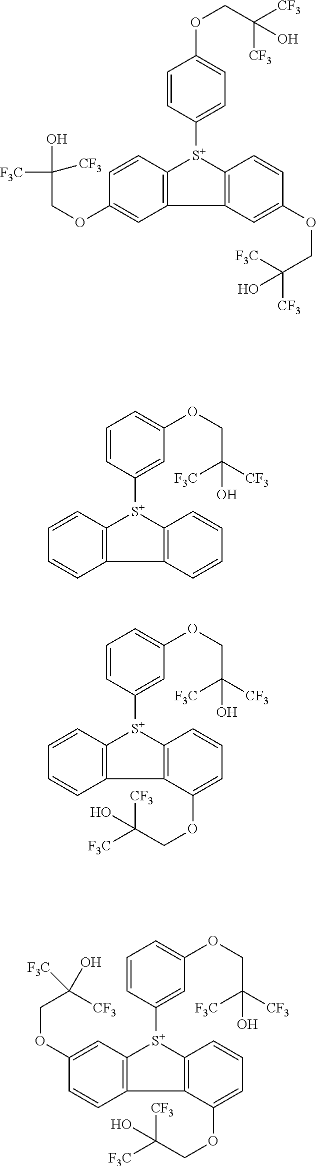 [SMILES: OC(COc1ccc(-[s+]2c3ccc(OCC(O)(C(F)(F)F)C(F)(F)F)cc3c3cc(OCC(O)(C(F)(F)F)C(F)(F)F)ccc32)cc1)(C(F)(F)F)C(F)(F)F ; OC(COc1cccc(-[s+]2c3ccccc3c3ccccc32)c1)(C(F)(F)F)C(F)(F)F ; OC(COc1cccc(-[s+]2c3ccccc3c3c(OCC(O)(C(F)(F)F)C(F)(F)F)cccc32)c1)(C(F)(F)F)C(F)(F)F ; OC(COc1cccc(-[s+]2c3cc(OCC(O)(C(F)(F)F)C(F)(F)F)ccc3c3c(OCC(O)(C(F)(F)F)C(F)(F)F)cccc32)c1)(C(F)(F)F)C(F)(F)F]

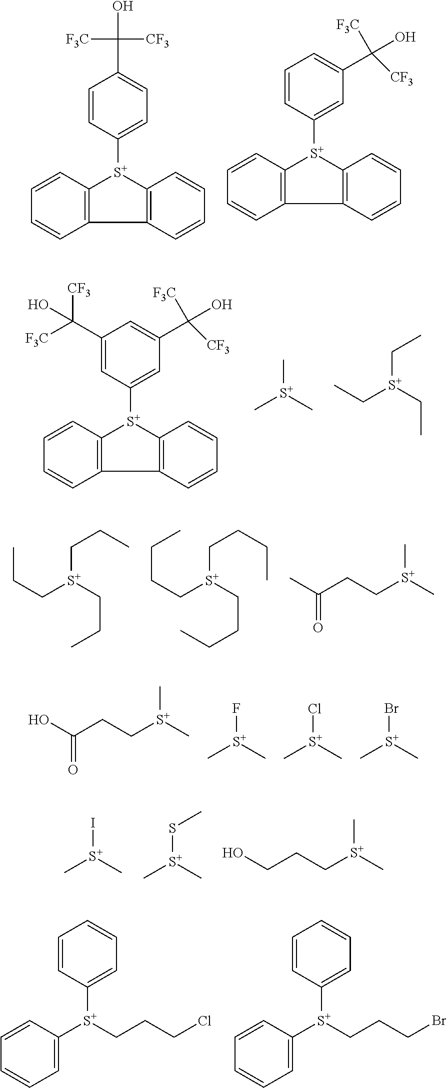

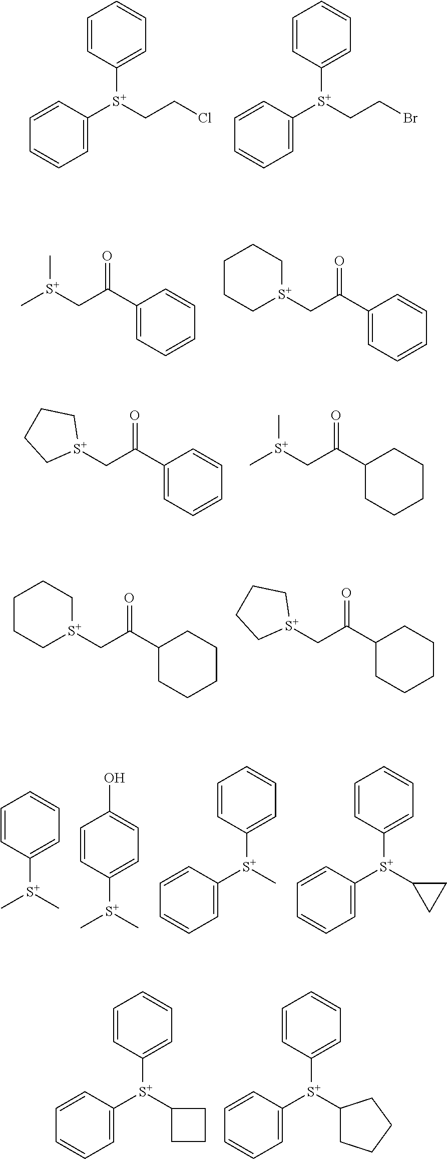

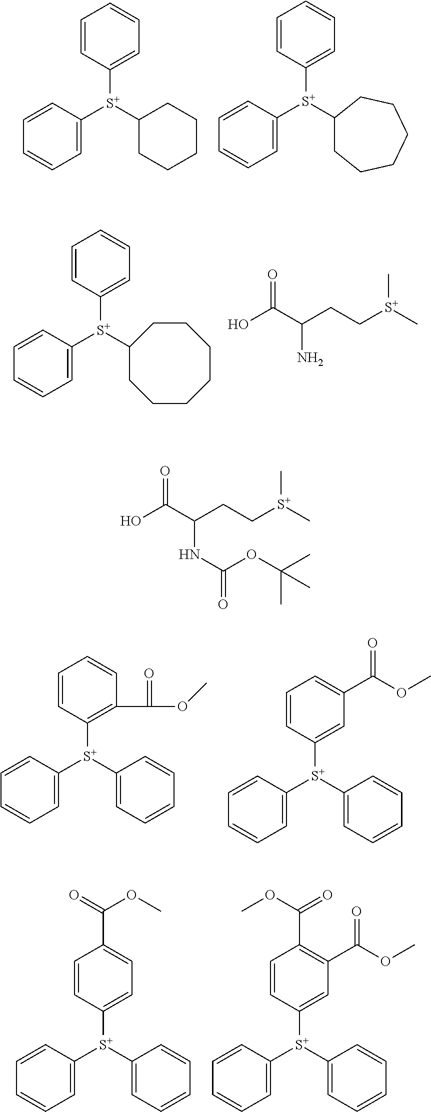

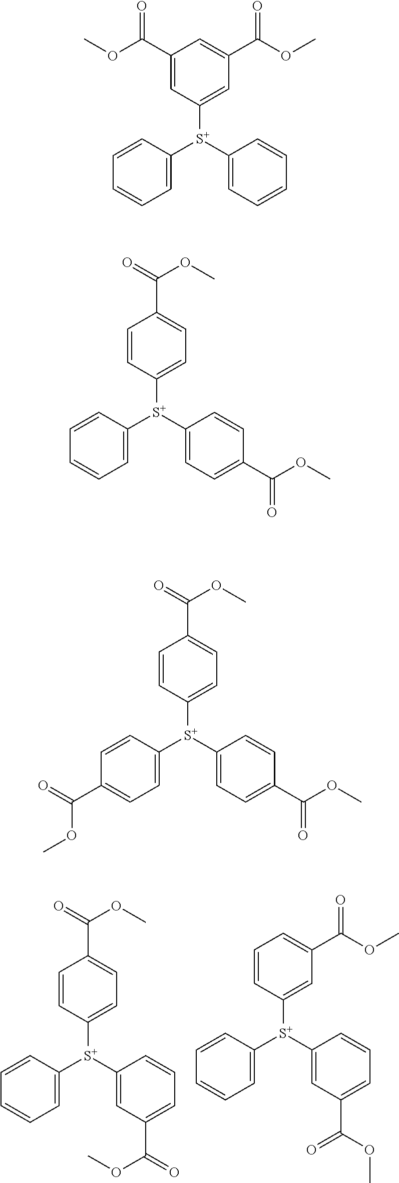

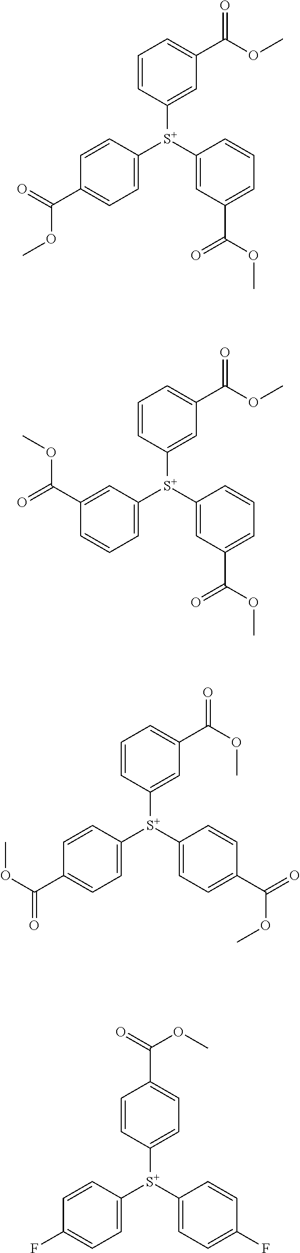 [SMILES: COC(=O)c1ccc([S+](c2cccc(C(=O)OC)c2)c2cccc(C(=O)OC)c2)cc1 ; COC(=O)c1cccc([S+](c2cccc(C(=O)OC)c2)c2cccc(C(=O)OC)c2)c1 ; COC(=O)c1ccc([S+](c2ccc(C(=O)OC)cc2)c2cccc(C(=O)OC)c2)cc1 ; COC(=O)c1ccc([S+](c2ccc(F)cc2)c2ccc(F)cc2)cc1]

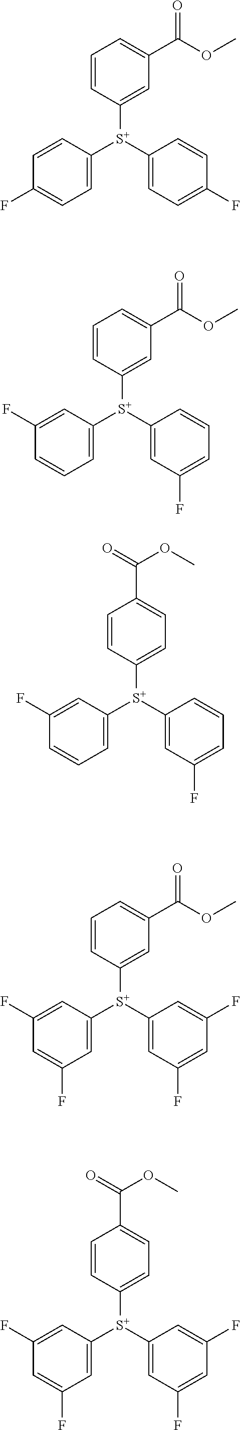 [SMILES: COC(=O)c1cccc([S+](c2ccc(F)cc2)c2ccc(F)cc2)c1 ; COC(=O)c1cccc([S+](c2cccc(F)c2)c2cccc(F)c2)c1 ; COC(=O)c1ccc([S+](c2cccc(F)c2)c2cccc(F)c2)cc1 ; COC(=O)c1cccc([S+](c2cc(F)cc(F)c2)c2cc(F)cc(F)c2)c1 ; COC(=O)c1ccc([S+](c2cc(F)cc(F)c2)c2cc(F)cc(F)c2)cc1]

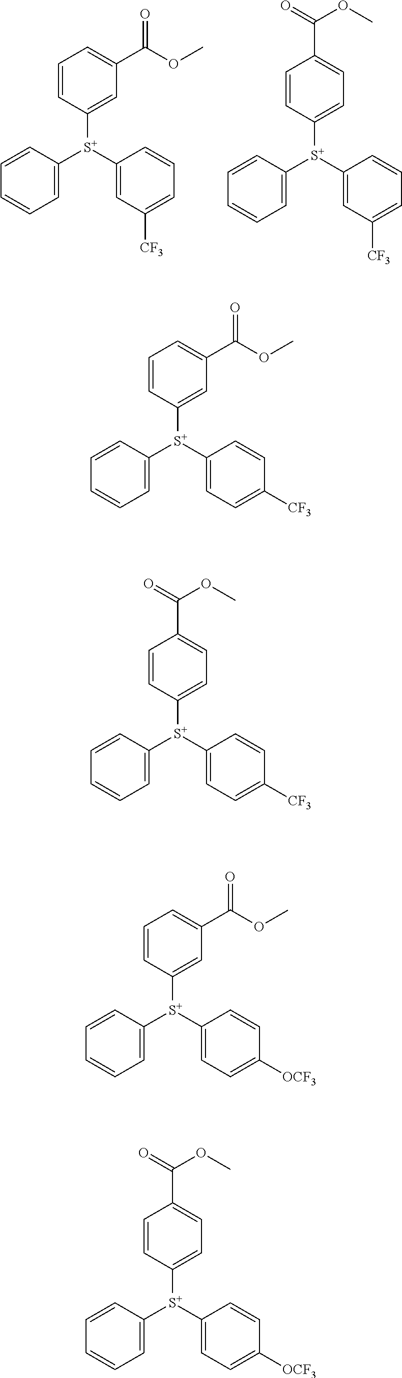 [SMILES: COC(=O)c1cccc([S+](c2ccccc2)c2cccc(C(F)(F)F)c2)c1 ; COC(=O)c1ccc([S+](c2ccccc2)c2cccc(C(F)(F)F)c2)cc1 ; COC(=O)c1cccc([S+](c2ccccc2)c2ccc(C(F)(F)F)cc2)c1 ; COC(=O)c1ccc([S+](c2ccccc2)c2ccc(C(F)(F)F)cc2)cc1 ; COC(=O)c1cccc([S+](c2ccccc2)c2ccc(OC(F)(F)F)cc2)c1 ; COC(=O)c1ccc([S+](c2ccccc2)c2ccc(OC(F)(F)F)cc2)cc1]

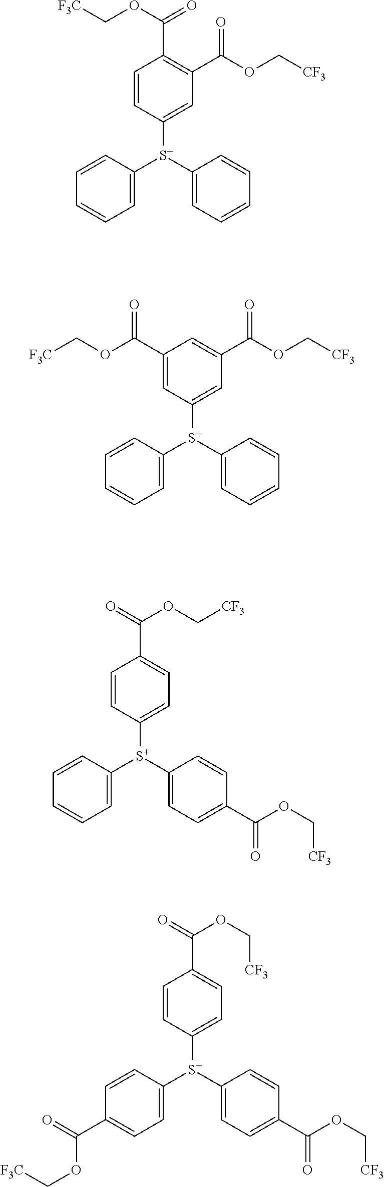 [SMILES: O=C(OCC(F)(F)F)c1ccc([S+](c2ccccc2)c2ccccc2)cc1C(=O)OCC(F)(F)F ; O=C(OCC(F)(F)F)c1cc(C(=O)OCC(F)(F)F)cc([S+](c2ccccc2)c2ccccc2)c1 ; O=C(OCC(F)(F)F)c1ccc([S+](c2ccccc2)c2ccc(C(=O)OCC(F)(F)F)cc2)cc1 ; O=C(OCC(F)(F)F)c1ccc([S+](c2ccc(C(=O)OCC(F)(F)F)cc2)c2ccc(C(=O)OCC(F)(F)F)cc2)cc1]

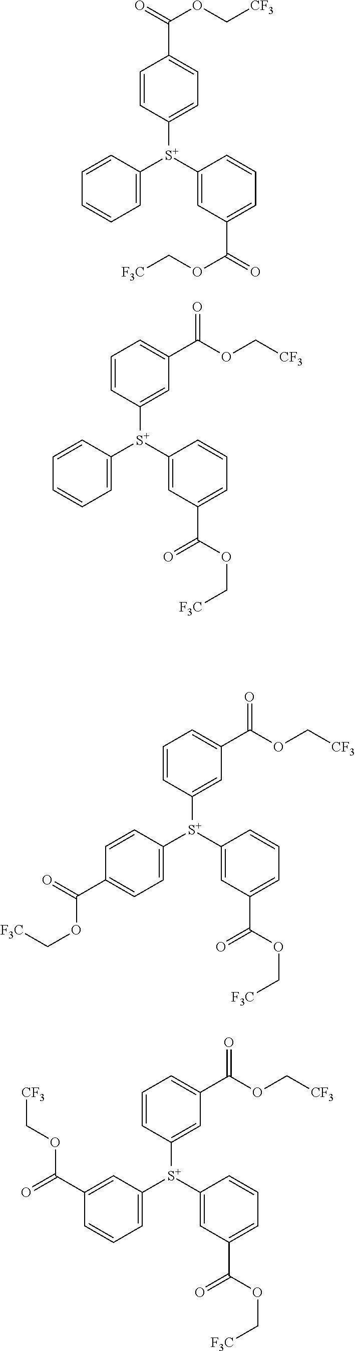 [SMILES: O=C(OCC(F)(F)F)c1ccc([S+](c2ccccc2)c2cccc(C(=O)OCC(F)(F)F)c2)cc1 ; O=C(OCC(F)(F)F)c1cccc([S+](c2ccccc2)c2cccc(C(=O)OCC(F)(F)F)c2)c1 ; O=C(OCC(F)(F)F)c1ccc([S+](c2cccc(C(=O)OCC(F)(F)F)c2)c2cccc(C(=O)OCC(F)(F)F)c2)cc1 ; O=C(OCC(F)(F)F)c1cccc([S+](c2cccc(C(=O)OCC(F)(F)F)c2)c2cccc(C(=O)OCC(F)(F)F)c2)c1]

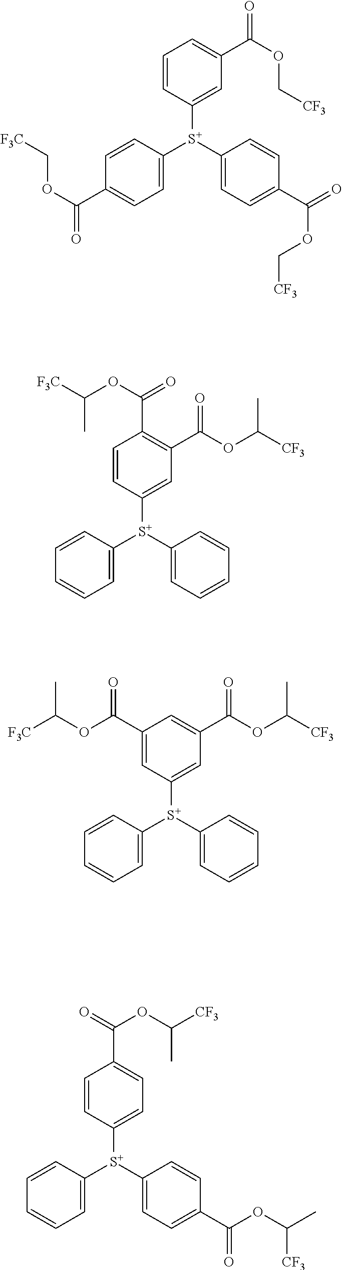 [SMILES: O=C(OCC(F)(F)F)c1ccc([S+](c2ccc(C(=O)OCC(F)(F)F)cc2)c2cccc(C(=O)OCC(F)(F)F)c2)cc1 ; CC(OC(=O)c1ccc([S+](c2ccccc2)c2ccccc2)cc1C(=O)OC(C)C(F)(F)F)C(F)(F)F ; CC(OC(=O)c1cc(C(=O)OC(C)C(F)(F)F)cc([S+](c2ccccc2)c2ccccc2)c1)C(F)(F)F ; CC(OC(=O)c1ccc([S+](c2ccccc2)c2ccc(C(=O)OC(C)C(F)(F)F)cc2)cc1)C(F)(F)F]

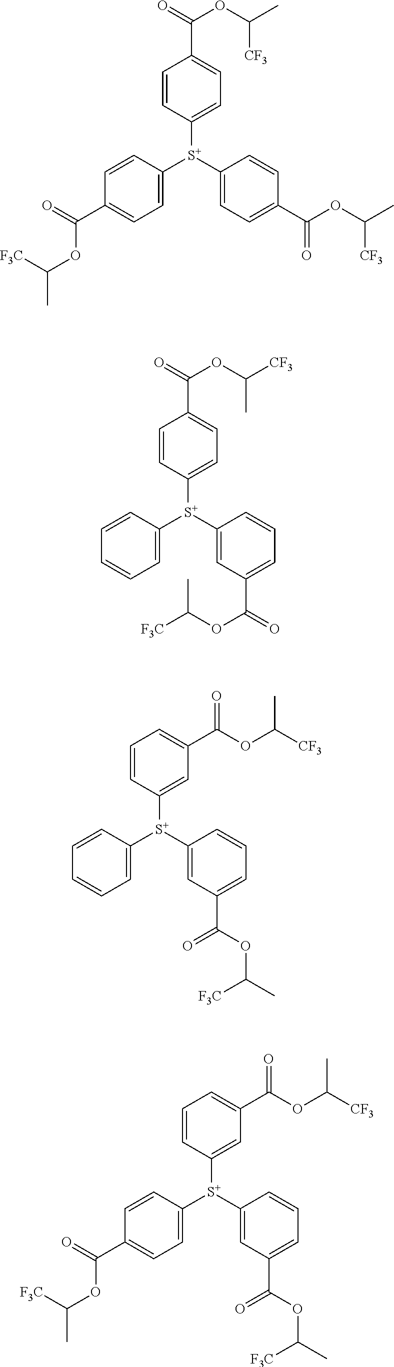 [SMILES: CC(OC(=O)c1ccc([S+](c2ccc(C(=O)OC(C)C(F)(F)F)cc2)c2ccc(C(=O)OC(C)C(F)(F)F)cc2)cc1)C(F)(F)F ; CC(OC(=O)c1ccc([S+](c2ccccc2)c2cccc(C(=O)OC(C)C(F)(F)F)c2)cc1)C(F)(F)F ; CC(OC(=O)c1cccc([S+](c2ccccc2)c2cccc(C(=O)OC(C)C(F)(F)F)c2)c1)C(F)(F)F ; CC(OC(=O)c1ccc([S+](c2cccc(C(=O)OC(C)C(F)(F)F)c2)c2cccc(C(=O)OC(C)C(F)(F)F)c2)cc1)C(F)(F)F]

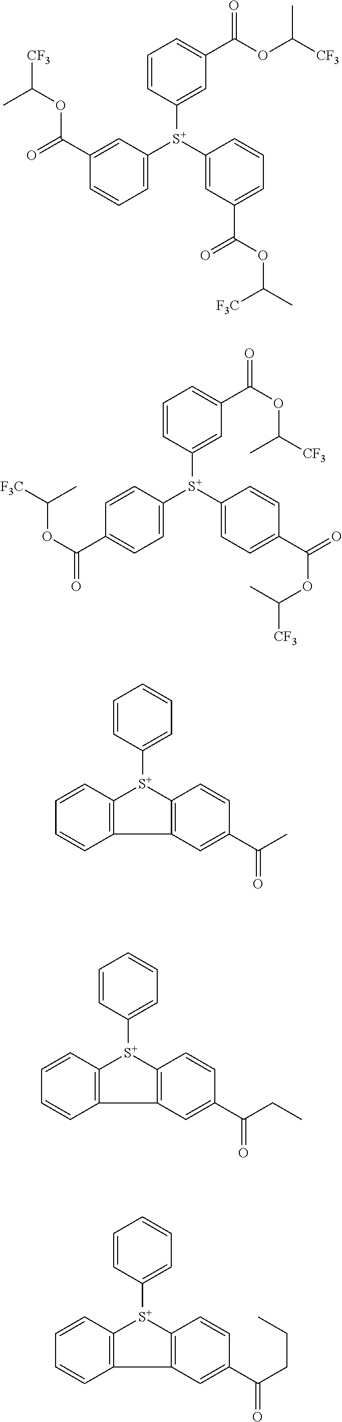 [SMILES: CC(OC(=O)c1cccc([S+](c2cccc(C(=O)OC(C)C(F)(F)F)c2)c2cccc(C(=O)OC(C)C(F)(F)F)c2)c1)C(F)(F)F ; CC(OC(=O)c1ccc([S+](c2ccc(C(=O)OC(C)C(F)(F)F)cc2)c2cccc(C(=O)OC(C)C(F)(F)F)c2)cc1)C(F)(F)F ; CC(=O)c1ccc2c(c1)c1ccccc1[s+]2-c1ccccc1 ; CCC(=O)c1ccc2c(c1)c1ccccc1[s+]2-c1ccccc1 ; CCCC(=O)c1ccc2c(c1)c1ccccc1[s+]2-c1ccccc1]

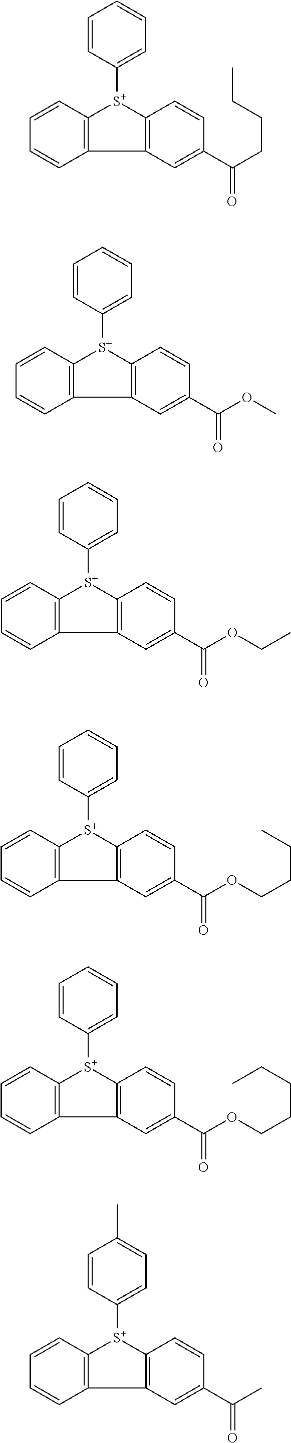 [SMILES: CCCCC(=O)c1ccc2c(c1)c1ccccc1[s+]2-c1ccccc1 ; COC(=O)c1ccc2c(c1)c1ccccc1[s+]2-c1ccccc1 ; CCOC(=O)c1ccc2c(c1)c1ccccc1[s+]2-c1ccccc1 ; CCCCOC(=O)c1ccc2c(c1)c1ccccc1[s+]2-c1ccccc1 ; CCCCCOC(=O)c1ccc2c(c1)c1ccccc1[s+]2-c1ccccc1 ; CC(=O)c1ccc2c(c1)c1ccccc1[s+]2-c1ccc(C)cc1]

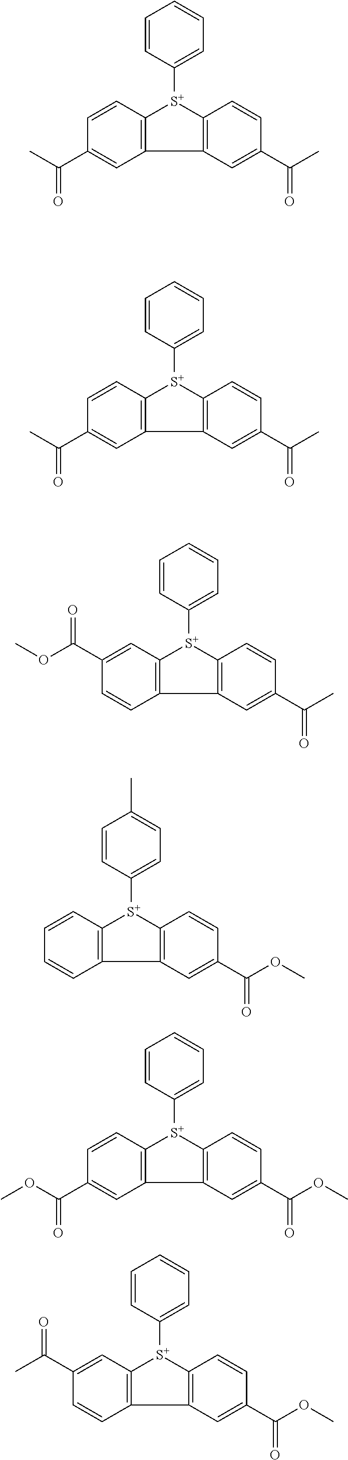

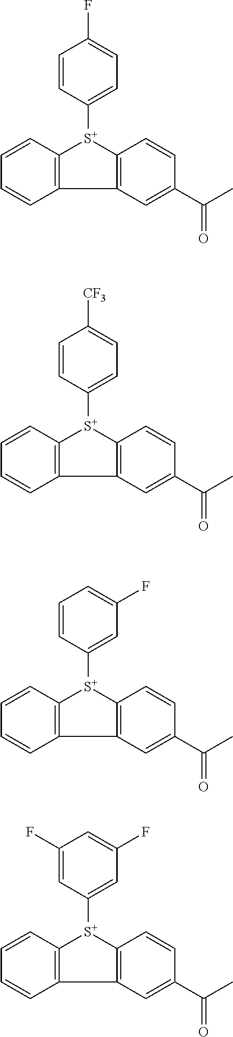 [SMILES: CC(=O)c1ccc2c(c1)c1ccccc1[s+]2-c1ccc(F)cc1 ; CC(=O)c1ccc2c(c1)c1ccccc1[s+]2-c1ccc(C(F)(F)F)cc1 ; CC(=O)c1ccc2c(c1)c1ccccc1[s+]2-c1cccc(F)c1 ; CC(=O)c1ccc2c(c1)c1ccccc1[s+]2-c1cc(F)cc(F)c1]

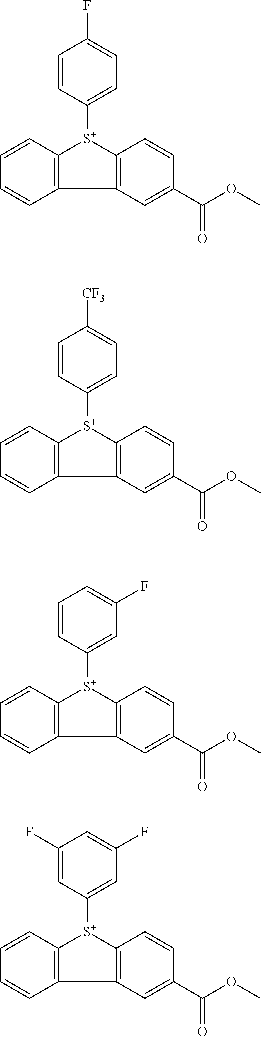 [SMILES: COC(=O)c1ccc2c(c1)c1ccccc1[s+]2-c1ccc(F)cc1 ; COC(=O)c1ccc2c(c1)c1ccccc1[s+]2-c1ccc(C(F)(F)F)cc1 ; COC(=O)c1ccc2c(c1)c1ccccc1[s+]2-c1cccc(F)c1 ; COC(=O)c1ccc2c(c1)c1ccccc1[s+]2-c1cc(F)cc(F)c1]

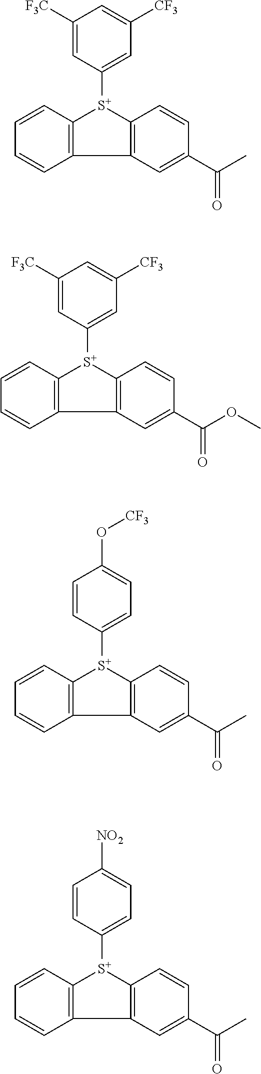 [SMILES: CC(=O)c1ccc2c(c1)c1ccccc1[s+]2-c1cc(C(F)(F)F)cc(C(F)(F)F)c1 ; COC(=O)c1ccc2c(c1)c1ccccc1[s+]2-c1cc(C(F)(F)F)cc(C(F)(F)F)c1 ; CC(=O)c1ccc2c(c1)c1ccccc1[s+]2-c1ccc(OC(F)(F)F)cc1 ; CC(=O)c1ccc2c(c1)c1ccccc1[s+]2-c1ccc([N+](=O)[O-])cc1]

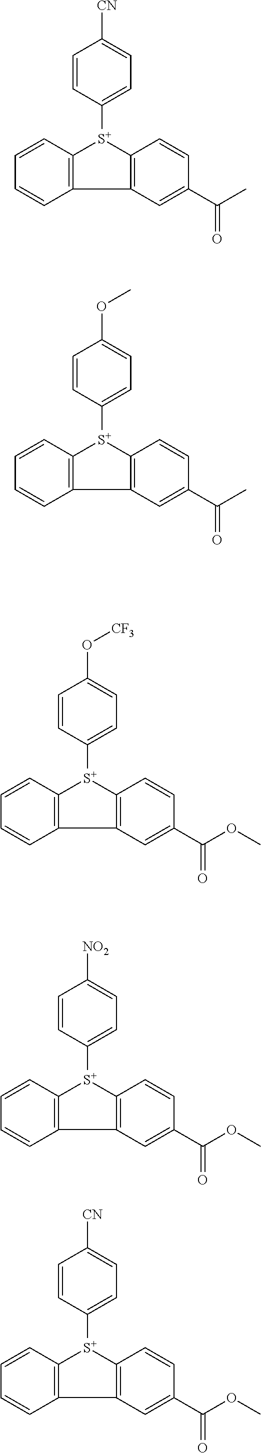 [SMILES: CC(=O)c1ccc2c(c1)c1ccccc1[s+]2-c1ccc(C#N)cc1 ; COc1ccc(-[s+]2c3ccccc3c3cc(C(C)=O)ccc32)cc1 ; COC(=O)c1ccc2c(c1)c1ccccc1[s+]2-c1ccc(OC(F)(F)F)cc1 ; COC(=O)c1ccc2c(c1)c1ccccc1[s+]2-c1ccc([N+](=O)[O-])cc1 ; COC(=O)c1ccc2c(c1)c1ccccc1[s+]2-c1ccc(C#N)cc1]

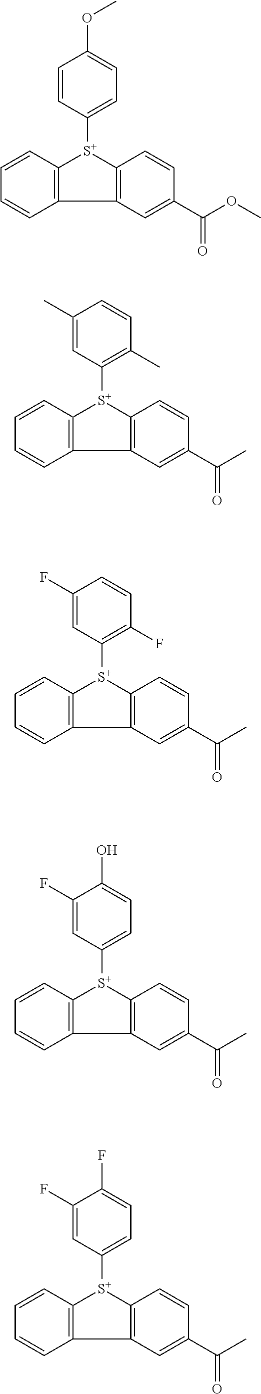 [SMILES: COC(=O)c1ccc2c(c1)c1ccccc1[s+]2-c1ccc(OC)cc1 ; CC(=O)c1ccc2c(c1)c1ccccc1[s+]2-c1cc(C)ccc1C ; CC(=O)c1ccc2c(c1)c1ccccc1[s+]2-c1cc(F)ccc1F ; CC(=O)c1ccc2c(c1)c1ccccc1[s+]2-c1ccc(O)c(F)c1 ; CC(=O)c1ccc2c(c1)c1ccccc1[s+]2-c1ccc(F)c(F)c1]

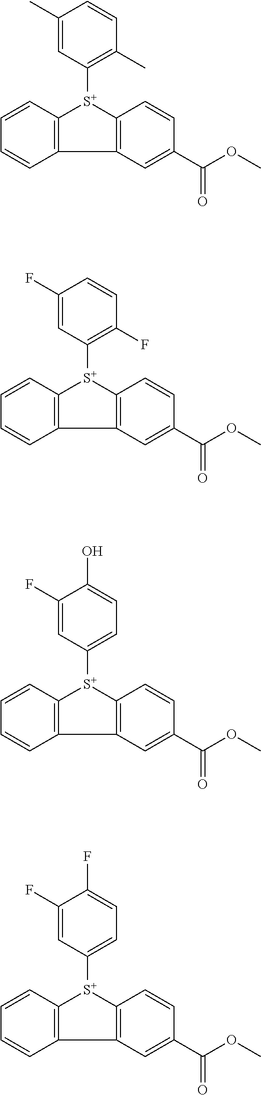 [SMILES: COC(=O)c1ccc2c(c1)c1ccccc1[s+]2-c1cc(C)ccc1C ; COC(=O)c1ccc2c(c1)c1ccccc1[s+]2-c1cc(F)ccc1F ; COC(=O)c1ccc2c(c1)c1ccccc1[s+]2-c1ccc(O)c(F)c1 ; COC(=O)c1ccc2c(c1)c1ccccc1[s+]2-c1ccc(F)c(F)c1]

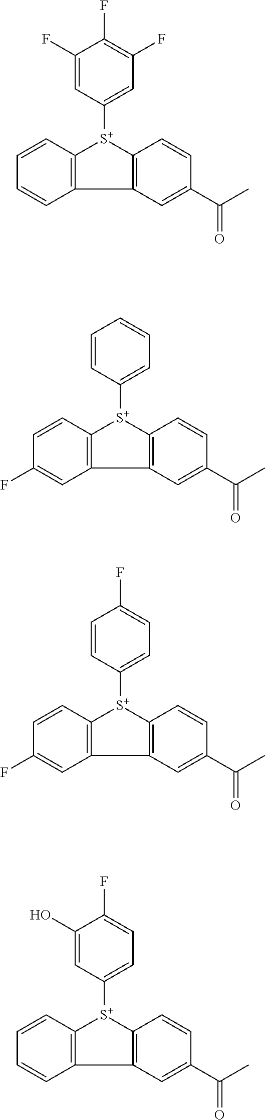 [SMILES: CC(=O)c1ccc2c(c1)c1ccccc1[s+]2-c1cc(F)c(F)c(F)c1 ; CC(=O)c1ccc2c(c1)c1cc(F)ccc1[s+]2-c1ccccc1 ; CC(=O)c1ccc2c(c1)c1cc(F)ccc1[s+]2-c1ccc(F)cc1 ; CC(=O)c1ccc2c(c1)c1ccccc1[s+]2-c1ccc(F)c(O)c1]

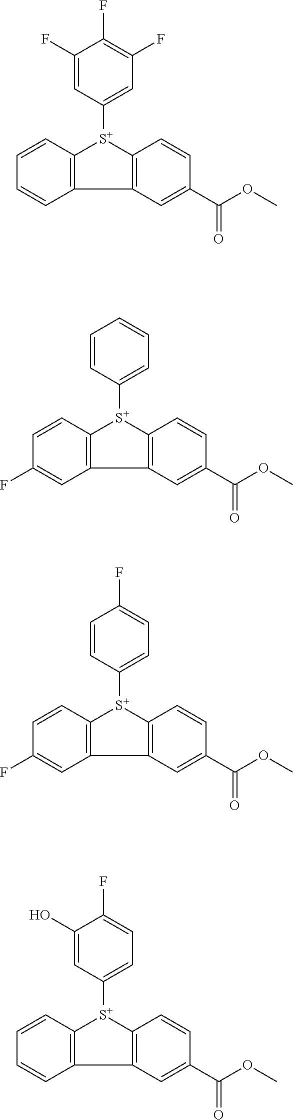 [SMILES: COC(=O)c1ccc2c(c1)c1ccccc1[s+]2-c1cc(F)c(F)c(F)c1 ; COC(=O)c1ccc2c(c1)c1cc(F)ccc1[s+]2-c1ccccc1 ; COC(=O)c1ccc2c(c1)c1cc(F)ccc1[s+]2-c1ccc(F)cc1 ; COC(=O)c1ccc2c(c1)c1ccccc1[s+]2-c1ccc(F)c(O)c1]

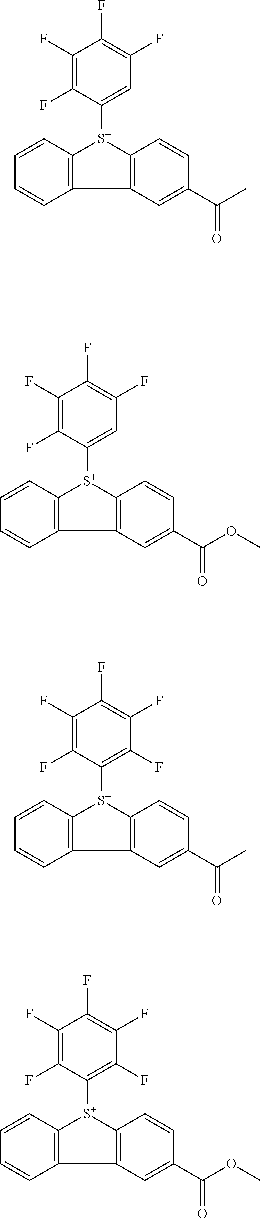 [SMILES: CC(=O)c1ccc2c(c1)c1ccccc1[s+]2-c1cc(F)c(F)c(F)c1F ; COC(=O)c1ccc2c(c1)c1ccccc1[s+]2-c1cc(F)c(F)c(F)c1F ; CC(=O)c1ccc2c(c1)c1ccccc1[s+]2-c1c(F)c(F)c(F)c(F)c1F ; COC(=O)c1ccc2c(c1)c1ccccc1[s+]2-c1c(F)c(F)c(F)c(F)c1F]

Examples of the iodonium cation M⁺ are shown below, but not limited thereto.

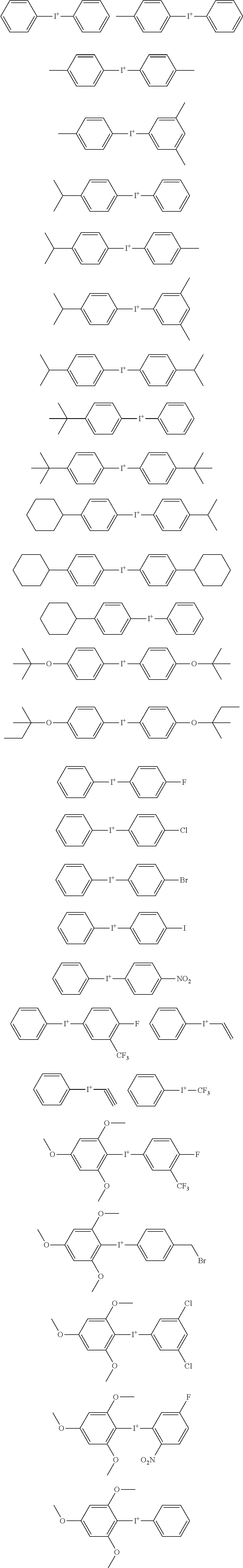

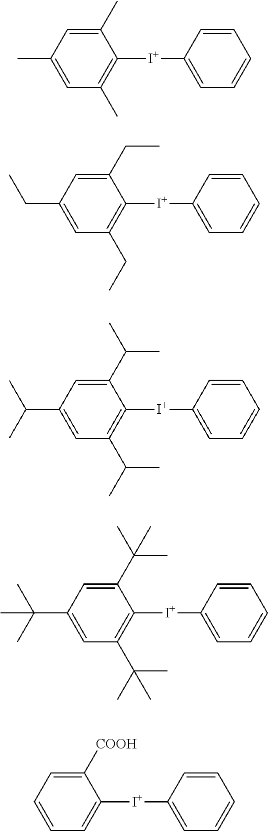 [SMILES: Cc1cc(C)c([I+]c2ccccc2)c(C)c1 ; CCc1cc(CC)c([I+]c2ccccc2)c(CC)c1 ; CC(C)c1cc(C(C)C)c([I+]c2ccccc2)c(C(C)C)c1 ; CC(C)(C)c1cc(C(C)(C)C)c([I+]c2ccccc2)c(C(C)(C)C)c1 ; O=C(O)c1ccccc1[I+]c1ccccc1]

The bis-onium salt can be synthesized, for example, by salt exchange between a sulfonium or iodonium salt containing a halide anion and an ammonium salt containing a divalent anion having a sulfonic acid anion structure directly bonded to an iodized or brominated aromatic group and a sulfonimide or sulfonamide anion structure bonded to the iodized or brominated aromatic group via a linking group of one or more carbon atoms.

In the resist composition, the bis-onium salt is preferably present in an amount of 0.01 to 1,000 parts by weight, and more preferably 0.05 to 500 parts by weight per 100 parts by weight of a base polymer to be described just below, as viewed from sensitivity and acid diffusion suppressing effect.

### [Base Polymer]

In a preferred embodiment, the resist composition contains a base polymer. In the case of a positive resist composition, the base polymer comprises repeat units containing an acid labile group. The preferred repeat units containing an acid labile group are repeat units having the formula (a1) or repeat units having the formula (a2). These repeat units are also referred to as repeat units (a1) and (a2).

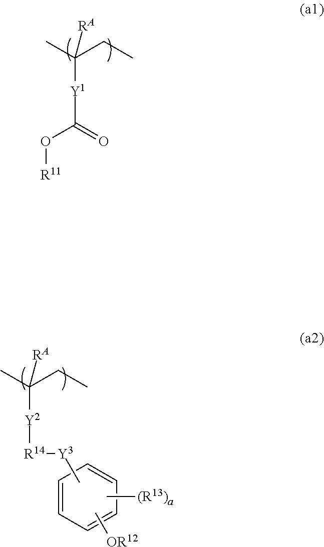

In formulae (a1) and (a2), R^{A} is each independently hydrogen or methyl. Y¹ is a single bond, phenylene group, naphthylene group, or a C₁-C₁₂ linking group containing at least one moiety selected from ester bond, ether bond and lactone ring. The phenylene group, naphthylene group, and linking group may contain at least one moiety selected from hydroxy, C₁-C₈ saturated hydrocarbyloxy moiety, and C₂-C₈ saturated hydrocarbylcarbonyloxy moiety. Y² is a single bond or ester bond. Y³ is a single bond, ether bond or ester bond. R¹¹ and R¹² are each independently an acid labile group. R¹³ is a C₁-C₄ saturated hydrocarbyl group, halogen, C₂-C₅ saturated hydrocarbylcarbonyl group, cyano, or C₂-C₅ saturated hydrocarbyloxycarbonyl group. R¹⁴ is a single bond or C₁-C₆ alkanediyl group which may contain an ether bond or ester bond. The subscript “a” is an integer of 0 to 4.

Examples of the monomer from which repeat units (a1) are derived are shown below, but not limited thereto. Herein R^{A} and R¹¹ are as defined above.

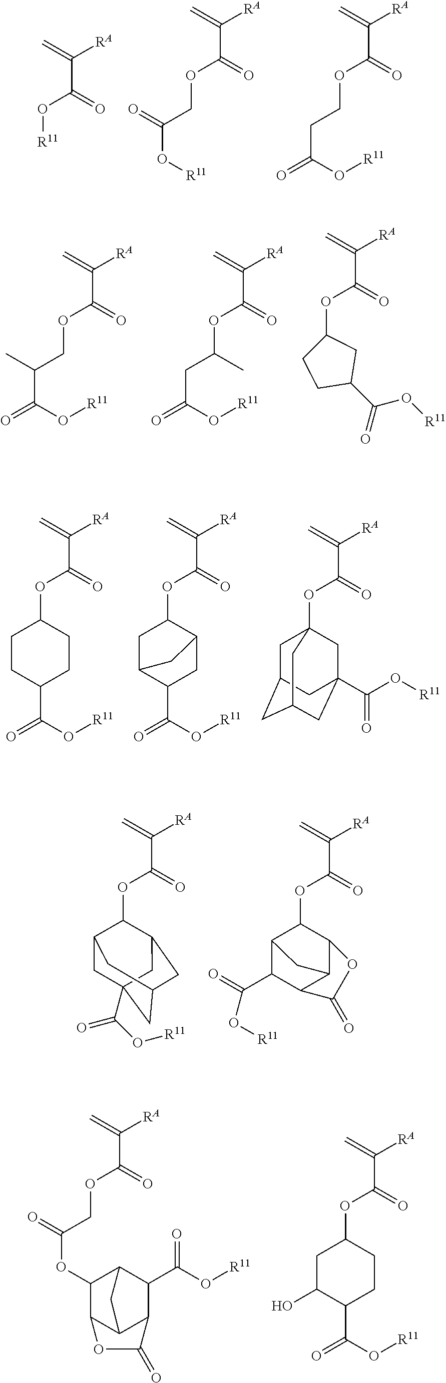

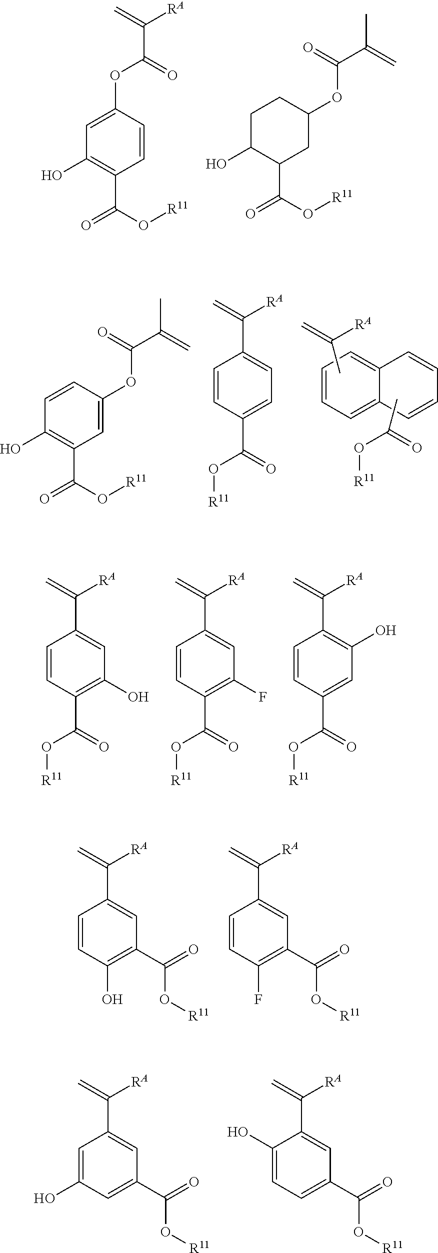

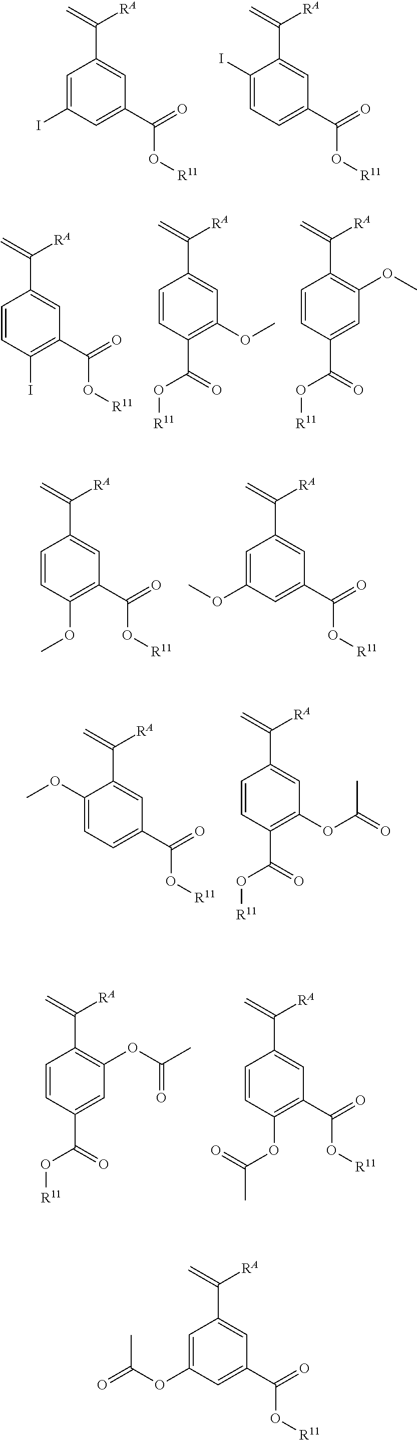

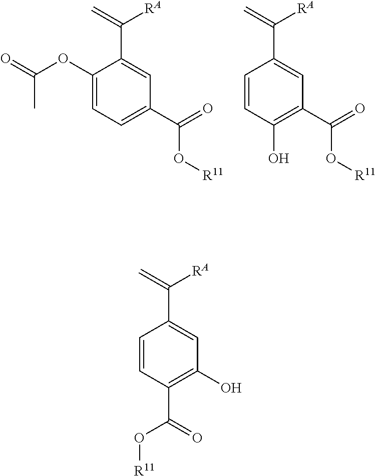

Examples of the monomer from which repeat units (a2) are derived are shown below, but not limited thereto. Herein R^{A} and R¹² are as defined above.

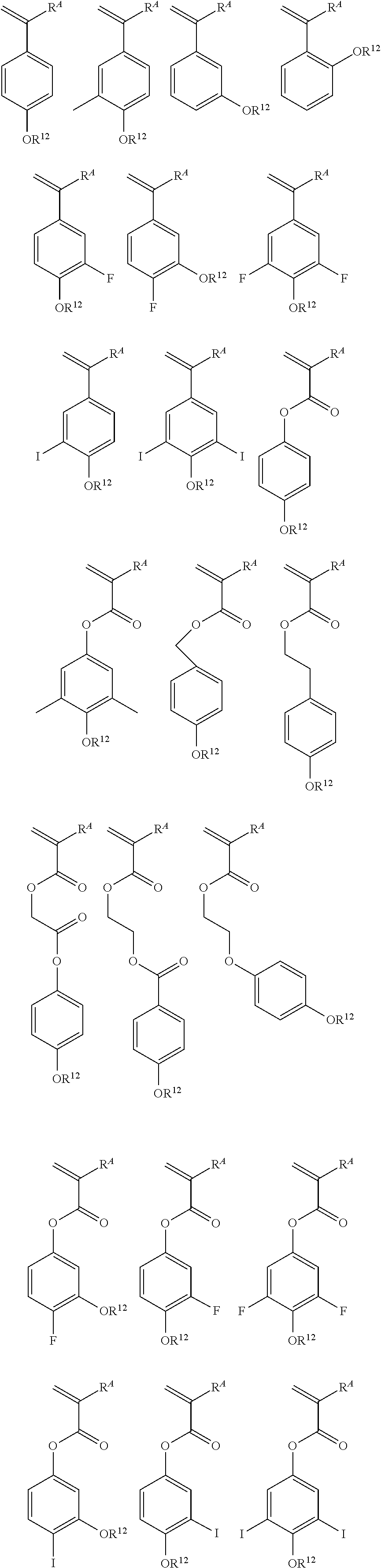

In formulae (a1) and (a2), R¹¹ and R¹² are each independently an acid labile group. The acid labile group may be selected from a variety of such groups, for example, the groups described in U.S. Pat. No. 8,574,817 (JP-A 2013-080033) and U.S. Pat. No. 8,846,303 (JP-A 2013-083821).

Typical of the acid labile group are groups having the following formulae (AL-1) to (AL-3).

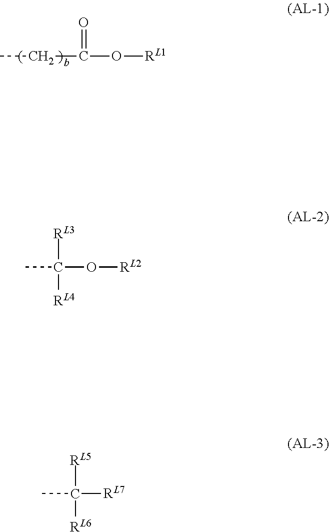

In formulae (AL-1) and (AL-2), R^{L1} and R^{L2} are each independently a C₁-C₄₀ hydrocarbyl group which may contain a heteroatom such as oxygen, sulfur, nitrogen or fluorine. The hydrocarbyl group may be saturated or unsaturated and straight, branched or cyclic. Inter alia, C₁-C₄₀ saturated hydrocarbyl groups are preferred, with C₁-C₂₀ saturated hydrocarbyl groups being more preferred.

In formula (AL-1), b is an integer of 0 to 10, preferably 1 to 5.

In formula (AL-2), R^{L3} and R^{L4} are each independently hydrogen or a C₁-C₂₀ hydrocarbyl group which may contain a heteroatom such as oxygen, sulfur, nitrogen or fluorine. The hydrocarbyl group may be saturated or unsaturated and straight, branched or cyclic. Inter alia, C₁-C₂₀ saturated hydrocarbyl groups are preferred. Any two of R^{L2}, R^{L3} and R^{L4} may bond together to form a C₃-C₂₀ ring with the carbon atom or carbon and oxygen atoms to which they are attached. Rings of 4 to 16 carbon atoms are preferred, with aliphatic rings being more preferred.

In formula (AL-3), R^{L5}, R^{L6} and R^{L7} are each independently a C₁-C₂₀ hydrocarbyl group which may contain a heteroatom such as oxygen, sulfur, nitrogen or fluorine. The hydrocarbyl group may be saturated or unsaturated and straight, branched or cyclic. Inter alia, C₁-C₂₀ saturated hydrocarbyl groups are preferred. Any two of R^{L5}, R^{L6} and R^{L7} may bond together to form a C₃-C₂₀ ring with the carbon atom to which they are attached. Rings of 4 to 16 carbon atoms are preferred, with aliphatic rings being more preferred.

The base polymer may comprise repeat units (b) having a phenolic hydroxy group as an adhesive group. Examples of suitable monomers from which repeat units (b) are derived are given below, but not limited thereto. Herein R^{A} is as defined above.

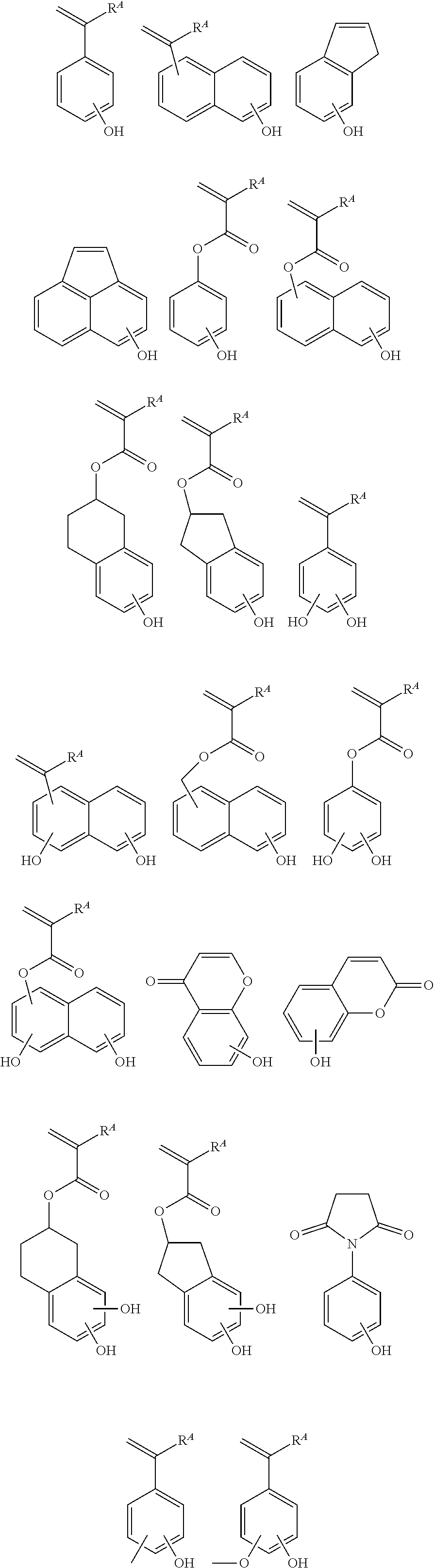

The base polymer may further comprise repeat units (c) having another adhesive group selected from hydroxy group (other than the foregoing phenolic hydroxy), lactone ring, sultone ring, ether bond, ester bond, sulfonate ester bond, carbonyl group, sulfonyl group, cyano group, and carboxy group. Examples of suitable monomers from which repeat units (c) are derived are given below, but not limited thereto. Herein R^{A} is as defined above.

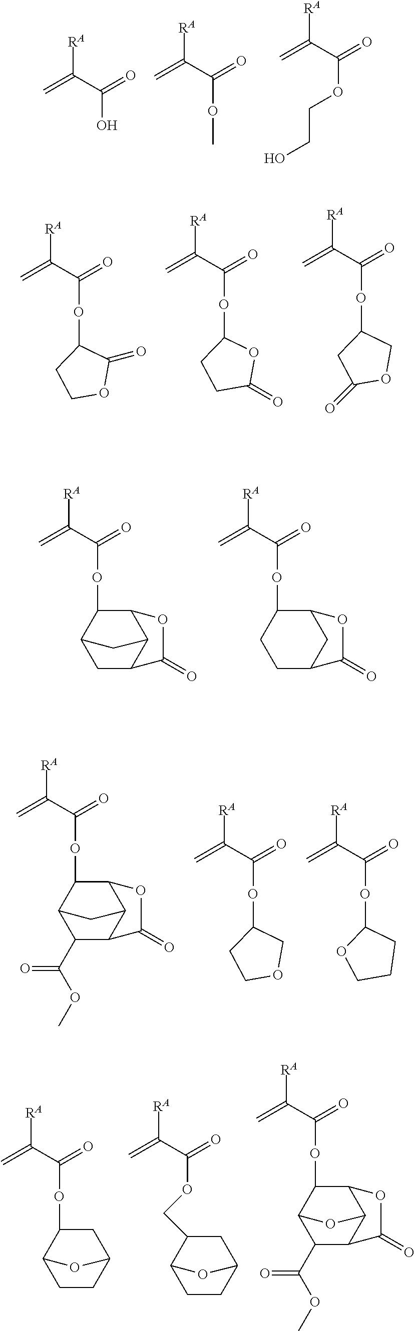

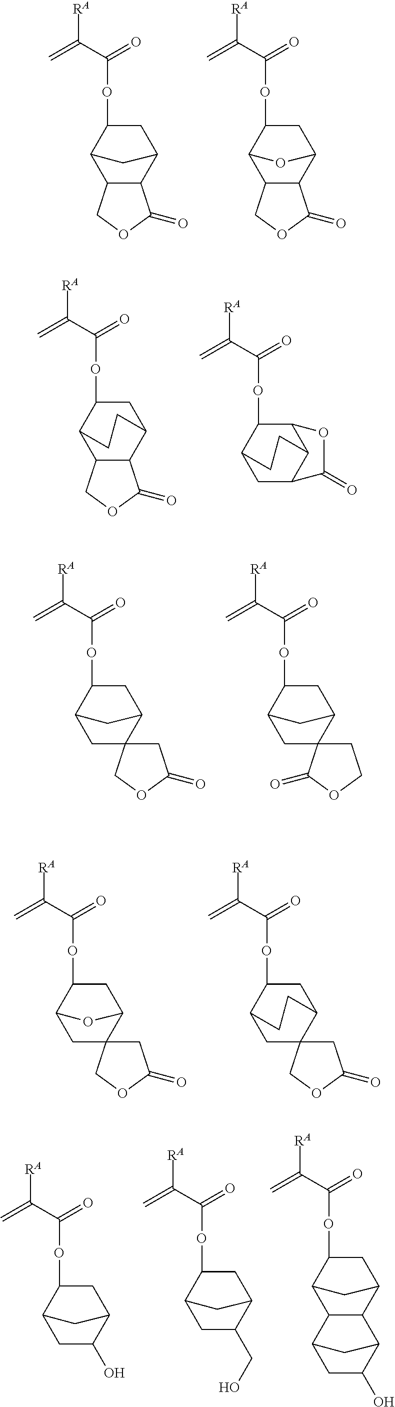

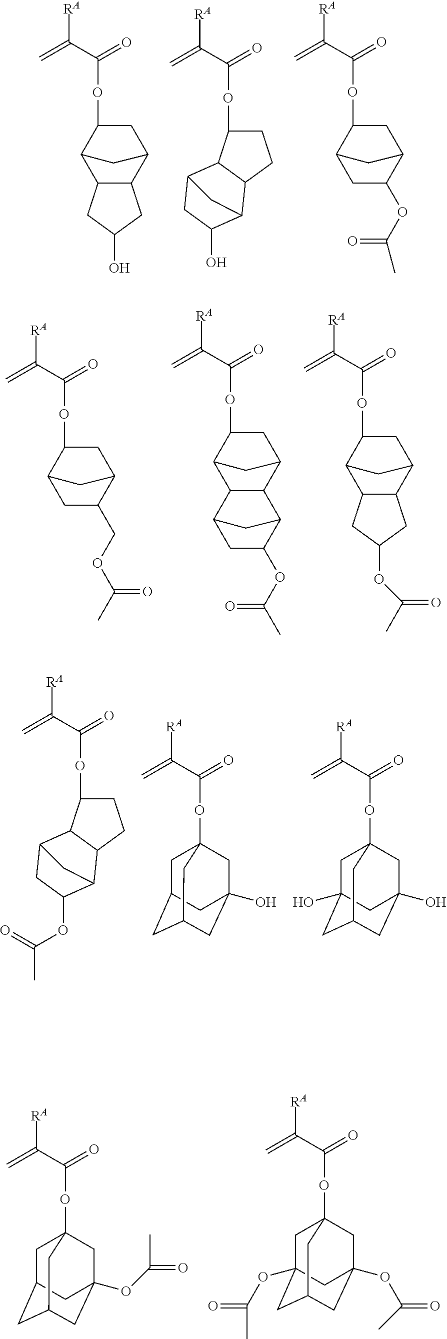

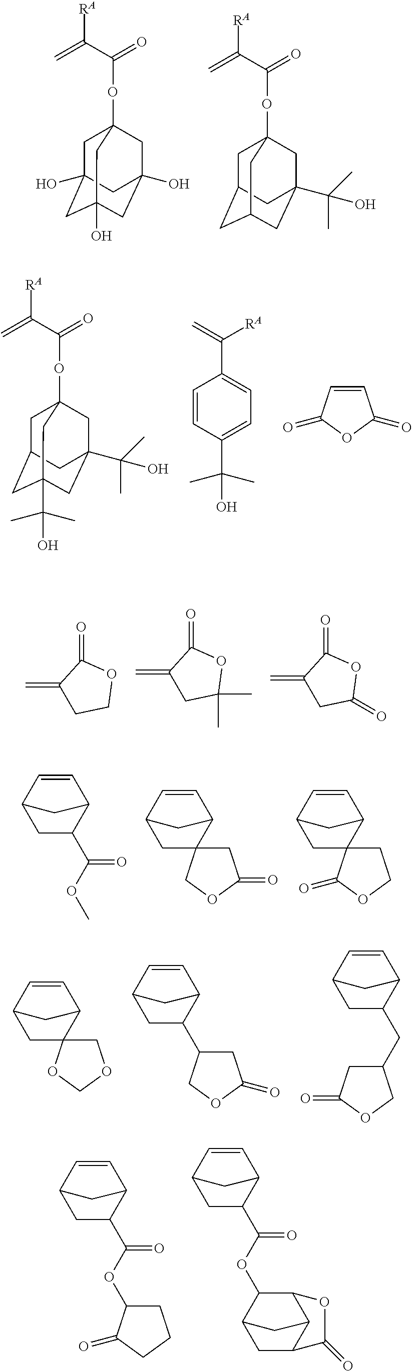

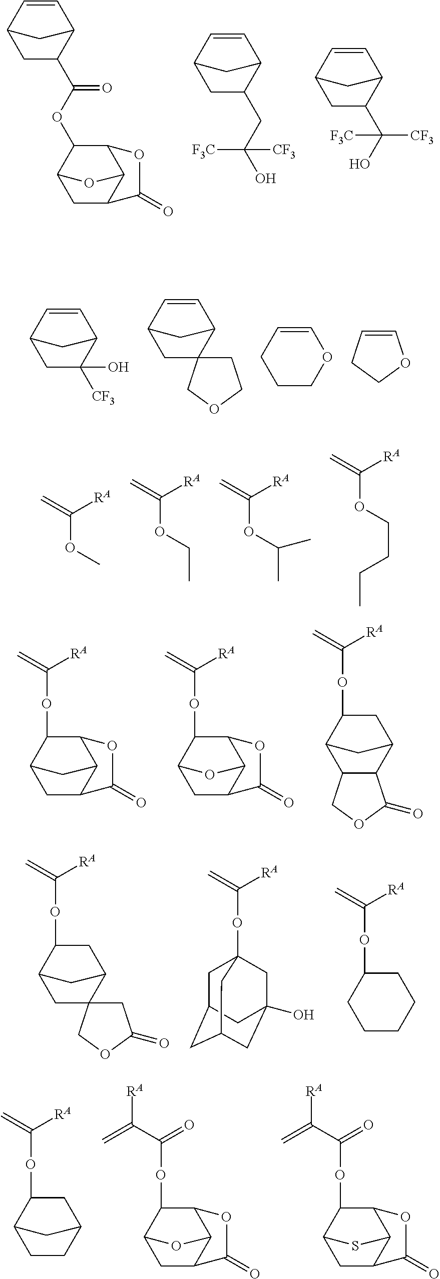

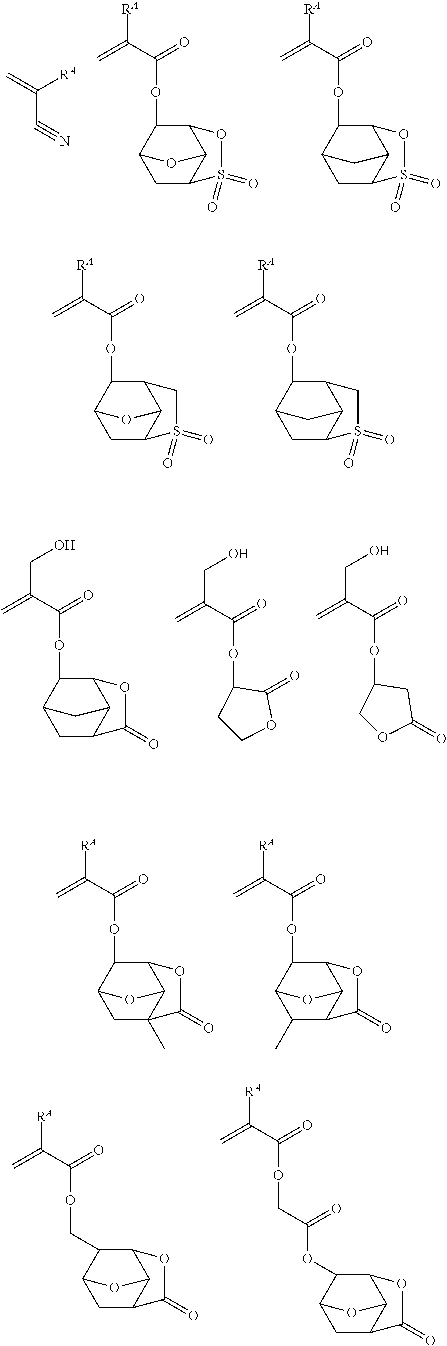

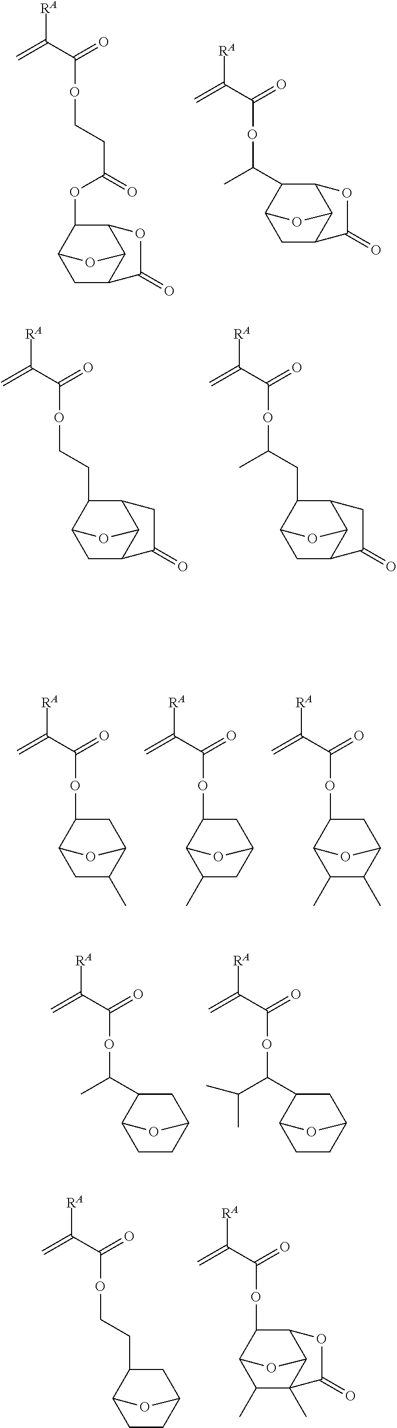

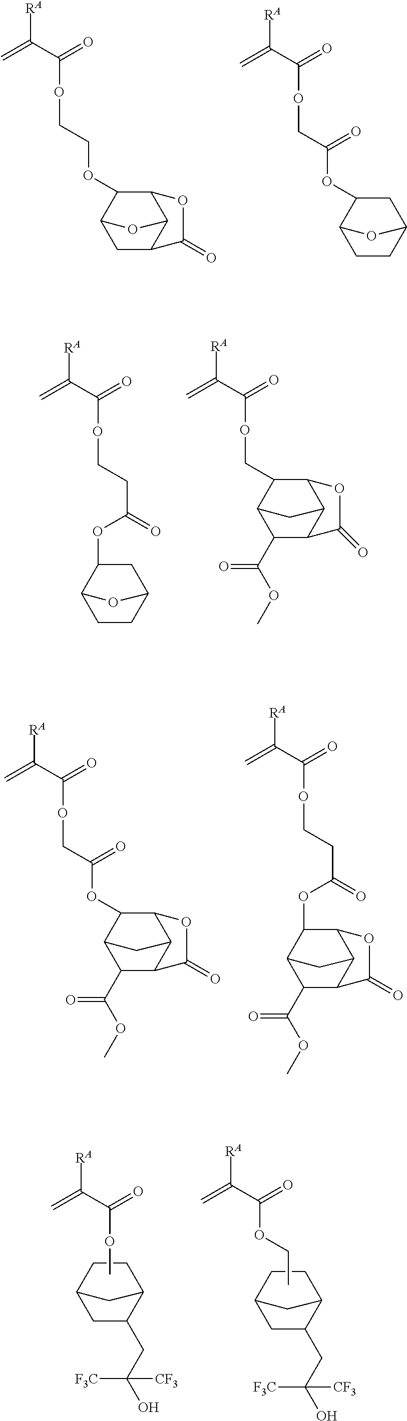

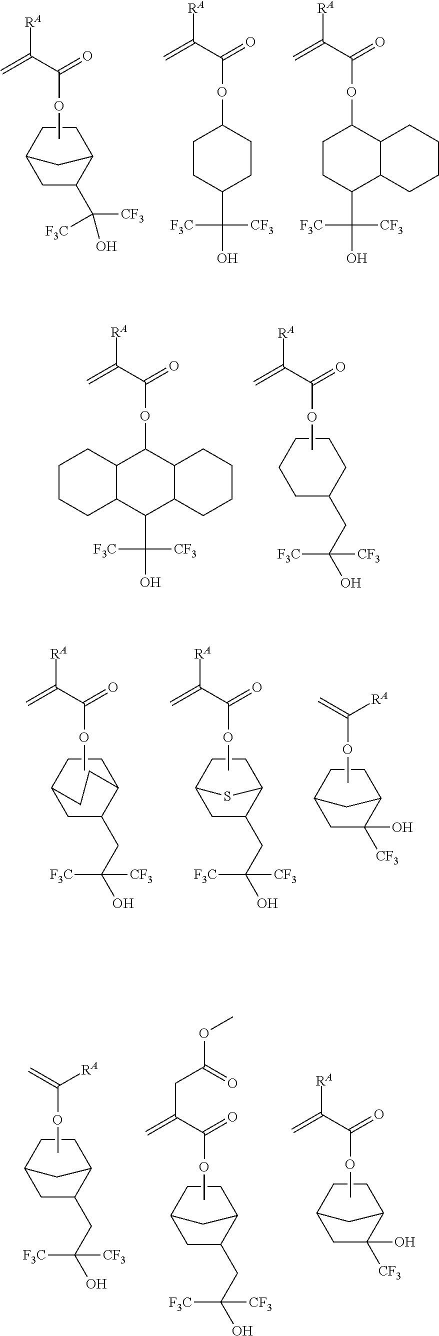

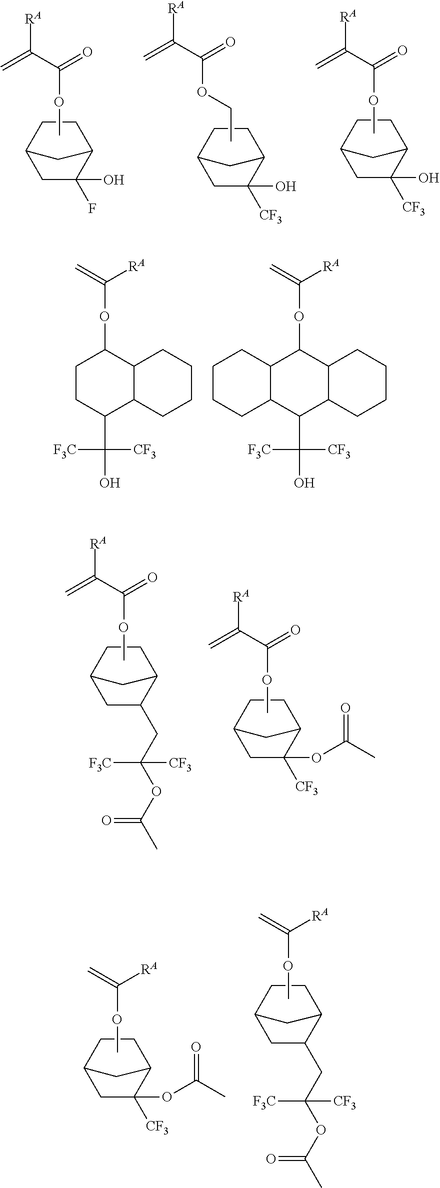

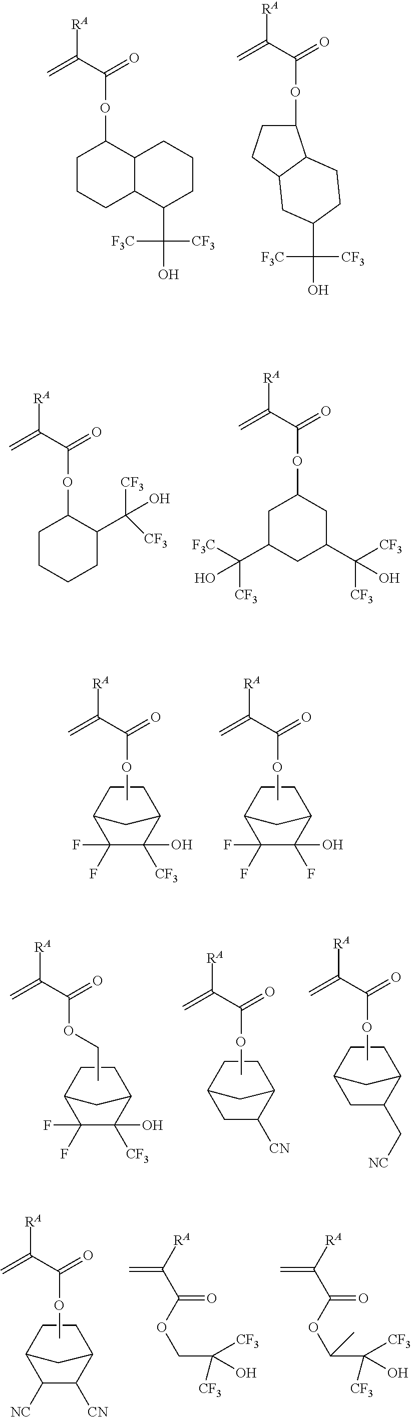

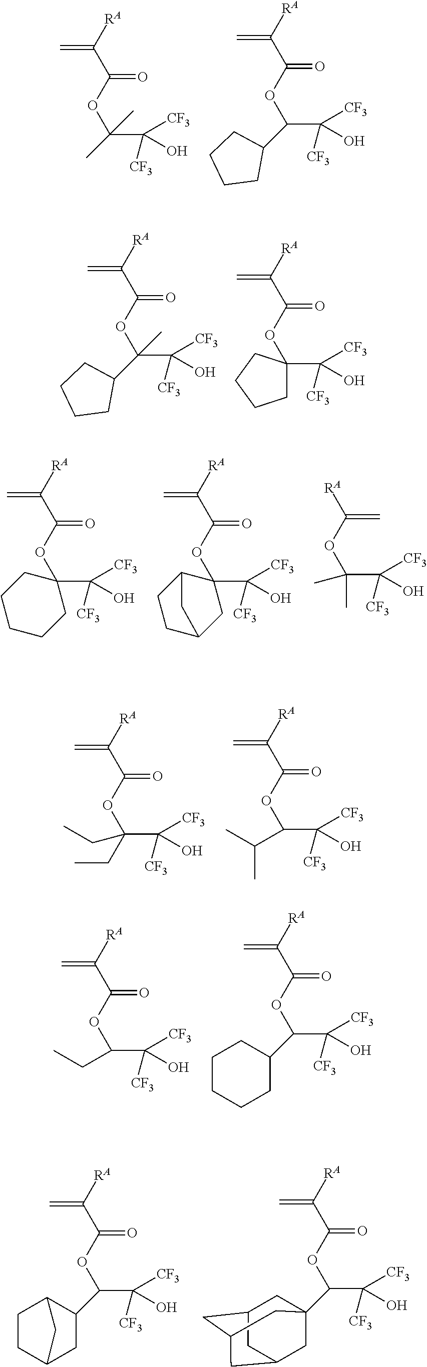

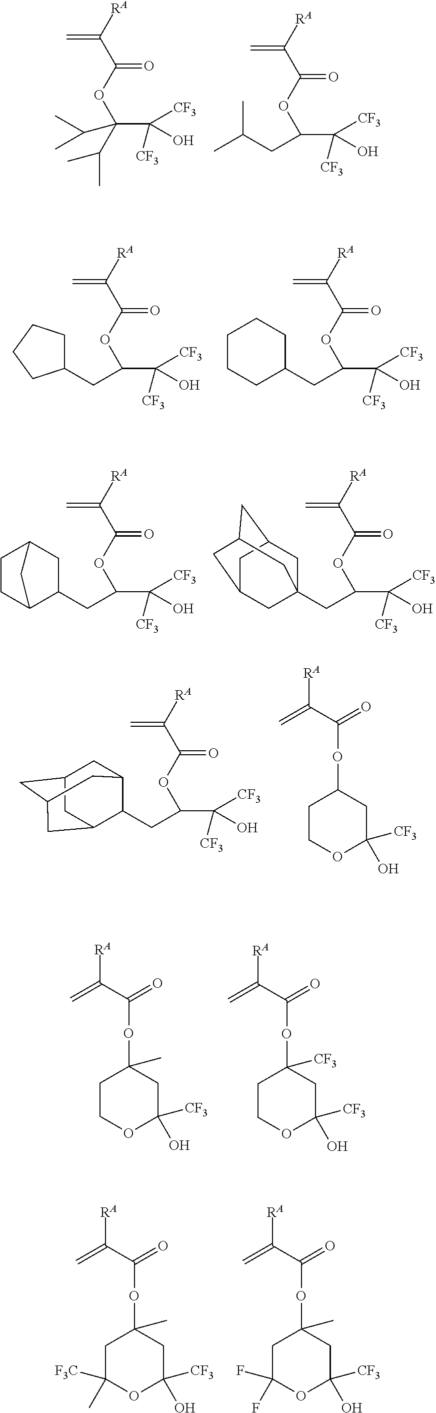

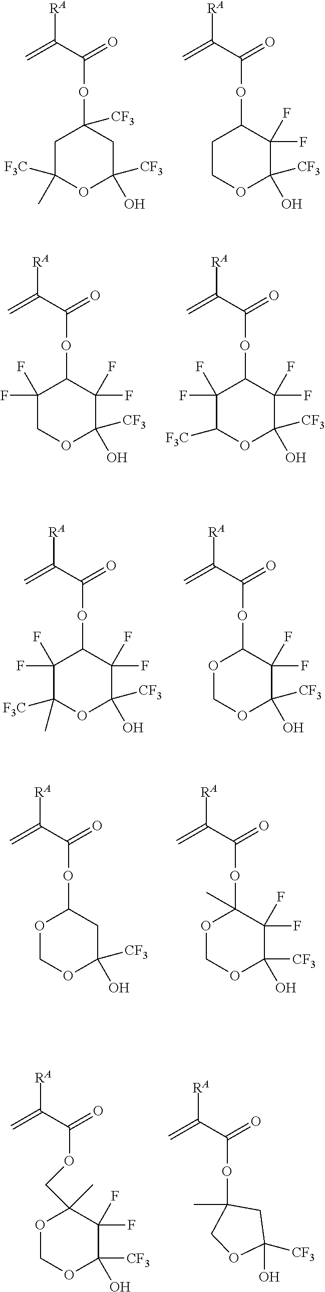

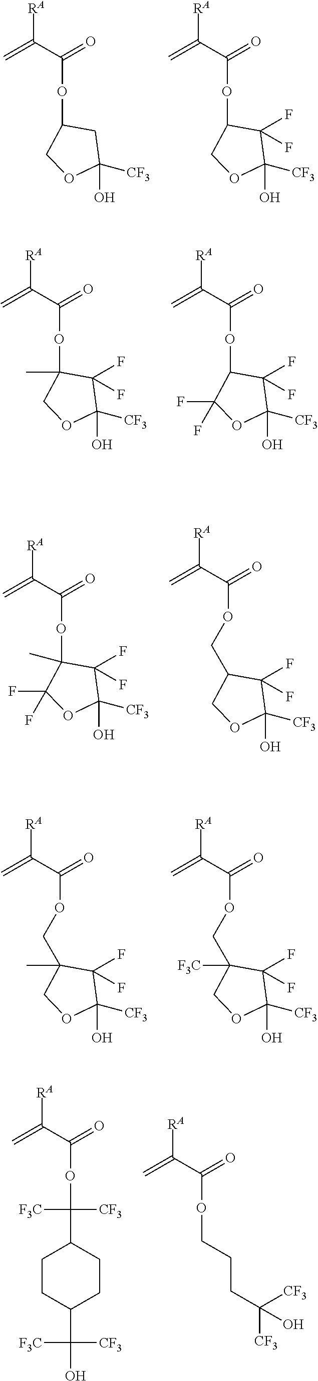

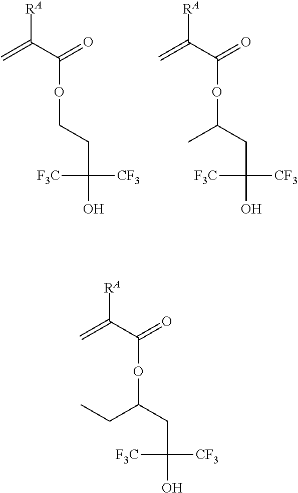

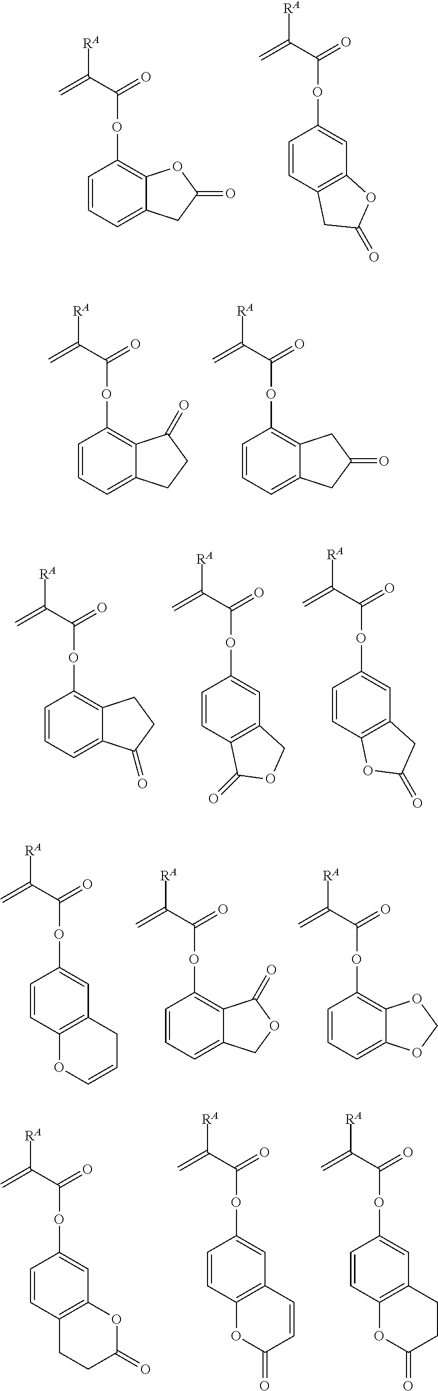

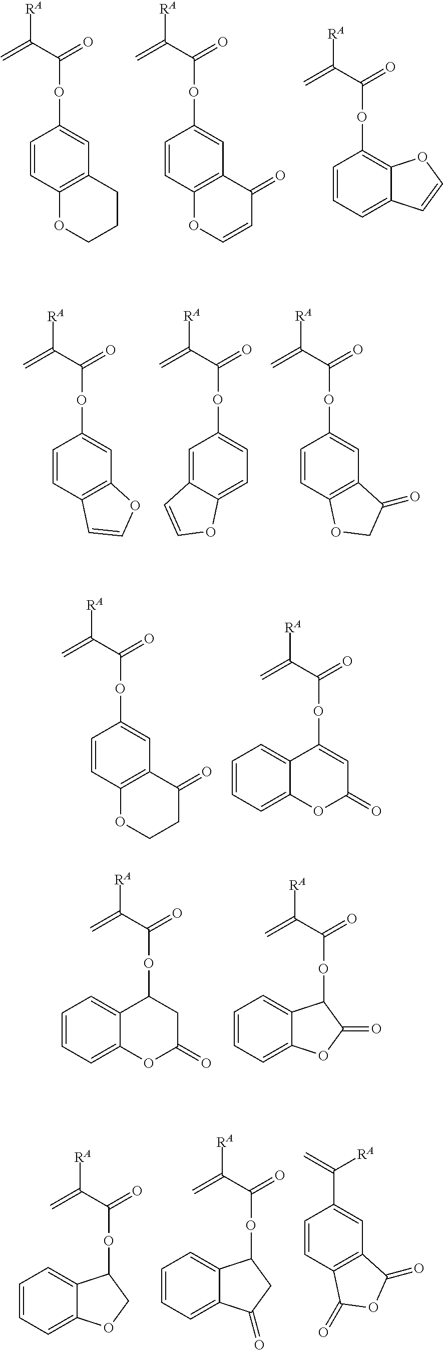

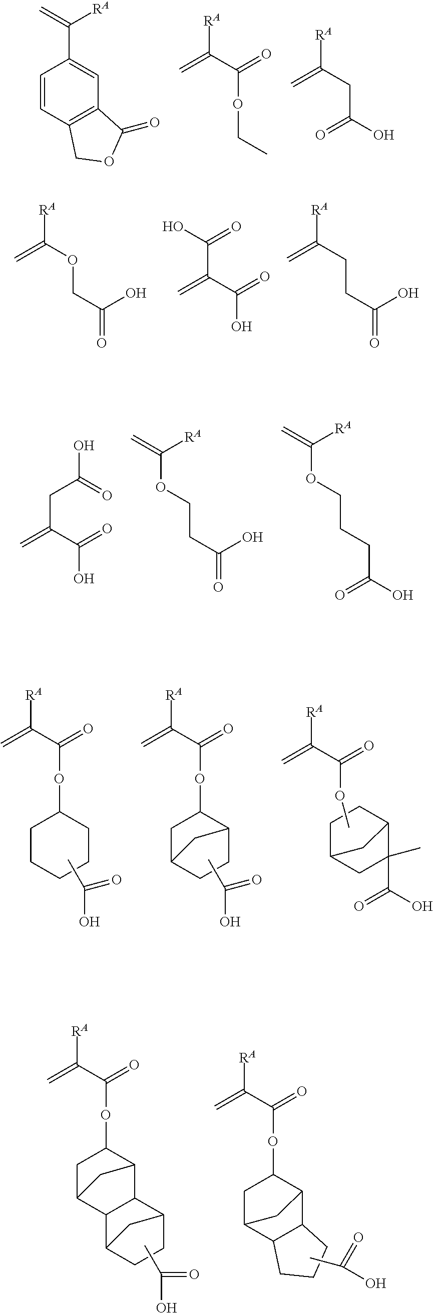

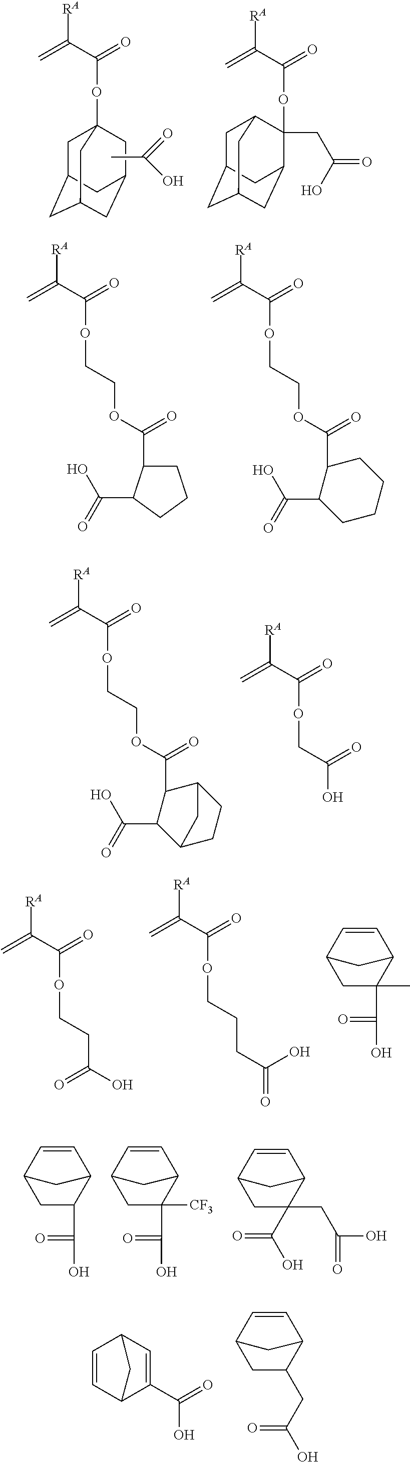

In another preferred embodiment, the base polymer may further comprise repeat units (d) derived from indene, benzofuran, benzothiophene, acenaphthylene, chromone, coumarin, and norbornadiene, or derivatives thereof. Suitable monomers are exemplified below.

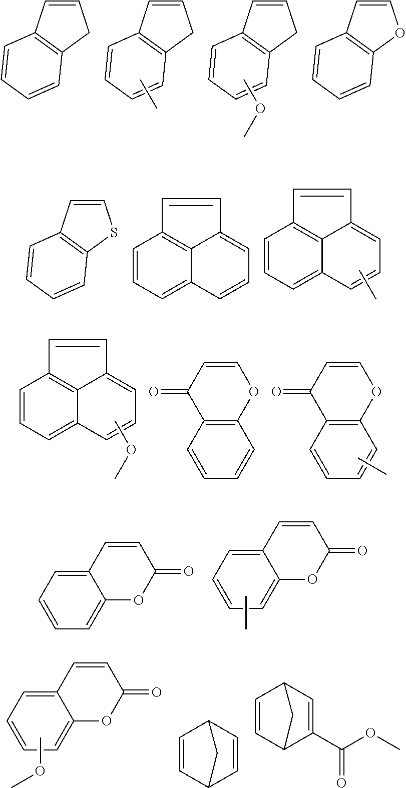

The base polymer may further comprise repeat units (e) which are derived from styrene, vinylnaphthalene, vinylanthracene, vinylpyrene, methyleneindene, vinylpyridine, vinylcarbazole, or derivatives thereof.

The base polymer may further comprise repeat units (f) which are derived from an onium salt having a polymerizable unsaturated bond. JP-A 2005-084365 discloses sulfonium and iodonium salts having polymerizable olefin capable of generating a specific sulfonic acid. JP-A 2006-178317 discloses a sulfonium salt having sulfonic acid directly attached to the main chain.

The preferred repeat units (f) are repeat units having formulae (f1), (f2) and (f3). These units are simply referred to as repeat units (f1), (f2) and (f3), which may be used alone or in combination of two or more types.

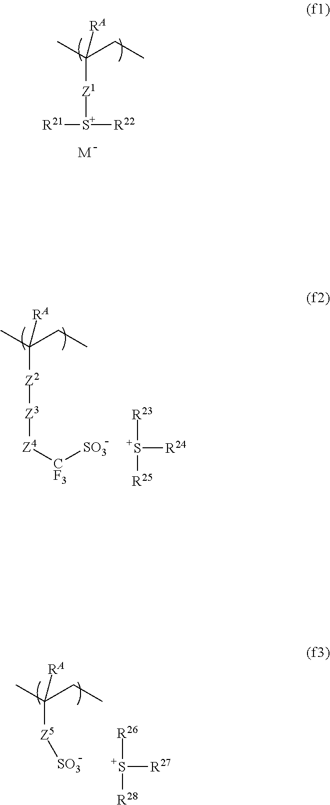

In formulae (f1) to (f3), R^{A} is each independently hydrogen or methyl. Z¹ is a single bond, C₁-C₆ aliphatic hydrocarbylene group, phenylene group, naphthylene group, or C₇-Cis group obtained by combining the foregoing, —O—Z¹¹—, —C(═O)—O—Z¹¹—, or —C(═O)—NH—Z¹¹—. Z¹¹ is a C₁-C₆ aliphatic hydrocarbylene group, phenylene group, naphthylene group, or C₇-C₁₈ group obtained by combining the foregoing, which may contain a carbonyl moiety, ester bond, ether bond or hydroxy moiety. Z² is a single bond or ester bond. Z³ is a single bond, —Z³¹—C(═O)—O—, —Z³¹—O— or —Z³¹—O—C(═O)—. Z³¹ is a C₁-C₁₂ aliphatic hydrocarbylene group, phenylene group or C₇-C₁₈ group obtained by combining the foregoing, which may contain a carbonyl moiety, ester bond, ether bond, iodine or bromine. Z⁴ is methylene, 2,2,2-trifluoro-1,1-ethanediyl or carbonyl. Z⁵ is a single bond, methylene, ethylene, phenylene, fluorinated phenylene, trifluoromethyl-substituted phenylene, —O—Z⁵¹—, —C(═O)—O—Z⁵¹—, or —C(═O)—NH—Z⁵¹—. Z⁵¹ is a C₁-C₆ aliphatic hydrocarbylene group, phenylene group, fluorinated phenylene group, or trifluoromethyl-substituted phenylene group, which may contain a carbonyl moiety, ester bond, ether bond, hydroxy moiety or halogen.

In formulae (f1) to (f3), R²¹ to R²⁸ are each independently halogen or a C₁-C₂₀ hydrocarbyl group which may contain a heteroatom. Suitable halogen atoms include fluorine, chlorine, bromine and iodine. The hydrocarbyl group may be saturated or unsaturated and straight, branched or cyclic. Examples thereof are as exemplified above for R⁶ to R¹⁰ in formulae (2) and (3). In the hydrocarbyl group, some or all of the hydrogen atoms may be substituted by a moiety containing a heteroatom such as oxygen, sulfur, nitrogen or halogen, or some —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonic ester bond, carbonate bond, lactone ring, sultone ring, carboxylic anhydride (—C(═O)—O—C(═O)—) or haloalkyl. A pair of R²³ and R²⁴, or R²⁶ and R²⁷ may bond together to form a ring with the sulfur atom to which they are attached. Examples of the ring are as exemplified above for the ring that R⁶ and R⁷ in formula (2), taken together, form with the sulfur atom to which they are attached.

In formula (f1), M⁻ is a non-nucleophilic counter ion. Examples of the non-nucleophilic counter ion include halide ions such as chloride and bromide ions; fluoroalkylsulfonate ions such as triflate, 1,1,1-trifluoroethanesulfonate, and nonafluorobutanesulfonate; arylsulfonate ions such as tosylate, benzenesulfonate, 4-fluorobenzenesulfonate, and 1,2,3,4,5-pentafluorobenzenesulfonate; alkylsulfonate ions such as mesylate and butanesulfonate; imide ions such as bis(trifluoromethylsulfonyl)imide, bis(perfluoroethylsulfonyl)imide and bis(perfluorobutylsulfonyl)imide; and methide ions such as tris(trifluoromethylsulfonyl)methide and tris(perfluoroethylsulfonyl)methide.

Also included are sulfonate ions having fluorine substituted at α-position as represented by the formula (f1-1), and sulfonate ions having fluorine substituted at α-position and trifluoromethyl at β-position as represented by the formula (f1-2).

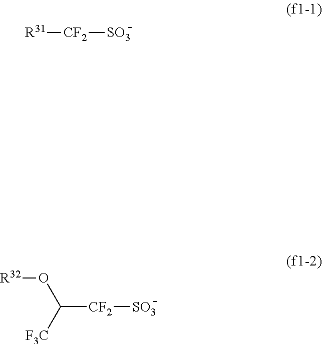

In formula (f1-1), R³¹ is hydrogen, or a C₁-C₂₀ hydrocarbyl group which may contain an ether bond, ester bond, carbonyl moiety, lactone ring, or fluorine atom.

In formula (f1-2), R³² is hydrogen, or a C₁-C₃₀ hydrocarbyl group or C₂-C₃₀ hydrocarbylcarbonyl group, which may contain an ether bond, ester bond, carbonyl moiety or lactone ring.

The hydrocarbyl group and hydrocarbyl moiety in the hydrocarbylcarbonyl group, represented by R³¹ or R³², may be saturated or unsaturated and straight, branched or cyclic. Examples of the hydrocarbyl group include alkyl groups such as methyl, ethyl, propyl, isopropyl, butyl, isobutyl, sec-butyl, tert-butyl, pentyl, neopentyl, hexyl, heptyl, 2-ethylhexyl, nonyl, undecyl, tridecyl, pentadecyl, heptadecyl, and icosyl; cyclic saturated hydrocarbyl groups such as cyclopentyl, cyclohexyl, 1-adamantyl, 2-adamantyl, 1-adamantylmethyl, norbornyl, norbornylmethyl, tricyclodecanyl, tetracyclododecanyl, tetracyclododecanylmethyl, and dicyclohexylmethyl; alkenyl groups such as allyl; cyclic unsaturated hydrocarbyl groups such as 3-cyclohexenyl; aryl groups such as phenyl, 1-naphthyl, 2-naphthyl; and aralkyl groups such as benzyl and diphenylmethyl.

In the hydrocarbyl group, some or all of the hydrogen atoms may be substituted by a moiety containing a heteroatom such as oxygen, sulfur, nitrogen or halogen, or some —CH₂— may be replaced by a moiety containing a heteroatom such as oxygen, sulfur or nitrogen, so that the group may contain a hydroxy, cyano, carbonyl, ether bond, ester bond, sulfonic ester bond, carbonate bond, lactone ring, sultone ring, carboxylic anhydride (—C(═O)—O—C(═O)—) or haloalkyl moiety. Examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, and 3-oxocyclohexyl.

Examples of the cation in the monomer from which repeat unit (f1) is derived are shown below, but not limited thereto. R^{A} is as defined above.

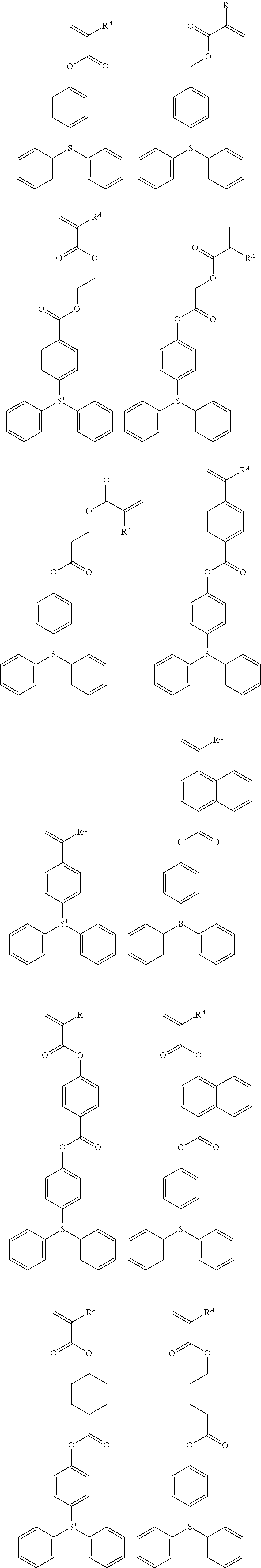

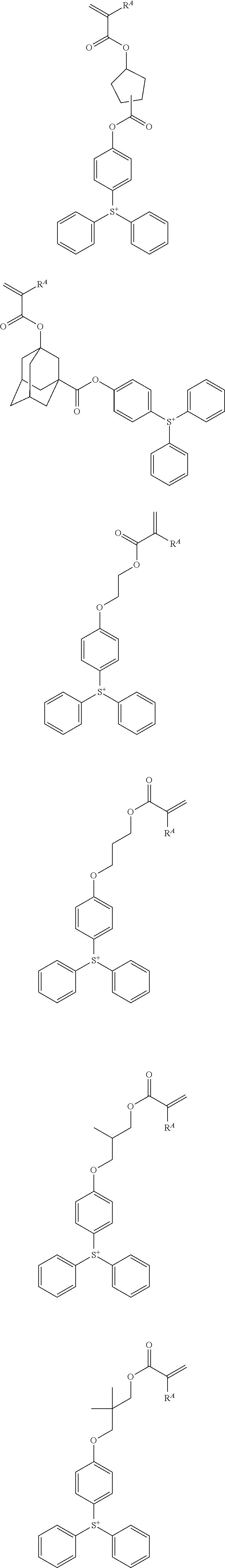

Examples of the cation in the monomer from which repeat unit (f2) or (f3) is derived are as exemplified above for the sulfonium cation M⁺ in formula (1).

Examples of the monomer from which repeat unit (f2) is derived are shown below, but not limited thereto. R^{A} is as defined above.

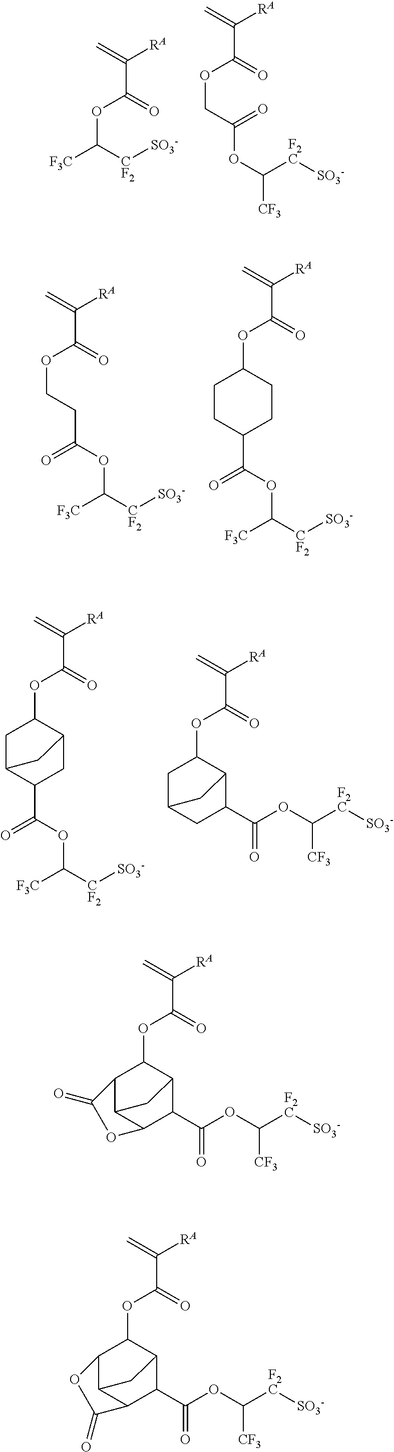

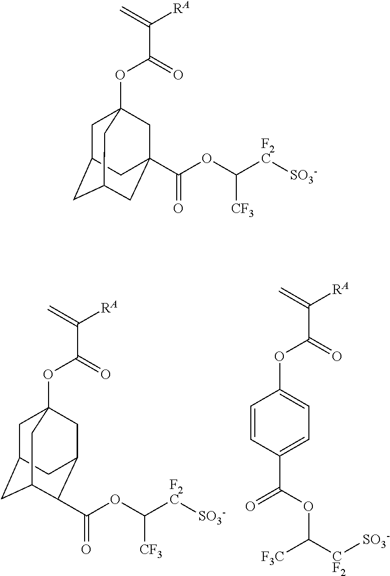

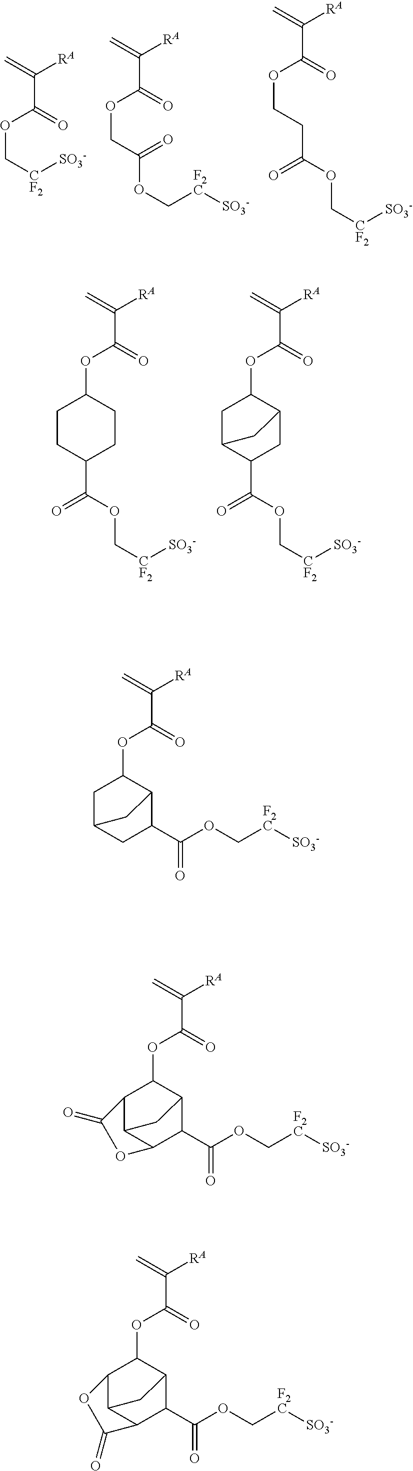

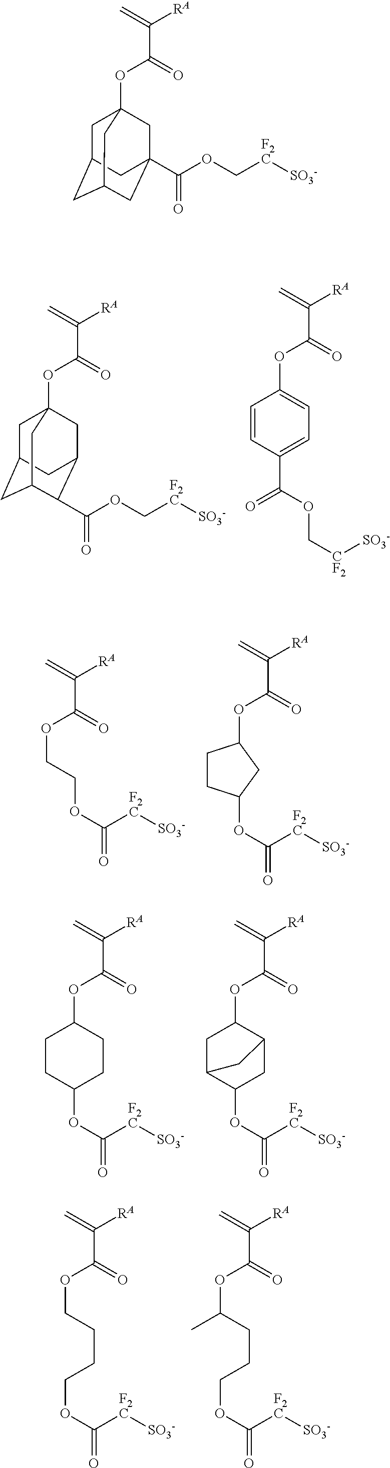

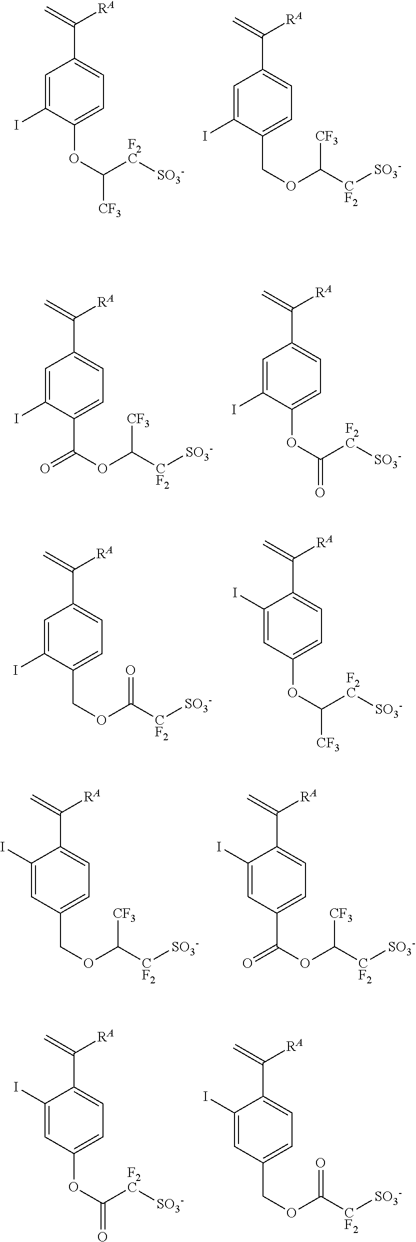

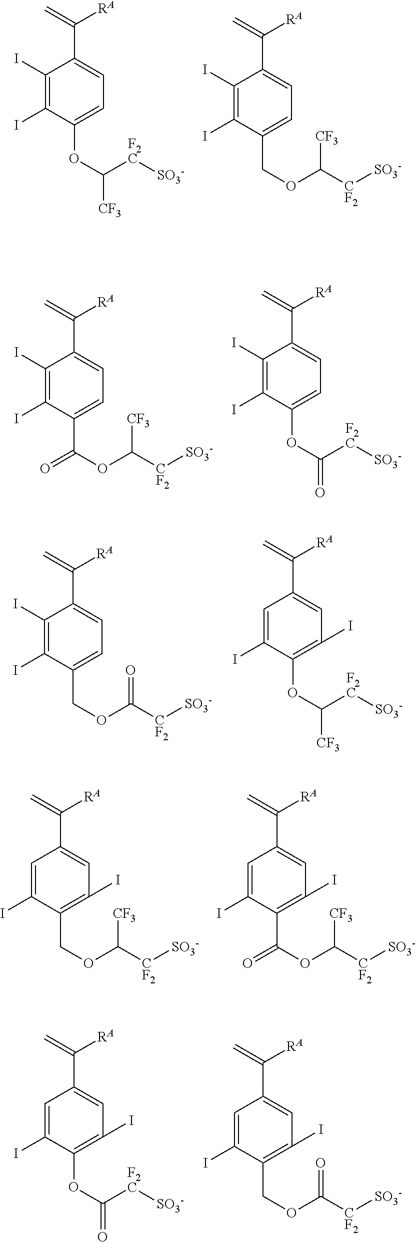

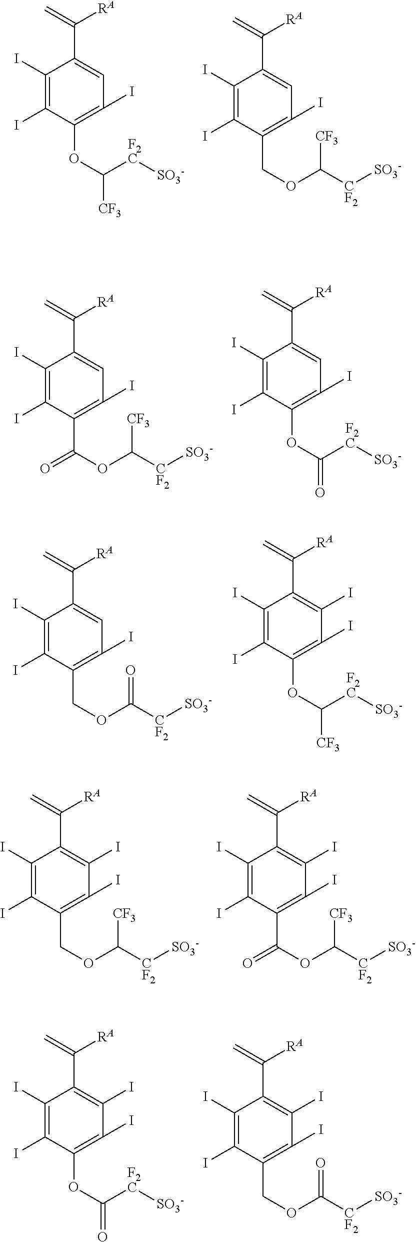

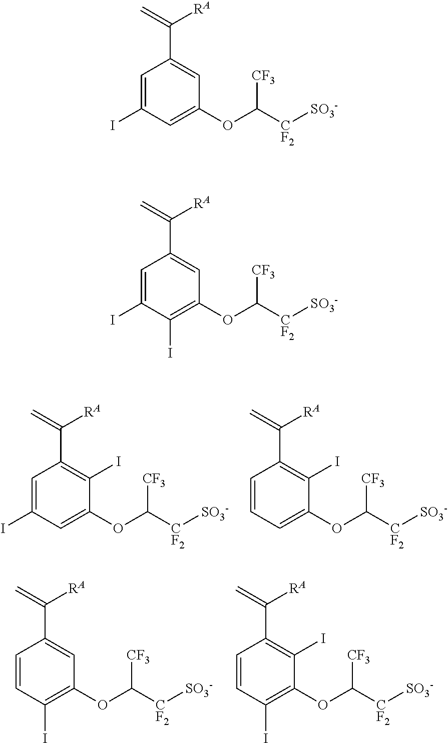

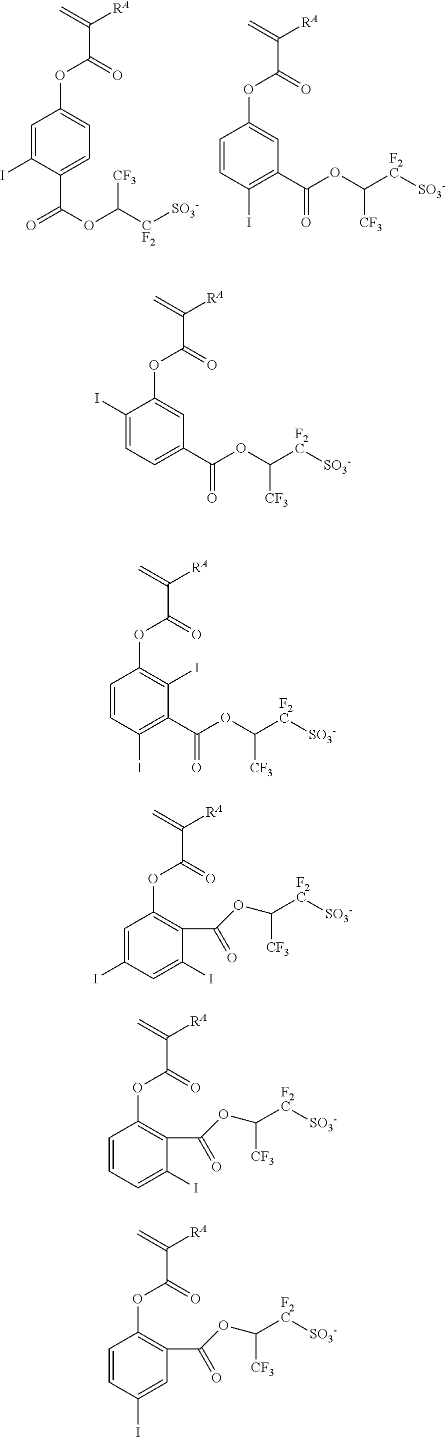

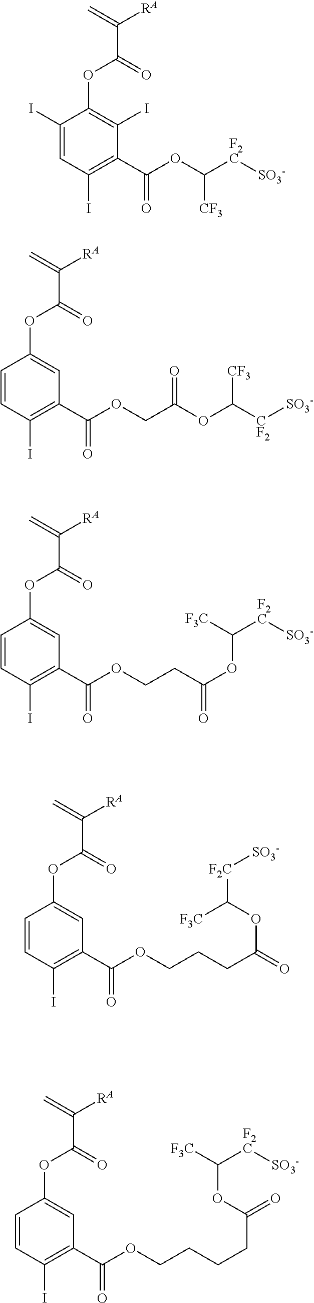

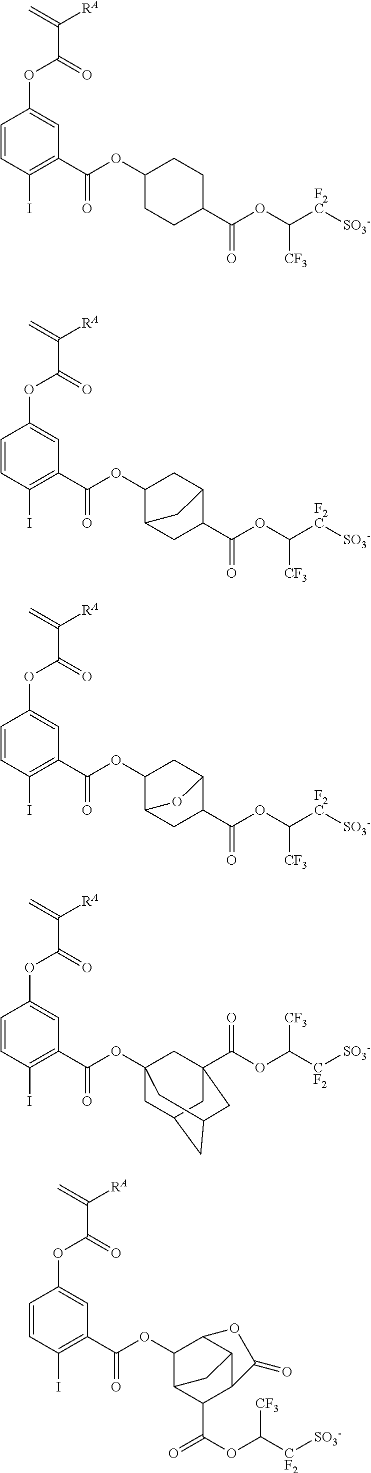

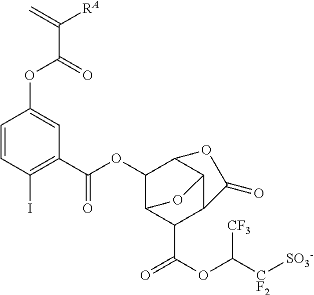

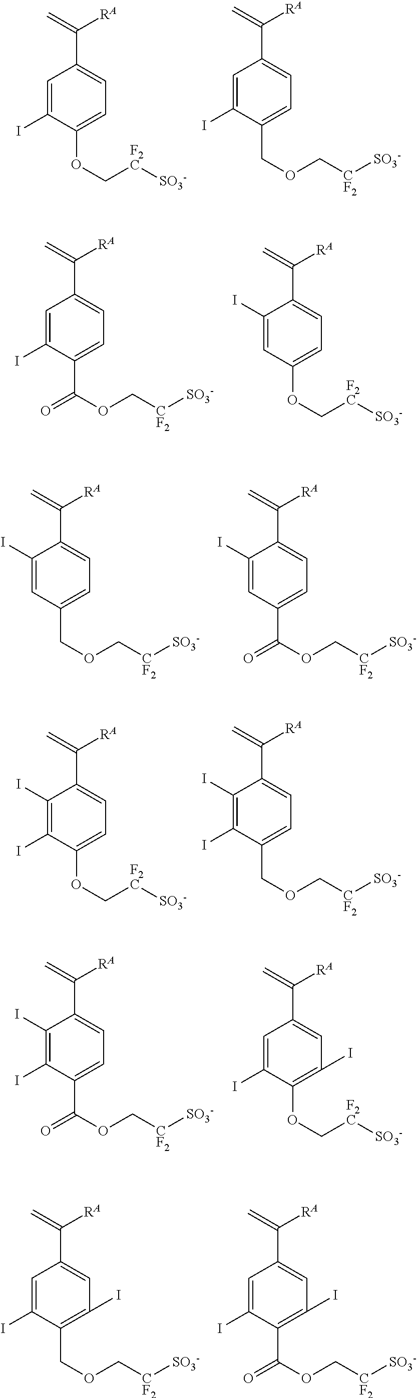

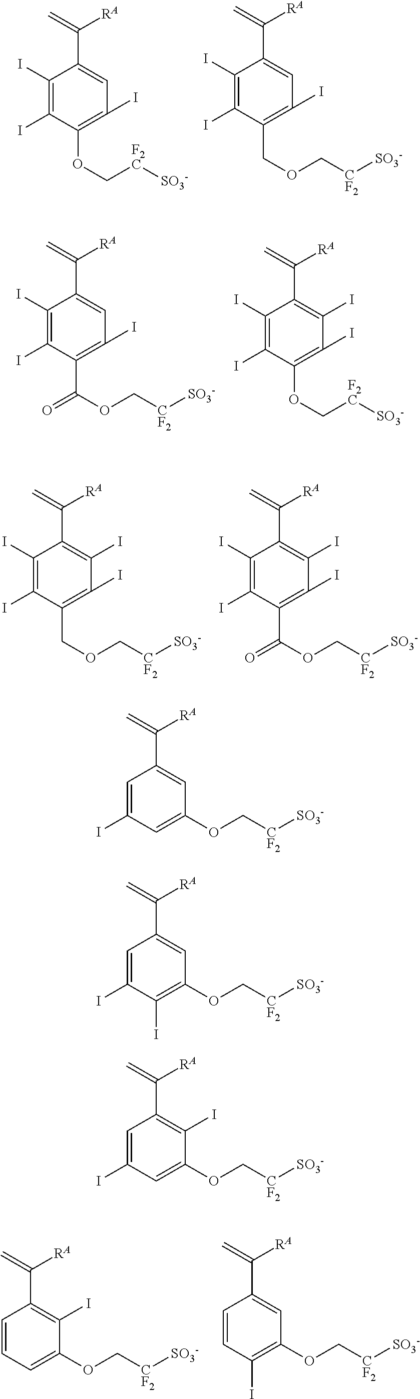

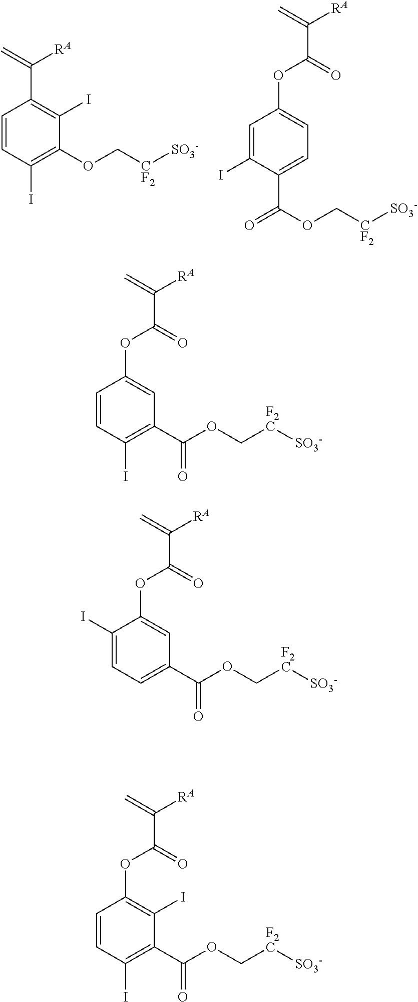

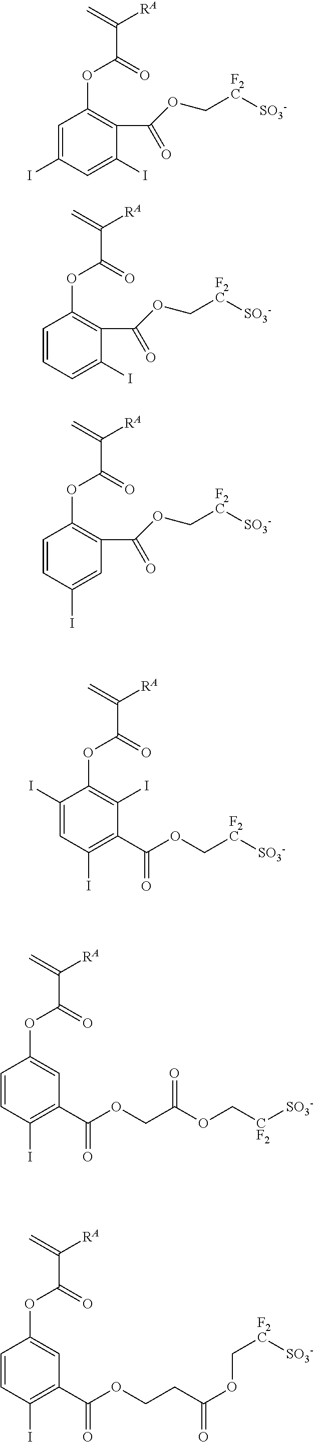

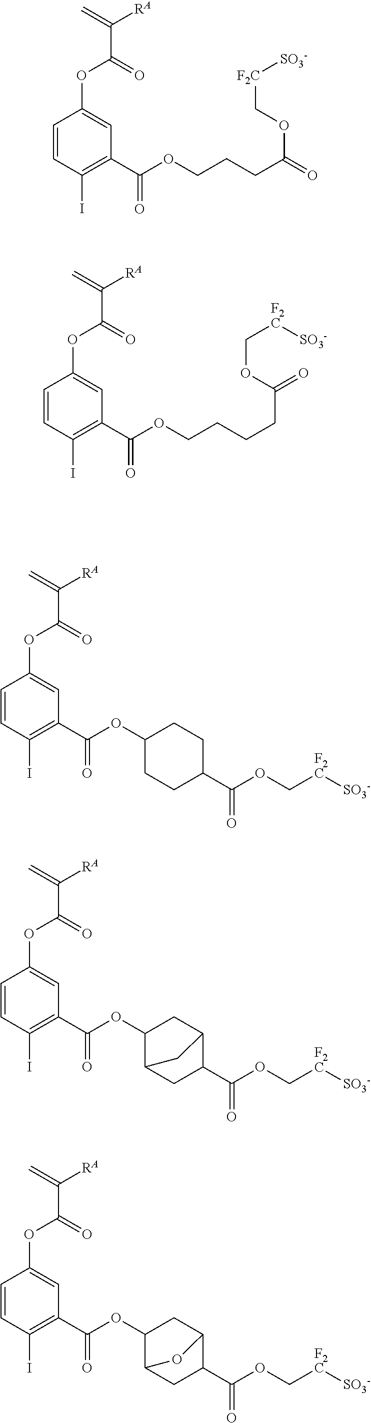

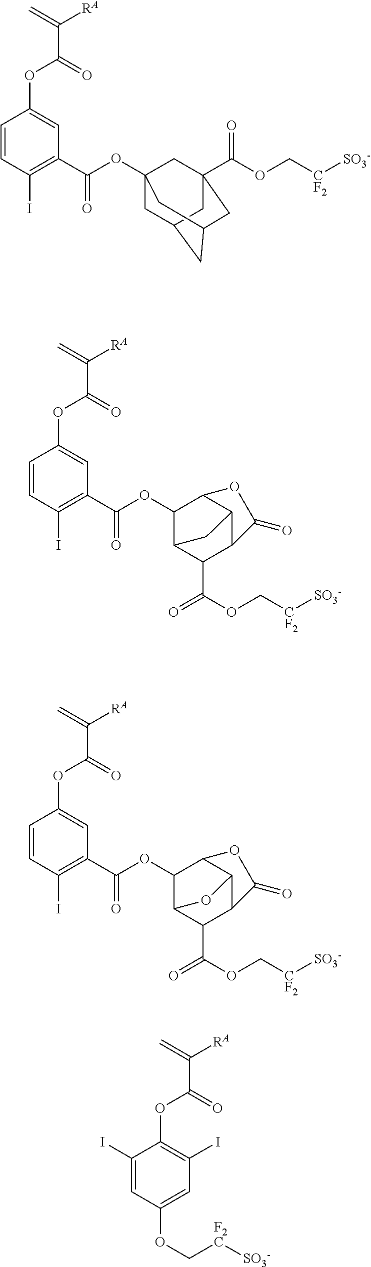

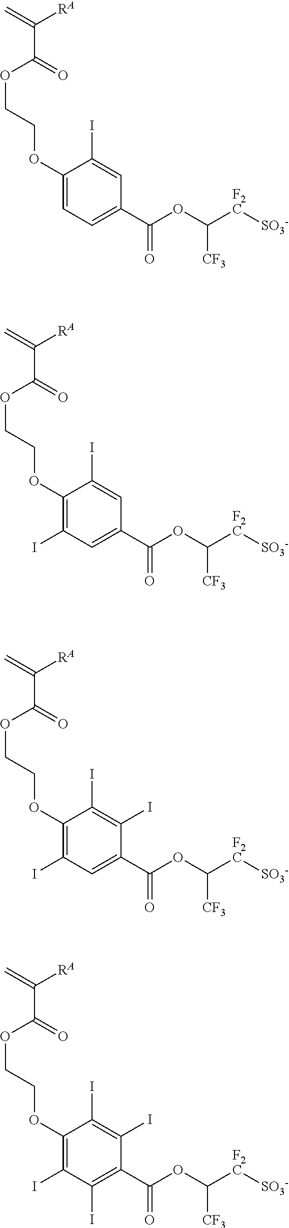

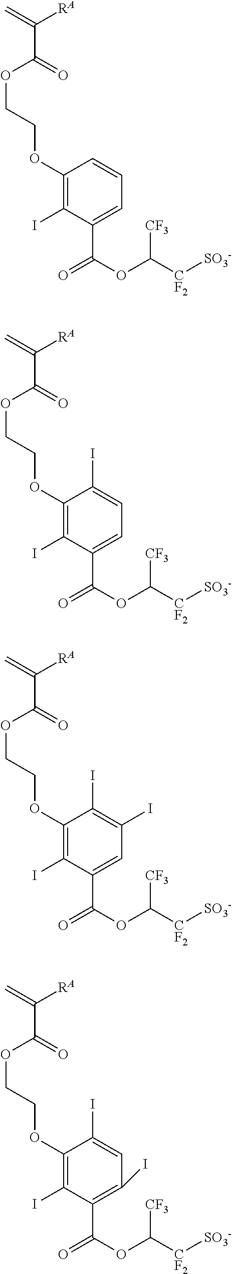

Examples of the monomer from which repeat unit (0) is derived are shown below, but not limited thereto. R^{A} is as defined above.

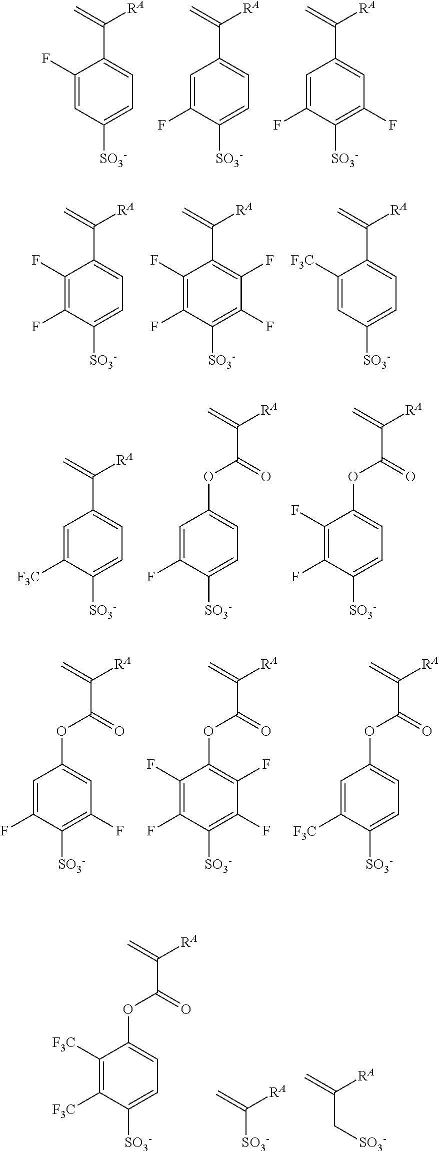

The repeat units (f1) to (f3) function as an acid generator. The binding of an acid generator to the polymer main chain is effective in restraining acid diffusion, thereby preventing a reduction of resolution due to blur by acid diffusion. Also, LWR or CDU is improved since the acid generator is uniformly distributed.

The base polymer for formulating the positive resist composition comprises repeat units (a1) or (a2) having an acid labile group as essential component and additional repeat units (b), (c), (d), (e), and (f) as optional components. A fraction of units (a1), (a2), (b), (c), (d), (e), and (f) is: preferably 0≤a1<1.0, 0≤a2<1.0, 0<a1+a2<1.0, 0≤b≤0.9, 0≤c≤0.9, 0≤d≤0.8, 0≤e≤0.8, and 0≤f≤0.5; more preferably 0≤a1≤0.9, 0≤a2≤0.9, 0.1≤a1+a2≤0.9, 0≤b≤0.8, 0≤c≤0.8, 0≤d≤0.7, 0≤e≤0.7, and 0≤f≤0.4; and even more preferably 0≤a1≤0.8, 0≤a2≤0.8, 0.1≤a1+a2≤0.8, 0≤b≤0.75, 0≤c<0.75, 0≤d≤0.6, 0≤e≤0.6, and 0≤f≤0.3. Notably, f=f1+f2+f3, meaning that unit (f) is at least one of units (f1) to (f3), and a1+a2+b+c+d+e+f=1.0.

For the base polymer for formulating the negative resist composition, an acid labile group is not necessarily essential. The base polymer comprises repeat units (b), and optionally repeat units (c), (d), (e), and/or (f). A fraction of these units is: preferably 0<b≤1.0, 0≤c≤0.9, 0≤d≤0.8, 0≤e≤0.8, and 0≤f≤0.5; more preferably 0.2≤b≤1.0, 0≤c≤0.8, 0≤d≤0.7, 0≤e≤0.7, and 0≤f≤0.4; and even more preferably 0.3≤b≤1.0, 0≤c≤0.75, 0≤d≤0.6, 0≤e≤0.6, and 0≤f≤0.3. Notably, f=f1+f2+f3, meaning that unit (f) is at least one of units (f1) to (f3), and b+c+d+e+f=1.0.

The base polymer may be synthesized by any desired methods, for example, by dissolving one or more monomers selected from the monomers corresponding to the foregoing repeat units in an organic solvent, adding a radical polymerization initiator thereto, and heating for polymerization. Examples of the organic solvent which can be used for polymerization include toluene, benzene, tetrahydrofuran (THF), diethyl ether, and dioxane. Examples of the polymerization initiator used herein include 2,2′-azobisisobutyronitrile (AIBN), 2,2′-azobis(2,4-dimethylvaleronitrile), dimethyl 2,2-azobis(2-methylpropionate), benzoyl peroxide, and lauroyl peroxide. Preferably, the reaction temperature is 50 to 80° C. and the reaction time is 2 to 100 hours, more preferably 5 to 20 hours.

Where a monomer having a hydroxy group is copolymerized, the hydroxy group may be replaced by an acetal group susceptible to deprotection with acid, typically ethoxyethoxy, prior to polymerization, and the polymerization be followed by deprotection with weak acid and water. Alternatively, the hydroxy group may be replaced by an acetyl, formyl, pivaloyl or similar group prior to polymerization, and the polymerization be followed by alkaline hydrolysis.

When hydroxystyrene or hydroxyvinylnaphthalene is copolymerized, an alternative method is possible. Specifically, acetoxystyrene or acetoxyvinylnaphthalene is used instead of hydroxystyrene or hydroxyvinylnaphthalene, and after polymerization, the acetoxy group is deprotected by alkaline hydrolysis, for thereby converting the polymer product to hydroxystyrene or hydroxyvinylnaphthalene. For alkaline hydrolysis, a base such as aqueous ammonia or triethylamine may be used. Preferably the reaction temperature is −20° C. to 100° C., more preferably 0° C. to 60° C., and the reaction time is 0.2 to 100 hours, more preferably 0.5 to 20 hours.

The base polymer should preferably have a weight average molecular weight (Mw) in the range of 1,000 to 500,000, and more preferably 2,000 to 30,000, as measured by GPC versus polystyrene standards using tetrahydrofuran (THF) solvent. A Mw in the range ensures that the resist film has heat resistance and high solubility in alkaline developer.

If a base polymer has a wide molecular weight distribution or dispersity (Mw/Mn), which indicates the presence of lower and higher molecular weight polymer fractions, there is a possibility that foreign matter is left on the pattern or the pattern profile is degraded. The influences of Mw and Mw/Mn become stronger as the pattern rule becomes finer. Therefore, the base polymer should preferably have a narrow dispersity (Mw/Mn) of 1.0 to 2.0, especially 1.0 to 1.5, in order to provide a resist composition suitable for micropatterning to a small feature size.

It is understood that a blend of two or more polymers which differ in compositional ratio, Mw or Mw/Mn is acceptable.

### [Organic Solvent]

An organic solvent may be added to the resist composition. The organic solvent used herein is not particularly limited as long as the foregoing and other components are soluble therein. Examples of the organic solvent are described in JP-A 2008-111103, paragraphs [0144]-[0145](U.S. Pat. No. 7,537,880). Exemplary solvents include ketones such as cyclohexanone, cyclopentanone, methyl-2-n-pentyl ketone and 2-heptanone; alcohols such as 3-methoxybutanol, 3-methyl-3-methoxybutanol, 1-methoxy-2-propanol, 1-ethoxy-2-propanol, and diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as γ-butyrolactone, which may be used alone or in admixture.

The organic solvent is preferably added in an amount of 100 to 10,000 parts, and more preferably 200 to 8,000 parts by weight per 100 parts by weight of the base polymer.

### [Quencher]

The resist composition may comprise a quencher. As used herein, the “quencher” refers to a compound capable of trapping the acid generated from the acid generator for thereby preventing the acid from diffusing to the unexposed region.

The quencher is typically selected from conventional basic compounds. Conventional basic compounds include primary, secondary, and tertiary aliphatic amines, mixed amines, aromatic amines, heterocyclic amines, nitrogen-containing compounds with carboxy group, nitrogen-containing compounds with sulfonyl group, nitrogen-containing compounds with hydroxy group, nitrogen-containing compounds with hydroxyphenyl group, alcoholic nitrogen-containing compounds, amide derivatives, imide derivatives, and carbamate derivatives. Also included are primary, secondary, and tertiary amine compounds, specifically amine compounds having a hydroxy group, ether bond, ester bond, lactone ring, cyano group, or sulfonic ester bond as described in U.S. Pat. No. 7,537,880 (JP-A 2008-111103, paragraphs [0146]-[0164]), and compounds having a carbamate bond as described in JP 3790649. Addition of a basic compound is effective for further suppressing the diffusion rate of acid in the resist film or correcting the pattern profile.

Suitable quenchers also include onium salts such as sulfonium salts, iodonium salts and ammonium salts of sulfonic acids which are not fluorinated at α-position, carboxylic acids or fluorinated alkoxides, as described in U.S. Pat. No. 8,795,942 (JP-A 2008-158339). While an α-fluorinated sulfonic acid, imide acid, and methide acid are necessary to deprotect the acid labile group of carboxylic acid ester, an α-non-fluorinated sulfonic acid, carboxylic acid or fluorinated alcohol is released by salt exchange with an α-non-fluorinated onium salt. The α-non-fluorinated sulfonic acid, carboxylic acid and fluorinated alcohol function as a quencher because they do not induce deprotection reaction.

Exemplary such quenchers include a compound (onium salt of α-non-fluorinated sulfonic acid) having the formula (4), a compound (onium salt of carboxylic acid) having the formula (5), and a compound (onium salt of alkoxide) having the formula (6).

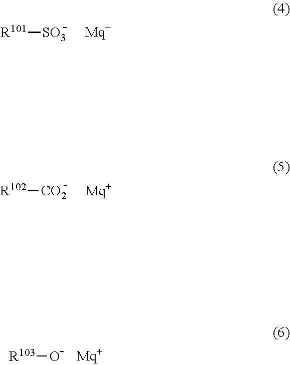

In formula (4), R¹⁰¹ is hydrogen or a C₁-C₄₀ hydrocarbyl group which may contain a heteroatom, exclusive of the hydrocarbyl group in which the hydrogen bonded to the carbon atom at α-position of the sulfo group is substituted by fluorine or fluoroalkyl moiety.

The C₁-C₄₀ hydrocarbyl group may be saturated or unsaturated and straight, branched or cyclic. Examples thereof include C₁-C₄₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, n-decyl; C₃-C₄₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decyl, adamantyl, and adamantylmethyl; C₂-C₄₀ alkenyl groups such as vinyl, allyl, butenyl and hexenyl; C₃-C₄₀ cyclic unsaturated aliphatic hydrocarbyl groups such as cyclohexenyl; C₆-C₄₀ aryl groups such as phenyl, naphthyl, alkylphenyl groups (e.g., 2-methylphenyl, 3-methylphenyl, 4-methylphenyl, 4-ethylphenyl, 4-tert-butylphenyl, 4-n-butylphenyl), di- or trialkylphenyl groups (e.g., 2,4-dimethylphenyl and 2,4,6-triisopropylphenyl), alkylnaphthyl groups (e.g., methylnaphthyl and ethylnaphthyl), dialkylnaphthyl groups (e.g., dimethylnaphthyl and diethylnaphthyl); and C₇-C₄₀ aralkyl groups such as benzyl, 1-phenylethyl and 2-phenylethyl.

In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, carbonyl moiety, ether bond, ester bond, sulfonic ester bond, carbonate bond, lactone ring, sultone ring, carboxylic anhydride (—C(═O)—O—C(═O)—), or haloalkyl moiety. Suitable heteroatom-containing hydrocarbyl groups include heteroaryl groups such as thienyl, 4-hydroxyphenyl, alkoxyphenyl groups such as 4-methoxyphenyl, 3-methoxyphenyl, 2-methoxyphenyl, 4-ethoxyphenyl, 4-tert-butoxyphenyl, 3-tert-butoxyphenyl; alkoxynaphthyl groups such as methoxynaphthyl, ethoxynaphthyl, n-propoxynaphthyl and n-butoxynaphthyl; dialkoxynaphthyl groups such as dimethoxynaphthyl and diethoxynaphthyl; and aryloxoalkyl groups, typically 2-aryl-2-oxoethyl groups such as 2-phenyl-2-oxoethyl, 2-(1-naphthyl)-2-oxoethyl and 2-(2-naphthyl)-2-oxoethyl.

In formula (5), R¹⁰² is a C₁-C₄₀ hydrocarbyl group which may contain a heteroatom. Examples of the hydrocarbyl group R¹⁰² are as exemplified above for the hydrocarbyl group R¹⁰¹. Also included are fluorinated alkyl groups such as trifluoromethyl, trifluoroethyl, 2,2,2-trifluoro-1-methyl-1-hydroxyethyl, 2,2,2-trifluoro-1-(trifluoromethyl)-1-hydroxyethyl, and fluorinated aryl groups such as pentafluorophenyl and 4-trifluoromethylphenyl.

In formula (6), R¹⁰³ is a C₁-C₈ saturated hydrocarbyl group containing at least 3 fluorine atoms or a C₆-C₁₀ aryl group containing at least 3 fluorine atoms, the hydrocarbyl and aryl groups optionally containing a nitro moiety.

In formulae (4), (5) and (6), Mq⁺ is an onium cation. The onium cation is preferably a sulfonium, iodonium or ammonium cation, with the sulfonium cation being more preferred. Suitable sulfonium cations are as exemplified for the sulfonium cation M⁺ in formula (1).

A sulfonium salt of iodized benzene ring-containing carboxylic acid having the formula (7) is also useful as the quencher.

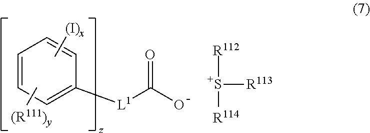

In formula (7), x is an integer of 1 to 5, y is an integer of 0 to 3, and z is an integer of 1 to 3.

In formula (7), R¹¹¹ is hydroxy, fluorine, chlorine, bromine, amino, nitro, cyano, or a C₁-C₆ saturated hydrocarbyl, C₁-C₆ saturated hydrocarbyloxy, C₂-C₆ saturated hydrocarbylcarbonyloxy, or C₁-C₄ saturated hydrocarbylsulfonyloxy group, in which some or all hydrogen may be substituted by halogen, or —N(R^{111A})—C(═O)—R^{111B}, —N(R^{111A})—C(═O)—O—R^{111B}. R^{111A} is hydrogen or a C₁-C₆ saturated hydrocarbyl group. R^{111B} is a C₁-C₆ saturated hydrocarbyl or C₂-C₈ unsaturated aliphatic hydrocarbyl group. A plurality of R¹¹¹ may be identical or different when y and/or z is 2 or 3.

In formula (7), L¹ is a single bond, or a C₁-C₂₀ (z+1)-valent linking group which may contain at least one moiety selected from ether bond, carbonyl, ester bond, amide bond, sultone ring, lactam ring, carbonate bond, halogen, hydroxy and carboxy moiety. The saturated hydrocarbyl, saturated hydrocarbyloxy, saturated hydrocarbylcarbonyloxy and saturated hydrocarbylsulfonyloxy groups may be straight, branched or cyclic.

In formula (7), R¹¹², R¹¹³ and R¹¹⁴ are each independently halogen or a C₁-C₂₀ hydrocarbyl group which may contain a heteroatom. The hydrocarbyl group may be saturated or unsaturated and straight, branched or cyclic. Examples thereof are as exemplified for the hydrocarbyl groups R⁶ to R¹⁰ in formulae (2) and (3).

Examples of the compound having formula (7) include those described in U.S. Pat. No. 10,295,904 (JP-A 2017-219836) and US 20210188770 (JP-A 2021-091666).

Also useful are quenchers of polymer type as described in U.S. Pat. No. 7,598,016 (JP-A 2008-239918). The polymeric quencher segregates at the resist film surface and thus enhances the rectangularity of resist pattern. When a protective film is applied as is often the case in the immersion lithography, the polymeric quencher is also effective for preventing a film thickness loss of resist pattern or rounding of pattern top.

Other useful quenchers include sulfonium salts of betaine structure as described in JP 6848776 and JP-A 2020-037544, fluorine-free methide acids as described in JP-A 2020-055797, sulfonium salts of sulfonamide as described in JP 5807552, and sulfonium salts of iodized sulfonamide, phenols, halogens or acid generators capable of generating carbonic acid as described in JP-A 2019-211751.

The quencher is preferably added in an amount of 0 to 5 parts, more preferably 0 to 4 parts by weight per 100 parts by weight of the base polymer. The quencher may be used alone or in admixture.

### [Other Components]

In addition to the foregoing components, the resist composition may contain other components such as an acid generator other than the salt having formula (1), surfactant, dissolution inhibitor, crosslinker, water repellency improver and acetylene alcohol. Each of the other components may be used alone or in admixture.

The other acid generator is typically a compound (PAG) capable of generating an acid upon exposure to actinic ray or radiation. Although the PAG used herein may be any compound capable of generating an acid upon exposure to high-energy radiation, those compounds capable of generating sulfonic acid, imide acid (imidic acid) or methide acid are preferred. Suitable PAGs include sulfonium salts, iodonium salts, sulfonyldiazomethane, N-sulfonyloxyimide, and oxime-O-sulfonate acid generators. Exemplary PAGs are described in JP-A 2008-111103, paragraphs [0122]-[0142](U.S. Pat. No. 7,537,880), JP-A 2018-005224, and JP-A 2018-025789. The other acid generator is preferably used in an amount of 0 to 200 parts, more preferably 0.1 to 100 parts by weight per 100 parts by weight of the base polymer.

Exemplary surfactants are described in JP-A 2008-111103, paragraphs [0165]-[0166]. Inclusion of a surfactant may improve or control the coating characteristics of the resist composition. The surfactant is preferably added in an amount of 0.0001 to 10 parts by weight per 100 parts by weight of the base polymer.

In the embodiment wherein the resist composition is of positive tone, the inclusion of a dissolution inhibitor may lead to an increased difference in dissolution rate between exposed and unexposed areas and a further improvement in resolution. The dissolution inhibitor is typically a compound having at least two phenolic hydroxy groups on the molecule, in which an average of from 0 to 100 mol % of all the hydrogen atoms on the phenolic hydroxy groups are replaced by acid labile groups or a compound having at least one carboxy group on the molecule, in which an average of 50 to 100 mol % of all the hydrogen atoms on the carboxy groups are replaced by acid labile groups, both the compounds having a molecular weight of 100 to 1,000, and preferably 150 to 800. Typical are bisphenol A, trisphenol, phenolphthalein, cresol novolac, naphthalenecarboxylic acid, adamantanecarboxylic acid, and cholic acid derivatives in which the hydrogen atom on the hydroxy or carboxy group is replaced by an acid labile group, as described in U.S. Pat. No. 7,771,914 (JP-A 2008-122932, paragraphs [0155]-[0178]).

When the resist composition is of positive tone and contains a dissolution inhibitor, the dissolution inhibitor is preferably added in an amount of 0 to 50 parts, more preferably 5 to 40 parts by weight per 100 parts by weight of the base polymer.

In the case of negative resist compositions, a negative pattern may be formed by adding a crosslinker to reduce the dissolution rate of exposed area. Suitable crosslinkers which can be used herein include epoxy compounds, melamine compounds, guanamine compounds, glycoluril compounds and urea compounds having substituted thereon at least one group selected from among methylol, alkoxymethyl and acyloxymethyl groups, isocyanate compounds, azide compounds, and compounds having a double bond such as an alkenyloxy group. These compounds may be used as an additive or introduced into a polymer side chain as a pendant. Hydroxy-containing compounds may also be used as the crosslinker.

Suitable epoxy compounds include tris(2,3-epoxypropyl) isocyanurate, trimethylolmethane triglycidyl ether, trimethylolpropane triglycidyl ether, and triethylolethane triglycidyl ether. Examples of the melamine compound include hexamethylol melamine, hexamethoxymethyl melamine, hexamethylol melamine compounds having 1 to 6 methylol groups methoxymethylated and mixtures thereof, hexamethoxyethyl melamine, hexaacyloxymethyl melamine, hexamethylol melamine compounds having 1 to 6 methylol groups acyloxymethylated and mixtures thereof. Examples of the guanamine compound include tetramethylol guanamine, tetramethoxymethyl guanamine, tetramethylol guanamine compounds having 1 to 4 methylol groups methoxymethylated and mixtures thereof, tetramethoxyethyl guanamine, tetraacyloxyguanamine, tetramethylol guanamine compounds having 1 to 4 methylol groups acyloxymethylated and mixtures thereof. Examples of the glycoluril compound include tetramethylol glycoluril, tetramethoxyglycoluril, tetramethoxymethyl glycoluril, tetramethylol glycoluril compounds having 1 to 4 methylol groups methoxymethylated and mixtures thereof, tetramethylol glycoluril compounds having 1 to 4 methylol groups acyloxymethylated and mixtures thereof. Examples of the urea compound include tetramethylol urea, tetramethoxymethyl urea, tetramethylol urea compounds having 1 to 4 methylol groups methoxymethylated and mixtures thereof, and tetramethoxyethyl urea.

Suitable isocyanate compounds include tolylene diisocyanate, diphenylmethane diisocyanate, hexamethylene diisocyanate and cyclohexane diisocyanate. Suitable azide compounds include 1,1′-biphenyl-4,4′-bisazide, 4,4′-methylidenebisazide, and 4,4′-oxybisazide. Examples of the alkenyloxy group-containing compound include ethylene glycol divinyl ether, triethylene glycol divinyl ether, 1,2-propanediol divinyl ether, 1,4-butanediol divinyl ether, tetramethylene glycol divinyl ether, neopentyl glycol divinyl ether, trimethylol propane trivinyl ether, hexanediol divinyl ether, 1,4-cyclohexanediol divinyl ether, pentaerythritol trivinyl ether, pentaerythritol tetravinyl ether, sorbitol tetravinyl ether, sorbitol pentavinyl ether, and trimethylol propane trivinyl ether.

When the resist composition is of negative tone and contains a crosslinker, the crosslinker is preferably added in an amount of 0.1 to 50 parts, more preferably 1 to 40 parts by weight per 100 parts by weight of the base polymer.

To the resist composition, a water repellency improver may also be added for improving the water repellency on surface of a resist film. The water repellency improver may be used in the topcoatless immersion lithography. Suitable water repellency improvers include polymers having a fluoroalkyl group and polymers having a specific structure with a 1,1,1,3,3,3-hexafluoro-2-propanol residue and are described in JP-A 2007-297590 and JP-A 2008-111103, for example. The water repellency improver to be added to the resist composition should be soluble in alkaline developers and organic solvent developers. The water repellency improver of specific structure with a 1,1,1,3,3,3-hexafluoro-2-propanol residue is well soluble in the developer. A polymer having an amino group or amine salt copolymerized as repeat units may serve as the water repellent additive and is effective for preventing evaporation of acid during PEB, thus preventing any hole pattern opening failure after development. An appropriate amount of the water repellency improver is 0 to 20 parts, preferably 0.5 to 10 parts by weight per 100 parts by weight of the base polymer.

Also, an acetylene alcohol may be blended in the resist composition. Suitable acetylene alcohols are described in JP-A 2008-122932, paragraphs [0179]-[0182]. An appropriate amount of the acetylene alcohol blended is 0 to 5 parts by weight per 100 parts by weight of the base polymer.

### [Process]

The resist composition is used in the fabrication of various integrated circuits. Pattern formation using the resist composition may be performed by well-known lithography processes. The process generally involves the steps of applying the resist composition onto a substrate to form a resist film thereon, exposing the resist film to high-energy radiation, and developing the exposed resist film in a developer. If necessary, any additional steps may be added.

Specifically, the resist composition is first applied onto a substrate on which an integrated circuit is to be formed (e.g., Si, SiO₂, SiN, SiON, TiN, WSi, BPSG, SOG, or organic antireflective coating) or a substrate on which a mask circuit is to be formed (e.g., Cr, CrO, CrON, MoSi₂, or SiO₂) by a suitable coating technique such as spin coating, roll coating, flow coating, dipping, spraying or doctor coating. The coating is prebaked on a hotplate preferably at a temperature of 60 to 150° C. for 10 seconds to 30 minutes, more preferably at 80 to 120° C. for 30 seconds to 20 minutes. The resulting resist film is generally 0.01 to 2 μm thick.

The resist film is then exposed to a desired pattern of high-energy radiation such as UV, deep-UV, EB, EUV of wavelength 3 to 15 nm, x-ray, soft x-ray, excimer laser light, γ-ray or synchrotron radiation. When UV, deep-UV, EUV, x-ray, soft x-ray, excimer laser light, γ-ray or synchrotron radiation is used as the high-energy radiation, the resist film is exposed thereto directly or through a mask having a desired pattern in a dose of preferably about 1 to 200 mJ/cm², more preferably about 10 to 100 mJ/cm². When EB is used as the high-energy radiation, the resist film is exposed thereto directly or through a mask having a desired pattern in a dose of preferably about 0.1 to 300 μC/cm², more preferably about 0.5 to 200 μC/cm². It is appreciated that the inventive resist composition is suited in micropatterning using KrF excimer laser, ArF excimer laser, EB, EUV, x-ray, soft x-ray, γ-ray or synchrotron radiation, especially in micropatterning using EB or EUV.

After the exposure, the resist film may be baked (PEB) on a hotplate or in an oven preferably at 30 to 150° C. for 10 seconds to 30 minutes, more preferably at 50 to 120° C. for 30 seconds to 20 minutes.

After the exposure or PEB, the resist film is developed in a developer in the form of an aqueous base solution for 3 seconds to 3 minutes, preferably 5 seconds to 2 minutes by conventional techniques such as dip, puddle and spray techniques. A typical developer is a 0.1 to 10 wt %, preferably 2 to 5 wt % aqueous solution of tetramethylammonium hydroxide (TMAH), tetraethylammonium hydroxide (TEAH), tetrapropylammonium hydroxide (TPAH), or tetrabutylammonium hydroxide (TBAH). In the case of positive tone, the resist film in the exposed area is dissolved in the developer whereas the resist film in the unexposed area is not dissolved. In this way, the desired positive pattern is formed on the substrate. In the case of negative tone, inversely the resist film in the exposed area is insolubilized whereas the resist film in the unexposed area is dissolved away.

In an alternative embodiment, a negative pattern can be obtained from the positive resist composition comprising a base polymer containing acid labile groups by effecting organic solvent development. The developer used herein is preferably selected from among 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate, and mixtures thereof.

At the end of development, the resist film is rinsed. As the rinsing liquid, a solvent which is miscible with the developer and does not dissolve the resist film is preferred. Suitable solvents include alcohols of 3 to 10 carbon atoms, ether compounds of 8 to 12 carbon atoms, alkanes, alkenes, and alkynes of 6 to 12 carbon atoms, and aromatic solvents. Specifically, suitable alcohols of 3 to 10 carbon atoms include n-propyl alcohol, isopropyl alcohol, 1-butyl alcohol, 2-butyl alcohol, isobutyl alcohol, t-butyl alcohol, 1-pentanol, 2-pentanol, 3-pentanol, t-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, and 1-octanol. Suitable ether compounds of 8 to 12 carbon atoms include di-n-butyl ether, diisobutyl ether, di-s-butyl ether, di-n-pentyl ether, diisopentyl ether, di-s-pentyl ether, di-t-pentyl ether, and di-n-hexyl ether. Suitable alkanes of 6 to 12 carbon atoms include hexane, heptane, octane, nonane, decane, undecane, dodecane, methylcyclopentane, dimethylcyclopentane, cyclohexane, methylcyclohexane, dimethylcyclohexane, cycloheptane, cyclooctane, and cyclononane. Suitable alkenes of 6 to 12 carbon atoms include hexene, heptene, octene, cyclohexene, methylcyclohexene, dimethylcyclohexene, cycloheptene, and cyclooctene. Suitable alkynes of 6 to 12 carbon atoms include hexyne, heptyne, and octyne. Suitable aromatic solvents include toluene, xylene, ethylbenzene, isopropylbenzene, t-butylbenzene and mesitylene.

Rinsing is effective for minimizing the risks of resist pattern collapse and defect formation. However, rinsing is not essential. If rinsing is omitted, the amount of solvent used may be reduced.

A hole or trench pattern after development may be shrunk by the thermal flow, RELACS® or DSA process. A hole pattern is shrunk by coating a shrink agent thereto, and baking such that the shrink agent may undergo crosslinking at the resist surface as a result of the acid catalyst diffusing from the resist layer during bake, and the shrink agent may attach to the sidewall of the hole pattern. The bake is preferably at a temperature of 70 to 180° C., more preferably 80 to 170° C., for a time of 10 to 300 seconds. The extra shrink agent is stripped and the hole pattern is shrunk.

### EXAMPLES

Examples of the invention are given below by way of illustration and not by way of limitation. All parts are by weight (pbw). THF stands for tetrahydrofuran.

Bis-onium salts PAG-PDQ-1 to PAG-PDQ-17 used in resist compositions as acid generator/quencher have the structure shown below.

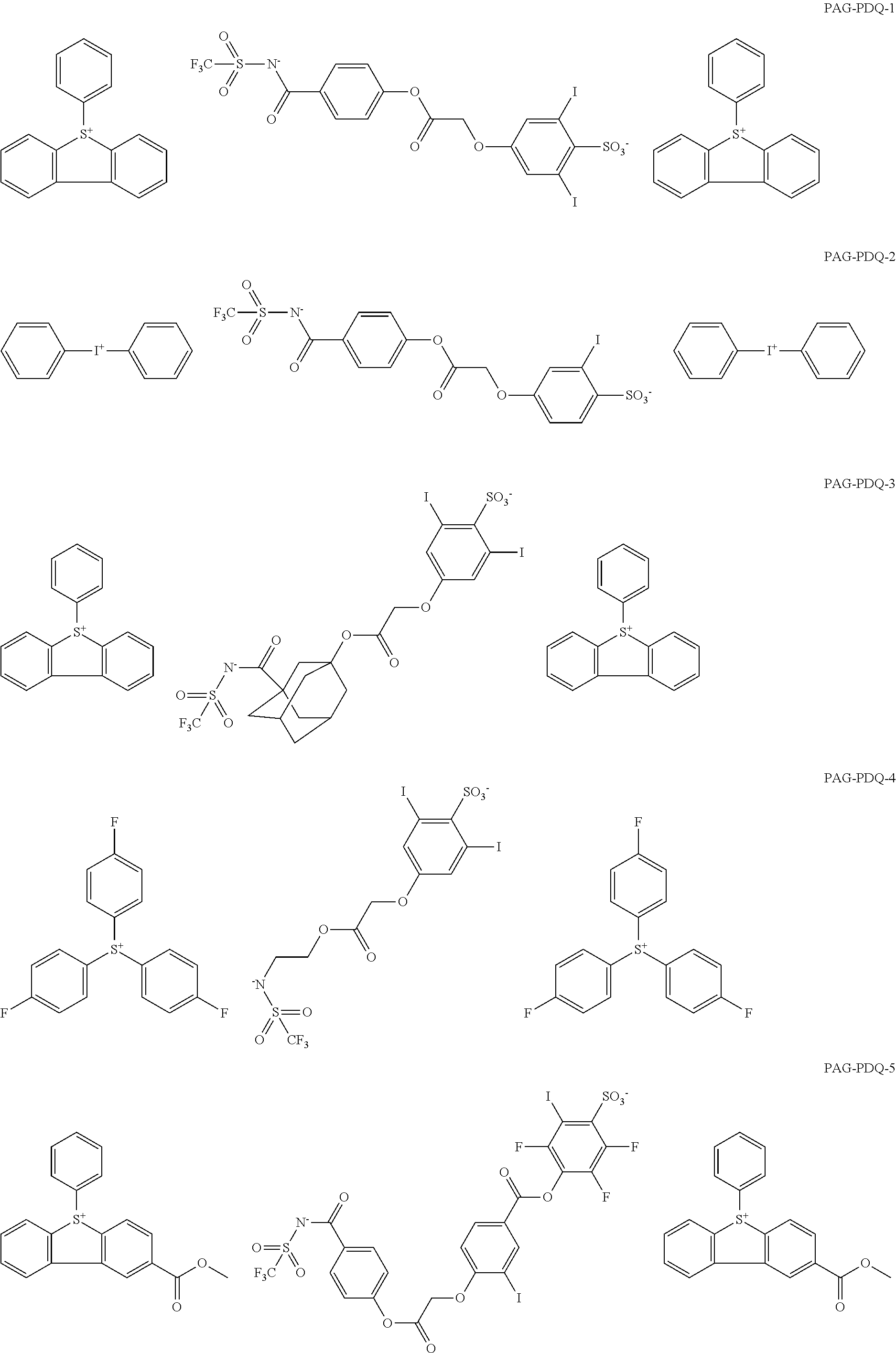

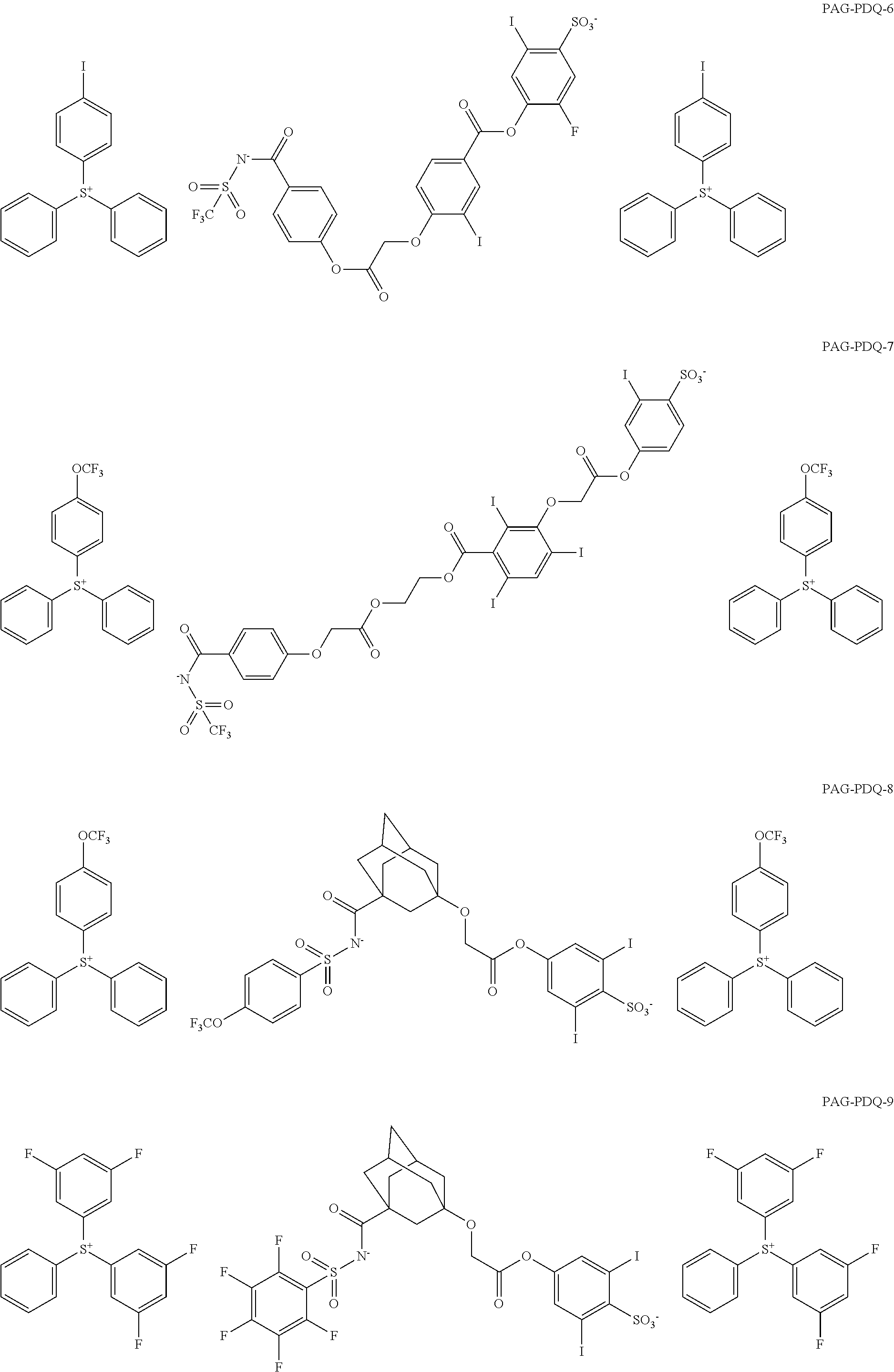

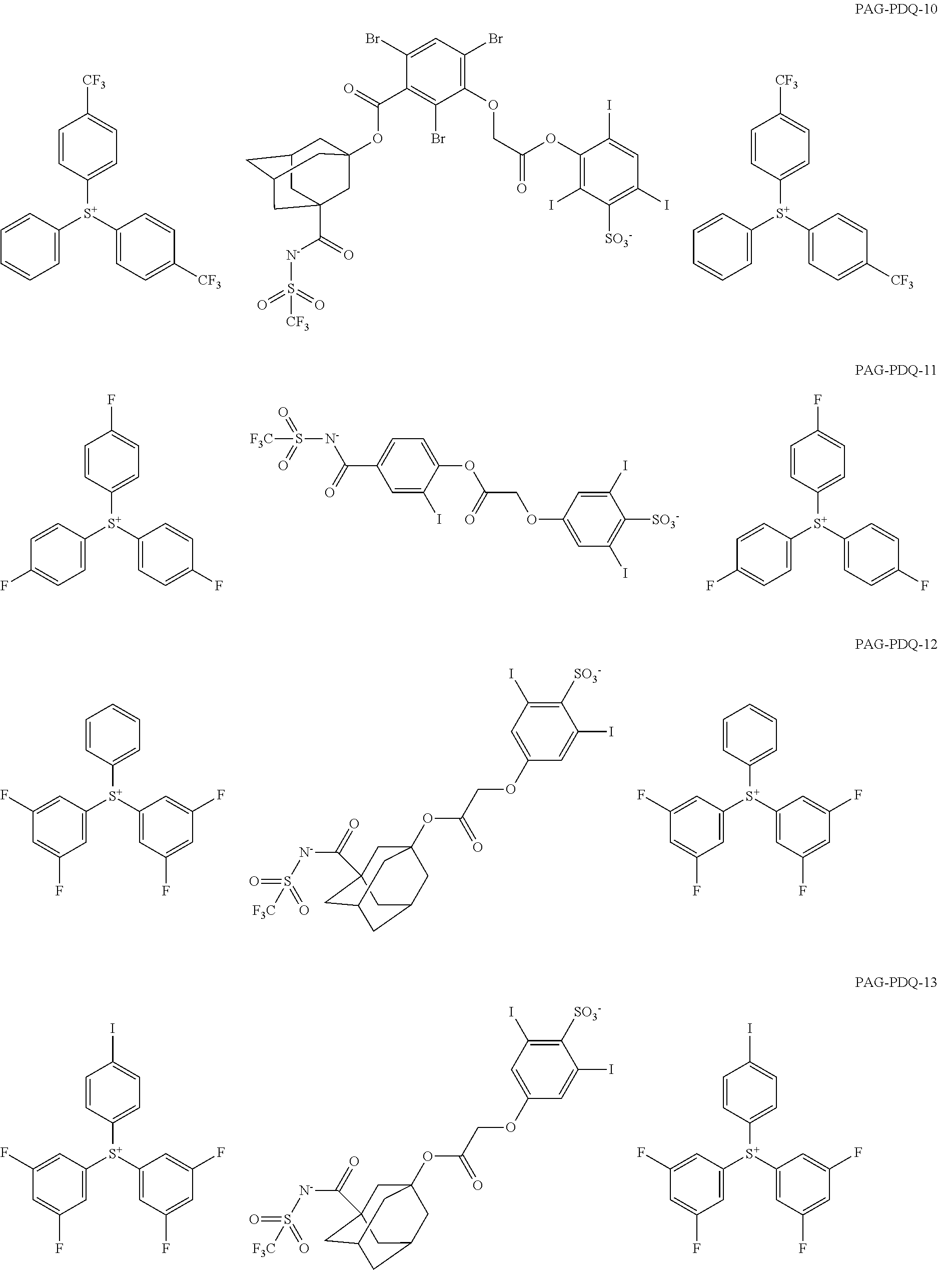

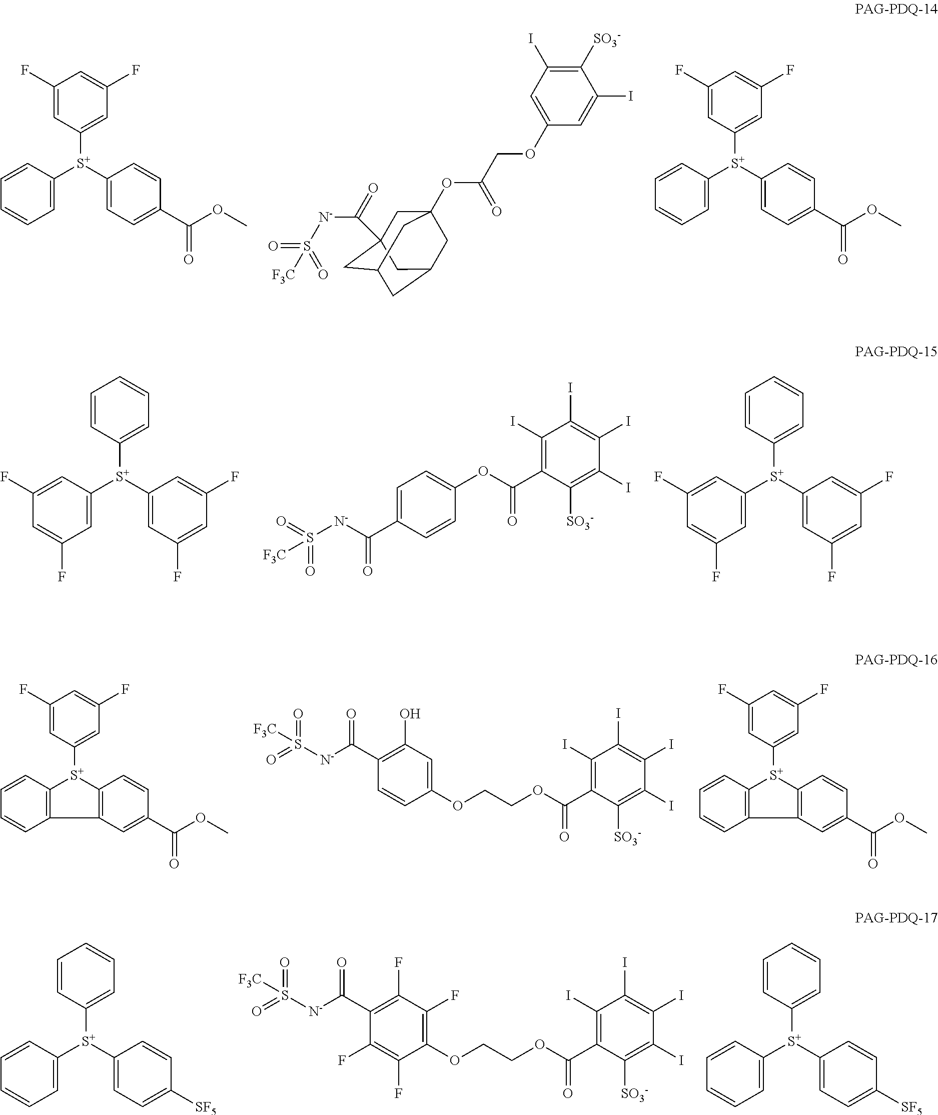

### [Synthesis Example] Synthesis of Base Polymers (Polymers P-1 to P-4)

Base polymers (Polymers P-1 to P-4) of the structure shown below were synthesized by combining selected monomers, effecting copolymerization reaction in THF solvent, pouring the reaction solution into methanol, washing the solid precipitate with hexane, isolating, and drying. The base polymers were analyzed for composition by ¹H-NMR spectroscopy and for Mw and Mw/Mn by GPC versus polystyrene standards using THF solvent.

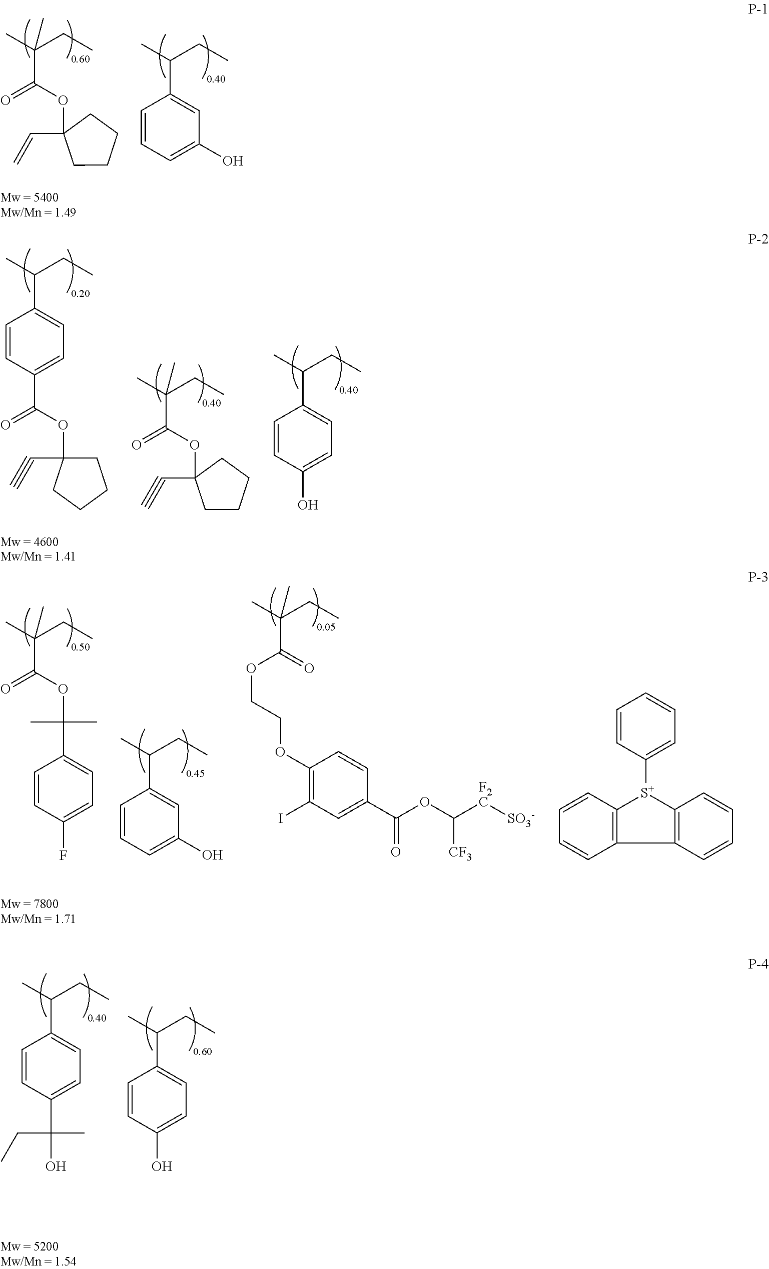

### [Examples 1 to 22 and Comparative Examples 1 to 3] Preparation and Evaluation of Resist Compositions

### (1) Preparation of Resist Compositions

Resist compositions were prepared by dissolving components in a solvent in accordance with the recipe shown in Table 1, and filtering the solution through a filter having a pore size of 0.2 μm.

The components in Table 1 are identified below.

### Organic Solvents:

PGMEA (propylene glycol monomethyl ether acetate)
EL (ethyl lactate)
DAA (diacetone alcohol)
   Comparative Acid Generators: cPAG-1 and cPAG-PDQ-1

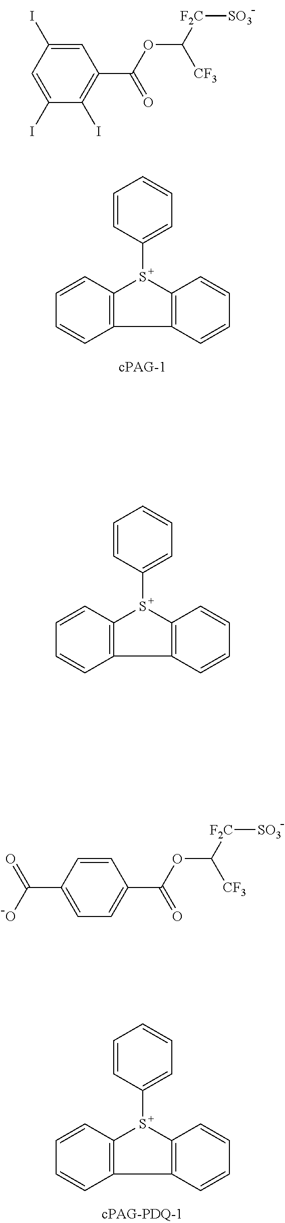

Blending Acid Generator: bPAG-1

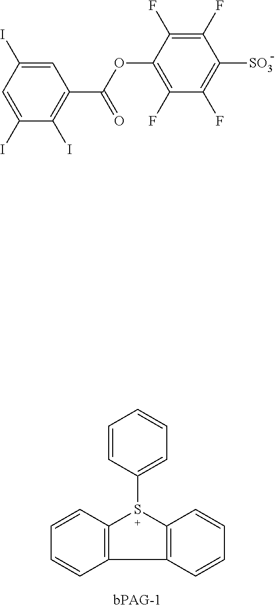

Comparative Quencher: cPDQ-1

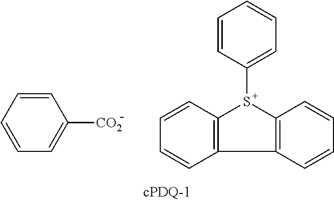

### (2) EUV Lithography Test

Each of the resist compositions in Table 1 was spin coated on a silicon substrate having a 20-nm coating of silicon-containing spin-on hard mask SHB-A940 (Shin-Etsu Chemical Co., Ltd., Si content 43 wt %) and prebaked on a hotplate at 105° C. for 60 seconds to form a resist film of 40 nm thick. Using an EUV scanner NXE3400 (ASML, NA 0.33, σ 0.9/0.7, dipole illumination), the resist film was exposed to EUV. The resist film was baked (PEB) on a hotplate at the temperature shown in Table 1 for 60 seconds and developed in a 2.38 wt % TMAH aqueous solution for 30 seconds to form a line-and-space pattern having a pitch of 32 nm and a line width of 16 nm. Examples 1 to 21 and Comparative Examples 1 and 2 were of positive tone and Example 22 and Comparative Example 3 were of negative tone.

The resist pattern was observed under CD-SEM (CG6300, Hitachi High-Technologies Corp.). The exposure dose that provides a L/S pattern having a size of 16±1.6 nm is reported as sensitivity. The LWR of a L/S pattern at this dose was measured.

The resist compositions are shown in Table 1 together with the sensitivity and LWR of EUV lithography.

TABLE 1

| | | Polymer | Additive | Organic solvent | PEB temp. | Sensitivity | LWR |
|---|---|---|---|---|---|---|---|
| | | (pbw) | (pbw) | (pbw) | (° C.) | (mJ/cm²) | (nm) |
| Example | 1 | P-1 | PAG-PDQ-1 | PGMEA (500) | 85 | 38 | 2.4 |
| | | (100) | (18.8) | EL (2500) | | | |
| | 2 | P-1 | PAG-PDQ-2 | PGMEA (500) | 85 | 36 | 2.6 |
| | | (100) | (17.5) | EL (2500) | | | |
| | 3 | P-1 | PAG-PDQ-3 | PGMEA (500) | 85 | 38 | 2.5 |
| | | (100) | (19.7) | EL (2500) | | | |
| | 4 | P-1 | PAG-PDQ-4 | PGMEA (2500) | 85 | 38 | 2.6 |
| | | (100) | (19.4) | DAA (500) | | | |
| | 5 | P-1 | PAG-PDQ-5 | PGMEA (2500) | 85 | 35 | 2.6 |
| | | (100) | (23.2) | DAA (500) | | | |
| | 6 | P-1 | PAG-PDQ-6 | PGMEA (2500) | 85 | 38 | 2.5 |
| | | (100) | (24.7) | DAA (500) | | | |
| | 7 | P-1 | PAG-PDQ-7 | PGMEA (2500) | 85 | 36 | 2.5 |
| | | (100) | (28.5) | DAA (500) | | | |
| | 8 | P-1 | PAG-PDQ-8 | PGMEA (2500) | 85 | 37 | 2.5 |
| | | (100) | (22.8) | DAA (500) | | | |
| | 9 | P-1 | PAG-PDQ-9 | PGMEA (2500) | 85 | 37 | 2.4 |
| | | (100) | (23.4) | DAA (500) | | | |
| | 10 | P-1 | PAG-PDQ-10 | PGMEA (2500) | 85 | 36 | 2.5 |
| | | (100) | (31.1) | DAA (500) | | | |
| | 11 | P-1 | PAG-PDQ-11 | PGMEA (500) | 85 | 37 | 2.5 |
| | | (100) | (24.0) | EL (2500) | | | |
| | 12 | P-1 | PAG-PDQ-12 | PGMEA (2500) | 85 | 37 | 2.4 |
| | | (100) | (29.2) | DAA (500) | | | |
| | 13 | P-1 | PAG-PDQ-13 | PGMEA (2500) | 85 | 37 | 2.5 |
| | | (100) | (34.3) | DAA (500) | | | |
| | 14 | P-1 | PAG-PDQ-14 | PGMEA (500) | 85 | 36 | 2.5 |
| | | (100) | (32.2) | EL (2500) | | | |
| | 15 | P-1 | PAG-PDQ-15 | PGMEA (2500) | 85 | 33 | 2.4 |
| | | (100) | (24.4) | DAA (500) | | | |
| | 16 | P-1 | PAG-PDQ-16 | PGMEA (2500) | 85 | 35 | 2.5 |
| | | (100) | (25.9) | DAA (500) | | | |
| | 17 | P-1 | PAG-PDQ-17 | PGMEA (500) | 85 | 36 | 2.5 |
| | | (100) | (27.7) | EL (2500) | | | |
| | 18 | P-1 | PAG-PDQ-1 (18.8) | PGMEA (500) | 85 | 35 | 2.7 |
| | | (100) | bPDQ-1 (1.5) | EL (2500) | | | |
| | 19 | P-1 | PAG-PDQ-1 (18.8) | PGMEA (2500) | 85 | 41 | 2.3 |
| | | (100) | bPDQ-1 (1.2) | DAA (500) | | | |
| | 20 | P-2 | PAG-PDQ-1 | PGMEA (2500) | 85 | 40 | 2.6 |
| | | (100) | (18.8) | DAA (500) | | | |
| | 21 | P-3 | PAG-PDQ-1 | PGMEA (2500) | 90 | 33 | 2.5 |
| | | (100) | (18.8) | DAA (500) | | | |
| | 22 | P-4 | PAG-PDQ-1 | PGMEA (2500) | 125 | 41 | 3.4 |
| | | (100) | (18.8) | DAA (500) | | | |
| Comparative | 1 | P-1 | cPAG-1 (14.5) | PGMEA (2500) | 80 | 45 | 3. |
| Example | | (100) | cPDQ-1 (5.7) | DAA (500) | | | |
| | 2 | P-1 | cPAG-PDQ1 | PGMEA (1000) | 80 | 46 | 3.4 |
| | | (100) | (13.5) | DAA (500) | | | |
| | | | | EL (1500) | | | |
| | 3 | P-4 | cPAG-1 (14.5) | PGMEA (2500) | 120 | 50 | 4.6 |
| | | (100) | cPDQ-1 (5.7) | DAA (500) | | | |

It is demonstrated in Table 1 that resist compositions comprising a bis-onium salt consisting of a divalent anion having a sulfonic acid anion structure directly bonded to an iodized aromatic group and a sulfonimide or sulfonamide anion structure bonded to the aromatic group directly or via a linking group containing at least one atom, and onium cations as the acid generator/quencher offer a high sensitivity and excellent LWR.

Japanese Patent Application No. 2023-193456 is incorporated herein by reference. Although some preferred embodiments have been described, many modifications and variations may be made thereto in light of the above teachings. It is therefore to be understood that the invention may be practiced otherwise than as specifically described without departing from the scope of the appended claims.

1. A resist composition comprising a bis-onium salt consisting of a divalent anion having a sulfonic acid anion structure directly bonded to an iodized aromatic group and a sulfonimide or sulfonamide anion structure bonded to the aromatic group directly or via a linking group containing at least one atom, and onium cations.

2. The resist composition of claim 1 wherein the bis-onium salt has the formula (1): 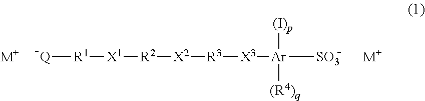
wherein p is an integer of 1 to 5, q is an integer of 0 to 7,
X¹ to X³ are each independently a single bond, ether bond, ester bond or amide bond,
R¹ to R³ are each independently a single bond or C₁-C₃₀ hydrocarbylene group which may contain at least one element selected from oxygen, nitrogen, sulfur and halogen, the total number of carbon atoms in R¹ to R³ being up to 30,
R⁴ is hydrogen, hydroxy, carboxy, fluorine, chlorine, bromine, amino, nitro, cyano, C₁-C₂₀ hydrocarbyl group, C₁-C₂₀ hydrocarbyloxy group, C₂-C₂₀ hydrocarbyloxycarbonyl group, C₂-C₂₀ hydrocarbylcarbonyloxy group, C₁-C₂₀ hydrocarbylsulfonyloxy group, —N(R^{4A})—C(═O)—R^{4B}, —N(R^{4A})—C(═O)—O—R^{4B}, or —N(R^{4A})—S(═O)₂—R^{4B}, the hydrocarbyl, hydrocarbyloxy, hydrocarbyloxycarbonyl, hydrocarbylcarbonyloxy, and hydrocarbylsulfonyloxy groups may contain at least one moiety selected from fluorine, chlorine, bromine, iodine, hydroxy, amino, ester bond, ether bond, urethane bond, urea bond, carbonate bond, amide bond, sulfonate ester bond, carbonyl, sulfide, and sulfonyl, R^{4A} is hydrogen or a C₁-C₆ saturated hydrocarbyl group which may contain halogen, hydroxy, a C₁-C₆ saturated hydrocarbyloxy moiety, C₂-C₆ saturated hydrocarbylcarbonyl moiety, or C₂-C₆ saturated hydrocarbylcarbonyloxy moiety, R^{4B} is a C₁-C₁₆ aliphatic hydrocarbyl group or C₆-C₁₂ aryl group, which may contain halogen, hydroxy, a C₁-C₆ saturated hydrocarbyloxy moiety, C₂-C₆ saturated hydrocarbylcarbonyl moiety, or C₂-C₆ saturated hydrocarbylcarbonyloxy moiety,
Ar is a C₆-C₁₆ (p+q+1)-valent aromatic hydrocarbon group,
Q⁻ is a group having a sulfonimide or sulfonamide anion structure, and
M⁺ is a sulfonium or iodonium cation.

3. The resist composition of claim 2 wherein Q⁻ is a group having any one of the following formulae (Q-1) to (Q-4): 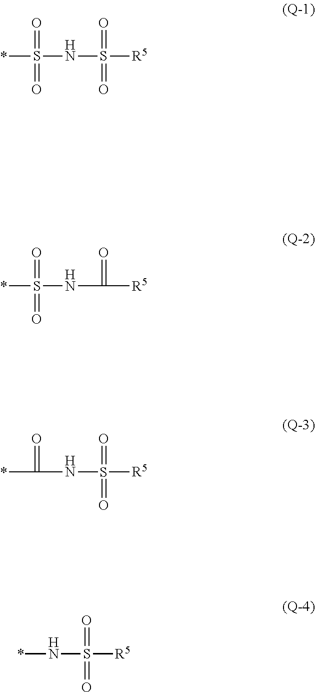
wherein R⁵ is each independently a C₁-C₁₂ saturated hydrocarbyl group or C₆-C₁₂ aryl group, which may be substituted with at least one moiety selected from halogen, cyano, nitro, hydroxy, C₁-C₈ saturated hydrocarbyl moiety, C₂-C₈ saturated hydrocarbylcarbonyloxy moiety, C₂-C₈ saturated hydrocarbyloxycarbonyl moiety, trifluoromethoxy, difluoromethoxy, trifluoromethylthio, and trifluoromethyl, and * designates a point of attachment to X¹.

4. The resist composition of claim 1, further comprising a base polymer.

5. The resist composition of claim 4 wherein the base polymer comprises repeat units having formula (a1) or (a2): 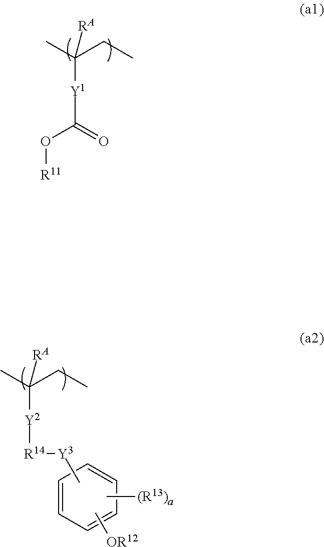
wherein R^{A} is each independently hydrogen or methyl,
Y¹ is a single bond, phenylene group, naphthylene group or a C₁-C₁₂ linking group which contains at least one moiety selected from an ester bond, ether bond and lactone ring, the phenylene group, naphthylene group and linking group may contain at least one moiety selected from hydroxy, C₁-C₈ saturated hydrocarbyloxy moiety and C₂-C₈ saturated hydrocarbylcarbonyloxy moiety,
Y² is a single bond or ester bond,
Y³ is a single bond, ether bond or ester bond,
R¹¹ and R¹² are each independently an acid labile group,
R¹³ is a C₁-C₄ saturated hydrocarbyl group, halogen, C₂-C₅ saturated hydrocarbylcarbonyl group, cyano, or C₂-C₅ saturated hydrocarbyloxycarbonyl group,
R¹⁴ is a single bond or a C₁-C₆ alkanediyl group which may contain an ether bond or ester bond, and
a is an integer of 0 to 4.

6. The resist composition of claim 5 which is a chemically amplified positive resist composition.

7. The resist composition of claim 4 wherein the base polymer is free of an acid labile group.

8. The resist composition of claim 7 which is a chemically amplified negative resist composition.

9. The resist composition of claim 1, further comprising an organic solvent.

10. The resist composition of claim 1, further comprising a quencher.

11. The resist composition of claim 1, further comprising an acid generator.

12. The resist composition of claim 1, further comprising a surfactant.

13. A pattern forming process comprising the steps of applying the resist composition of claim 1 onto a substrate to form a resist film thereon, exposing the resist film to high-energy radiation, and developing the exposed resist film in a developer.

14. The process of claim 13 wherein the high-energy radiation is ArF excimer laser of wavelength 193 nm, KrF excimer laser of wavelength 248 nm, EB or EUV of wavelength 3 to 15 nm.
